(19) United States
(12) Patent Application Publication
(10) Pub. No.: US 2019/0292167 A1
(43) Pub. Date: Sep. 26, 2019

(54) HETEROAROMATIC COMPOUNDS USEFUL FOR THE TREATMENT OF PROLIFERATIVE DISEASES

(71) Applicant: Syros Pharmaceuticals, Inc., Cambridge, MA (US)

(72) Inventors: Stephane Ciblat, Montreal (CA); Patrick Deroy, Blainville (CA); Melissa Leblanc, Laval (CA); Jason J. Marineau, Franklin, MA (US); Joel Moore, Lexington, MA (US); Stephanie Roy, Lachine (CA); M. Arshad Siddiqui, Newton, MA (US); Kevin Sprott, Needham, MA (US); Dana K. Winter, Rigaud (CA); Anzhelika Kabro, Montreal (CA); Serge Leger, Notre-Dame-De-L'ile-Perrot (CA); Tom Miller, Wakefield, MA (US); Darby Schmidt, Arlington, MA (US); Michael Bradley, Allston, MA (US)

(21) Appl. No.: 16/230,353

(22) Filed: Dec. 21, 2018

Related U.S. Application Data
(63) Continuation of application No. 15/030,265, filed on Apr. 18, 2016, now abandoned.
(60) Provisional application No. 61/975,467, filed on Apr. 4, 2014.
(60) Provisional application No. 61/892,888, filed on Oct. 18, 2013.

(51) Int. Cl. C07D 401/14; C07D 451/04; C07D 417/14; A61K 31/506; A61K 31/5377; A61K 31/635; C07D 413/14; C07D 417/04; C07D 413/04; C07D 403/14; C07D 403/04; C07D 401/04; A61K 45/06

(52) U.S. Cl. CPC C07D 401/14; C07D 451/04; C07D 417/14; A61K 31/506; A61K 31/5377; A61K 31/635; A61K 45/06; C07D 417/04; C07D 413/04; C07D 403/14; C07D 403/04; C07D 401/04; C07D 413/14

(57) ABSTRACT

The present invention provides novel compounds of Formula (I), and pharmaceutically acceptable salts, solvates, hydrates, tautomers, stereoisomers, isotopically labeled derivatives, and compositions thereof. Also provided are methods and kits involving the compounds or compositions for treating or preventing proliferative diseases (e.g., cancers (e.g., leukemia, melanoma, multiple myeloma), benign neoplasms, angiogenesis, inflammatory diseases, autoinflammatory diseases, and autoimmune diseases) in a subject. Treatment of a subject with a proliferative disease using a compound or composition of the invention may inhibit the aberrant activity of cyclin-dependent kinase 7 (CDK7), and therefore, induce cellular apoptosis and/or inhibit transcription in the subject.

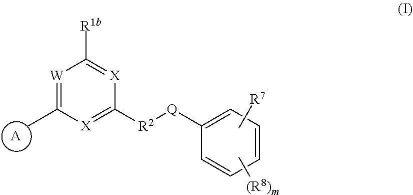

### CLAIM OF PRIORITY

The present application is a continuation of U.S. patent application Ser. No. 15/030,265, filed Apr. 18, 2016, which is a U.S. National Stage Application under 35 U.S.C. § 371 of International Application No. PCT/US2014/061264, filed Oct. 17, 2014, which claims priority under 35 U.S.C. § 119(e) to U.S. Provisional Application No. 61/892,888, filed Oct. 18, 2013, and U.S. Provisional Application No. 61/975,467, filed Apr. 4, 2014. The entire contents of each of the foregoing applications are incorporated herein by reference.

### BACKGROUND OF THE INVENTION

The members of the cyclin-dependent kinase (CDK) family play critical regulatory roles in proliferation. Unique among the mammalian CDKs, CDK7 has consolidated kinase activities, regulating both the cell cycle and transcription. In the cytosol, CDK7 exists as a heterotrimeric complex and is believed to function as a CDK½-activating kinase (CAK), whereby phosphorylation of conserved residues in CDK½ by CDK7 is required for full catalytic CDK activity and cell cycle progression. In the nucleus, CDK7 forms the kinase core of the RNA polymerase (RNAP) II general transcription factor complex and is charged with phosphorylating the C-terminal domain (CTD) of RNAP II, a requisite step in gene transcriptional initiation Together, the two functions of CDK7, i.e., CAK and CTD phosphorylation, support critical facets of cellular proliferation, cell cycling, and transcription.

Disruption of RNAP II CTD phosphorylation has been shown to preferentially affect proteins with short half-lives, including those of the anti-apoptotic BCL-2 family. Cancer cells have demonstrated ability to circumvent pro-cell death signaling through upregulation of BCL-2 family members. Therefore, inhibition of human CDK7 kinase activity is likely to result in anti-proliferative activity.

The discovery of selective inhibitors of CDK7 has been hampered by the high sequence and structural similarities of the kinase domain of CDK family members. Therefore, there is a need for the discovery and development of selective CDK7 inhibitors. Such CKD7 inhibitors hold promise as a therapeutic agent for the treatment of CLL and other cancers.

### SUMMARY OF THE INVENTION

The present invention provides CDK inhibitors, more particularly CDK7, CDK12 and CDK13 inhibitors, and in particular selective CDK7 inhibitors of Formula (I) or Formula (II), and pharmaceutically acceptable salts, solvates, hydrates, tautomers, stereoisomers, isotopically labeled derivatives, and compositions thereof. The present invention further provides methods of using the compounds of the invention, and pharmaceutically acceptable salts, solvates, hydrates, tautomers, stereoisomers, isotopically labeled derivatives, and compositions thereof, to study the inhibition of CDK7 and other CDK family members, and as therapeutics for the prevention and/or treatment of diseases associated with overexpression and/or aberrant activity of CDK7 and other CDK family members. In certain embodiments, the inventive compounds are used for the prevention and/or treatment of proliferative diseases (e.g., cancers (e.g., leukemia, melanoma, multiple myeloma), benign neoplasms, angiogenesis, inflammatory diseases, autoinflammatory diseases, and autoimmune diseases) in a subject.

In one aspect, the present invention provides compounds of Formula (I):

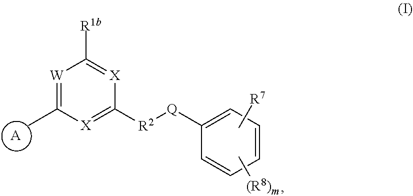

and pharmaceutically acceptable salts, solvates, hydrates, tautomers, stereoisomers, and isotopically labeled derivatives thereof, wherein Ring A, W, X, R^{1b}, R², Q, R⁷, R⁸, m and subvariables thereof are as defined herein.

In another aspect, the present invention provides compounds of Formula (II):

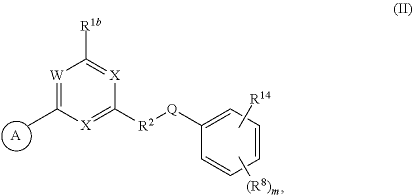

and pharmaceutically acceptable salts, solvates, hydrates, tautomers, stereoisomers, and isotopically labeled derivatives thereof, wherein Ring A, W, X, R^{1b}, R², Q, R¹⁴, R⁸, and subvariables thereof are as defined herein.

In another aspect, the present invention provides pharmaceutical compositions comprising a compound of Formula (I) or Formula (II), or a pharmaceutically acceptable salt, solvate, hydrate, tautomer, stereoisomer, or isotopically labeled derivative thereof, and optionally a pharmaceutically acceptable excipient. In certain embodiments, the pharmaceutical compositions described herein include a therapeutically effective amount of a compound of Formula (I) or Formula (II), or a pharmaceutically acceptable salt, solvate, hydrate, tautomer, stereoisomer, or isotopically labeled derivative thereof. The pharmaceutical composition may be useful for treating and/or preventing a proliferative or infectious disease.

In another aspect, the present invention provides methods for treating and/or preventing proliferative diseases. Exemplary proliferative diseases include cancer (e.g., leukemia, melanoma, multiple myeloma), benign neoplasm, angiogenesis, inflammatory diseases, autoinflammatory diseases, and autoimmune diseases. In other embodiments, the present invention provides methods for treating and/or preventing an infectious disease (e.g., a viral infection).

In still another aspect, the present invention provides methods of down-regulating the expression of CDK7 in a biological sample or subject.

Another aspect of the invention relates to methods of inhibiting the activity of CDK7 in a biological sample or subject.

The present invention also provides methods of inhibiting cell growth in a biological sample or subject.

In still another aspect, the present invention provides methods of inducing apoptosis of a cell in a biological sample or a subject.

In yet another aspect, the present invention provides compounds of Formula (I) or Formula (II), and pharmaceutically acceptable salts, solvates, hydrates, tautomers, stereoisomers, isotopically labeled derivatives, and compositions thereof, for use in the treatment of a proliferative disease in a subject.

In yet another aspect, the present invention provides compounds of Formula (I) or Formula (II), and pharmaceutically acceptable salts, solvates, hydrates, tautomers, stereoisomers, isotopically labeled derivatives, and compositions thereof, for use in the treatment or prevention of an infectious disease in a subject. In certain embodiments, the infectious disease is a viral infection.

Another aspect of the present invention relates to kits comprising a container with a compound of Formula (I) or Formula (II), or a pharmaceutically acceptable salt, solvate, hydrate, tautomer, stereoisomer, or isotopically labeled derivative thereof, or a pharmaceutical composition thereof. In certain embodiments, the kits described herein further include instructions for administering the compound of Formula (I) or Formula (II), or the pharmaceutically acceptable salt, solvate, hydrate, tautomer, stereoisomer, or isotopically labeled derivative thereof, or the pharmaceutical composition thereof.

The details of one or more embodiments of the invention are set forth herein. Other features, objects, and advantages of the invention will be apparent from the Detailed Description, the Figures, the Examples, and the Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions, p. 268 (E. L. Eliel, Ed., Univ. of Notre Dame Press, Notre Dame, Ind. 1972).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂ alkyl, C₁-C₁₀ alkyl, and C₁-C₆ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” are art-recognized and refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH═CH₂.

The term “alkylene” refers to the diradical of an alkyl group.

The terms “alkenylene” and “alkynylene” refer to the diradicals of an alkenyl and an alkynyl group, respectively.

The term “methylene unit” refers to a divalent —CH₂— group present in an alkyl, alkenyl, alkynyl, alkylene, alkenylene, or alkynylene moiety.

The term “carbocyclic ring system”, as used herein, means a monocyclic, bicyclic or polycyclic hydrocarbon ring system, wherein each ring is either completely saturated or contains one or more units of unsaturation, but where no ring is aromatic.

The term “carbocyclyl” refers to a radical of a carbocyclic ring system. Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like).

The term “aromatic ring system” is art-recognized and refers to a monocyclic, bicyclic or polycyclic hydrocarbon ring system, wherein at least one ring is aromatic.

The term “aryl” refers to a radical of an aromatic ring system. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like.

The term “heteroaromatic ring system” is art-recognized and refers to monocyclic, bicyclic or polycyclic ring system wherein at least one ring is both aromatic and comprises a heteroatom; and wherein no other rings are heterocyclyl (as defined below). In certain instances, a ring which is aromatic and comprises a heteroatom contains 1, 2, 3, or 4 independently selected ring heteroatoms in such ring.

The term “heteroaryl” refers to a radical of a heteroaromatic ring system. Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, 5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

The term “heterocyclic ring system” refers to monocyclic, bicyclic and polycyclic ring systems where at least one ring is saturated or partially unsaturated (but not aromatic) and comprises a heteroatom. A heterocyclic ring system can be attached to its pendant group at any heteroatom or carbon atom that results in a stable structure and any of the ring atoms can be optionally substituted.

The term “heterocyclyl” refers to a radical of a heterocyclic ring system. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine, and 1,2,3,4-tetrahydro-2,6-naphthyridine. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

The term “saturated heterocyclyl” refers to a radical of heterocyclic ring system wherein every ring is saturated, e.g., tetrahydrofuran, tetrahydro-2H-pyran, pyrrolidine, piperidine and piperazine.

“Partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group (such as an alkyl, alkenyl, alkynyl, alkylene, alkenylene, alkynylene or the carbon atom of a carbocyclyl, aryl, heterocyclyl or heteroaryl) are independently deuterium; halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph (where “Ph” is phenyl), which may be substituted with R^{∘}; —(CH₂)₀₋₄O(CH₂)₀₋₁Ph which may be substituted with R^{∘}; —CH═CHPh, which may be substituted with —R^{∘}; —NO₂; —CN; —N₃; —(CH₂)₀₋₄N(R^{∘})₂; —(CH₂)₀₋₄N(R)C(O)R; —N(R^{∘})C(S)R^{∘}; —(CH₂)₀₋₄N(R)C(O)NR^{∘}₂; —N(R^{∘})C(S)NR^{∘}₂; —(CH₂)₀₋₄N(R^{∘})C(O)OR^{∘}; —N(R)N(R^{∘})C(O)R^{∘}; —N(R)N(R^{∘})C(O)NR^{∘}₂; —N(R^{∘})N(R^{∘})C(O)OR^{∘}; —(CH₂)₀₋₄C(O)R; —C(S)R; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄—C(O)—N(R^{∘})—S(O)₂—R^{∘}, —(CH₂)₀₋₄OC(O)R^{∘}; —OC(O)(CH₂)₀₋₄SR^{∘}—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, deuterium, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently deuterium, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include deuterium, halogen, —R^{•}, -(haloR^{•}), —OH, —OR^{•}, —O(haloR^{•}), —CN, —C(O)OH, —C(O)OR^{•}, —NH₂, —NHR^{•}, —NR^{∘}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently deuterium, halogen, —R^{•}—, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

“Halo” or “halogen” refers to fluorine (fluoro, —F), chlorine (chloro, —Cl), bromine (bromo, —Br), or iodine (iodo, —I).

The term “one or more methylene units of the alkylene, alkenylene or alkynylene is optionally replaced with —O—, —S—, —S(═O)₂, or —NR^{X}—” as used herein means that none, one, more than one, or all of the methylene units present may be so replaced. Thus, for example, the moieties, —O—, —S—, and —NR^{X}— are included in this definition because in each case they represent a C₁ alkylene (i.e., methylene) replaced with —O—, —S—, or —NR^{X}—, respectively.

It should also be understood that reference to a variable or subvariable in Formula I (e.g., R², R⁴ or R⁵) being “an optionally substituted C₁-C₄ alkylene, and an optionally substituted C₂-C₄ alkenylene or alkynylene, wherein: one or more methylene units of the alkylene, alkenylene or alkynylene other than a methylene unit bound to a nitrogen atom is optionally and independently replaced with —O—, —S—, —N(R⁶)—, or —S(═O)₂—” is only intended to encompass chemically stable combinations of optionally substitutions and replacements.

As used herein, the term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, -OTs), methanesulfonate (mesylate, -OMs), p-bromobenzenesulfonyloxy (brosylate, -OBs), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

In still another aspect, the present invention provides methods of inhibiting other CDKs, specifically CDK12 or CDK13, with a compound of Formula (I) or Formula (II).

These and other exemplary substituents are described in more detail in the Detailed Description, Figures, Examples, and Claims. The invention is not intended to be limited in any manner by the above exemplary listing of substituents.

### Other Definitions

The following definitions are more general terms used throughout the present application:

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) or Formula (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. The invention includes all enantiomers, stereoisomers, racemic mixtures and combinations thereof for any compound depicted. The invention also includes both E- and Z-forms of any carbon-carbon bond, regardless of whether one particular form is depicted in a structure.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

As used herein, the terms “condition,” “disease,” and “disorder” are used interchangeably.

An “effective amount” of a compound of Formula (I) or Formula (II) refers to an amount sufficient to elicit the desired biological response, i.e., treating the condition. As will be appreciated by those of ordinary skill in this art, the effective amount of a compound of Formula (I) or Formula (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) or Formula (II) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound of Formula (I) or Formula (II) is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases).

As used herein, an “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis.

As used herein, an “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barré syndrome, Hashimoto's thyroiditis, and cardiomyopathy.

The term “autoinflammatory disease” refers to a category of diseases that are similar but different from autoimmune diseases. Autoinflammatory and autoimmune diseases share common characteristics in that both groups of disorders result from the immune system attacking a subject's own tissues and result in increased inflammation. In autoinflammatory diseases, a subject's innate immune system causes inflammation for unknown reasons. The innate immune system reacts even though it has never encountered autoantibodies or antigens in the subject. Autoinflammatory disorders are characterized by intense episodes of inflammation that result in such symptoms as fever, rash, or joint swelling. These diseases also carry the risk of amyloidosis, a potentially fatal buildup of a blood protein in vital organs. Autoinflammatory diseases include, but are not limited to, familial Mediterranean fever (FMF), neonatal onset multisystem inflammatory disease (NOMID), tumor necrosis factor (TNF) receptor-associated periodic syndrome (TRAPS), deficiency of the interleukin-1 receptor antagonist (DIRA), and Behçet's disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus.

### BRIEF DESCRIPTION OF THE DRAWINGS

FIG. 1A-FIG. 1H depicts the structure of representative compounds of Formula (I). The “*” annotation indicates that the absolute stereochemistry of the compound was not determined, but the relative stereochemistry of the indicated chiral bonds is known. In each of the compounds so annotated, the relative stereochemistry of the chiral bonds is opposite (e.g., R,S or S,R, but not S,S or R,R). The “**” annotation indicates that the absolute stereochemistry of the compound was not determined, but the relative stereochemistry of the indicated chiral bonds is known. In each of the compounds so annotated, the relative stereochemistry of the chiral bonds is the same (e.g., R,R or S,S, but not S,R or R,S).

FIG. 2A-FIG. 2E depicts the structure of representative compounds of Formula (II). The “**” annotation in FIG. 2A-FIG. 2E is defined as described above for FIG. 1A-FIG. 1H.

### DETAILED DESCRIPTION OF CERTAIN EMBODIMENTS OF THE INVENTION

### Compounds

In one aspect of the present invention, provided are compounds of Formula (I): compound having the structural formula I:

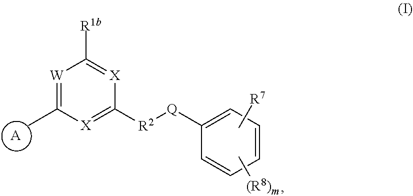

(I), or a pharmaceutically acceptable salt, solvate, hydrate, tautomer, stereoisomer, or isotopically labeled derivative thereof, wherein:

ring A is an optionally substituted heteroaryl ring of any one of the Formulae (i-1)-(i-5):

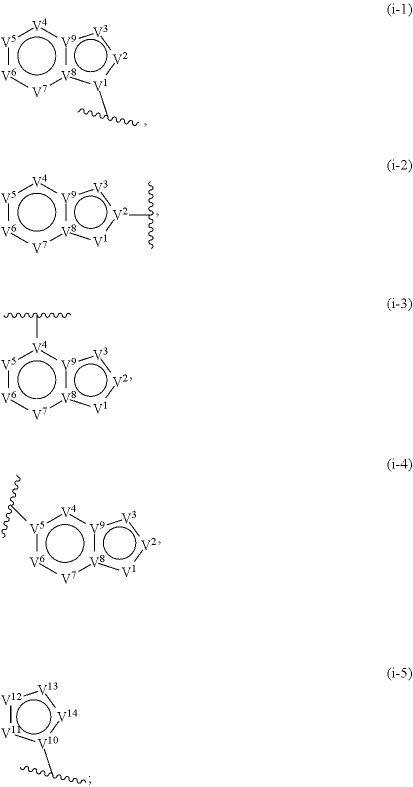

each instance of V¹, V², V³, V⁴, V⁵, V⁶, V⁷, V⁸, V⁹, V¹⁰, V¹¹, V¹², V¹³, and V¹⁴ is independently O, S, N, N(R^{A1}), C, or C(R^{A2});

each instance of R^{A1} is independently selected from hydrogen, deuterium, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;

each instance of R^{A2} is independently selected from hydrogen, deuterium, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a}, wherein each occurrence of R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring;

each X is independently selected from N and CH, wherein at least one X is N;

W is selected from N and C(R^{1a});

each of R^{1a}, if present, and R^{1b} is independently selected from hydrogen, deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B1a}, —N(R^{B1a})₂, and —SR^{B1a}, wherein each occurrence of R^{B1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

R^{1a} and R^{1b} are joined to form an optionally substituted carbocyclic, optionally substituted heterocyclic, optionally substituted aryl, or optionally substituted heteroaryl ring;

R² is an optionally substituted C₁-C₄ alkylene or an optionally substituted C₂-C₄ alkenylene or alkynylene, wherein one or more methylene units of the alkylene, alkenylene or alkynylene are optionally and independently replaced with —O—, —S—, or —N(R⁶)—;

Q is selected from R⁵,

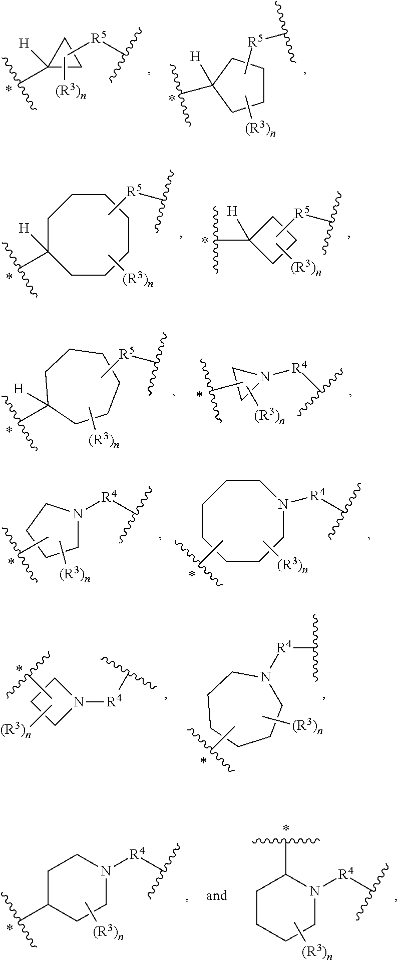

wherein each [symbol] represents a portion of Q bound to the rest of the compound; and “*” represents a portion of Q bound to R²;

each instance of R³, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1})₂, and —SR^{C1}, wherein each occurrence of R^{C1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or

two R³ groups bound to the same ring carbon atom are taken together to form ═O, or

two R³ groups bound to the same or different ring carbon atoms are joined to form an optionally substituted carbocyclyl, optionally substituted heterocyclyl, optionally substituted aryl, or optionally substituted heteroaryl ring;

R3a is selected from hydrogen, optionally substituted acyl, optionally substituted alkyl, optionally substituted carbocyclyl, optionally substituted heterocyclyl, optionally substituted aryl, optionally substituted heteroaryl,

R⁴ is selected from a bond, an optionally substituted C₁-C₄ alkylene, and an optionally substituted C₂-C₄ alkenylene or alkynylene, wherein:
one or more methylene units of the alkylene, alkenylene or alkynylene other than a methylene unit bound to a nitrogen atom is optionally and independently replaced with —O—, —S—, —N(R⁶)—, or —S(═O)₂—, and
two substituents on either the same or adjacent carbon atoms in the alkylene, alkenylene or alkynylene are taken together to form an optionally substituted carbocyclic or optionally substituted heterocyclic ring;

R⁵ is selected from a bond, an optionally substituted C₁-C₄ alkylene, and an optionally substituted C₂-C₄ alkenylene or alkynylene, wherein:
one or more methylene units of the alkylene, alkenylene or alkynylene is optionally and independently replaced with —O—, —S—, —N(R⁶)—, or —S(═O)₂—, and
two substituents on either the same or adjacent carbon atoms in the alkylene, alkenylene or alkynylene are optionally taken together to form an optionally substituted carbocyclic or optionally substituted heterocyclic ring;

each R⁶ is independently selected from hydrogen, and —C₁-C₆ alkyl;

R⁷ is any one of the Formulae (ii-1)-(ii-17):

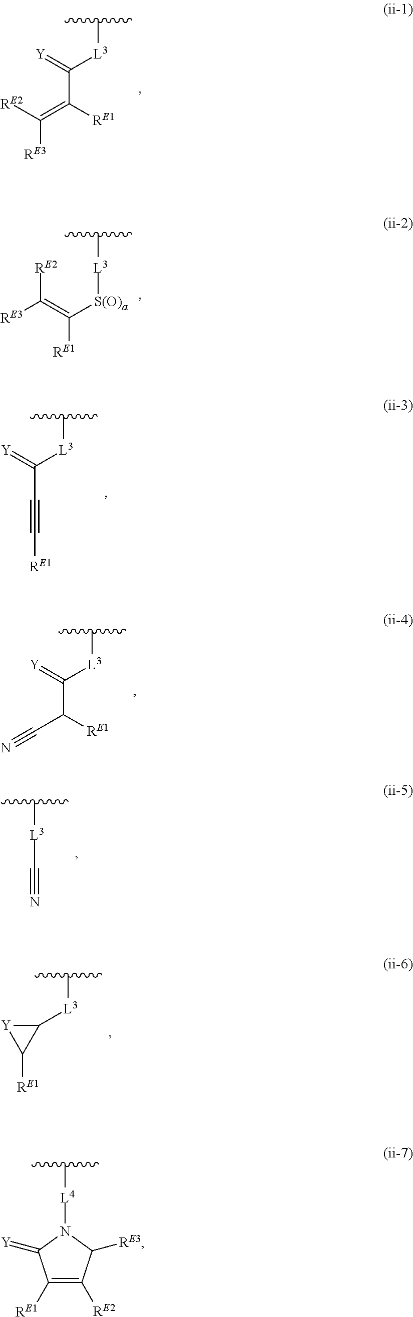

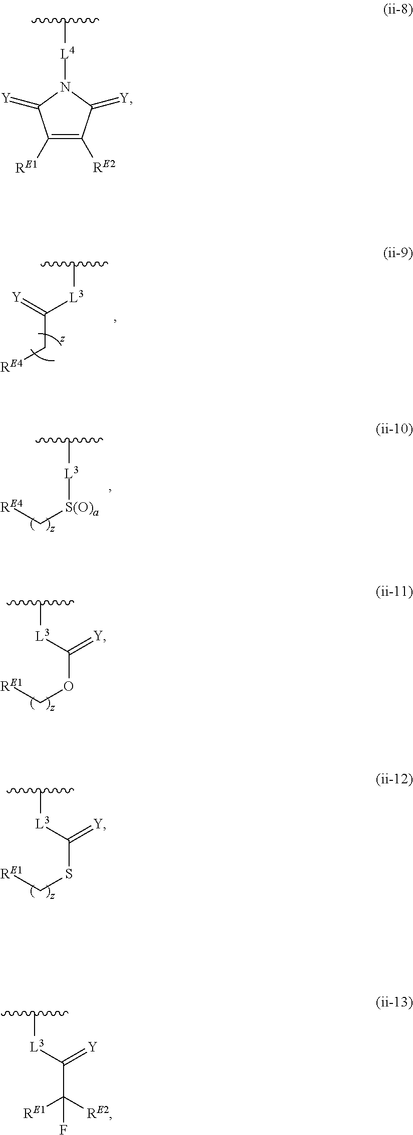

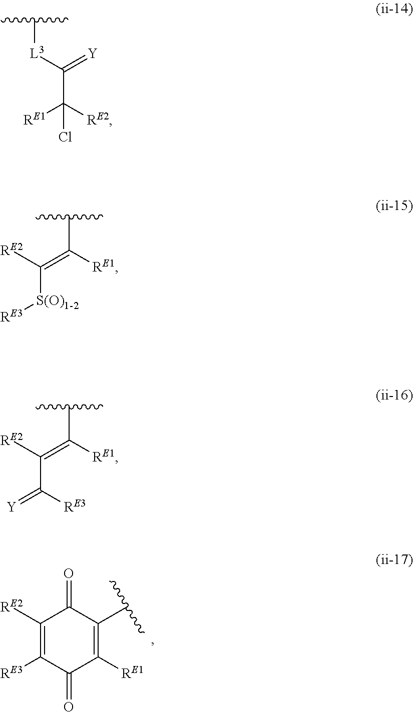

wherein:
R⁷ and Q are para or meta to each other;
L³ is a bond, an optionally substituted C₁-C₄ alkylene, or an optionally substituted C₂-C₄ alkenylene or alkynylene, wherein one or more methylene units of the alkylene, alkenylene or alkynylene are optionally and independently replaced with —O—, —S—, or —N(R⁶)—;
L⁴ is a bond, an optionally substituted C₁-C₄ alkylene, or an optionally substituted C₂-C₄ alkenylene or alkynylene;
each of R^{E1}, R^{E2} and R^{E3} is independently selected from hydrogen, deuterium, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CH₂OR⁹, —CH₂N(R⁹)₂, —CH₂SR⁹, —CN, —OR⁹, —N(R⁹)₂, and —SR⁹, wherein each occurrence of R⁹ is independently selected from hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
Y is O, S, or N(R^{E6}); wherein R^{E6} is hydrogen, substituted or unsubstituted C₁₋₆ alkyl, or a nitrogen protecting group;
z is 0, 1, 2, 3, 4, 5, or 6;

each instance of R⁸, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D1}, —N(R^{D1})₂, and —SR^{D1}, wherein each occurrence of R^{D1} is independently selected from hydrogen, optionally substituted acyl, optionally substituted alkyl, optionally substituted alkenyl, optionally substituted alkynyl, optionally substituted carbocyclyl, optionally substituted heterocyclyl, and optionally substituted aryl, optionally substituted heteroaryl, or

two R⁸ groups are joined to form an optionally substituted carbocyclyl, optionally substituted heterocyclyl, optionally substituted aryl, or optionally substituted heteroaryl ring;

m is 0, 1, 2, 3 or 4; and

n is 0, 1, 2, 3, 4, 5 or 6.

It will be understood by those of skill in the art that the value of n is limited by the number of hydrogen atoms bound to Q.

In some embodiments, ring A is an optionally substituted heteroaryl ring additionally of Formula (i-6):

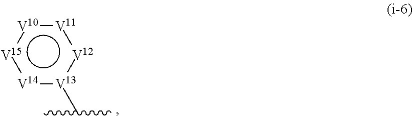

wherein each of V¹⁰, V¹¹, V¹², V¹³, V¹⁴ is defined as above and V¹⁵ is O, S, N, N(R^{A1}), C, or C(R^{A2}), wherein R^{A1} and R^{A2} are as defined above.

In certain embodiments, n is additionally selected from 7, 8, 9, 10, 11, 12, 13 or 14.

In some embodiments Q is additionally selected from R⁵;

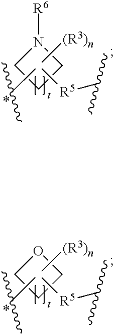

and a 4-14 membered, divalent, fused or spirofused bicyclic ring system comprising a total of 0 to 4 ring heteroatoms independently selected from N, O and S, and optionally substituted with 1 to 6 independently selected R³, wherein:

each ring in the bicyclic ring system is independently selected from heterocyclyl, carbocyclyl, aromatic or heteroaromatic,

one atom in each ring of the bicyclic ring system is attached to the rest of the compound,

t is 0, 1, 2, 3, or 4,

R³, R⁵, R⁶ are as defined above, and

n is 0, 1, 2, 3, 4, 5, 6, 7, 8, 9, 10, 11, 12, 13 or 14.

In certain embodiments, one or more methylene units of the alkylene, alkenylene or alkynylene in L³ are additionally optionally and independently replaced with —S(O)— or —S(O)₂.

In certain embodiments L³ is additionally selected from an optionally substituted C₅-C₇ alkylene, or an optionally substituted C₅ alkenylene or alkynylene, wherein one or more methylene units of the alkylene, alkenylene or alkynylene are optionally and independently replaced with —O—, —S—, —S(O)—, —S(O)₂ or —N(R⁶)—.

In certain embodiments, R⁷ is additionally selected from formulae (ii-18)-(ii-20):

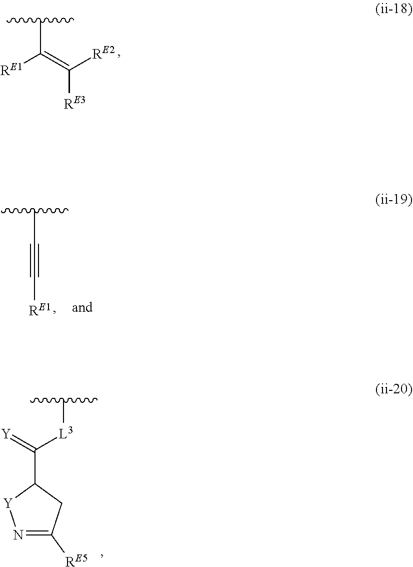

wherein R^{E5} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E5a}, —CH₂N(R^{E5a})₂, —CH₂SR^{E5a}, —OR^{E5a}, —N(R^{E5a})₂, and —SR^{E5a}, wherein each occurrence of R^{E5a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E5a} groups are joined to form an optionally substituted heterocyclic ring;

In certain embodiments, the compound is other than:

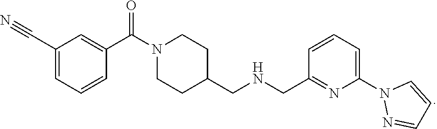 [SMILES: N#Cc1cccc(C(=O)N2CCC(CNCc3cccc(-n4cccn4)n3)CC2)c1]

In certain embodiments, provided in the present invention are compounds of Formula (I) and pharmaceutically acceptable salts thereof.

In certain embodiments, no more than three of V¹, V², V³, V⁴, V⁵, V⁶, V⁷, V⁸, and V⁹ are each independently selected from the group consisting of O, S, N, and N(R^{A1}).

In certain embodiments, two of V¹, V², V³, V⁴, V⁵, V⁶, V⁷, V⁸, and V⁹ are each independently selected from the group consisting of N and N(R^{A1}) and the rest of V¹, V², V³, V⁴, V⁵, V⁶, V⁷, V⁸, and V⁹ are each independently C or C(R^{A2}). In one aspect of these embodiments, one of V¹, V², or V³ is N(R^{A1}); one of V¹, V², or V³ is C; one of V¹, V², and V³ is C(R^{A2}); one of V⁴, V⁵, V⁶, or V⁷ is N, the rest of V⁴, V⁵, V⁶, and V⁷ are C(R^{A2}); and V⁸ and V⁹ are C.

In certain embodiments, one of V¹, V², V³, V⁴, V⁵, V⁶, V⁷, V⁸, and V⁹ is N or N(R^{A1}) and the rest of V¹, V², V³, V⁴, V⁵, V⁶, V⁷, V⁸, and V⁹ are each independently C or C(R^{A2}). In one aspect of these embodiments, one of V¹, V², or V³ is N(R^{A1}); one of V¹, V², or V³ is C; one of V¹, V², and V³ is C(R^{A2}); each of V⁴, V⁵, V⁶, and V⁷ are C(R^{A2}); and V⁸ and V⁹ are C.

In certain embodiments ring A is

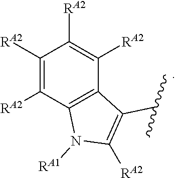

In one aspect of these embodiments, ring A is

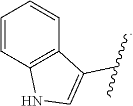 [SMILES: *c1c[nH]c2ccccc12]

In certain embodiments ring A is

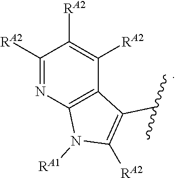

In one aspect of these embodiments, ring A is

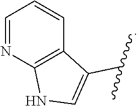 [SMILES: *c1c[nH]c2ncccc12]

In certain embodiments, ring A is selected from:

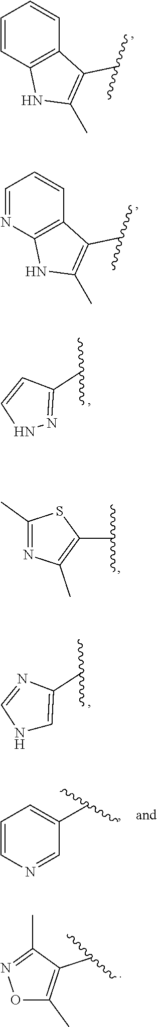

In a more specific aspect of these embodiments, ring A is selected from

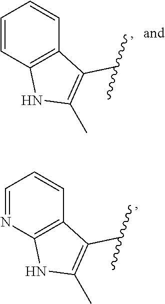 [SMILES: *c1c(C)[nH]c2ccccc12 ; *c1c(C)[nH]c2ncccc12]

In certain embodiments, each R^{A1} is independently selected from hydrogen, or C₁₋₆ alkyl. In certain embodiments, all instances of R^{A1} are hydrogen.

In certain embodiments, each R^{A2} is independently selected from hydrogen, halogen, and optionally substituted C₁-C₆ alkyl, and optionally substituted aryl. In one aspect of these embodiments, all instances of R^{A2} are hydrogen.

In certain embodiments, W is N.

In certain embodiments, W is C(R^{1a}). In one aspect of these embodiments, each X is N.

In certain embodiments, R^{1a} is selected from selected from hydrogen, halo, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂. In one aspect of these embodiments, R^{1a} is selected from halo, —CN and C₁-C₃ alkyl. In a more specific aspect of these embodiments, R^{1a} is selected from chloro, —CN and —CH₃. In an even more specific aspect of these embodiments, R^{1a} is selected from chloro and —CN.

In certain embodiments, R^{1b} is selected from selected from hydrogen, halo, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂. In one aspect of these embodiments, R^{1b} is hydrogen.

In certain embodiments, R² is selected from —N(R⁶)—, —N(R⁶)—CH₂—*, —N(R⁶)—CH₂—CH(CH₃)₂—*, —N(R⁶)—CH₂—CH(CH₃)₂—CH₂—*, and C₁-C₂ alkylene optionally substituted with 1 to 4 substituents independently selected from halo, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂, wherein “*” represents a portion of R² bound to Q. In certain aspects of these embodiments, R² is selected from —NH—; —N(C₁-C₃ alkyl)-; —NH—CH₂—*; and C₁-C₂ alkylene optionally substituted with 1 to 4 substituents independently selected from halo, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂. In a more specific aspect of these embodiments, R² is selected from —NH— and —NH—CH₂—*.

In certain embodiments, Q is selected from:

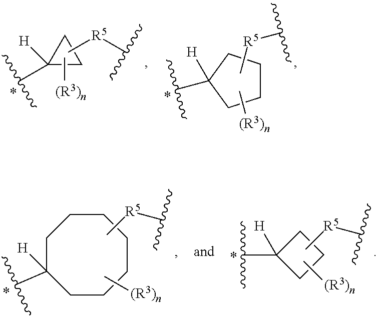

In a more specific aspect on these embodiments, Q is

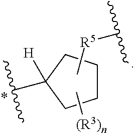

In one aspect of these embodiments, R⁵ is selected from —N(R⁶)—, †—N(R⁶)—(C₁-C₃ alkylene)- and †—N(R⁶)—(C₂-C₃ alkenylene or alkynylene)-, wherein:

one or more methylene units in the alkylene, alkenylene or alkynylene other than one bound to a nitrogen atom is optionally and independently replaced with —O—, —S—, —N(R⁶)—, or —S(═O)₂—,

two substituents on either the same or adjacent carbon atoms in the alkylene, alkenylene or alkynylene are taken together to form an optionally substituted carbocyclic or optionally substituted heterocyclic ring, and

“†” represents a portion of R⁵ bound to Q.

In another aspect of these embodiments, R⁵ is a bond. In a more specific aspect of these embodiments, R⁵ is selected from —NH—, †—NH—CH₂—, †—NH—C(═O)—, and †—NH—S(═O)₂—. In another more specific aspect of these embodiments, R⁵ is selected from †—NH—C(═O)— and a bond.

In certain embodiments, Q is selected from:

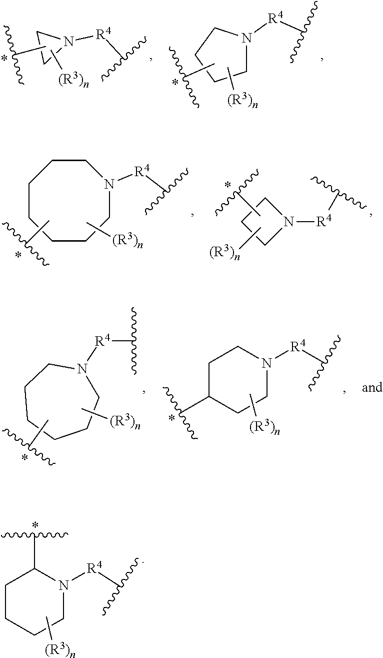

In one aspect of these embodiments, R⁴ is selected from —S(═O)₂—, or C₁-C₂ alkylene optionally substituted with 1 to 4 substituents independently selected from halo, ═O, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂. In a more specific aspect of these embodiments, R⁴ is selected from —C(O)—, —S(O)₂— and —CH₂—. In another more specific aspect of these embodiments, Q is selected from

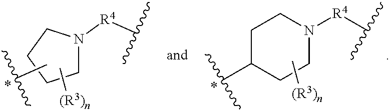

In a still more specific aspect of these embodiments, Q is selected from

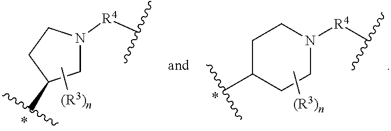

In still other embodiments, Q is selected from R⁵,

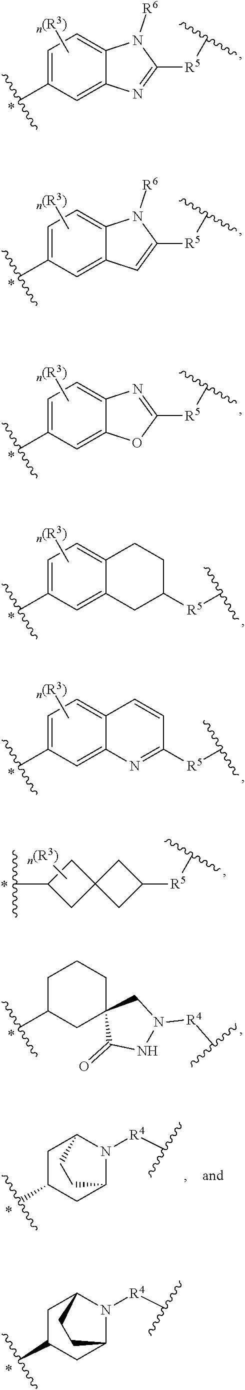

wherein:

“*” represents a portion of Q bound to R²; and

n is 0, 1, 2, 3, 4, 5, 6, 7, 8, 9, or 10.

In one aspect of these embodiments, R⁵ is selected from a bond, †—N(R⁶)—C(O)—, and †—CH₂—N(R⁶)—C(O)—, wherein “†” represents a portion of R⁵ bound to Q. In another aspect of these embodiments, R⁴ is a bond.

In still other embodiments, Q is selected from a bond, *—C(CH₃)₂CH₂NHC(O)—,

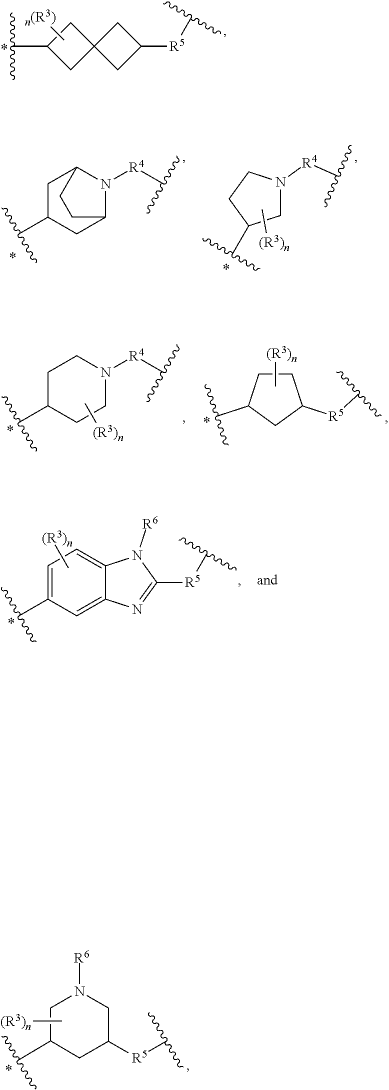

wherein:

“*” represents a portion of Q bound to R²; and

n is 0, 1, 2, 3, 4, 5, 6, 7, 8, 9, or 10.

In one aspect of these embodiments, R⁵, when present, is selected from a bond, †—N(R⁶)—C(O)—, †—C(O)—N(R⁶)—, and †—CH₂—, wherein “†” represents a portion of R⁵ bound to Q. In another aspect of these embodiments, R⁴, when present, is selected from —C(O)—, —S(O)₂ and —CH₂—. In a more specific aspect of these embodiments, Q is selected from: a bond,

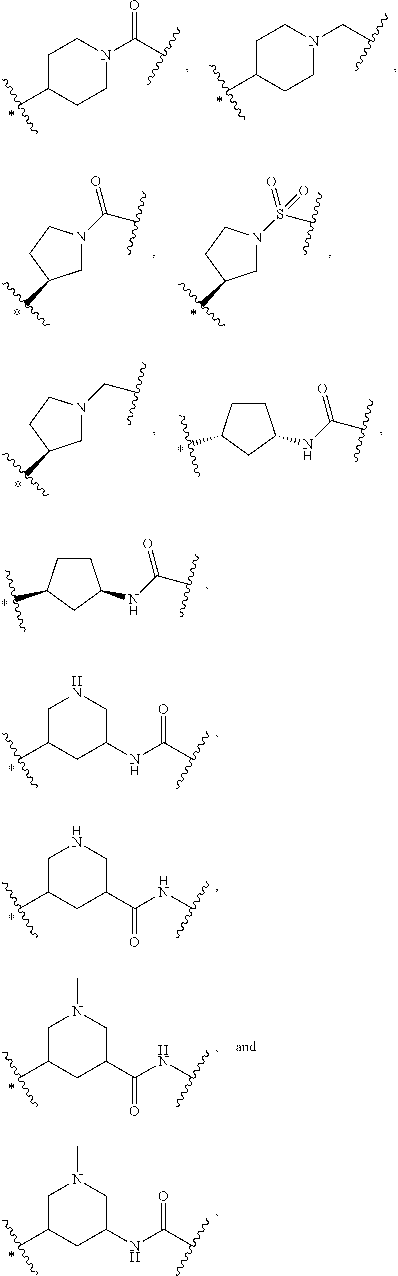

wherein where no stereochemistry is depicted in a structure, all enantiomers and stereoisomers are included.

In one aspect of all embodiments of Q, R³ is absent (i.e., n is 0), or is selected from halo, —OH, —C₁-C₃ alkyl, halo-substituted —C₁-C₃ alkyl, —O—C₁-C₃ alkyl, halo-substituted —O—C₁-C₃ alkyl, —CN, —NH₂, —NH(C₁-C₃ alkyl), and —N(C₁-C₃ alkyl)₂, or two R³ bound to the same ring carbon atom are taken together to form ═O. In a more specific aspect of all embodiments of Q, R³ is absent (i.e., n is 0)

In certain embodiments, each R⁶ present in a compound of Formula (I) is selected from hydrogen and —CH₃. In a more specific aspect of these embodiments, each R⁶ is hydrogen. In another more specific aspect of these embodiments, at least one R⁶ is —CH₃.

In certain embodiments, R⁷ is located para to Q. In one aspect of these embodiments, R⁷ comprises L³ and L³ is —NR^{L3a}—. In a more specific aspect of these embodiments, R⁷ comprises L³ and L³ is —NH—. In another aspect of these embodiments, R⁷ comprises Y, and Y is ═O. In still another aspect of these embodiments, R⁷ comprises at least one of R^{E1}, R^{E2} and R^{E3} and one of the R^{E1}, R^{E2} or R^{E3} that is present is —CH₂N(R^{E1a})₂. In a more specific aspect of these embodiments, R⁷ comprises at least one of R^{E1}, R^{E2} and R^{E3}; one of the R^{E1}, R^{E2} or R^{E3} that is present is —CH₂N(R^{E1a})₂; and each R^{E1a} is independently an optionally substituted C₁-C₄ alkyl, or the two R^{E1a} are taken together with the nitrogen atom to which they are bound to form an optionally substituted heterocyclyl or an optionally substituted heteroaryl.

In certain embodiments, R⁷ is

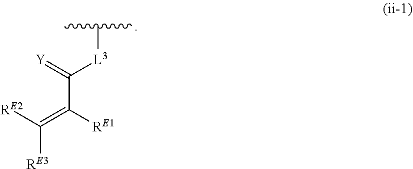

In one aspect of these embodiments, L³ is selected from —NH—, and —NH—C(O)—(CH₂)₁₋₄—NH—**, wherein “**” represents a portion of L³ bound to —C(═Y)—. In a more specific aspect of these embodiments, R⁷ is

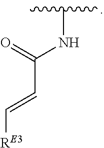

In an even more specific aspect of these embodiments, R⁷ is

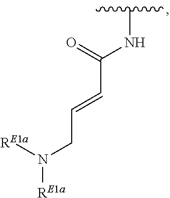

wherein each R^{E1a} is independently an optionally substituted C₁-C₄ alkyl, or the two R^{E1a} are taken together with the nitrogen atom to which they are bound to form an optionally substituted heterocyclyl or an optionally substituted heteroaryl. In a further more specific aspect of these embodiments, R⁷ is para to Q and is selected from 4-dimethylaminobut-2-enamido, 4-morpholin-4-ylbut-2-enamido, 4-pyrrolidin-1-ylbut-2-enamido, 4-1H-imidazo-1-ylbut-2-enamido, 4-(4-methylpiperazin-1-yl)but-2-enamido, and 4-(2-hydroxyethyl)(methyl)aminobut-2-enamido. In still another more specific aspect of these embodiments, R⁷ is selected from: 4-hydroxybut-2-enamido, 5-(4-(dimethylamino)but-2-enamido)pentanamido, 3-(4-(dimethylamino)but-2-enamido)propanamido, and 2-(4-(dimethylamino)but-2-enamido)ethanamido.

In still another specific embodiment, R⁷ is selected from —CH₂N(CH₃)C(O)CH═CHN(CH₃)₂, —CH₂NHC(O)CH═CHN(CH₃)₂, —N(CH₃)C(O)CH═CHCH₂N(CH₃)₂, —NHC(O)(CH₂)₄NHC(O)CH═CHCH₂N(CH₃)₂, —NHC(O)CH═CH₂, —NHC(O)CH═CHCH₂N(CH₃)₂, —NHC(O)CH═CHCH₂N(CH₃)CH₂CH(OH)CH₂OH, —NHC(O)CH═CHCH₂N(CH₃)CH₂CH₂OH, —NHC(O)CH═CHCH₂N(CH₃)CH₂C(O)NH₂, —NHC(O)CH═CHCH₂N(CH₃)CH₂C(O)OH, —NHC(O)CH═CHCH₂NHC(O)CF₃, —NHC(O)CH═CHCH₂NHS(O)₂CH₃, —NHC(O)CH═CHCH₂OH, —NHC(O)CH═CHN(CH₃)₂, —NHC(O)CH═CHNHCH₃, —NHC(O)CH₂CH₂NHC(O)CH═CHCH₂N(CH₃)₂, —NHC(O)CH₂NHC(O)CH═CHCH₂N(CH₃)₂,

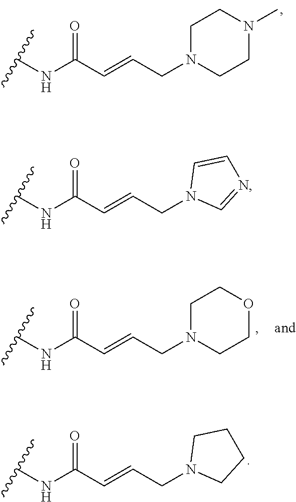 [SMILES: *NC(=O)/C=C/CN1CCN(C)CC1 ; *NC(=O)/C=C/Cn1ccnc1 ; *NC(=O)/C=C/CN1CCOCC1 ; *NC(=O)/C=C/CN1CCCC1]

In certain embodiments, m is 0 or 1; and the single R⁸, if present, is selected C₁-C₄ alkyl and halogen. In a more specific aspect of these embodiments, R⁸ is absent (i.e., m is 0), or selected from 2-methyl, 3-methyl and 3-fluoro.

Although, as indicated above, various embodiments and aspects thereof for a variable in Formula (I) may be selected from a group of chemical moieties, the invention also encompasses as further embodiments and aspects thereof situations where such variable is: a) selected from any subset of chemical moieties in such a group; and b) any single member of such a group.

Although various embodiments and aspects thereof are set forth (or implied, as discussed in the preceding paragraph) individually for each variable in Formula (I) above, the invention encompasses all possible combinations of the different embodiments and aspects for each of the variables in Formula (I).

Thus, in certain embodiments, the compound of Formula (I) is of Formula (Ia):

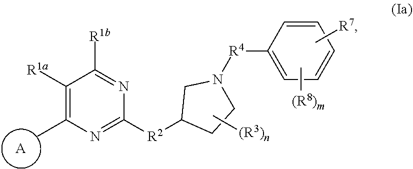

or a pharmaceutically acceptable salt, solvate, hydrate, tautomer, stereoisomer, or isotopically labeled derivative thereof, wherein ring A, R^{1a}, R^{1b}, R², R³, R⁴, R⁷, R⁸, m, n and all subvariables thereof are selected from any of the embodiments or aspects thereof set forth above for such variable and subvariable. In one aspect of these embodiments, the compound has the structural formula Ia-1:

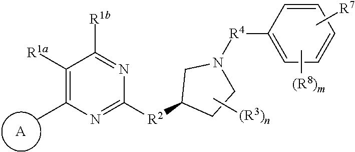

or a pharmaceutically acceptable salt, solvate, hydrate, tautomer, or isotopically labeled derivative thereof, wherein ring A, R^{1a}, R^{1b}, R², R³, R⁴, R⁷, R⁸, m, n and all subvariables thereof are selected from any of the embodiments or aspects thereof set forth above for such variable and subvariable.

In certain other embodiments, the compound of Formula (I) is of Formula (Ib):

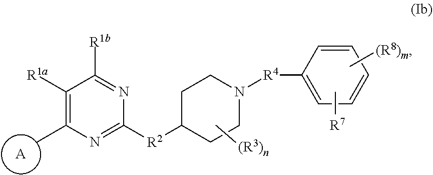

or a pharmaceutically acceptable salt, solvate, hydrate, tautomer, stereoisomer, or isotopically labeled derivative thereof, wherein ring A, R^{1a}, R^{1b}, R², R³, R⁴, R⁷, R⁸, m, n and all subvariables thereof are selected from any of the embodiments or aspects thereof set forth above for such variable and subvariable.

In certain other embodiments, the compound of Formula (I) is of Formula (Ic):

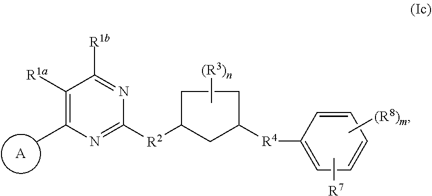

or a pharmaceutically acceptable salt, solvate, hydrate, tautomer, stereoisomer, or isotopically labeled derivative thereof, wherein ring A, R^{1a}, R^{1b}, R², R³, R⁴, R⁷, R⁸, m, n and all subvariables thereof are selected from any of the embodiments or aspects thereof set forth above for such variable and subvariable.

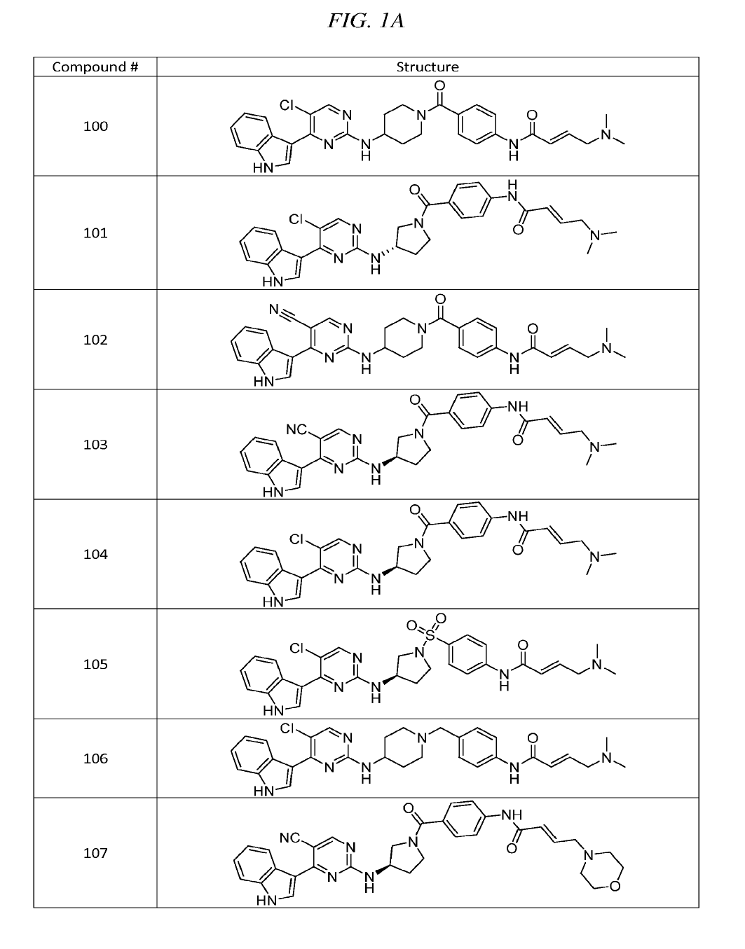

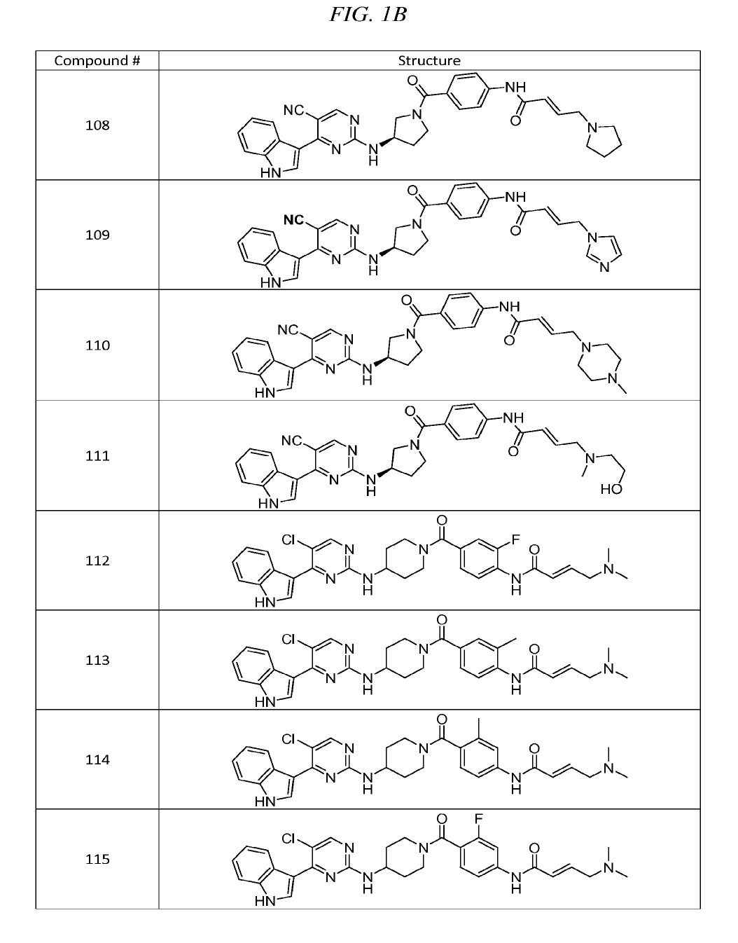

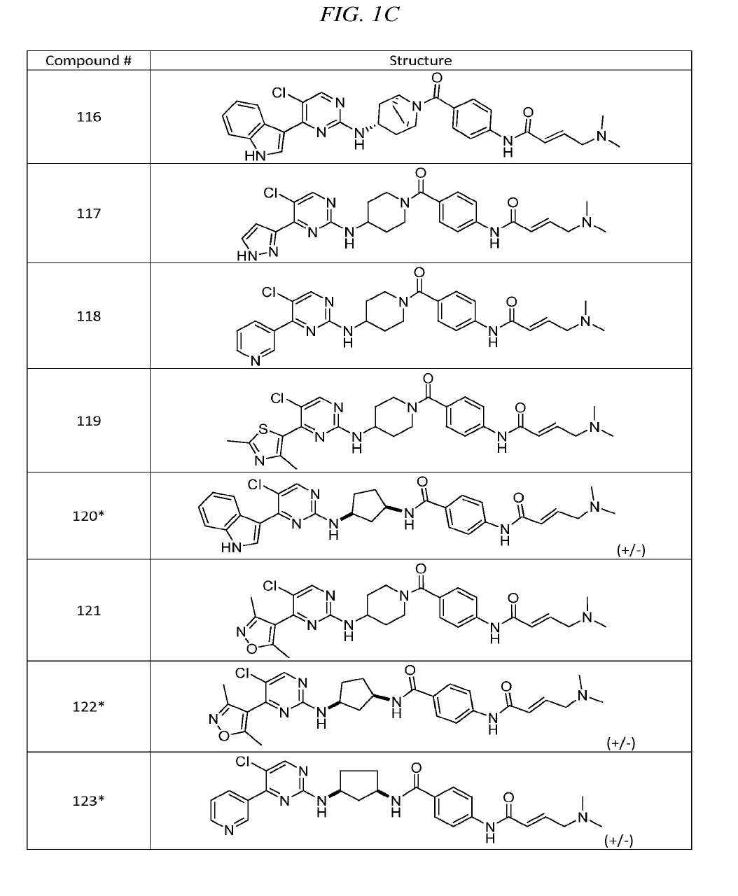

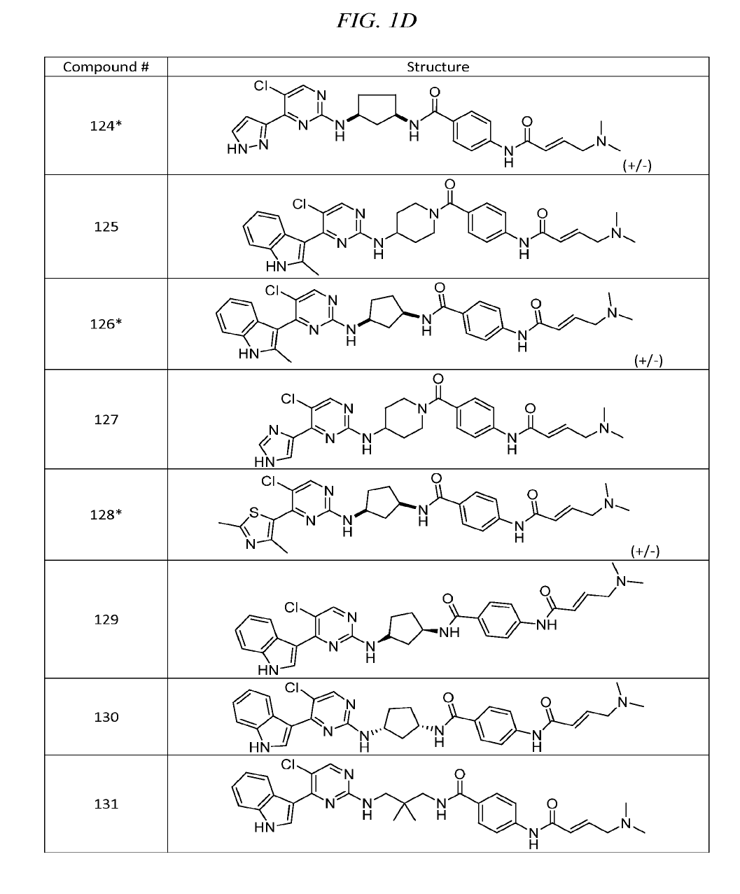

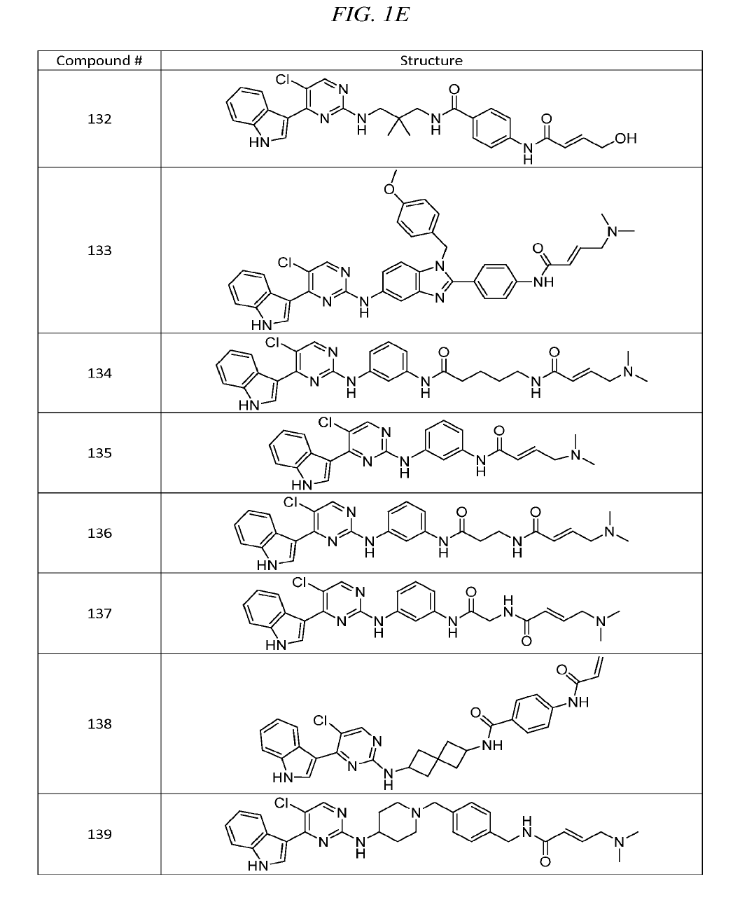

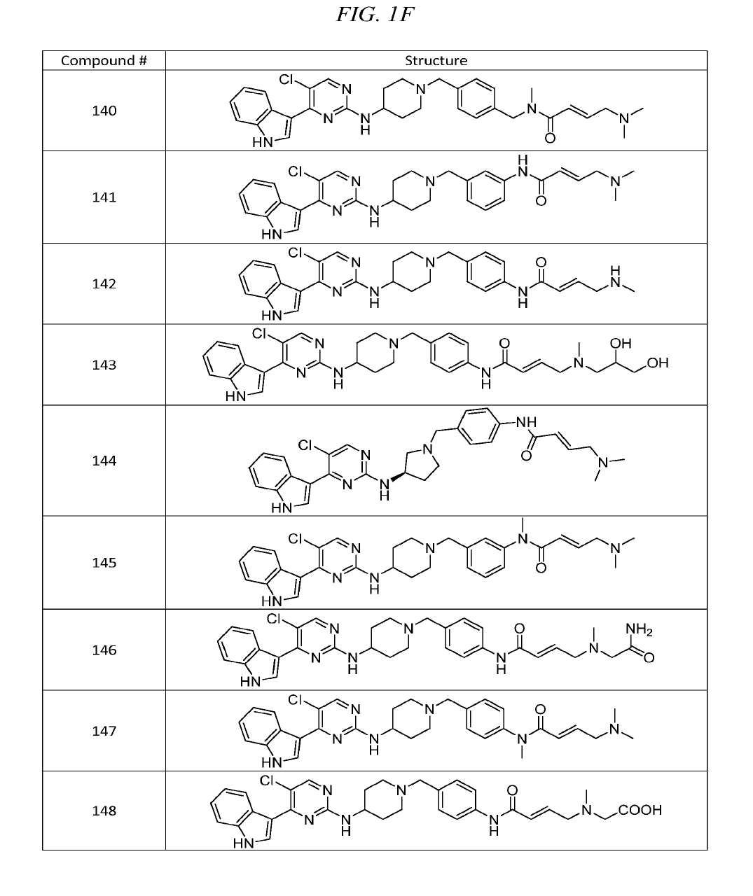

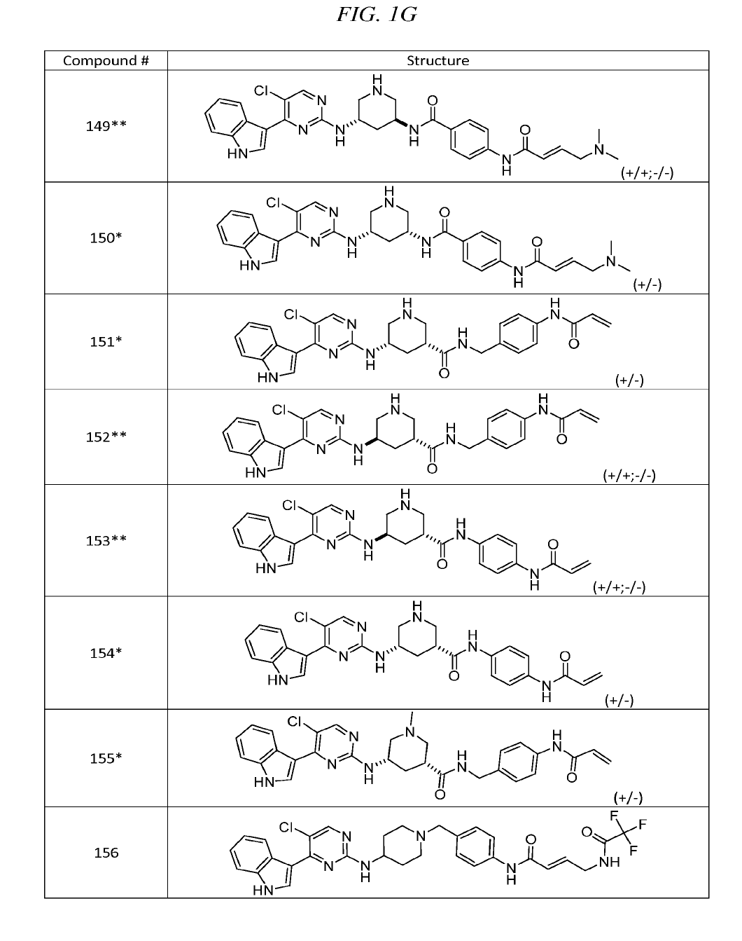

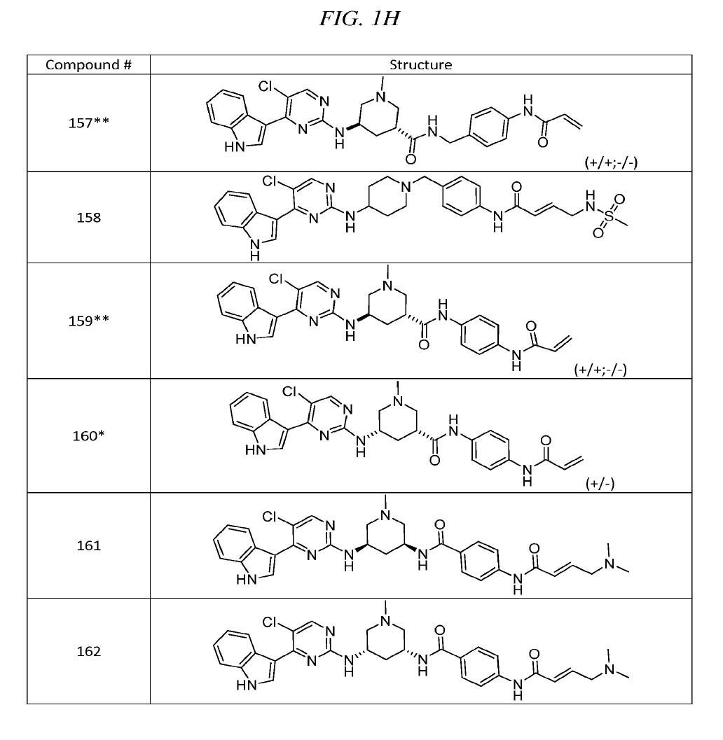

In certain embodiments, the compound of Formula (I) is selected from the group consisting of any one of the compounds in FIG. 1A-FIG. 1H and pharmaceutically acceptable salts, solvates, hydrates, tautomers, stereoisomers, and isotopically labeled derivatives thereof.

In another aspect, the present invention provides compounds of Formula (II):

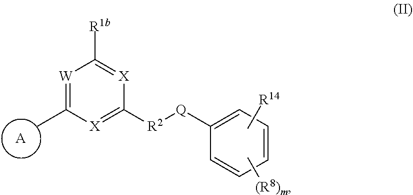

and pharmaceutically acceptable salts, solvates, hydrates, tautomers, stereoisomers, and isotopically labeled derivatives thereof, wherein m, Ring A, W, X, R^{1b}, R², Q, R⁸, and subvariables thereof are as defined herein for Formula (I) and embodiments and specific aspects thereof set forth above; and wherein R¹⁴ is selected from —C₁-C₈ alkyl, —O—C₁-C₈ alkyl, —NH₂, —NH(C₁-C₈ alkyl), —N(C₁-C₈ alkyl)₂, wherein each alkyl in R¹⁴ is independently selected and optionally and independently substituted.

In some embodiments, R¹⁴ is selected from —(C₁-C₄ alkyl), —C(O)—(C₁-C₄ alkylene)-NH₂, —(C₁-C₄ alkylene)-NH₂, —NH₂, —NH—C(O)—(C₁-C₄ alkylene)-NH₂, —NH—C(O)—(C₁-C₄ alkylene)-NH—(C₁-C₄ alkyl), —NH—C(O)—(C₁-C₄ alkylene)-N—(C₁-C₄ alkyl)₂, —NH—C(O)—C(O)—(C₀-C₄ alkylene)-NH₂, —NH—C(O)—C(O)—(C₀-C₄ alkylene)-NH(C₁-C₄ alkyl), —NH—C(O)—C(O)—(C₀-C₄ alkylene)-N(C₁-C₄ alkyl)₂, and —NH—C(O)—(C₁-C₄ alkyl). In another aspect of these embodiments, R¹⁴ is selected from —NH₂, —NH—CH₃, —NH—C(O)—CH₃, —NH—C(O)—(CH₂)₃—N(CH₃)₂, —NH—C(O)—(CH₂)₄—NH₂, —NH—C(O)—(CH₂)₂—NH₂, and —NH—C(O)—CH₂—NH₂.

Although, as indicated above, various embodiments and aspects thereof for a variable in Formula (II) may be selected from a group of chemical moieties set forth for the same variables in Formula (I), the invention also encompasses as further embodiments and aspects thereof situations where such variable in Formula (II) is: a) selected from any subset of chemical moieties in such a group; and b) any single member of such a group.

Although various embodiments and aspects thereof are set forth (or implied, as discussed in the preceding paragraphs) individually for each variable in Formula (II) above, the invention encompasses all possible combinations of the different embodiments and aspects for each of the variables in Formula (II).

Thus, in certain embodiments, the compound of Formula (II) is of Formula (IIa):

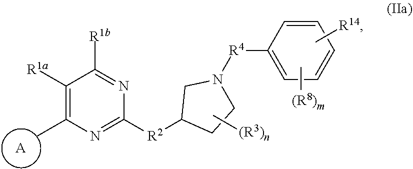

or a pharmaceutically acceptable salt, solvate, hydrate, tautomer, stereoisomer, or isotopically labeled derivative thereof, wherein ring A, R^{1a}, R^{1b}, R², R³, R⁴, R⁸, R¹⁴, m, n and all subvariables thereof are selected from any of the embodiments or aspects thereof set forth above for such variable and subvariable. In one aspect of these embodiments, the compound has the structural formula IIa-1:

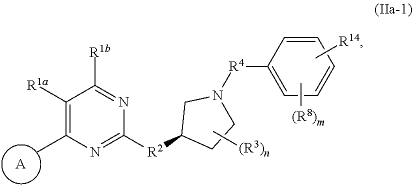

or a pharmaceutically acceptable salt, solvate, hydrate, tautomer, or isotopically labeled derivative thereof, wherein ring A, R^{1a}, R^{1b}, R², R³, R⁴, R⁸, R¹⁴, m, n and all subvariables thereof are selected from any of the embodiments or aspects thereof set forth above for such variable and subvariable.

In certain other embodiments, the compound of Formula (II) is of Formula (IIb):

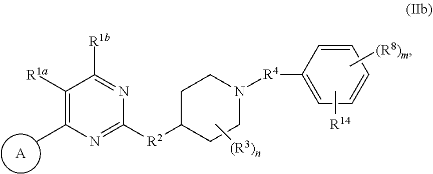

or a pharmaceutically acceptable salt, solvate, hydrate, tautomer, stereoisomer, or isotopically labeled derivative thereof, wherein ring A, R^{1a}, R^{1b}, R², R³, R⁴, R⁸, R¹⁴, m, n and all subvariables thereof are selected from any of the embodiments or aspects thereof set forth above for such variable and subvariable.

In certain other embodiments, the compound of Formula (II) is of Formula (IIc):

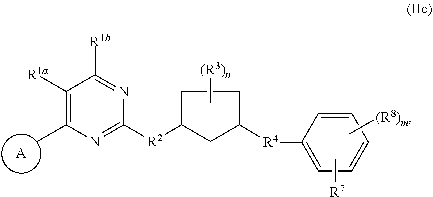

or a pharmaceutically acceptable salt, solvate, hydrate, tautomer, stereoisomer, or isotopically labeled derivative thereof, wherein ring A, R^{1a}, R^{1b}, R², R³, R⁴, R⁸, R¹⁴, m, n and all subvariables thereof are selected from any of the embodiments or aspects thereof set forth above for such variable and subvariable.

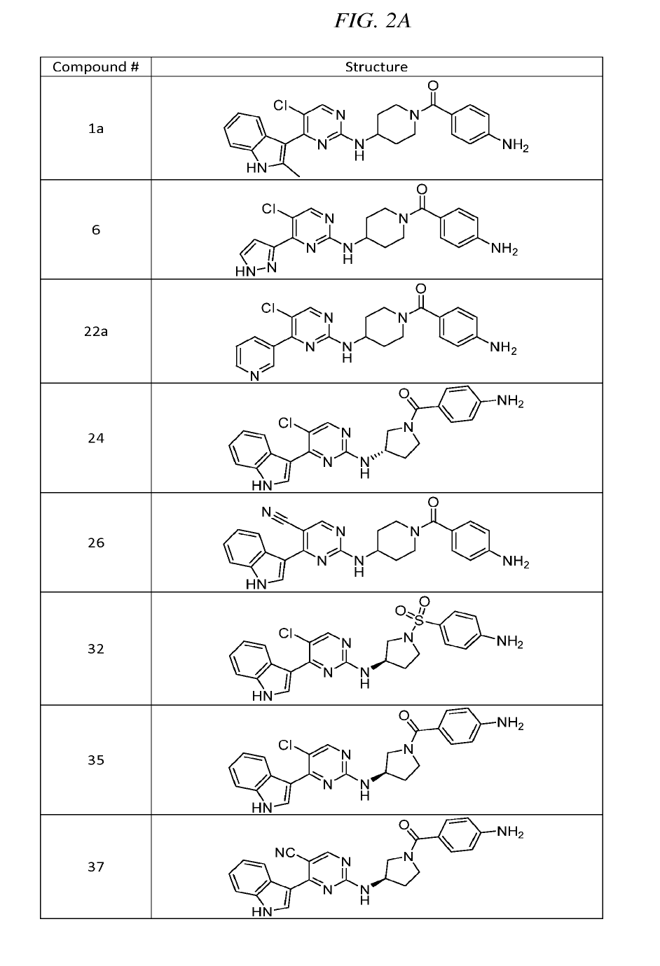

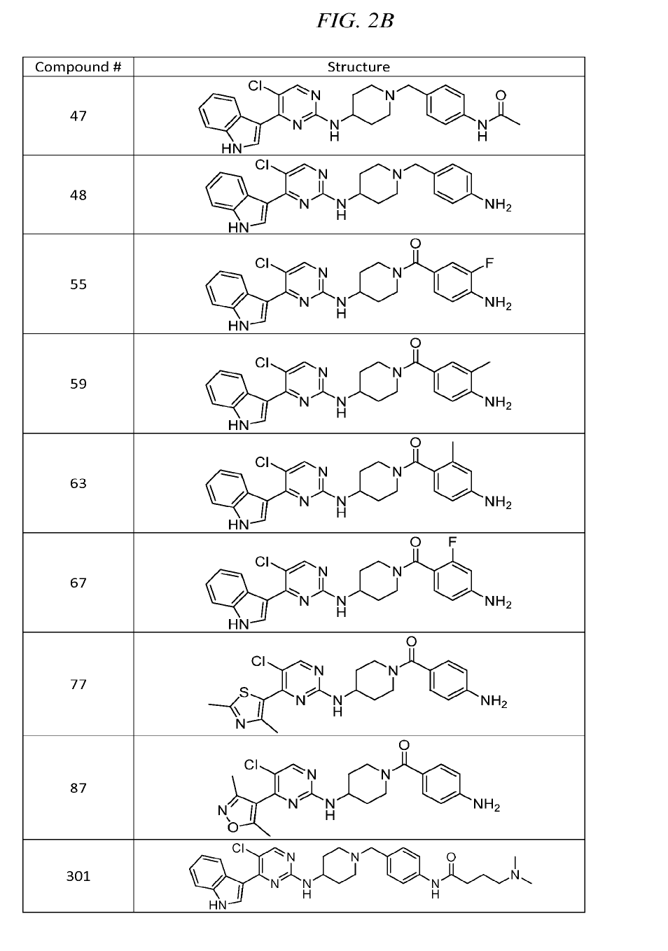

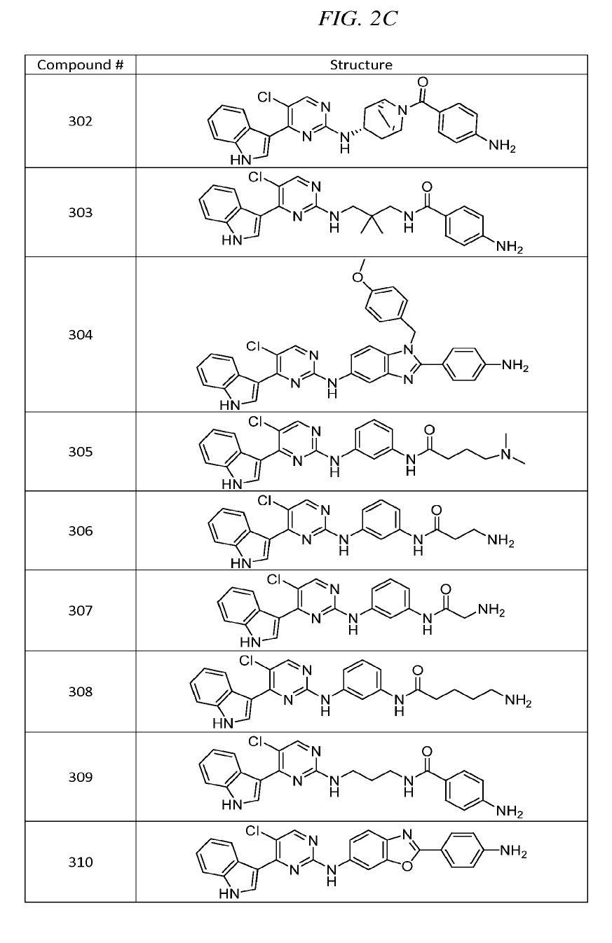

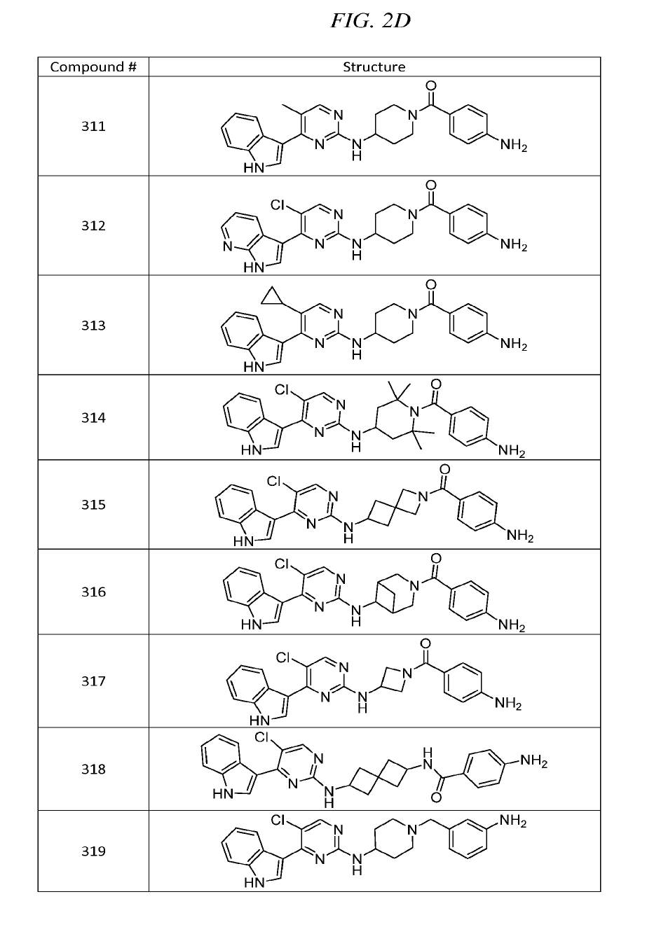

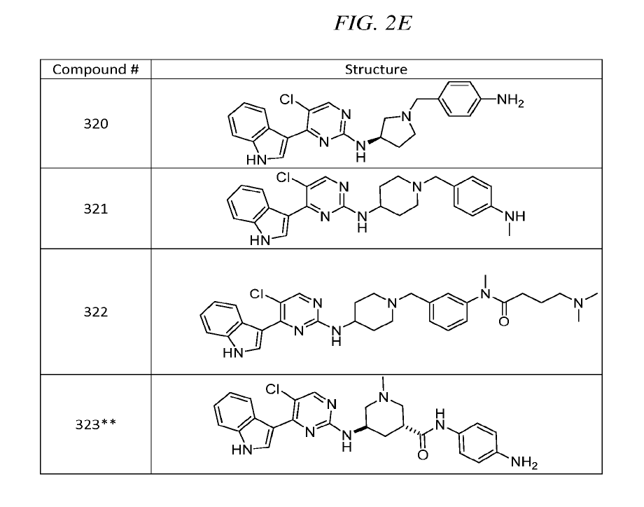

In certain embodiments, the compound of Formula (II) is selected from the group consisting of any one of the compounds in FIG. 2A-FIG. 2E and pharmaceutically acceptable salts, solvates, hydrates, tautomers, stereoisomers, and isotopically labeled derivatives thereof.

### Pharmaceutical Compositions, Kits, and Administration

The present invention provides pharmaceutical compositions comprising a compound of Formula (I) or Formula (II), e.g., a compound of Formula (I) or Formula (II), or a pharmaceutically acceptable salt, solvate, hydrate, tautomer, stereoisomer, or isotopically labeled derivative thereof, as described herein, and optionally a pharmaceutically acceptable excipient. In certain embodiments, the pharmaceutical composition of the invention comprises a compound of Formula (I) or Formula (II), or a pharmaceutically acceptable salt thereof, and optionally a pharmaceutically acceptable excipient. In certain embodiments, the compound of Formula (I) or Formula (II), or a pharmaceutically acceptable salt, solvate, hydrate, tautomer, stereoisomer, or isotopically labeled derivative thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or Formula (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For ophthalmic use, provided pharmaceutically acceptable compositions may be formulated as micronized suspensions or in an ointment such as petrolatum.

Pharmaceutically acceptable compositions of this invention may also be administered by nasal aerosol or inhalation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or Formula (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease (e.g., cancer (e.g., leukemia, melanoma, multiple myeloma), benign neoplasm, angiogenesis, inflammatory disease, autoinflammatory disease, or autoimmune disease). The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Thus, in one aspect, provided are kits including a first container comprising a compound described herein, or a pharmaceutically acceptable salt, solvate, hydrate, tautomer, stereoisomer, and isotopically labeled derivative, or a pharmaceutical composition thereof. In certain embodiments, the kit of the invention includes a first container comprising a compound described herein, or a pharmaceutically acceptable salt thereof, or a pharmaceutical composition thereof. In certain embodiments, the kits are useful in preventing and/or treating a proliferative disease in a subject. In certain embodiments, the kits further include instructions for administering the compound, or a pharmaceutically acceptable salt, solvate, hydrate, tautomer, stereoisomer, isotopically and labeled derivative thereof, or a pharmaceutical composition thereof, to a subject to prevent and/or treat a proliferative disease.

### Methods of Treatment and Uses

The present invention also provides methods for the treatment or prevention of a proliferative disease (e.g., cancer, benign neoplasm, angiogenesis, inflammatory disease, autoinflammatory disease, or autoimmune disease) or an infectious disease (e.g., a viral disease) in a subject. Such methods comprise the step of administering to the subject in need thereof an effective amount of a compound of Formula (I) or Formula (II), or a pharmaceutically acceptable salt, solvate, hydrate, tautomer, stereoisomer, or isotopically labeled derivative thereof, or a pharmaceutical composition thereof. In certain embodiments, the methods described herein include administering to a subject an effective amount of a compound of Formula (I) or Formula (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

The proliferative disease to be treated or prevented using the compounds of Formula (I) or Formula (II) will typically be associated with aberrant activity of CDK7. Aberrant activity of CDK7 may be an elevated and/or an inappropriate (e.g., abnormal) activity of CDK7. In certain embodiments, CDK7 is not overexpressed, and the activity of CDK7 is elevated and/or inappropriate. In certain other embodiments, CDK7 is overexpressed, and the activity of CDK7 is elevated and/or inappropriate. The compounds of Formula (I) or Formula (II), and pharmaceutically acceptable salts, solvates, hydrates, tautomers, stereoisomers, isotopically labeled derivatives, and compositions thereof, may inhibit the activity of CDK7 and be useful in treating and/or preventing proliferative diseases.

In other embodiments, the proliferative disease to be treated or prevented using the compounds of Formula (I) or Formula (II) will typically be associated with aberrant activity of a CDK family member, e.g., CDK12 or CDK13. Aberrant activity of CDK12 or CDK13 may be an elevated and/or an inappropriate (e.g., abnormal) activity of CDK12 or CDK13. In certain embodiments, CDK12 or CDK13 is not overexpressed, and the activity of CDK12 or CDK13 is elevated and/or inappropriate. In certain other embodiments, CDK12 or CDK13 is overexpressed, and the activity of CDK12 or CDK13 is elevated and/or inappropriate. The compounds of Formula (I) or Formula (II), and pharmaceutically acceptable salts, solvates, hydrates, tautomers, stereoisomers, isotopically labeled derivatives, and compositions thereof, may inhibit the activity of CDK12 or CDK13 and be useful in treating and/or preventing proliferative diseases.

A proliferative disease may also be associated with inhibition of apoptosis of a cell in a biological sample or subject. All types of biological samples described herein or known in the art are contemplated as being within the scope of the invention. Inhibition of the activity of CDK7 is expected to cause cytotoxicity via induction of apoptosis. The compounds of Formula (I) or Formula (II), and pharmaceutically acceptable salts, solvates, hydrates, tautomers, stereoisomers, isotopically labeled derivatives, and compositions thereof, may induce apoptosis, and therefore, be useful in treating and/or preventing proliferative diseases.

In certain embodiments, the proliferative disease to be treated or prevented using the compounds of Formula (I) or Formula (II) is cancer. All types of cancers disclosed herein or known in the art are contemplated as being within the scope of the invention. In certain embodiments, the proliferative disease is a cancer associated with dependence on BCL-2 anti-apoptotic proteins (e.g., MCL-1 and/or XIAP). In certain embodiments, the proliferative disease is a cancer associated with overexpression of MYC (a gene that codes for a transcription factor). In certain embodiments, the proliferative disease is a hematological malignancy. In certain embodiments, the proliferative disease is a blood cancer. In certain embodiments, the proliferative disease is leukemia. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myelogenous leukemia (AML). In certain embodiments, the proliferative disease is lymphoma. In certain embodiments, the proliferative disease is melanoma. In certain embodiments, the proliferative disease is multiple myeloma. In certain embodiments, the proliferative disease is a bone cancer. In certain embodiments, the proliferative disease is osteosarcoma. In some embodiments, the proliferative disease is Ewing's sarcoma. In some embodiments, the proliferative disease is triple-negative breast cancer (TNBC). In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is large cell lung cancer. In some embodiments, the proliferative disease is a benign neoplasm. All types of benign neoplasms disclosed herein or known in the art are contemplated as being within the scope of the invention.

In some embodiments, the proliferative disease is associated with angiogenesis. All types of angiogenesis disclosed herein or known in the art are contemplated as being within the scope of the invention.

In certain embodiments, the proliferative disease is an inflammatory disease. All types of inflammatory diseases disclosed herein or known in the art are contemplated as being within the scope of the invention. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the invention. In some embodiments, the proliferative disease is an autoimmune disease. All types of autoimmune diseases disclosed herein or known in the art are contemplated as being within the scope of the invention.

The cell described herein may be an abnormal cell. The cell may be in vitro or in vivo. In certain embodiments, the cell is a proliferative cell. In certain embodiments, the cell is a blood cell. In certain embodiments, the cell is a lymphocyte. In certain embodiments, the cell is a cancer cell. In certain embodiments, the cell is a leukemia cell. In certain embodiments, the cell is a CLL cell. In certain embodiments, the cell is a melanoma cell. In certain embodiments, the cell is a multiple myeloma cell. In certain embodiments, the cell is a benign neoplastic cell. In certain embodiments, the cell is an endothelial cell. In certain embodiments, the cell is an immune cell.

In another aspect, the present invention provides methods of down-regulating the expression of a CDK (e.g., CDK7, CDK1, CDK2, CDK5, CDK8, CDK9, CDK12, CDK13) in a biological sample or subject. In certain embodiments, the present invention provides methods of down-regulating the expression of CDK7 in a biological sample or subject. In another aspect, the present invention provides methods of down-regulating the expression of IRAK1, JNK1, JNK2, or MLK3 in a biological sample or subject.

Another aspect of the invention relates to methods of inhibiting the activity of a kinase in a biological sample or subject. In certain embodiments, the kinase is CDK. In certain embodiments, the kinase is CDK7. In other embodiments, the kinase is CDK12 or CDK13. In certain embodiments, the activity of the kinase is aberrant activity of the kinase. In certain embodiments, the inhibition of the activity of the kinase is irreversible. In other embodiments, the inhibition of the activity of the kinase is reversible. In certain embodiments, the methods of inhibiting the activity of the kinase include attaching a compound of Formula (I) to the kinase.

In certain embodiments, the methods described herein comprise the additional step of administering one or more additional pharmaceutical agents in combination with the compound of Formula (I) or Formula (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment inhibition of CDK7, CDK12, or CDK13 induced by the inventive compounds or compositions of this invention in the biological sample or subject. In certain embodiments, the additional pharmaceutical agent is flavopiridol, triptolide, SNS-032 (BMS-387032), PHA-767491, PHA-793887, BS-181, (S)-CR8, (R)-CR8, or NU6140. In certain embodiments, the additional pharmaceutical agent is an inhibitor of a mitogen-activated protein kinase (MAPK). In certain embodiments, the additional pharmaceutical agent is an inhibitor of a glycogen synthase kinase 3 (GSK3). In certain embodiments, the additional pharmaceutical agent is an inhibitor of an AGC kinase. In certain embodiments, the additional pharmaceutical agent is an inhibitor of a CaM kinase. In certain embodiments, the additional pharmaceutical agent is an inhibitor of a casein kinase 1. In certain embodiments, the additional pharmaceutical agent is an inhibitor of a STE kinase. In certain embodiments, the additional pharmaceutical agent is an inhibitor of a tyrosine kinase. Thus, the combination of the inventive compounds or compositions and the additional pharmaceutical agent(s) may be useful in treating proliferative diseases resistant to a treatment using the additional pharmaceutical agent(s) without the inventive compounds or compositions.

In some embodiments, the one or more additional pharmaceutical agents are independently selected from a topoisomerase inhibitor, a MCL1 inhibitor, a BCL-2 inhibitor, a BCL-xL inhibitor, a BRD4 inhibitor, a CDK9 inhibitor, a Jumonji histone demethylase inhibitor, and a DNA damage inducer. In a more specific aspect of these embodiments, the one or more additional agents is selected from etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04 and cisplatin. In an even more specific aspect of these embodiments, the additional agent is selected from etoposide, obatoclax, and navitoclax and the disease to be treated is breast cancer, e.g., triple-negative breast cancer, HER2 positive breast cancer, ER-positive breast cancer, or ER/PR-positive breast cancer. In another even more specific aspect of these embodiments, the additional agent is selected from etoposide, JIB04 and cisplatin and the disease to be treated is Ewing's sarcoma. In still another even more specific aspect of these embodiments, the additional agent is selected from JQ1 and 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, and the disease to be treated is leukemia, e.g., acute myelogenous leukemia, myeloblastic leukemia, promyelocytic leukemia, myelomonocytic leukemia, monocytic leukemia, monoblastic leukemia, or megakaryoblastic leukemia.

In yet another aspect, the present invention provides the compounds of Formula (I) or Formula (II), and pharmaceutically acceptable salts, solvates, hydrates, tautomers, stereoisomers, isotopically labeled derivatives, and compositions thereof, for use in the treatment of a proliferative disease in a subject. In certain embodiments, provided by the invention are the compounds described herein, and pharmaceutically acceptable salts and compositions thereof, for use in the treatment of a proliferative disease in a subject. In certain embodiments, provided by the invention are the compounds described herein, and pharmaceutically acceptable salts and compositions thereof, for use in inhibiting cell growth. In certain embodiments, provided by the invention are the compounds described herein, and pharmaceutically acceptable salts and compositions thereof, for use in inducing apoptosis in a cell. In certain embodiments, provided by the invention are the compounds described herein, and pharmaceutically acceptable salts and compositions thereof, for use in inhibiting transcription.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

| | |
|---|---|
| Ac | acetyl |
| ACN | acetonitrile |
| aq. | aqueous |
| atm | atmospheres |
| Boc | tert-butoxy carbonyl |
| Boc₂O | Di-t-butyl dicarbonate |
| DCC | N,N′-Dicyclohexylcarbodiimide |
| DCM | dichloromethane |
| DIAD | Diisopropyl azodicarboxylate |
| DIPEA | N,N-Diisopropyl ethylamine |
| DMA | Dimethyl adipate |
| DMF | Dimethylformamide |
| DMSO | dimethylsulfoxide |
| DPPA | Diphenoxyphosphoryl azide |
| EDTA | ethylenediamine tetraacetic acid |
| eq(s). | equivalent(s) |
| EtOAc | ethyl acetate |
| Et | Ethyl |
| EtOH | ethanol |
| Et₃N | triethylamine |
| g | gram(s) |
| h | hour(s) |
| HATU | (Dimethylamino)-N,N-dimethyl(3H-[1,2,3]triazolo[4,5- |
| | b]pyridin-3-yloxy)methaniminium hexafluorophosphate |
| HBTU | O-Benzotriazole-N,N,N′,N′-tetramethyl-uronium- |
| | hexafluoro-phosphate |
| Hex | hexane |
| HOBt | 1-Hydroxybenzotriazole |
| HPLC | High pressure liquid chromatography |
| IPA | isopropanol |
| LCMS; LC- | liquid chromatography mass spectrometry |
| MS | |
| MeOH | methanol |
| mg | milligram(s) |
| min | Minute(s) |
| mL; ml | milliliter(s) |
| MS | mass spectrometry |
| mW | megawatt |
| NMe | N-methyl |
| NMP | N-Methyl-2-pyrrolidone |
| NMR | Nuclear magnetic resonance |
| Pd₂dba₃ | Tris(dibenzylideneacetone) dipalladium(0) |
| Ph | phenyl |
| r.t.; rt; RT | Room temperature |
| S. | saturated |
| TEA | triethylamine |
| TFA | trifluoroacetic acid |
| THF | tetrahydrofuran |
| TLC | Thin layer chromatography |
| X-Phos | 2-Dicyclohexylphosphino-2′,4′,6′-triisopropylbiphenyl |

### Example 1. Synthesis of (E)-N-(4-(4-(5-chloro-4-(1H-indol-3-yl)pyrimidin-2-ylamino)piperidine-1-carbonyl)phenyl)-4-(dimethylamino)but-2-enamide (Compound 100)

### p-{[4-(Benzyloxycarbonylamino)-1-piperidyl]carbonyl}phenylamino 2,2-dimethylpropionate

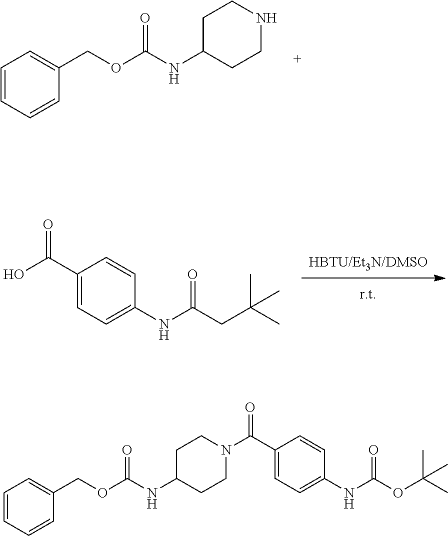 [SMILES: O=C(NC1CCNCC1)OCc1ccccc1 ; CC(C)(C)CC(=O)Nc1ccc(C(=O)O)cc1 ; CC(C)(C)OC(=O)Nc1ccc(C(=O)N2CCC(NC(=O)OCc3ccccc3)CC2)cc1]

To a solution of 4-(tert-butoxycarbonylamino)benzoic acid (500 mg, 2 mmol), 4-CBz-aminopiperidine (500 mg, 2 mmol) and Et₃N (0.89 ml, 6 mmol) in DMSO (10 mL) was added HBTU (1.2 g, 3 mmol) and the mixture was stirred 12 h at rt. The reaction was then diluted with EtOAc (100 ml) and water (100 mL). The layers were separated and the organic layer was washed with brine (3×100 mL). The organic layer was dried (MgSO₄), filtered and concentrated under reduced pressure. The residue was purified by SiO₂ chromatography (DCM/MeOH, 0 to 10% gradient) and afforded the title compound as a white solid (850 mg, 87.8%)

### tert-butyl 4-(4-aminopiperidine-1-carbonyl)phenylcarbamate

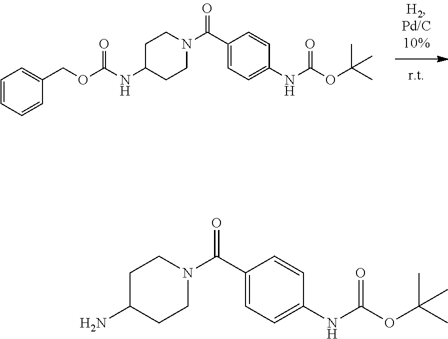 [SMILES: CC(C)(C)OC(=O)Nc1ccc(C(=O)N2CCC(NC(=O)OCc3ccccc3)CC2)cc1 ; CC(C)(C)OC(=O)Nc1ccc(C(=O)N2CCC(N)CC2)cc1]

To a degassed solution of p-{[4-(Benzyloxycarbonylamino)-1-piperidyl]carbonyl}phenylamino 2,2-dimethylpropionate (75 mg, 0.165 mmol) in MeOH (5 mL) was added 10% Pd/C (0.1 g). The resulting mixture was stirred 1 h under H₂ (1 atm) before being filtered over Celite (MeOH). The volatile were removed under reduced pressure and afford the title compound (50 mg, 0.156 mmol, 94.7%) as a white solid.

### tert-butyl 4-(4-(5-chloro-4-(1-(phenylsulfonyl)-1H-indol-3-yl)pyrimidin-2-ylamino)piperidine-1-carbonyl)phenylcarbamate

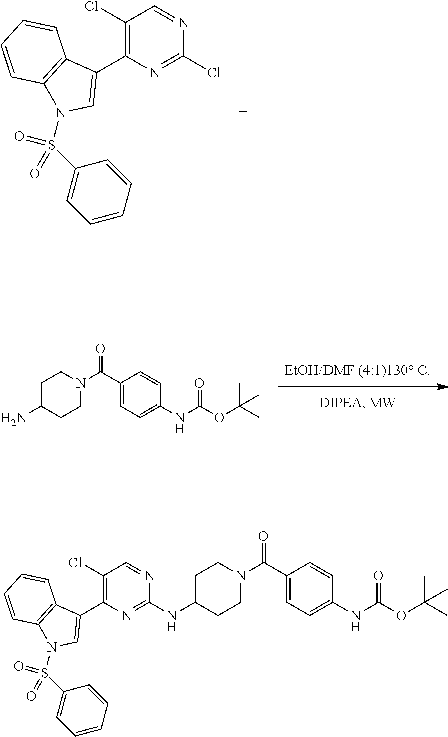 [SMILES: O=S(=O)(c1ccccc1)n1cc(-c2nc(Cl)ncc2Cl)c2ccccc21 ; CC(C)(C)OC(=O)Nc1ccc(C(=O)N2CCC(N)CC2)cc1 ; CC(C)(C)OC(=O)Nc1ccc(C(=O)N2CCC(Nc3ncc(Cl)c(-c4cn(S(=O)(=O)c5ccccc5)c5ccccc45)n3)CC2)cc1]

A suspension of 3-(2,5-dichloropyrimidin-4-yl)-1-(phenylsulfonyl)-1H-indole (63 mg, 0.160 mmol), tert-butyl 4-(4-aminopiperidine-1-carbonyl)phenylcarbamate (50 mg, 0.160 mmol) and DIPEA (54 μL, 0.31 mmol) in EtOH/DMF (4/1, 3 mL) was heated at 130° C. (mW) for 20 min. After being cooled to room temperature, the mixture was diluted with EtOAc (10 mL), washed with sat NaHCO₃ (5 mL), brine (5 mL), dried (MgSO₄), filtered and concentrated under reduced pressure. The crude mixture was purified by SiO₂ chromatography (Hex/EtOAc, 20 to 100% gradient) and afforded the title compound (100 mg, 0.146 mmol, 93.4%) as a beige solid.

### (4-aminophenyl)(4-(5-chloro-4-(1-(phenylsulfonyl)-1H-indol-3-yl)pyrimidin-2-ylamino)piperidin-1-yl)methanone TFA Salt

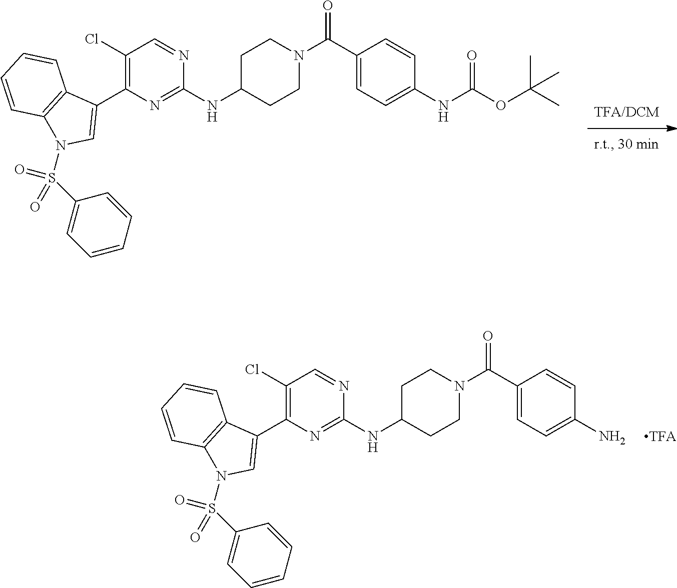 [SMILES: CC(C)(C)OC(=O)Nc1ccc(C(=O)N2CCC(Nc3ncc(Cl)c(-c4cn(S(=O)(=O)c5ccccc5)c5ccccc45)n3)CC2)cc1 ; Nc1ccc(C(=O)N2CCC(Nc3ncc(Cl)c(-c4cn(S(=O)(=O)c5ccccc5)c5ccccc45)n3)CC2)cc1]

To a stirred solution of tert-butyl 4-(4-(5-chloro-4-(1-(phenylsulfonyl)-1H-indol-3-yl)pyrimidin-2-ylamino)piperidine-1-carbonyl)phenylcarbamate (107 mg, 0.156 mmol) in DCM (3 mL) was added TFA (1 mL). The mixture was stirred 30 min at rt before being concentrated under reduced pressure and afforded the title compound (109 mg, 0.155 mmol, 99.6%) as a colorless glue which was used in the next step without further purification.

### (E)-N-(4-(4-(5-chloro-4-(1-(phenylsulfonyl)-1H-indol-3-yl)pyrimidin-2-ylamino)piperidine-1-carbonyl)phenyl)-4-(dimethylamino)but-2-enamide

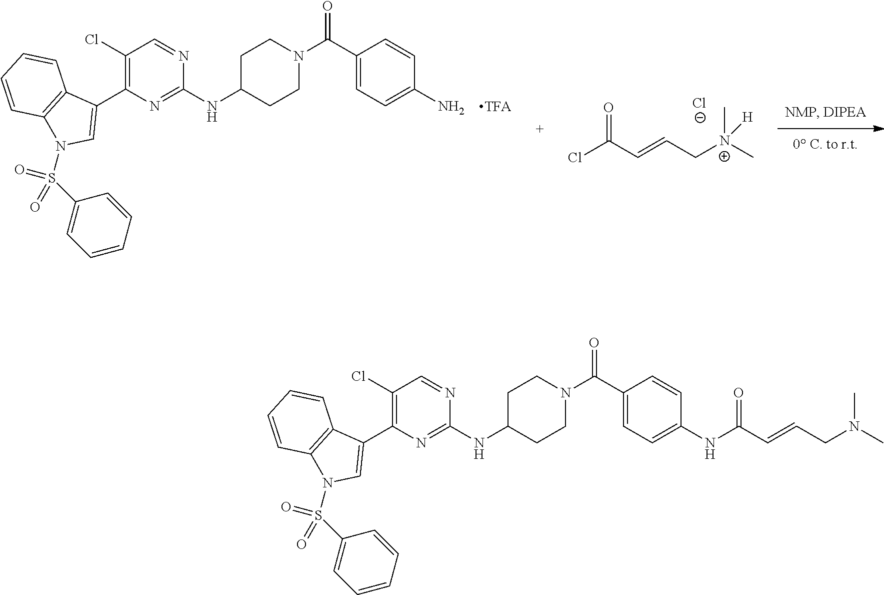

To a 0° C. solution of (4-aminophenyl)(4-(5-chloro-4-(1-(phenylsulfonyl)-1H-indol-3-yl)pyrimidin-2-ylamino)piperidin-1-yl)methanone.TFA (107 mg, 0.181 mmol) and DIPEA (95 μL, 0.54 mmol) in NMP (2 mL) was slowly added a 0.1M solution of (E)-4-chloro-N,N-dimethyl-4-oxobut-2-en-1-aminium chloride in DCM (1.81 mL, 0.181 mmol). After 1 hour the reaction was diluted with EtOAc (25 ml) before being washed with water (25 mL), sat NaHCO₃ (10 mL) and brine (3×10 mL). The organic layer was dried (MgSO₄), filtered and concentrated under reduced pressure. The crude mixture was triturated with Et₂O/Hexane (10:1) and afforded the title compound (130 mg, 0.143 mmol, 79%) as clean brown solid

### (E)-N-(4-(4-(5-chloro-4-(1H-indol-3-yl)pyrimidin-2-ylamino)piperidine-1-carbonyl)phenyl)-4-(dimethylamino)but-2-enamide (Compound 100)

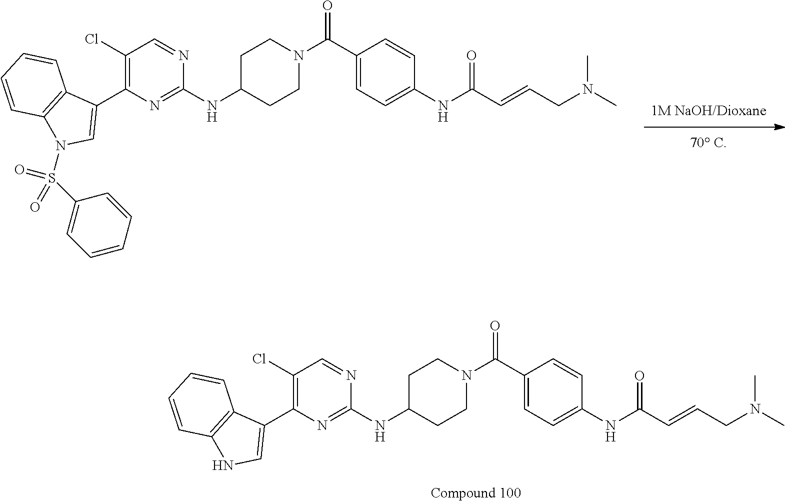 [SMILES: CN(C)C/C=C/C(=O)Nc1ccc(C(=O)N2CCC(Nc3ncc(Cl)c(-c4cn(S(=O)(=O)c5ccccc5)c5ccccc45)n3)CC2)cc1 ; CN(C)C/C=C/C(=O)Nc1ccc(C(=O)N2CCC(Nc3ncc(Cl)c(-c4c[nH]c5ccccc45)n3)CC2)cc1]

A solution of (E)-N-(4-(4-(5-chloro-4-(1-(phenylsulfonyl)-1H-indol-3-yl)pyrimidin-2-ylamino)piperidine-1-carbonyl)phenyl)-4-(dimethylamino)but-2-enamide (130 mg, 0.186 mmol) in dioxane (2 mL) and 1M NaOH (400 μL) was heated at 70° C. for 3 h. After being cooled to rt, the solution was treated with 10% HCl until pH=3-4. The mixture was extracted with EtOAc (3×10 mL) and the combined organic layers were washed with sat NaHCO₃ (10 mL). The organic layer was dried (MgSO₄), filtered and concentrated under reduced pressure. The crude mixture was purified by reverse phase chromatography (C18, H₂O/MeOH 0 to 100% gradient) and afforded Compound 100 (12 mg, 0.022 mmol, 11.4%) as a white solid after lyophilization. ¹H NMR (500 MHz, d₆-DMSO) δ 11.87 (s, 1H), 10.45 (s, 1H), 8.49 (d, J=3.1 Hz, 1H), 8.29 (s, 1H), 8.15 (s, 1H), 7.76 (d, J=8.6 Hz, 2H), 7.51 (d, J=8.0 Hz, 1H), 7.42 (d, J=8.5 Hz, 2H), 7.32 (d, J=7.6 Hz, 1H), 7.28-7.14 (m, 2H), 6.88-6.69 (m, 1H), 6.43 (d, J=15.5 Hz, 1H), 4.25-3.99 (m, 2H), 3.68 (m, 2H), 2.60 (s, 2H), 2.56 (s, 6H), 2.18-1.92 (m, 2H), 1.67-1.35 (m, 2H); MS (m/z): 558.66 [M+1]⁺.

### Example 2. Synthesis of (E)-N-(4-((S)-3-(5-chloro-4-(1H-indol-3-yl)pyrimidin-2-ylamino)pyrrolidine-1-carbonyl)phenyl)-4-(dimethylamino)but-2-enamide (Compound 101)

### (3S)-tert-butyl 3-(5-chloro-4-(1-(phenylsulfonyl)-1H-indol-3-yl)pyrimidin-2-ylamino)pyrrolidine-1-carboxylate

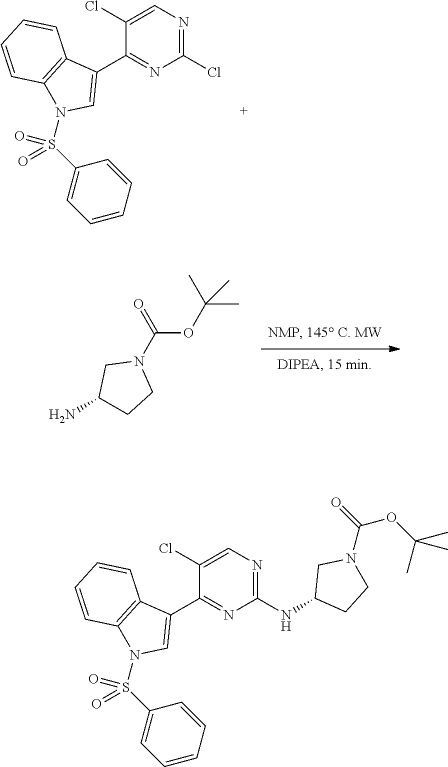 [SMILES: O=S(=O)(c1ccccc1)n1cc(-c2nc(Cl)ncc2Cl)c2ccccc21 ; CC(C)(C)OC(=O)N1CC[C@H](N)C1 ; CC(C)(C)OC(=O)N1CC[C@H](Nc2ncc(Cl)c(-c3cn(S(=O)(=O)c4ccccc4)c4ccccc34)n2)C1]

A solution of 3-(2,5-dichloropyrimidin-4-yl)-1-(phenylsulfonyl)-1H-indole (400 mg, 0.99 mmol), (S)-tert-butyl 3-aminopyrrolidine-1-carboxylate (193 mg, 1.04 mmol) and DIPEA (172 μL, 0.99 mmol) in NMP (2.64 mL) was heated at 135° C. (mW) for 15 min. After being cooled to rt, the reaction mixture was diluted with EtOAc (10 mL), washed with water (5 mL), brine (5 mL), dried (MgSO₄), filtered and concentrated under reduced pressure. The residue was purified by SiO₂ flash chromatography (Hex/EtOAc 0 to 100% gradient) and afforded the title compound (492 mg, 0.89 mmol, 85%) as a white solid.

### 5-chloro-4-(1-(phenylsulfonyl)-H-indol-3-yl)-N—((S)-pyrrolidin-3-yl)pyrimidin-2-amine.TFA

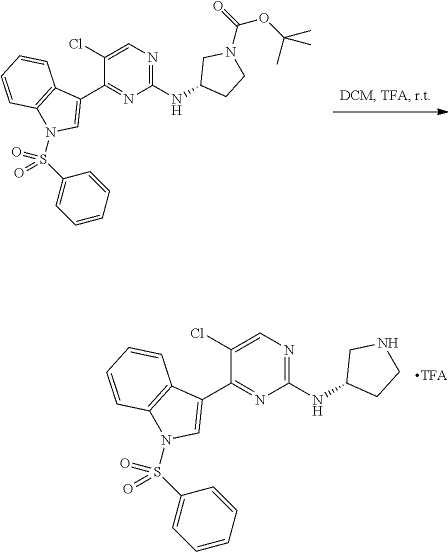 [SMILES: CC(C)(C)OC(=O)N1CC[C@H](Nc2ncc(Cl)c(-c3cn(S(=O)(=O)c4ccccc4)c4ccccc34)n2)C1 ; O=S(=O)(c1ccccc1)n1cc(-c2nc(N[C@H]3CCNC3)ncc2Cl)c2ccccc21]

A solution of (3S)-tert-butyl 3-(5-chloro-4-(1-(phenylsulfonyl)-1H-indol-3-yl)pyrimidin-2-ylamino)pyrrolidine-1-carboxylate (492 mg, 0.89 mmol) in DCM (4.0 mL) was treated with TFA (0.68 mL, 8.9 mmol). The mixture was stirred 90 min at rt before being concentrated under reduced pressure and afforded the title compound as a colorless oil (506 mg, 0.89 mmol, 100%) which was used in the next step without further purification.

### tert-butyl 4-((S)-3-(5-chloro-4-(1-(phenylsulfonyl)-1H-indol-3-yl)pyrimidin-2-ylamino)pyrrolidine-1-carbonyl)phenylcarbamate

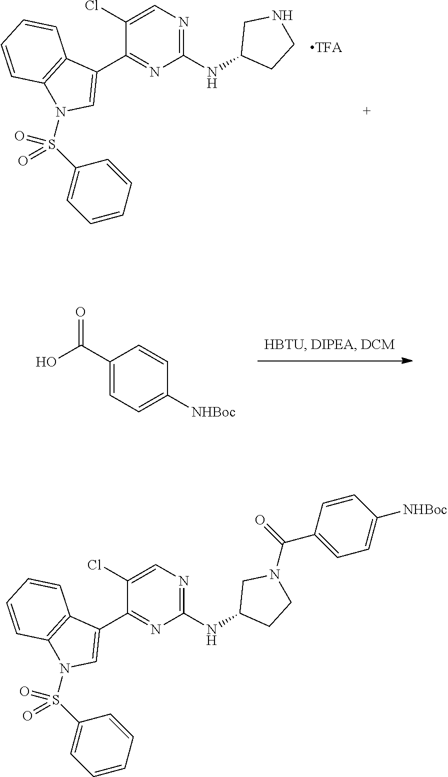 [SMILES: O=S(=O)(c1ccccc1)n1cc(-c2nc(N[C@H]3CCNC3)ncc2Cl)c2ccccc21 ; CC(C)(C)OC(=O)Nc1ccc(C(=O)O)cc1 ; CC(C)(C)OC(=O)Nc1ccc(C(=O)N2CC[C@H](Nc3ncc(Cl)c(-c4cn(S(=O)(=O)c5ccccc5)c5ccccc45)n3)C2)cc1]

A solution of 5-chloro-4-(1-(phenylsulfonyl)-1H-indol-3-yl)-N—((S)-pyrrolidin-3-yl)pyrimidin-2-amine.TFA (504 mg, 0.89 mmol), 4-(tert-butoxycarbonylamino)benzoic acid (211 mg, 0.89 mmol), HBTU (505 mg, 1.33 mmol) and diisopropylethylamine (0.46 mL, 2.66 mmol) was stirred overnight at rt. The reaction mixture was concentrated under reduced pressure and the mixture was purified by SiO₂ chromatography (Hex/EtOAc 0 to 100% gradient) and afforded the title compound (597 mg, 0.89 mmol, 100%) as a white solid.

### (4-aminophenyl)((S)-3-(5-chloro-4-(1-(phenylsulfonyl)-1H-indol-3-yl)pyrimidin-2-ylamino)pyrrolidin-1-yl)methanone.TFA

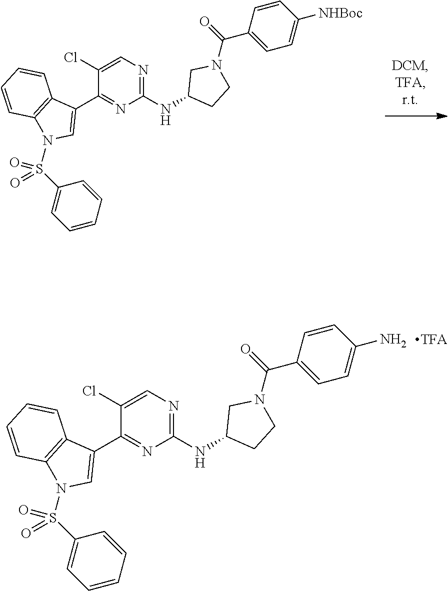 [SMILES: CC(C)(C)OC(=O)Nc1ccc(C(=O)N2CC[C@H](Nc3ncc(Cl)c(-c4cn(S(=O)(=O)c5ccccc5)c5ccccc45)n3)C2)cc1 ; Nc1ccc(C(=O)N2CC[C@H](Nc3ncc(Cl)c(-c4cn(S(=O)(=O)c5ccccc5)c5ccccc45)n3)C2)cc1]

A solution of ter-butyl 4-((S)-3-(5-chloro-4-(1-(phenylsulfonyl)-1H-indol-3-yl)pyrimidin-2-ylamino)pyrrolidine-1-carbonyl)phenylcarbamate (597 mg, 0.89 mmol) in DCM (3.9 mL) was treated with TFA (0.68 mL, 8.8 mmol). The mixture was stirred overnight at r before being diluted with dichloromethane (10 mL), washed with sat. NaHCO₃ (3×5 mL), brine (5 mL), dried (MgSO₄), filtered and concentrated under reduced pressure and afforded the title compound (446 mg, 0.65 mmol, 73%).

### (4-aminophenyl)((S)-3-(5-chloro-4-(1H-indol-3-yl)pyrimidin-2-ylamino)pyrrolidin-1-yl)methanone (Compound 24)

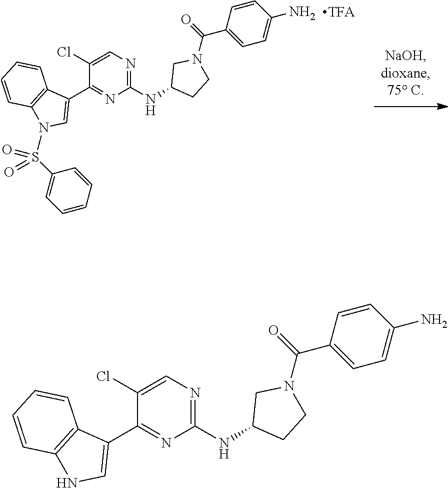 [SMILES: Nc1ccc(C(=O)N2CC[C@H](Nc3ncc(Cl)c(-c4cn(S(=O)(=O)c5ccccc5)c5ccccc45)n3)C2)cc1 ; Nc1ccc(C(=O)N2CC[C@H](Nc3ncc(Cl)c(-c4c[nH]c5ccccc45)n3)C2)cc1]

A solution of (4-aminophenyl)((S)-3-(5-chloro-4-(1-(phenylsulfonyl)-1H-indol-3-yl)pyrimidin-2-ylamino)pyrrolidin-1-yl)methanone.TFA (150 mg, 0.26 mmol) in 1,4-dioxane (1.2 mL) and 5M NaOH (0.52 mL, 2.6 mmol) was heated 2 h at 75° C. The cooled mixture was concentrated under reduced pressure, dissolved in DMF (1 mL) and injected on a reverse phase chromatography column (C18, H₂O/ACN 5 to 100%) to afford the title compound (96 mg, 0.22 mmol, 85%) as a white solid.

### (E)-N-(4-((S)-3-(5-chloro-4-(1H-indol-3-yl)pyrimidin-2-ylamino)pyrrolidine-1-carbonyl)phenyl)-4-(dimethylamino)but-2-enamide (Compound 101)

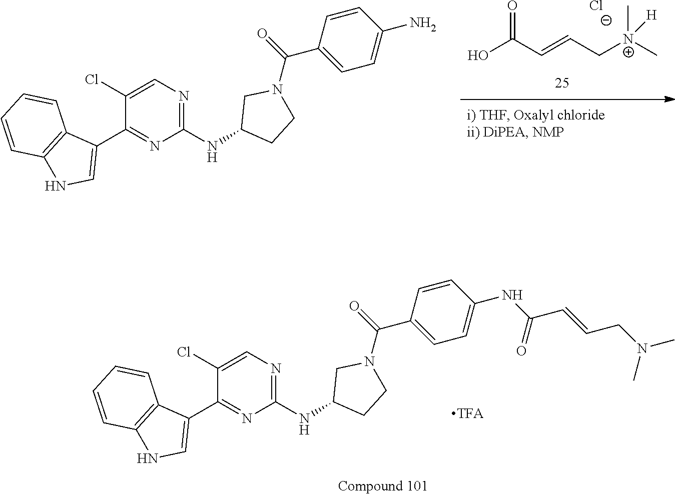

A solution of (E)-4-(dimethylamino)-2-butenoic acid hydrochloride (12.1 mg, 0.07 mmol) in THF (1.4 mL) and 1 drop of DMF was cooled to 0° C. Oxalyl chloride (10 eq, 59 μL, 0.7 mmol) was added dropwise and the mixture was warmed to rt. After 2 h at rt, the resulting suspension was concentrated under reduced pressure and co-evaporated 3 times with THF. The mixture was diluted with THF (2 mL), cooled to 0° C. and added to a solution of (4-aminophenyl)((S)-3-(5-chloro-4-(1H-indol-3-yl)pyrimidin-2-ylamino)pyrrolidin-1-yl)methanone (30 mg, 0.07 mmol) in THF (2 mL). After 5 h at rt, the mixture was concentrated under reduced pressure and the residue was purified by reverse phase preparative HPLC (MeCN/H₂O; 0.1% TFA 10 to 75% gradient) and afforded Compound 101 (14 mg, 0.021 mmol, 31%). ¹H NMR (500 MHz, d₆-DMSO) δ 11.89-11.79 (m, 1H), 11.09-10.87 (m, 1H), 10.46 (d, J=22.7 Hz, 1H), 9.70 (br s, 1H), 8.76-8.52 (m, 1H), 8.47 (dd, J=21.4, 2.8 Hz, 1H), 8.29 (d, J=36.0 Hz, 1H), 7.76-7.66 (m, 1H), 7.66-7.60 (m, 1H), 7.56 (dd, J=13.4, 8.4 Hz, 1H), 7.52-7.38 (m, 2H), 7.20 (dd, J=13.4, 7.5 Hz, 1H), 7.16-7.07 (m, 1H), 6.81-6.69 (m, 1H), 6.45 (t, J=15.7 Hz, 1H), 4.61-4.49 (m, 1H), 4.48-4.34 (m, 1H), 3.99-3.91 (m, 1H), 3.91-3.81 (m, 1H), 3.79-3.66 (m, 1H), 3.33 (s, 3H), 3.26-3.18 (m, 1H), 2.80 (s, 3H), 2.30-2.19 (m, 1H), 2.15-2.06 (m, 1H), 2.05-1.92 (m, 1H); MS (m/z): 544.61 [M+1]⁺.

### Example 3. (E)-N-(4-(4-(5-cyano-4-(1H-indol-3-yl)pyrimidin-2-ylamino)piperidine-1-carbonyl)phenyl)-4-(dimethylamino)but-2-enamide (Compound 102)

### 2-(1-(4-aminobenzoyl)piperidin-4-ylamino)-4-(1-(phenylsulfonyl)-1H-indol-3-yl)pyrimidine-5-carbonitrile

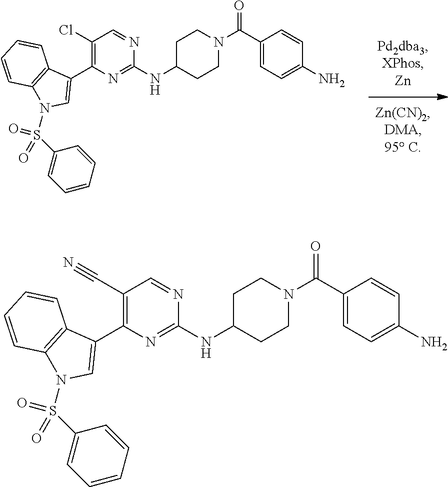 [SMILES: Nc1ccc(C(=O)N2CCC(Nc3ncc(Cl)c(-c4cn(S(=O)(=O)c5ccccc5)c5ccccc45)n3)CC2)cc1 ; N#Cc1cnc(NC2CCN(C(=O)c3ccc(N)cc3)CC2)nc1-c1cn(S(=O)(=O)c2ccccc2)c2ccccc12]

A suspension of (4-aminophenyl)(4-(5-chloro-4-(1-(phenylsulfonyl)-1H-indol-3-yl)pyrimidin-2-ylamino)piperidin-1-yl)methanone prepared as in Example 1 (187 mg, 0.319 mmol), zinc dust (2.1 mg, 0.03 mmol), Pd₂dba₃ (29.2 mg, 0.03 mmol), Xphos (30.4 mg, 0.06 mmol) and zinc cyanide (22.4 mg, 0.19 mmol) in degassed DMA (4.25 mL) was stirred 2 h at 95° C. The cooled mixture was diluted with EtOAc (20 mL) and washed with water (3×5 mL), brine (5 mL), dried (MgSO₄), filtered and concentrated under reduced pressure. The resulting compound was purified on SiO₂ chromatography (DCM/MeOH 1 to 10% gradient) and afforded the title compound (184 mg, 0.319 mmol, 100%) as a white solid.

### 2-(1-(4-aminobenzoyl)piperidin-4-ylamino)-4-(1H-indol-3-yl)pyrimidine-5-carbonitrile (Compound 26)

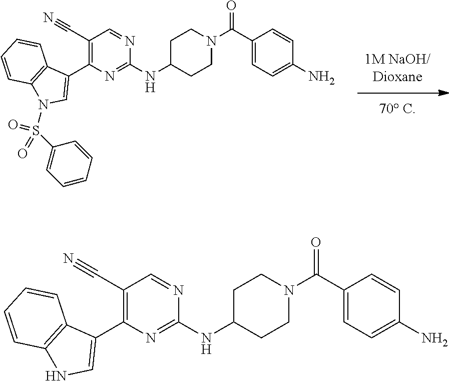 [SMILES: N#Cc1cnc(NC2CCN(C(=O)c3ccc(N)cc3)CC2)nc1-c1cn(S(=O)(=O)c2ccccc2)c2ccccc12 ; N#Cc1cnc(NC2CCN(C(=O)c3ccc(N)cc3)CC2)nc1-c1c[nH]c2ccccc12]

A solution of 2-(1-(4-aminobenzoyl)piperidin-4-ylamino)-4-(1-(phenylsulfonyl)-1H-indol-3-yl)pyrimidine-5-carbonitrile (184 mg, 0.319 mmol) and 1M NaOH (2 mL, 2 mmol) in dioxane (2 mL) was heated 3 h at 70° C. The cooled mixture was diluted with DCM (10 mL), washed with water (2 mL), dried (MgSO₄), filtered, concentrated under reduced pressure and afforded the title compound (130 mg, 0.297 mmol, 93.3%) as a yellowish solid which was used in the next step without further purification.

### (E)-N-(4-(4-(5-cyano-4-(1H-indol-3-yl)pyrimidin-2-ylamino)piperidine-1-carbonyl)phenyl)-4-(dimethylamino)but-2-enamid (Compound 102)

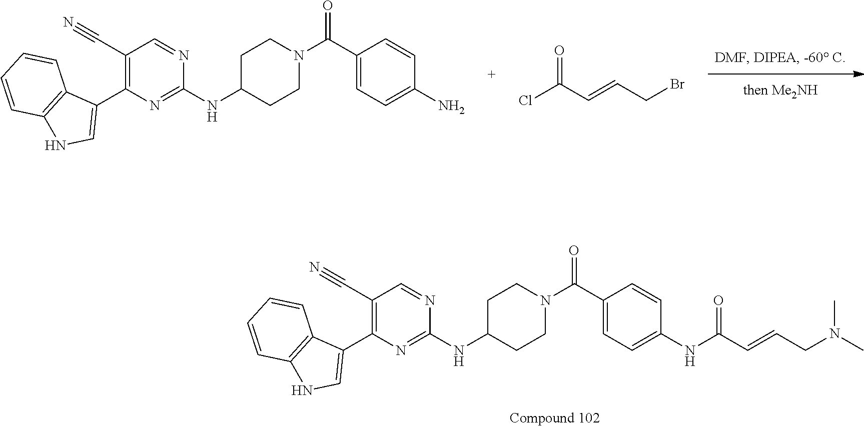 [SMILES: N#Cc1cnc(NC2CCN(C(=O)c3ccc(N)cc3)CC2)nc1-c1c[nH]c2ccccc12 ; O=C(Cl)/C=C/CBr ; CN(C)C/C=C/C(=O)Nc1ccc(C(=O)N2CCC(Nc3ncc(C#N)c(-c4c[nH]c5ccccc45)n3)CC2)cc1]

To a −60° C. solution of 2-(1-(4-aminobenzoyl)piperidin-4-ylamino)-4-(1H-indol-3-yl)pyrimidine-5-carbonitrile (130 mg, 0.31 mmol) and DIPEA (155 μL, 0.89 mmol) in DMF/THF 1:1 (2 mL) was added slowly a 54 mg/mL solution of (E)-4-bromobut-2-enoyl chloride in DCM (1 mL, 0.31 mmol). After 30 min at −60° C., a 2M solution of dimethylamine (450 μL, 0.93 mmol) was added and the resulting mixture was stirred 1 h at rt. The mixture was concentrated under reduced pressure and the resulting solution was purified by reverse phase chromatography (C18, water/ACN 15 to 60% gradient) and afforded Compound 102 (72.8 mg, 0.133 mmol, 45%) as a white solid after lyophilization. ¹H NMR (500 MHz, d₆-DMSO δ 12.06-11.93 (m, 1H), 10.33 (s, 1H), 8.71 (d, J=8.1 Hz, 1H), 8.63 (d, J=29.5 Hz, 1H), 8.50 (dt, J=8.8, 2.8 Hz, 1H), 8.22-8.10 (m, 1H), 7.73 (dd, J=8.5, 1.7 Hz, 1H), 7.58-7.47 (m, 1H), 7.45-7.34 (m, 1H), 7.28-7.21 (m, 1H), 7.22-7.14 (m, 1H), 6.82-6.69 (m, 1H), 6.35 (d, J=15.4 Hz, 1H), 4.62-4.31 (m, 2H), 4.33-4.06 (m, 2H), 4.00-3.52 (m, 2H), 3.25-2.79 (m, 4H), 2.45-2.26 (m, 4H), 2.16-1.82 (m, 3H), 1.65-1.42 (m, 2H). MS (m/z): 549.72 [M+1]⁺.

### Example 4. (E)-N-(4-((R)-3-(5-chloro-4-(1H-indol-3-yl)pyrimidin-2-ylamino)pyrrolidin-1-ylsulfonyl)phenyl)-4-(dimethylamino)but-2-enamide (Compound 105)

### (3R)-tert-butyl 3-(5-chloro-4-(1-(phenylsulfonyl)-1H-indol-3-yl)pyrimidin-2-ylamino)pyrrolidine-1-carboxylate

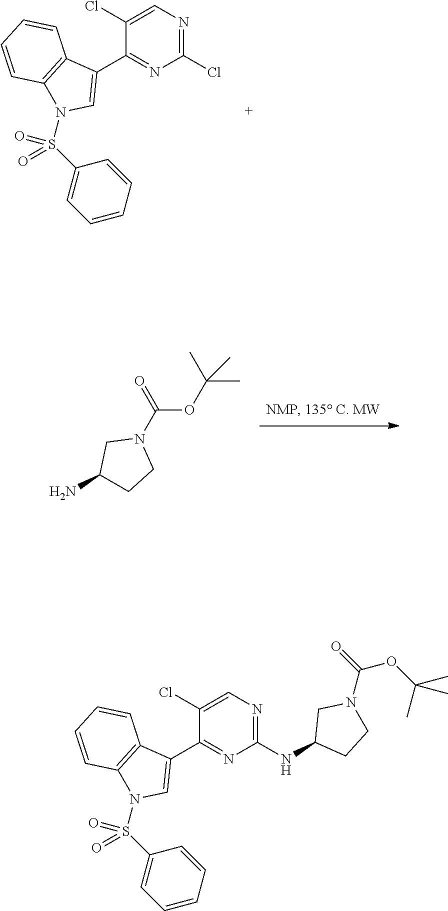 [SMILES: O=S(=O)(c1ccccc1)n1cc(-c2nc(Cl)ncc2Cl)c2ccccc21 ; CC(C)(C)OC(=O)N1CC[C@@H](N)C1 ; CC(C)(C)OC(=O)N1CC[C@@H](Nc2ncc(Cl)c(-c3cn(S(=O)(=O)c4ccccc4)c4ccccc34)n2)C1]

A solution of 3-(2,5-dichloropyrimidin-4-yl)-1-(phenylsulfonyl)-1H-indole (2.50 g, 6.18 mmol), (R)-tert-butyl 3-aminopyrrolidine-1-carboxylate (1.209 g, 6.49 mmol) and diisopropylethylamine (1.08 mL, 6.18 mmol) in NMP (16 mL) was heated 15 min at 135° C. (mW). The mixture was diluted with EtOAc (50 mL), washed with water (10 mL), brine (10 mL), dried (MgSO₄), filtered and concentrated under reduced pressure. The residue was purified by SiO₂ chromatography (DCM/EtOAc 0 to 40% gradient), and afforded the title compound (2.378 g, 4.29 mmol, 69%) as a white solid.

### 5-chloro-4-(1-(phenylsulfonyl)-H-indol-3-yl)-N—((R)-pyrrolidin-3-yl)pyrimidin-2-amine

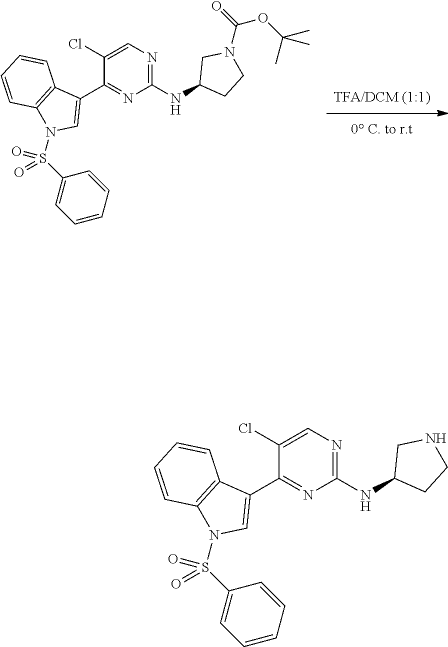 [SMILES: CC(C)(C)OC(=O)N1CC[C@@H](Nc2ncc(Cl)c(-c3cn(S(=O)(=O)c4ccccc4)c4ccccc34)n2)C1 ; O=S(=O)(c1ccccc1)n1cc(-c2nc(N[C@@H]3CCNC3)ncc2Cl)c2ccccc21]

Trifluoroacetic acid (7 mL, 85.8 mmol) was added to a stirring solution of (3R)-tert-butyl 3-(5-chloro-4-(1-(phenylsulfonyl)-1H-indol-3-yl)pyrimidin-2-ylamino)pyrrolidine-1-carboxylate (2.378 g, 4.292 mmoL) in DCM (7 mL) at 0° C. The resulting solution was stirred 2 h at rt, concentrated under reduced pressure, diluted with DCM (100 mL) and sat NaHCO₃ (15 mL). The phases were separated and aqueous extracted DCM (2×75 mL). The combined organic layers were dried (MgSO₄), filtered, concentrated and afforded the title compound (1.95 g, 4.29 mmol, 100%) as a yellow foam which was used in the next step without further purification.

### 5-chloro-N—((R)-1-(4-nitrophenylsulfonyl)pyrrolidin-3-yl)-4-(1-(phenylsulfonyl)-1H-indol-3-yl)pyrimidin-2-amine

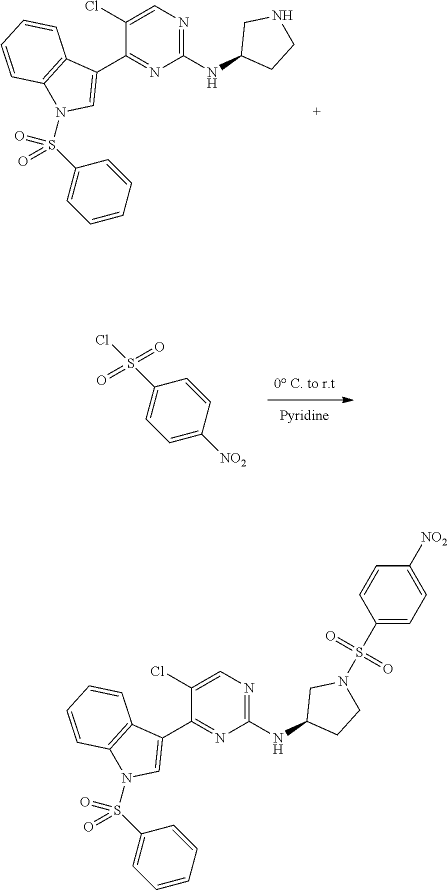 [SMILES: O=S(=O)(c1ccccc1)n1cc(-c2nc(N[C@@H]3CCNC3)ncc2Cl)c2ccccc21 ; O=[N+]([O-])c1ccc(S(=O)(=O)Cl)cc1 ; O=[N+]([O-])c1ccc(S(=O)(=O)N2CC[C@@H](Nc3ncc(Cl)c(-c4cn(S(=O)(=O)c5ccccc5)c5ccccc45)n3)C2)cc1]

To a cooled (0° C.) solution of 5-chloro-4-(1-(phenylsulfonyl)-1H-indol-3-yl)-N—((R)-pyrrolidin-3-yl)pyrimidin-2-amine (500 mg, 1.101 mmol) in pyridine (2.2 mL) was added 4-nitrobenzene-1-sulfonyl chloride (732 mg, 3.303 mmol). The mixture was stirred 12 h at rt and 5 h at 90° C. before being concentrated under reduced pressure. The crude residue was purified by SiO₂ chromatography (Hex/EtOAc 0 to 40% gradient) and afforded the title compound (286 mg, 0.449 mmol, 41%) as a yellow foam.

### N—((R)-1-(4-aminophenylsulfonyl)pyrrolidin-3-yl)-5-chloro-4-(1-(phenylsulfonyl)-1H-indol-3-yl)pyrimidin-2-amine

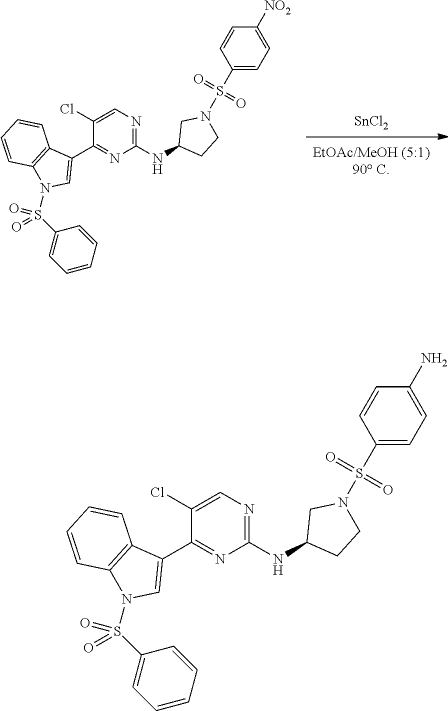 [SMILES: O=[N+]([O-])c1ccc(S(=O)(=O)N2CC[C@@H](Nc3ncc(Cl)c(-c4cn(S(=O)(=O)c5ccccc5)c5ccccc45)n3)C2)cc1 ; "SnCl2" ; Nc1ccc(S(=O)(=O)N2CC[C@@H](Nc3ncc(Cl)c(-c4cn(S(=O)(=O)c5ccccc5)c5ccccc45)n3)C2)cc1]

Tin (II) chloride dehydrate (64 mg, 0.2816 mmol) was added to a solution of 5-chloro-N—((R)-1-(4-nitrophenylsulfonyl)pyrrolidin-3-yl)-4-(1-(phenylsulfonyl)-1H-indol-3-yl)pyrimidin-2-amine (72 mg, 0.1127 mmol) in EtOAc/MeOH 5:1 (3 mL). The resulting solution was heated 3 h at 90° C. in a sealed tube. The cooled mixture was diluted with EtOAc (10 mL) and sat. NaHCO₃ (5 mL). The aqueous layer was extracted with CHCl₃/IPA 4:1 (3×10 mL) and the combined organic layers were dried (MgSO₄), filtered and concentrated under reduced pressure. The residue was purified by SiO₂ chromatography (DCM/EtOAc 0 to 35%) and afforded the title compound (43 mg, 0.071 mmol, 63%) as a yellow solid.

### N—((R)-1-(4-aminophenylsulfonyl)pyrrolidin-3-yl)-5-chloro-4-(1H-indol-3-yl)pyrimidin-2-amine (Compound 32)

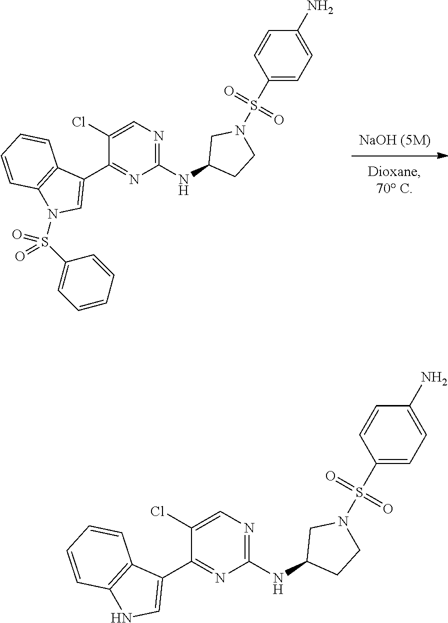 [SMILES: Nc1ccc(S(=O)(=O)N2CC[C@@H](Nc3ncc(Cl)c(-c4cn(S(=O)(=O)c5ccccc5)c5ccccc45)n3)C2)cc1 ; "NaOH (5M)" ; Nc1ccc(S(=O)(=O)N2CC[C@@H](Nc3ncc(Cl)c(-c4c[nH]c5ccccc45)n3)C2)cc1]

A solution of N—((R)-1-(4-aminophenylsulfonyl)pyrrolidin-3-yl)-5-chloro-4-(1-(phenylsulfonyl)-1H-indol-3-yl)pyrimidin-2-amine (43 mg, 0.0706 mmol) in dioxane (3 mL) and 5M NaOH solution (0.07 mL, 0.353 mmol) was heated 3 h at 70° C. The reaction mixture was neutralized with 1M HCl then extracted with EtOAc (3×10 mL). The combined organic layers were dried (MgSO₄), filtered and concentrated. The residue was purified by SiO₂ chromatography (DCM/EtOAc 0 to 50% gradient) and afforded the title compound (24 mg, 0.051 mmol, 73%) as a light yellow solid.

### (E)-N-(4-((R)-3-(5-chloro-4-(1H-indol-3-yl)pyrimidin-2-ylamino)pyrrolidin-1-ylsulfonyl)phenyl)-4-(dimethylamino)but-2-enamide (Compound 105)

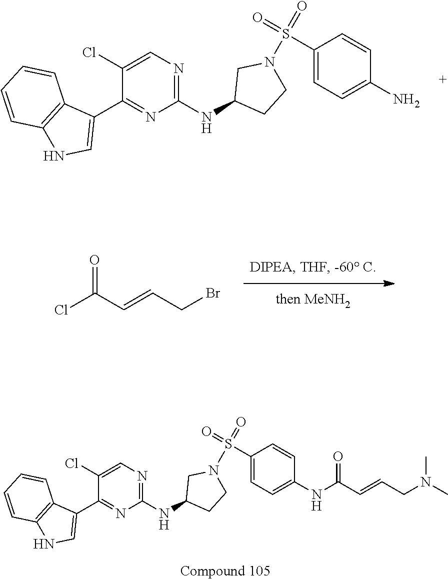 [SMILES: Nc1ccc(S(=O)(=O)N2CC[C@@H](Nc3ncc(Cl)c(-c4c[nH]c5ccccc45)n3)C2)cc1 ; O=C(Cl)/C=C/CBr ; CN(C)C/C=C/C(=O)Nc1ccc(S(=O)(=O)N2CC[C@@H](Nc3ncc(Cl)c(-c4c[nH]c5ccccc45)n3)C2)cc1]

To a −60° C. solution of N—((R)-1-(4-aminophenylsulfonyl)pyrrolidin-3-yl)-5-chloro-4-(1H-indol-3-yl)pyrimidin-2-amine (70 mg, 0.1493 mmol) and DIPEA (78 μL, 0.447 mmol) in THF (5 mL) was slowly added a 56 mg/mL solution of (E)-4-bromobut-2-enoyl chloride 27 in DCM (78 μL, 0.4478 mmol). After 1 h 15 min a 2M solution of dimethylamine in THF (0.22 mL, 0.5986 mmol) was added and the resulting mixture was warmed to room temp and stirred for 2 h. The mixture was concentrated under reduced pressure and the residue was purified by reverse phase chromatography (C18, water/ACN 0 to 100% gradient) and afforded Compound 105 (14 mg, 0.024 mmol, 16%) as a light yellow solid after lyophilization. ¹H NMR (500 MHz, d₆-DMSO) δ 11.86 (s, 1H), 10.75 (s, 1H), 8.48 (br s, 1H), 8.46 (d, J=3.0 Hz, 1H), 8.25 (s, 1H), 7.89 (d, J=8.7 Hz, 2H), 7.76 (d, J=8.0 Hz, 2H), 7.48 (d, J=8.1 Hz, 1H), 7.40 (br s, 1H), 7.20 (t, J=7.5 Hz, 1H), 7.10 (t, J=7.0 Hz, 1H), 6.82 (dt, J=14.4, 7.0 Hz, 1H), 6.48 (d, J=15.3 Hz, 1H), 4.29 (br s, 1H), 3.93 (d, J=6.9 Hz, 2H), 3.53-3.48 (m, 1H), 3.46-3.35 (m, 1H), 3.28-3.21 (m, 1H), 3.20-3.14 (m, 1H), 2.78 (s, 6H), 2.14-2.03 (m, 1H), 1.94-1.82 (m, 1H); MS (m/z): 580.59 [M+1]⁺.

### Example 5. (E)-N-(4-((R)-3-(5-chloro-4-(1H-indol-3-yl)pyrimidin-2-ylamino)pyrrolidine-1-carbonyl)phenyl)-4-(dimethylamino)but-2-enamide (Compound 104)

### tert-butyl 4-((R)-3-(5-chloro-4-(1-(phenylsulfonyl)-1H-indol-3-yl)pyrimidin-2-ylamino)pyrrolidine-1-carbonyl)phenylcarbamate

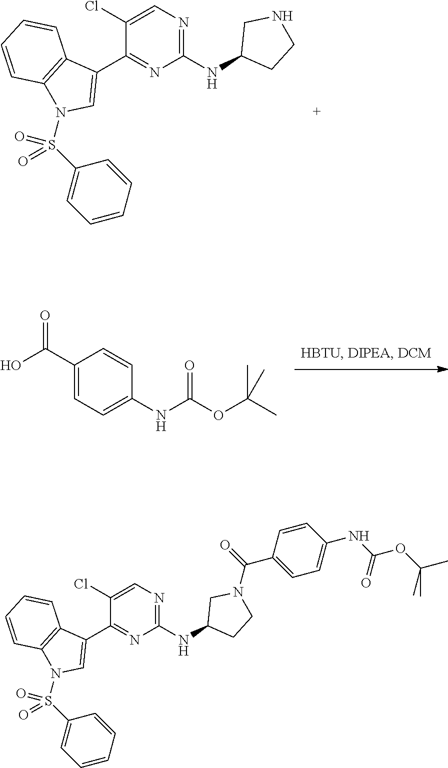 [SMILES: O=S(=O)(c1ccccc1)n1cc(-c2nc(N[C@@H]3CCNC3)ncc2Cl)c2ccccc21 ; CC(C)(C)OC(=O)Nc1ccc(C(=O)O)cc1 ; CC(C)(C)OC(=O)Nc1ccc(C(=O)N2CC[C@@H](Nc3ncc(Cl)c(-c4cn(S(=O)(=O)c5ccccc5)c5ccccc45)n3)C2)cc1]

A solution of 5-chloro-4-(1-(phenylsulfonyl)-1H-indol-3-yl)-N—((R)-pyrrolidin-3-yl)pyrimidin-2-amine (500 mg, 1.10 mmol), 4-(tert-butoxycarbonylamino)benzoic acid (261 mg, 1.10 mmol), HBTU (459 mg, 1.21 mmol), diisopropylethylamine (0.63 mL, 3.63 mmol) in DCM (3.9 mL) was stirred overnight at rt. The mixture was concentrated under reduced pressure and the residue was purified by SiO₂ chromatography (Hex/EtOAc 0 to 100% EtOAc gradient) to afford the title compound (659 mg, 0.98 mmol, 89%) as a white solid.

### (4-aminophenyl)((R)-3-(5-chloro-4-(1-(phenylsulfonyl)-1H-indol-3-yl)pyrimidin-2-ylamino)pyrrolidin-1-yl)methanone

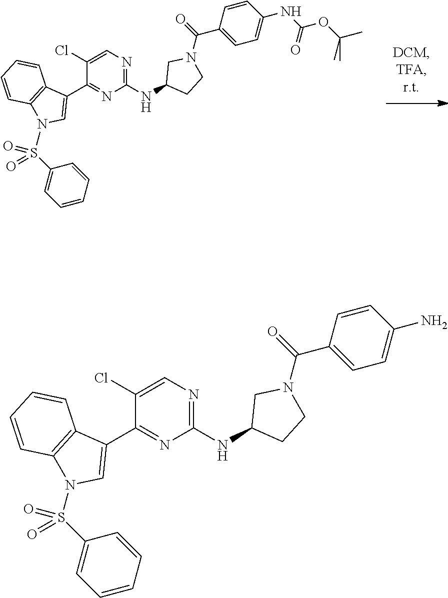 [SMILES: CC(C)(C)OC(=O)Nc1ccc(C(=O)N2CC[C@@H](Nc3ncc(Cl)c(-c4cn(S(=O)(=O)c5ccccc5)c5ccccc45)n3)C2)cc1 ; Nc1ccc(C(=O)N2CC[C@@H](Nc3ncc(Cl)c(-c4cn(S(=O)(=O)c5ccccc5)c5ccccc45)n3)C2)cc1]

A solution of tert-butyl 4-((R)-3-(5-chloro-4-(1-(phenylsulfonyl)-1H-indol-3-yl)pyrimidin-2-ylamino)pyrrolidine-1-carbonyl)phenylcarbamate (659 mg, 0.98 mmol) DCM (4 mL) was treated with TFA (1 mL) and stirred overnight at rt. The mixture was diluted with DCM (40 mL), and washed with sat. NaHCO₃ (3×5 mL) and with brine (5 mL), dried (MgSO₄), filtered, concentrated under reduced pressure and afforded the title compound (511 mg, 0.89 mmol, 91%) as a colorless glue which was used in the next step without further purification.

### (4-aminophenyl)((R)-3-(5-chloro-4-(1H-indol-3-yl)pyrimidin-2-ylamino)pyrrolidin-1-yl)methanone (Compound 35)

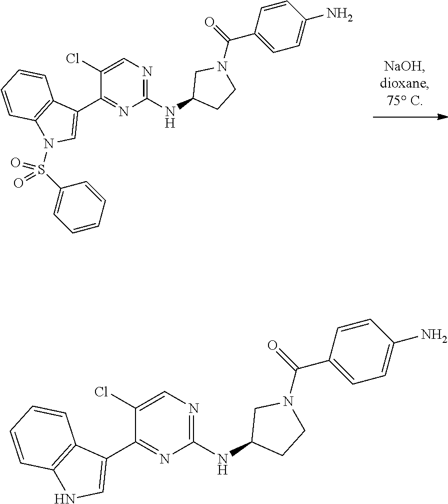 [SMILES: Nc1ccc(C(=O)N2CC[C@@H](Nc3ncc(Cl)c(-c4cn(S(=O)(=O)c5ccccc5)c5ccccc45)n3)C2)cc1 ; Nc1ccc(C(=O)N2CC[C@@H](Nc3ncc(Cl)c(-c4c[nH]c5ccccc45)n3)C2)cc1]

A solution of (4-aminophenyl)((R)-3-(5-chloro-4-(1-(phenylsulfonyl)-1H-indol-3-yl)pyrimidin-2-ylamino)pyrrolidin-1-yl)methanone (377 mg, 0.658 mmol) and 5M NaOH (1.32 mL, 6.58 mmol) in 1,4-dioxane (4.4 mL) was heated 2 h at 75° C. The cooled mixture was diluted with CHCl₃/IPA 10:1 (15 mL), washed with water (5 mL), dried (MgSO₄), filtered, evaporated under reduced pressure and afforded the title compound (285 mg, 0.658 mmol, 100%) as a white solid which was used in the next step without further purification.

### (E)-N-(4-((R)-3-(5-chloro-4-(1H-indol-3-yl)pyrimidin-2-ylamino)pyrrolidine-1-carbonyl)phenyl)-4-(dimethylamino)but-2-enamide.TFA (Compound 104)

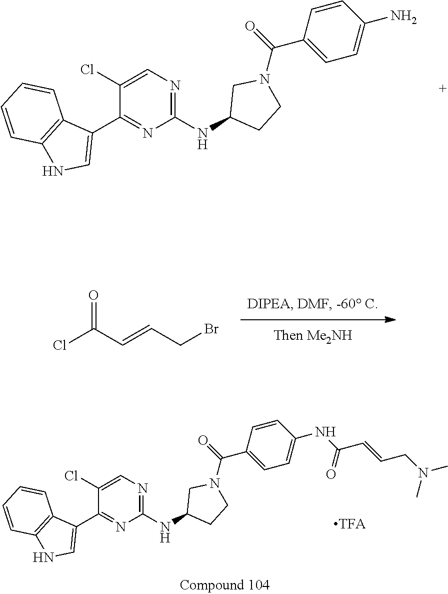 [SMILES: Nc1ccc(C(=O)N2CC[C@@H](Nc3ncc(Cl)c(-c4c[nH]c5ccccc45)n3)C2)cc1 ; O=C(Cl)/C=C/CBr ; CN(C)C/C=C/C(=O)Nc1ccc(C(=O)N2CC[C@@H](Nc3ncc(Cl)c(-c4c[nH]c5ccccc45)n3)C2)cc1]

To a cooled (−60° C.) solution of (4-aminophenyl)((R)-3-(5-chloro-4-(1H-indol-3-yl)pyrimidin-2-ylamino)pyrrolidin-1-yl)methanone (100 mg, 0.23 mmol) in THF (1.15 mL) was added a 55.6 mg/mL solution of (E)-4-bromobut-2-enoyl chloride in THF (821 μL, 0.23 mmol). The mixture was stirred 4 h at −60° C. before addition of a 2M solution of dimethylamine in THF (346 μL, 0.69 mmol). The mixture was stirred 24 h at rt and concentrated under vacuum. The residue was purified by reverse phase chromatography (C18, water/ACN +0.1% TFA 10 to 95% gradient) and afforded Compound 104 (16 mg, 0.025 mmol, 11%) as a white solid after lyophilization. ¹H NMR (500 MHz, d₆-DMSO) δ 11.93-11.79 (m, 1H), 10.57-10.38 (m, 1H), 9.74 (s (br), 1H), 8.68 (s (br), 1H), 8.47 (dd, J=27.4, 2.8 Hz, 1H), 8.29 (d, J=36.1 Hz, 1H), 7.73 (d, J=10.9 Hz, 1H), 7.72-7.61 (m, 2H), 7.56 (dd, J=13.4, 8.5 Hz, 1H), 7.49 (dd, J=13.7, 7.7 Hz, 1H), 7.20 (td, J=14.2, 7.1 Hz, 1H), 7.11 (s (br), 1H), 6.80-6.70 (m, 1H), 6.45 (t, J=16.2 Hz, 1H), 4.60-4.34 (m, 1H), 4.01-3.89 (m, 2H), 3.91-3.79 (m, 1H), 3.79-3.65 (m, 1H), 3.63-3.53 (m, 2H), 2.80 (s, 3H), 2.50 (s, 3H), 2.24 (s (br), 1H), 2.14-1.95 (m, 1H); MS (m/z): 544.65 [M+1]⁺.

### Example 6. (E)-N-(4-((R)-3-(5-cyano-4-(1H-indol-3-yl)pyrimidin-2-ylamino)pyrrolidine-1-carbonyl)phenyl)-4-(dimethylamino)but-2-enamide (Compound 103)

### 2-((R)-1-(4-aminobenzoyl)pyrrolidin-3-ylamino)-4-(1-(phenylsulfonyl)-1H-indol-3-yl)pyrimidine-5-carbonitrile

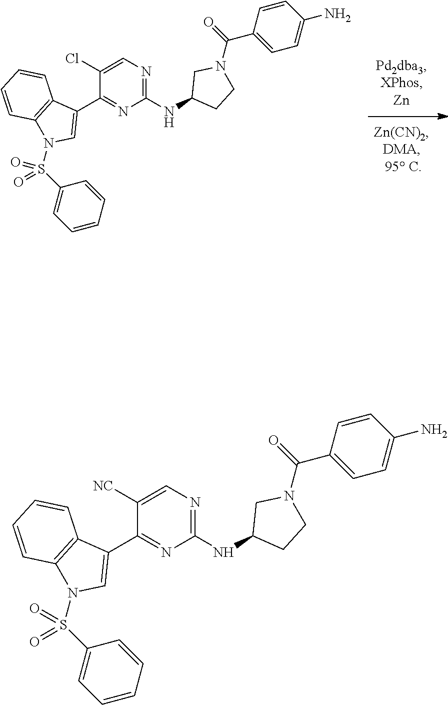 [SMILES: Nc1ccc(C(=O)N2CC[C@@H](Nc3ncc(Cl)c(-c4cn(S(=O)(=O)c5ccccc5)c5ccccc45)n3)C2)cc1 ; N#Cc1cnc(N[C@@H]2CCN(C(=O)c3ccc(N)cc3)C2)nc1-c1cn(S(=O)(=O)c2ccccc2)c2ccccc12]

A suspension of (4-aminophenyl)((R)-3-(5-chloro-4-(1-(phenylsulfonyl)-1H-indol-3-yl)pyrimidin-2-ylamino)pyrrolidin-1-yl)methanone (550 mg, 0.963 mmol), zinc dust (6.3 mg, 0.1 mmol), Pd₂dba₃ (87.9 mg, 0.1 mmol), Xphos (92.0 mg, 0.19 mmol) and zinc cyanide (113 mg, 0.96 mmol) in degassed DMA (19.2 mL) was heated 1 h 30 min at 95° C. The cooled mixture was diluted with EtOAc (50 mL), washed with water (3×10 mL), brine (10 mL), dried (MgSO₄), filtered, concentrated and afforded the title compound (543 mg, 0.963 mmol, 100%) which was used without further purification.

### 2-((R)-1-(4-aminobenzoyl)pyrrolidin-3-ylamino)-4-(1H-indol-3-yl)pyrimidine-5-carbonitrile (Compound 37)

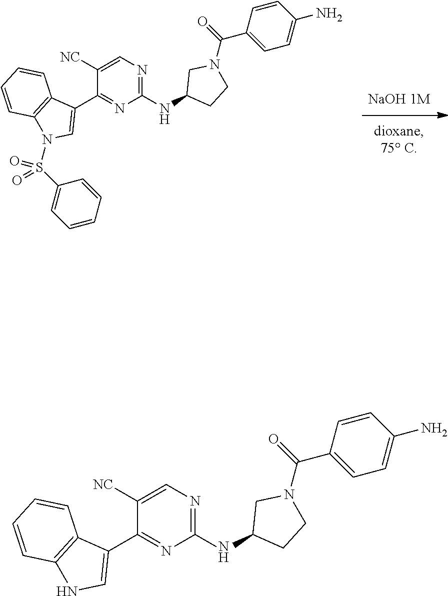 [SMILES: N#Cc1cnc(N[C@@H]2CCN(C(=O)c3ccc(N)cc3)C2)nc1-c1cn(S(=O)(=O)c2ccccc2)c2ccccc12 ; N#Cc1cnc(N[C@@H]2CCN(C(=O)c3ccc(N)cc3)C2)nc1-c1c[nH]c2ccccc12]

A solution of 2-((R)-1-(4-aminobenzoyl)pyrrolidin-3-ylamino)-4-(1-(phenylsulfonyl)-1H-indol-3-yl)pyrimidine-5-carbonitrile (541 mg, 0.961 mmol) and 5M NaOH (1.92 mL, 9.6 mmol) in 1,4-dioxane (6.4 mL) was heated 2 h at 75° C. The cooled mixture was concentrated under reduced pressure and the residue was purified by SiO₂ chromatography (Hex/EtOAc 80 to 100% gradient) to afford the title compound (379 mg, 0.896 mmol, 91%) as a white solid.

### (E)-N-(4-((R)-3-(5-cyano-4-(1H-indol-3-yl)pyrimidin-2-ylamino)pyrrolidine-1-carbonyl)phenyl)-4-(dimethylamino)but-2-enamide (Compound 103)

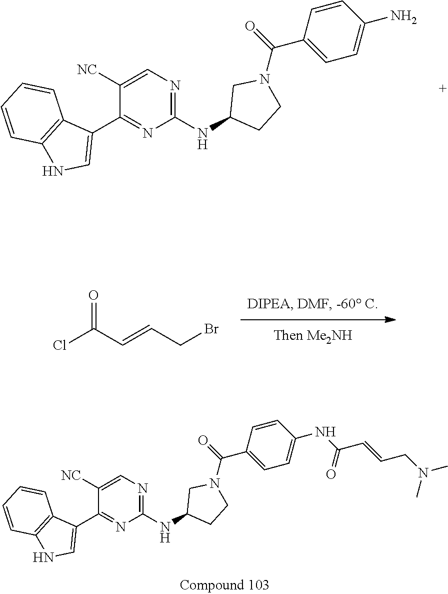 [SMILES: N#Cc1cnc(N[C@@H]2CCN(C(=O)c3ccc(N)cc3)C2)nc1-c1c[nH]c2ccccc12 ; O=C(Cl)/C=C/CBr ; CN(C)C/C=C/C(=O)Nc1ccc(C(=O)N2CC[C@@H](Nc3ncc(C#N)c(-c4c[nH]c5ccccc45)n3)C2)cc1]

To a cooled (−60° C.) solution of 2-((R)-1-(4-aminobenzoyl)pyrrolidin-3-ylamino)-4-(1H-indol-3-yl)pyrimidine-5-carbonitrile (79 mg, 0.187 mmol) in THF (9.3 mL) was added a 54.2 mg/mL solution of (E)-4-bromobut-2-enoyl chloride in THF (719 μL, 0.187 mmol). The mixture was stirred 4 h at (−60° C.) before addition of a 2M solution of dimethylamine in THE (280 μL, 0.56 mmol). After 24 h at rt, the mixture was concentrated under reduced pressure and the residue was purified by reverse phase chromatography (C18, water/ACN 15 to 65% gradient) and afforded Compound 103 (13 mg, 0.024 mmol, 13%) as a white solid after lyophilization. ¹H NMR (500 MHz, d₆-DMSO) δ 12.07-11.93 (m, 1H), 10.26-10.12 (m, 1H), 8.76-8.66 (m, 1H), 8.66-8.56 (m, 1H), 8.55-8.50 (m, 1H), 8.49-8.42 (m, 1H), 8.35 (dd, J=35.9, 7.5 Hz, 1H), 7.76-7.60 (m, 2H), 7.60-7.43 (m, 2H), 7.28-7.15 (m, 1H), 7.16-7.06 (m, 1H), 6.83-6.66 (m, 1H), 6.35-6.17 (m, 1H), 4.76-4.41 (m, 2H), 4.02-3.80 (m, 1H), 3.79-3.67 (m, 1H), 3.67-3.43 (m, 2H), 3.11-2.99 (m, 1H), 2.34-2.21 (m, 1H), 2.50 (s, 3H), 2.18 (s, 3H), 2.06-1.96 (m, 1H); MS (m/z): 535.60 [M+1]⁺.

### Example 7. (E)-N-(4-((4-(5-chloro-4-(1H-indol-3-yl)pyrimidin-2-ylamino)piperidin-1-yl)methyl)phenyl)-4-(dimethylamino)but-2-enamide (Compound 106)

### tert-butyl 1-(4-acetamidobenzyl)piperidin-4-ylcarbamate

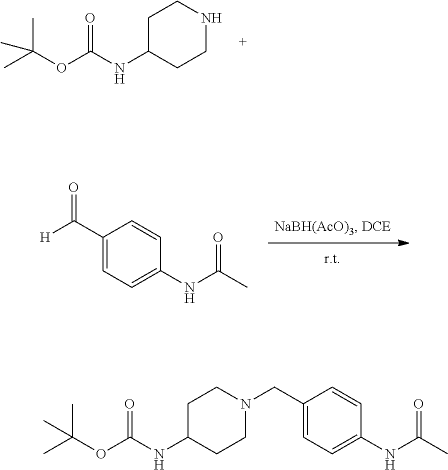 [SMILES: CC(C)(C)OC(=O)NC1CCNCC1 ; CC(=O)Nc1ccc(C=O)cc1 ; CC(=O)Nc1ccc(CN2CCC(NC(=O)OC(C)(C)C)CC2)cc1]

To a suspension of 4-Boc-aminopiperidine (500 mg, 2.5 mmol) in DCE (10 mL) was added AcOH (43 uL, 0.75 mmol) affording pale yellow solution which was treated with N-(4-formylphenyl)acetamide (407 mg, 2.5 mmol) followed by NaBH(OAc)₃ (794 mg, 3.74 mmol). The resulting solution was stirred 1 h at rt, diluted with DCM (50 ml), washed with sat. NaHCO₃, (60 mL), brine (3×30 mL), dried (MgSO₄), filtered, concentrated under reduced pressure and afforded the title compound (783 mg, 2.26 mmol, 90%) as a white solid which was used in the next step without further purification.

### N-(4-((4-aminopiperidin-1-yl)methyl)phenyl)acetamide.HCl

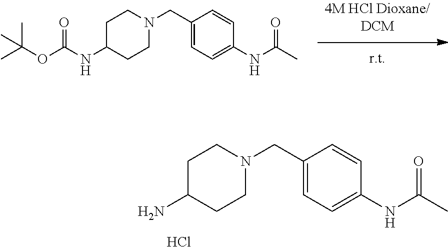 [SMILES: CC(=O)Nc1ccc(CN2CCC(NC(=O)OC(C)(C)C)CC2)cc1 ; CC(=O)Nc1ccc(CN2CCC(N)CC2)cc1 ; Cl]

A solution of tert-butyl 1-(4-acetamidobenzyl)piperidin-4-ylcarbamate (783 mg, 2.25 mmol) in DCM (7 mL) was treated with HCl and Dioxane (9 mL). The resulting mixture was stirred 30 min at rt and the resulting solid was filtered, washed with Et₂O (2×5 mL), dried overnight (high vacuum) and afforded the title compound (500 mg, 1.76 mmol, 78%) as white solid.

### N-(4-((4-(5-chloro-4-(1-(phenylsulfonyl)-1H-indol-3-yl)pyrimidin-2-ylamino)piperidin-1-yl)methyl)phenyl)acetamide

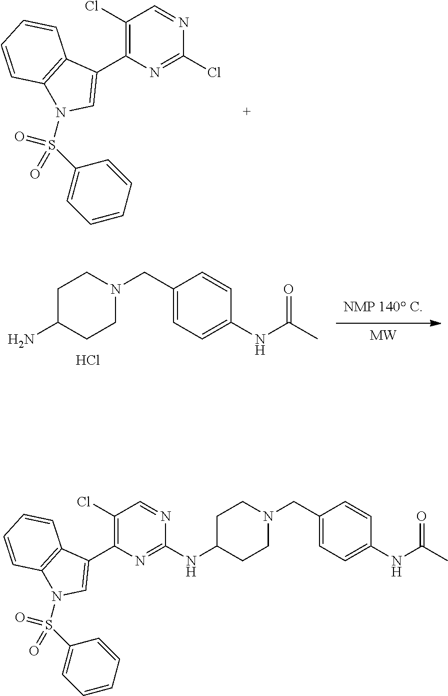

A suspension of 3-(2,5-dichloropyrimidin-4-yl)-1-(phenylsulfonyl)-1H-indole (400 mg, 0.99 mmol), N-(4-((4-aminopiperidin-1-yl)methyl)phenyl)acetamide.HCl (317 mg, 0.99 mmol) and DIPEA (1.03 mL, 5.94 mmol) in NMP (5 mL) was heated 20 min at 145° C. (mW). The cooled mixture was diluted with EtOAc (20 mL), washed with sat. NaHCO₃ (5 mL), brine (5 mL), dried (MgSO₄), filtered and concentrated under reduced pressure. The residue was purified by SiO₂ chromatography (DCM/MeOH 5 to 15% gradient) and afforded the title compound (331 mg, 0.534 mmol, 54%) as a pale beige solid.

### N-(4-((4-(5-chloro-4-(1H-indol-3-yl)pyrimidin-2-ylamino)piperidin-1-yl)methyl)phenyl)acetamide (Compound 47)

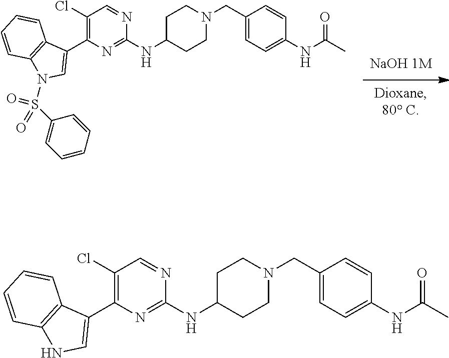 [SMILES: CC(=O)Nc1ccc(CN2CCC(Nc3ncc(Cl)c(-c4cn(S(=O)(=O)c5ccccc5)c5ccccc45)n3)CC2)cc1 ; "Dioxane,80° C." ; CC(=O)Nc1ccc(CN2CCC(Nc3ncc(Cl)c(-c4c[nH]c5ccccc45)n3)CC2)cc1]

A suspension of N-(4-((4-(5-chloro-4-(1-(phenylsulfonyl)-1H-indol-3-yl)pyrimidin-2-ylamino)piperidin-1-yl)methyl)phenyl)acetamide (331 mg, 0.534 mmol) and 1M NaOH (3 mL, 2.67 mmol) in dioxane (6 mL) was heated 3 h at 80° C. The cooled mixture was diluted with DCM/iPrOH 4/1 (10 mL), washed with water (5 mL), dried (MgSO₄), filtered and concentrated under reduced pressure. The residue was triturated with ACN and the solid filtered and dried (high vacuum) to afford the title compound (248 mg, 0.522 mmol, 98%) as a white solid.

### N-(1-(4-aminobenzyl)piperidin-4-yl)-5-chloro-4-(1H-indol-3-yl)pyrimidin-2-amine.HCl (Compound 48)

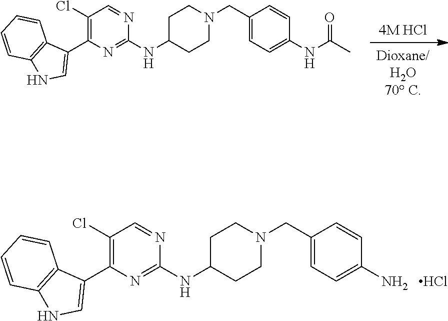 [SMILES: CC(=O)Nc1ccc(CN2CCC(Nc3ncc(Cl)c(-c4c[nH]c5ccccc45)n3)CC2)cc1 ; "4M HCl" ; "Dioxane/H2O70° C." ; Nc1ccc(CN2CCC(Nc3ncc(Cl)c(-c4c[nH]c5ccccc45)n3)CC2)cc1 ; Cl]

A suspension of N-(4-((4-(5-chloro-4-(1H-indol-3-yl)pyrimidin-2-ylamino)piperidin-1-yl)methyl)phenyl)acetamide (100 mg, 0.211 mmol) in 4M HCl dioxane (4 mL) and water (100 uL) was heated 3 h at 80° C. The cooled mixture was concentrated under reduced pressure and the residue purified by reverse phase chromatography (C18, water/ACN 10 to 70% gradient) and afforded the title compound (50 mg, 0.107 mmol, 51%) as a white solid.

### (E)-N-(4-((4-((5-chloro-4-(1H-indol-3-yl)pyrimidin-2-yl)amino)piperidin-1-yl)methyl)phenyl)-4-(dimethylamino)but-2-enamide (Compound 106)

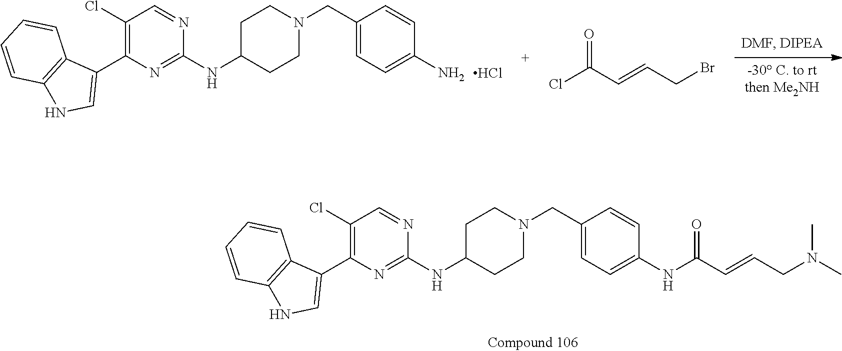 [SMILES: Nc1ccc(CN2CCC(Nc3ncc(Cl)c(-c4c[nH]c5ccccc45)n3)CC2)cc1 ; Cl ; O=C(Cl)/C=C/CBr ; "-30° C. to rtthen Me2NH" ; "DMF, DIPEA" ; CN(C)C/C=C/C(=O)Nc1ccc(CN2CCC(Nc3ncc(Cl)c(-c4c[nH]c5ccccc45)n3)CC2)cc1]

To a −30° C. solution of N-(1-(4-aminobenzyl)piperidin-4-yl)-5-chloro-4-(1H-indol-3-yl)pyrimidin-2-amine.HCl (59 mg, 0.136 mmol) and DIPEA (72 μL, 0.41 mmol) in DMF (2 mL) was slowly added a 66 mg/mL solution of (E)-4-bromobut-2-enoyl chloride in THF (378 μL, 0.136 mmol). The mixture was stirred 30 min at rt and a 2M solution of dimethylamine (680 μL, 0.68 mmol) was added. The mixture was stirred 30 min at rt and concentrated under reduced pressure, and the residue was purified by reverse phase chromatography (C18, water/ACN 5 to 40% gradient) to afford Compound 106 (44 mg, 0.081 mmol, 59%) as a white solid after lyophilization. ¹H NMR (500 MHz, d₆-DMSO) δ 11.87 (s, 1H), 10.55 (s, 1H), 8.81-8.41 (m, 2H), 8.28 (s, 1H), 7.77 (d, J=8.3 Hz, 2H), 7.55-7.43 (m, 3H), 7.21 (dd, J=21.0, 12.8 Hz, 2H), 6.92-6.68 (m, 2H), 6.49 (d, J=15.3 Hz, 2H), 4.35-4.18 (m, 3H), 4.04-3.84 (m, 3H), 3.17-2.96 (m, 3H), 2.77 (d, J=5.6 Hz, 4H), 2.30-2.04 (m, 3H), 1.90-1.64 (m, 2H); MS (m/z): 544.67 [M+1]⁺.

### Example 8. (E)-N-(4-((R)-3-(5-cyano-4-(1H-indol-3-yl)pyrimidin-2-ylamino)pyrrolidine-1-carbonyl)phenyl)-4-morpholinobut-2-enamide (Compound 107)

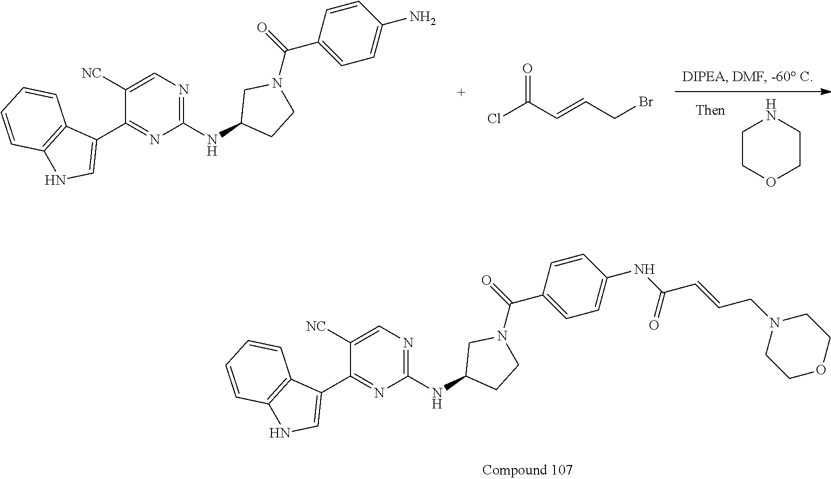

To a cooled (−60° C.) solution 2-((R)-1-(4-aminobenzoyl)pyrrolidin-3-ylamino)-4-(1H-indol-3-yl)pyrimidine-5-carbonitrile (40 mg, 0.094 mmol) and DIPEA (65 μL, 0.376 mmol) in THF (4.7 mL) was added a 54 mg/mL solution of (E)-4-bromobut-2-enoyl chloride in THF (625 μL, 0.184 mmol). The mixture was stirred 1 h 30 minutes at −60° C. before addition of morpholine (25 μL, 0.283 mmol). The mixture was stirred 24 h at rt and concentrated under reduced pressure. The residue was purified by preparative HPLC (water/ACN 0.1% NH₄HCO₃ 15 to 65% gradient) and afforded Compound 107 (5.6 mg, 0.010 mmol, 10%) as a white solid after lyophilization. ¹H NMR (500 MHz, d₆-DMSO) δ 10.28-10.16 (m, 1H), 8.70 (dd, J=23.5, 7.6 Hz, 1H), 8.58 (dd, J=16.2, 7.8 Hz, 1H), 8.53-8.48 (m, 1H), 8.44-8.37 (m, 1H), 8.33 (dd, J=27.9, 7.3 Hz, 1H), 7.73 (d, J=8.4 Hz, 1H), 7.67 (dd, J=25.7, 8.5 Hz, 1H), 7.53 (dd, J=14.1, 8.3 Hz, 2H), 7.25-7.05 (m, 2H), 6.79-6.67 (m, 1H), 6.33-6.21 (m, 1H), 4.76-4.40 (m, 1H), 4.03-3.79 (m, 1H), 3.72 (q, J=10 Hz, 1H), 3.66-3.44 (m, 6H), 3.15-3.07 (m, 2H), 2.43-2.33 (m, 3H), 2.33-2.21 (m, 1H), 2.21-1.96 (m, 2H); MS (m/z): 577.64 [M+1]⁺.

### Example 9. (E)-N-(4-((R)-3-(5-cyano-4-(1H-indol-3-yl)pyrimidin-2-ylamino)pyrrolidine-1-carbonyl)phenyl)-4-(pyrrolidin-1-yl)but-2-enamide (Compound 108)

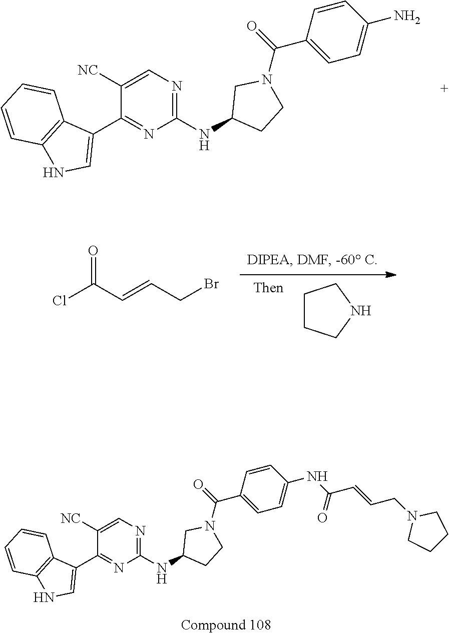

To a cooled (−60° C.) solution of 2-((R)-1-(4-aminobenzoyl)pyrrolidin-3-ylamino)-4-(1H-indol-3-yl)pyrimidine-5-carbonitrile (40 mg, 0.094 mmol) and DIPEA (65 μL, 0.376 mmol) in THF (4.7 mL) was added a 54 mg/mL solution of (E)-4-bromobut-2-enoyl chloride in THF (625 μL, 0.184 mmol). The mixture was stirred 1 h 30 min at −60° C. before addition of pyrolidine (24 μL, 0.283 mmol). The mixture was stirred 24 h at rt and concentrated under reduced pressure. The residue was purified by preparative HPLC (water/ACN 0.1% NH₄HCO₃ 15 to 60% gradient) and afforded Compound 108 (7.9 mg, 0.014 mmol, 15%) as a white solid after lyophilization. ¹H NMR (500 MHz, d₆-DMSO) δ 12.03 (s (br), 1H) 10.31-10.12 (m, 1H), 8.71 (dd, J=22.4, 9.1 Hz, 1H), 8.64-8.52 (m, 2H), 8.47 (dd, J=23.2, 5.5 Hz, 1H), 8.34 (dd, J=36.9, 6.8 Hz, 1H), 7.73 (d, J=8.5 Hz, 1H), 7.67 (dd, J=25.0, 8.3 Hz, 1H), 7.53 (dd, J=13.9, 8.1 Hz, 2H), 7.27-7.06 (m, 2H), 6.85-6.72 (m, 1H), 6.33-6.20 (m, 1H), 4.76-4.39 (m, 1H), 4.02-3.81 (m, 1H), 3.78-3.67 (m, 1H), 3.66-3.44 (m, 3H), 3.26-3.17 (m, 3H), 2.47 (s (br), 2H), 2.34-1.97 (m, 2H), 1.92-1.65 (m, 2H), 1.70 (s (br), 2H); MS (m/z): 561.68 [M+1]⁺.

### Example 10. (E)-N-(4-((R)-3-(5-cyano-4-(1H-indol-3-yl)pyrimidin-2-ylamino)pyrrolidine-1-carbonyl)phenyl)-4-(1H-imidazol-1-yl)but-2-enamide (Compound 109)

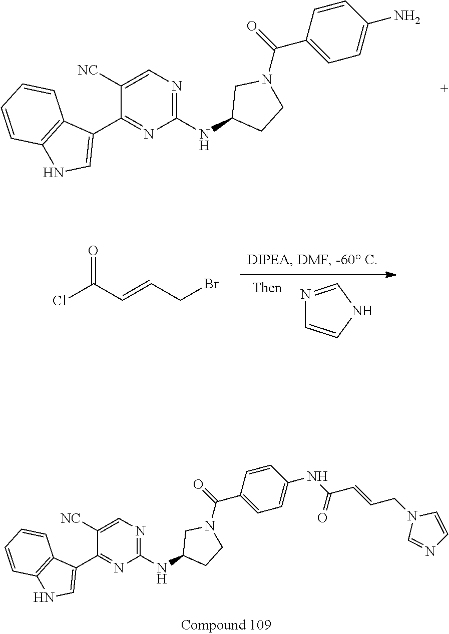

To a cooled (−60° C.) solution of 2-((R)-1-(4-aminobenzoyl)pyrrolidin-3-ylamino)-4-(1H-indol-3-yl)pyrimidine-5-carbonitrile (74 mg, 0.175 mmol) and DPEA (122 L, 0.700 mmol) in THF (3.5 mL) was added a 56 mg/mL solution of (E)-4-bromobut-2-enoyl chloride in THF (605 μL, 0.185 mmol). The mixture was stirred 1 h 30 min at −60° C. before addition of imidazole (36 mg, 0.524 mmol). The mixture was stirred 48 h at rt and concentrated under reduced pressure. The residue was purified by preparative HPLC (water/ACN 0.1% HCO₂H 15 to 50% gradient) and afforded Compound 109 (11.4 mg, 0.020 mmol, 12%) as a white solid after lyophilization. ¹H NMR (500 MHz, d₆-DMSO) δ 12.02 (s (br), 1H), 10.33-10.20 (m, 1H), 8.76-8.66 (m, 1H), 8.65-8.56 (m, 1H), 8.55-8.49 (m, 1H), 8.49-8.44 (m, 1H), 8.42-8.26 (m, 1H), 7.68 (dd, J=13.7, 8.2 Hz, 1H), 7.62 (d, J=8.8 Hz, 1H), 7.52 (dd, J=12.5, 8.1 Hz, 2H), 7.28-7.08 (m, 2H), 7.03-6.87 (m, 1H), 6.82-6.68 (m, 2H), 5.90-5.78 (m, 1H), 4.92-4.82 (m, 1H), 4.76-4.40 (m, 2H), 4.02-3.79 (m, 2H), 3.77-3.66 (m, 1H), 3.67-3.44 (m, 3H); MS (m/z): 558.63 [M+1]⁺.

### Example 11. (E)-N-(4-((R)-3-(5-cyano-4-(1H-indol-3-yl)pyrimidin-2-ylamino)pyrrolidine-1-carbonyl)phenyl)-4-(4-methylpiperazin-1-yl)but-2-enamide (Compound 110)

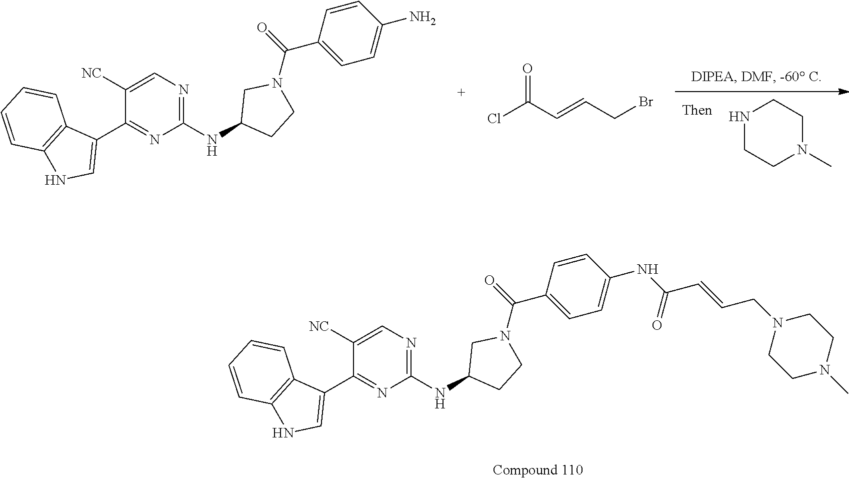

To a cooled (−60° C.) solution of 2-((R)-1-(4-aminobenzoyl)pyrrolidin-3-ylamino)-4-(1H-indol-3-yl)pyrimidine-5-carbonitrile (74 mg, 0.175 mmol) and DIPEA (122 μL, 0.700 mmol) in THF (3.5 mL) was added a 56 mg/mL solution of (E)-4-bromobut-2-enoyl chloride in THE (605 μL, 0.185 mmol). The mixture was stirred 3.5 h at −60° C. before addition of N-methylpiperazine (58 μL, 0.524 mmol). The mixture was stirred 24 h at rt and concentrated under reduced pressure. The residue was purified by preparative HPLC (water/ACN 0.1% HCO₂H 10 to 50% gradient) and afforded Compound 110 (16.0 mg, 0.027 mmol, 15.5%) as a white solid after lyophilization. ¹H NMR (500 MHz, d₆-DMSO) δ 12.10-11.94 (m, 1H), 10.27-10.15 (m, 1H), 8.76-8.66 (m, 1H), 8.65-8.58 (m, 1H), 8.55-8.44 (m, 1H), 8.35 (dd, J=44.7, 7.1 Hz, 1H), 8.25 (s (br), 1H), 7.71 (dd, J=14.1, 8.3 Hz, 1H), 7.64 (d, J=8.0 Hz, 1H), 7.57-7.48 (m, 2H), 7.29-7.08 (m, 2H), 6.79-6.66 (m, 1H), 6.31-6.18 (m, 1H), 4.74-4.41 (m, 2H), 4.02-3.78 (m, 2H), 3.77-3.67 (m, 1H), 3.66-3.47 (m, 3H), 3.13-3.05 (m, 2H), 2.5 (s, 3H), 2.46-2.21 (m, 4H), 2.16 (s, 2H), 2.08 (d, J=5.3 Hz, 1H); MS (m/z): 590.71 [M+1]⁺.

### Example 12. (E)-N-(4-((R)-3-(5-cyano-4-(1H-indol-3-yl)pyrimidin-2-ylamino)pyrrolidine-1-carbonyl)phenyl)-4-((2-hydroxyethyl)(methyl)amino)but-2-enamide (Compound 111)

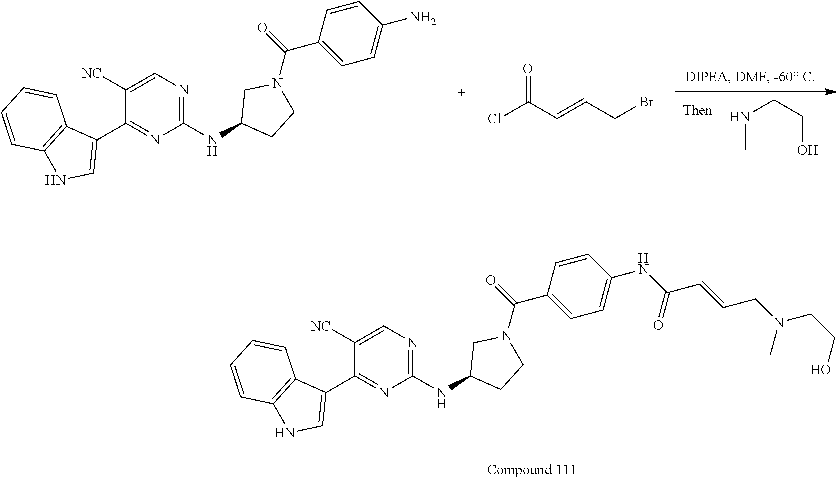

To a cold solution (−60° C.) of 2-((R)-1-(4-aminobenzoyl)pyrrolidin-3-ylamino)-4-(1H-indol-3-yl)pyrimidine-5-carbonitrile (74 mg, 0.175 mmol) in THE (3.5 mL) was added a 55.6 mg/mL solution of (E)-4-bromobut-2-enoyl chloride (605 μL, 0.185 mmol) in THF. After 4 h at (−60° C.), 2-(methylamino)ethanol was added (40 mg, 0.524 mmol) and the mixture was stirred 48 h at rt. NMP (2 mL) was added, and the THF was evaporated under reduced pressure, after which the residue was purified by prep HPLC (0.1% HCOOH, H₂O/ACN 15 to 50% gradient) to afford Compound 111 (6.4 mg, 0.011 mmol, 6.5%) as a white solid after lyophilization ¹H NMR (500 MHz, d₆-DMSO) δ 12.00 (s (br), 1H), 10.30-10.06 (m, 1H), 8.65 (dd, J=20.9, 9.0 Hz, 1H), 8.57-8.51 (m, 1H), 8.48-8.35 (m, 2H), 8.28 (dd, J=35.2, 7.0 Hz, 1H), 7.70-7.54 (m, 2H), 7.52-7.40 (m, 2H), 7.21-7.01 (m, 2H), 6.75-6.62 (m, 1H), 6.27-6.12 (m, 1H), 4.69-4.33 (m, 2H), 3.97-3.74 (m, 2H), 3.65 (dd, J=18.5, 10.2 Hz, 2H), 3.59-3.45 (m, 3H), 3.14-3.05 (m, 2H), 2.41-2.31 (m, 2H), 2.26-2.06 (m, 2H), 2.14 (s, 3H); MS (m/z): 565.66 [M+1]⁺.

### Example 13. (E)-N-(4-(4-(5-chloro-4-(1H-indol-3-yl)pyrimidin-2-ylamino)piperidine-1-carbonyl)-2-fluorophenyl)-4-(dimethylamino)but-2-enamide (Compound 112)

### tert-butyl 4-(5-chloro-4-(1-(phenylsulfonyl)-1H-indol-3-yl)pyrimidin-2-ylamino)piperidine-1-carboxylate

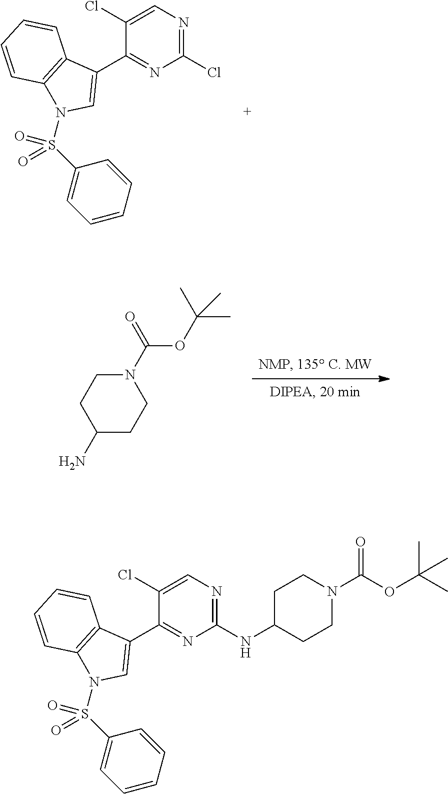 [SMILES: O=S(=O)(c1ccccc1)n1cc(-c2nc(Cl)ncc2Cl)c2ccccc21 ; CC(C)(C)OC(=O)N1CCC(N)CC1 ; "DIPEA, 20 min" ; "NMP, 135° C. MW" ; CC(C)(C)OC(=O)N1CCC(Nc2ncc(Cl)c(-c3cn(S(=O)(=O)c4ccccc4)c4ccccc34)n2)CC1]

A solution of 3-(2,5-dichloropyrimidin-4-yl)-1-(phenylsulfonyl)-1H-indole (1.5 g, 3.71 mmol), tert-butyl 4-aminopiperidine-1-carboxylate (780 mg, 3.9 mmol) and DIPEA (646 μL, 3.71 mmol) in NMP (6.2 mL) was heated at 135° C. (microwave) for 35 min. The cold mixture was diluted with EtOAc (100 mL), washed with water (20 mL), brine (20 mL), dried (MgSO₄), filtered and concentrated under reduced pressure. The residue was purified by flash chromatography (Hex/EtOAc 10 to 100% gradient) and afforded the title compound (1.58 g, 2.78 mmol, 75%) as a colorless glue.

### 5-chloro-4-(1-(phenylsulfonyl)-1H-indol-3-yl)-N-(piperidin-4-yl)pyrimidin-2-amine

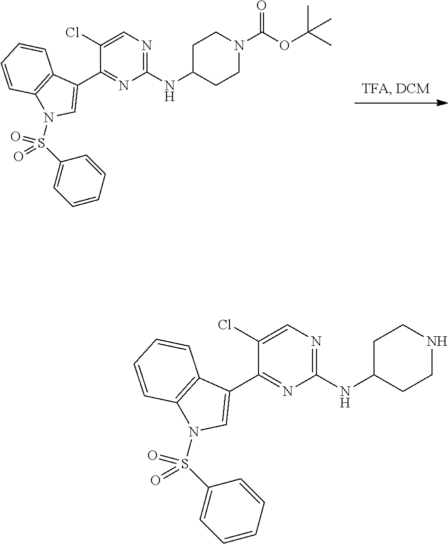

A solution of tert-butyl 4-(5-chloro-4-(1-(phenylsulfonyl)-1H-indol-3-yl)pyrimidin-2-ylamino)piperidine-1-carboxylate (1.58 g, 2.79 mmol) in DCM (12.4 mL) was treated with TFA (2.13 mL, 28 mmol). The mixture was stirred 1 h at rt before evaporation of the volatiles under reduced pressure. The residue was diluted with DCM (50 mL), washed with sat NaHCO₃ (3×10 mL), brine (10 mL), dried (MgSO₄), filtered and concentrated under reduced pressure. The residue was used in the next step without further purification.

### (4-amino-3-fluorophenyl)(4-(5-chloro-4-(1-(phenylsulfonyl)-1H-indol-3-yl)pyrimidin-2-ylamino)piperidin-1-yl)methanone

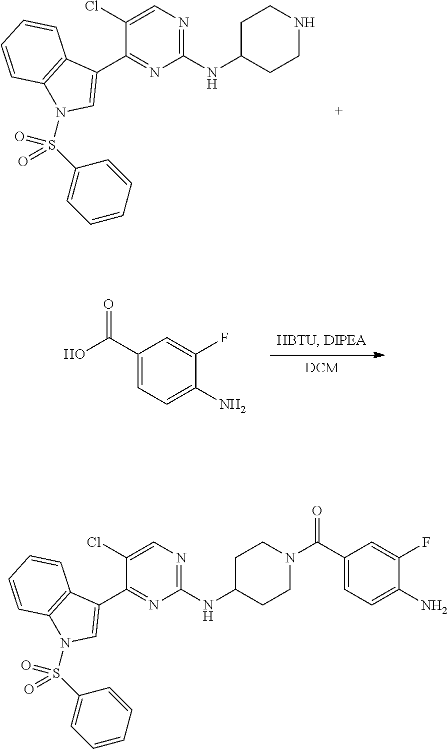

A solution of 5-chloro-4-(1-(phenylsulfonyl)-1H-indol-3-yl)-N-(piperidin-4-yl)pyrimidin-2-amine (100 mg, 0.21 mmol), 4-amino-3-fluorobenzoic acid (33 mg, 0.21 mmol), HBTU (162 mg, 0.43 mmol) and DIPEA (0.11 mL, 0.64 mmol) in DCM (1.42 mL) was stirred overnight at 23° C. before being concentrated under reduced pressure. The residue was purified by flash chromatography (Hex/EtOAc 0 to 100% gradient) and afforded the title compound (127 mg, 0.21 mmol, 100%) as a white solid.

### (4-amino-3-fluorophenyl)(4-(5-chloro-4-(1H-indol-3-yl)pyrimidin-2-ylamino)piperidin-1-yl)methanone (Compound 55)

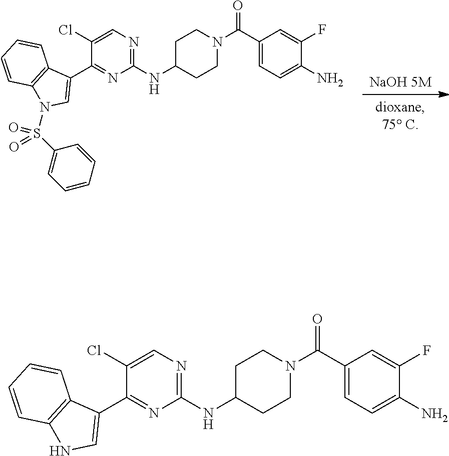 [SMILES: Nc1ccc(C(=O)N2CCC(Nc3ncc(Cl)c(-c4cn(S(=O)(=O)c5ccccc5)c5ccccc45)n3)CC2)cc1F ; "NaOH 5M" ; "dioxane,75° C." ; Nc1ccc(C(=O)N2CCC(Nc3ncc(Cl)c(-c4c[nH]c5ccccc45)n3)CC2)cc1F]

A solution of (4-amino-3-fluorophenyl)(4-(5-chloro-4-(1-(phenylsulfonyl)-1H-indol-3-yl)pyrimidin-2-ylamino)piperidin-1-yl)methanone (129 mg, 0.21 mmol) and 5M NaOH (0.43 mL, 2.13 mmol) in dioxane (1.4 mL) was heated 2 h at 75° C. The cold mixture was concentrated under reduced pressure and water was azeotropically removed with toluene (3×2 mL). The residue was purified by flash chromatography (iPrOH/DCM 0 to 15% gradient) and afforded the title compound (70 mg, 0.15 mmol, 71%) as a white solid.

### (E)-N-(4-(4-(5-chloro-4-(1H-indol-3-yl)pyrimidin-2-ylamino)piperidine-1-carbonyl)-2-fluorophenyl)-4-(dimethylamino)but-2-enamide (Compound 112)

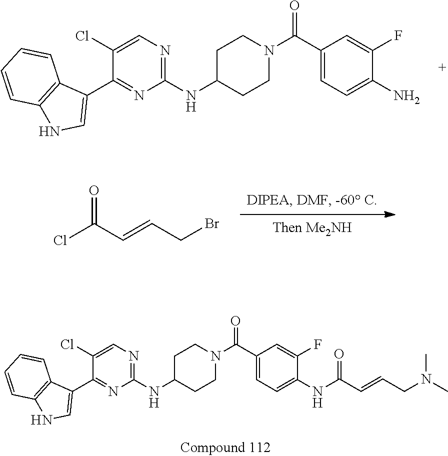 [SMILES: Nc1ccc(C(=O)N2CCC(Nc3ncc(Cl)c(-c4c[nH]c5ccccc45)n3)CC2)cc1F ; O=C(Cl)/C=C/CBr ; "DIPEA, DMF, -60° C." ; "Then Me2NH" ; CN(C)C/C=C/C(=O)Nc1ccc(C(=O)N2CCC(Nc3ncc(Cl)c(-c4c[nH]c5ccccc45)n3)CC2)cc1F]

To a cold solution (−60° C.) of (4-amino-3-fluorophenyl)(4-(5-chloro-4-(1H-indol-3-yl)pyrimidin-2-ylamino)piperidin-1-yl)methanone (63 mg, 0.136 mmol) and DIPEA (71 μL, 0.42 mmol) in THF (6.8 mL) was added a 55.6 mg/mL solution of (E)-4-bromobut-2-enoyl chloride (487 μL, 0.148 mmol) in THF. After 1 h30 at (−60° C.), a 2M solution of dimethylamine in THF (203 μL, 0.407 mmol) was added and the mixture was stirred 24 h at rt. NMP (2 mL) was added, THF was evaporated under reduced pressure and the residue was purified by prep HPLC (0.1% HCOOH, H₂O/ACN 15 to 50% gradient) and afforded the title compound (19.5 mg, 0.034 mmol, 25%) as a white solid after lyophilization. ¹H NMR (500 MHz, d₆-DMSO) δ 11.86 (s, 1H), 9.99 (s, 1H), 8.61 (s (br), 1H), 8.47 (d, J=2.8 Hz, 1H), 8.32 (s, 1H), 8.27 (s, 1H), 8.13 (t, J=8.2 Hz, 1H), 7.49 (d, J=7.8 Hz, 1H), 7.33 (d, J=11.0 Hz, 1H), 7.30 (d, J=7.7 Hz, 1H), 7.24-7.16 (m, 2H), 6.77 (dt, J=15.4, 5.9 Hz, 1H), 6.48 (d, J=15.4 Hz, 1H), 4.42 (s (br), 1H), 4.09 (s (br), 2H), 3.70 (s (br), 2H), 3.06 (d, J=4.9 Hz, 2H), 2.17 (s, 6H), 2.02 (s (br), 2H), 1.53 (s (br), 2H); MS (m/z): 576.56 [M+1]⁺.

### Example 14. (E)-N-(4-(4-(5-chloro-4-(1H-indol-3-yl)pyrimidin-2-ylamino)piperidine-1-carbonyl)-2-methylphenyl)-4-(dimethylamino)but-2-enamide (Compound 113)

### (4-(5-chloro-4-(1-(phenylsulfonyl)-1H-indol-3-yl)pyrimidin-2-ylamino)piperidin-1-yl)(3-methyl-4-nitrophenyl)methanone

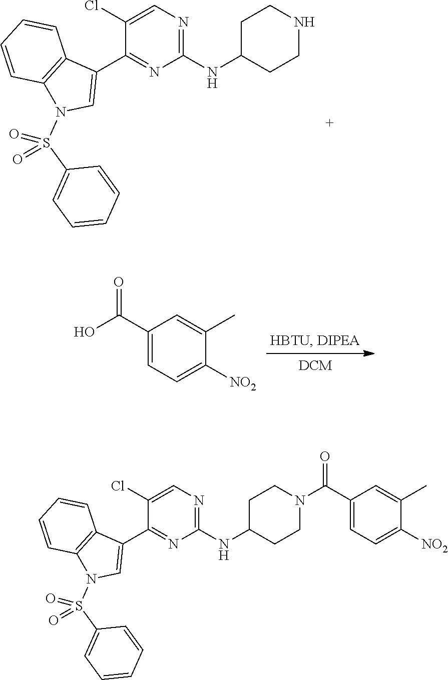 [SMILES: O=S(=O)(c1ccccc1)n1cc(-c2nc(NC3CCNCC3)ncc2Cl)c2ccccc21 ; "+" ; Cc1cc(C(=O)O)ccc1[N+](=O)[O-] ; Cc1cc(C(=O)N2CCC(Nc3ncc(Cl)c(-c4cn(S(=O)(=O)c5ccccc5)c5ccccc45)n3)CC2)ccc1[N+](=O)[O-]]

A solution of 5-chloro-4-(1-(phenylsulfonyl)-1H-indol-3-yl)-N-(piperidin-4-yl)pyrimidin-2-amine (220 mg, 0.47 mmol), 3-methyl-4-nitrobenzoic acid (85 mg, 0.47 mmol), HBTU (357 mg, 0.94 mmol) and DIPEA (0.25 mL, 1.41 mmol) in DCM (3.1 mL) was stirred overnight at 23° C. before being concentrated under reduced pressure. The residue was purified by flash chromatography (Hex/EtOAc 10 to 100% gradient) and afforded the title compound (271 mg, 0.43 mmol, 91%) as a yellowish solid.

### (4-amino-3-methylphenyl)(4-(5-chloro-4-(1-(phenylsulfonyl)-1H-indol-3-yl)pyrimidin-2-ylamino)piperidin-1-yl)methanone

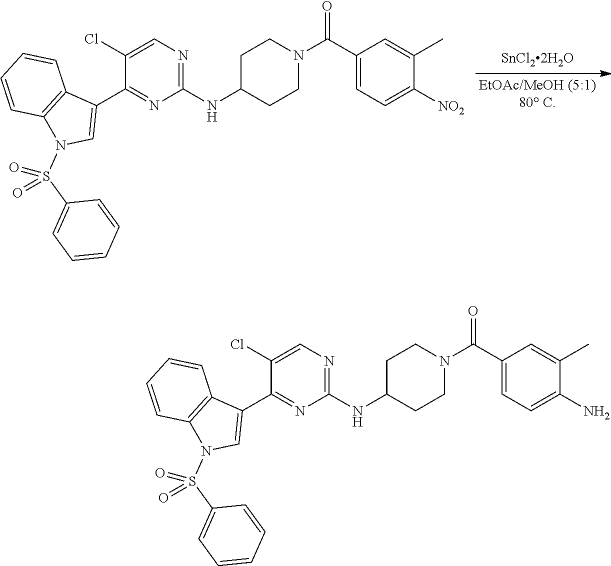 [SMILES: Cc1cc(C(=O)N2CCC(Nc3ncc(Cl)c(-c4cn(S(=O)(=O)c5ccccc5)c5ccccc45)n3)CC2)ccc1[N+](=O)[O-] ; Cc1cc(C(=O)N2CCC(Nc3ncc(Cl)c(-c4cn(S(=O)(=O)c5ccccc5)c5ccccc45)n3)CC2)ccc1N]

A solution of (4-(5-chloro-4-(1-(phenylsulfonyl)-1H-indol-3-yl)pyrimidin-2-ylamino)piperidin-1-yl)(3-methyl-4-nitrophenyl)methanone (271 mg, 0.43 mmol) in EtOAc/MeOH (5/1, 10 mL) was treated with Tin (II) chloride dihydrate (242 mg, 1.07 mmol) and the mixture was heated 3 h at 80° C. The cold mixture was poured into sat NaHCO₃ (10 mL) and the resulting mixture was stirred 20 min at rt before being extracted with EtOAc (3×20 ml). The combined organics layers were washed with water (10 mL), brine (10 mL), dried (MgSO₄), filtered and concentrated under reduced pressure and afforded the title compound (278 mg, 0.46 mmol, 108%) as a light orange solid which was used in the next step without further purification.

### (4-amino-3-methylphenyl)(4-(5-chloro-4-(1H-indol-3-yl)pyrimidin-2-ylamino)piperidin-1-yl)methanone (Compound 59)

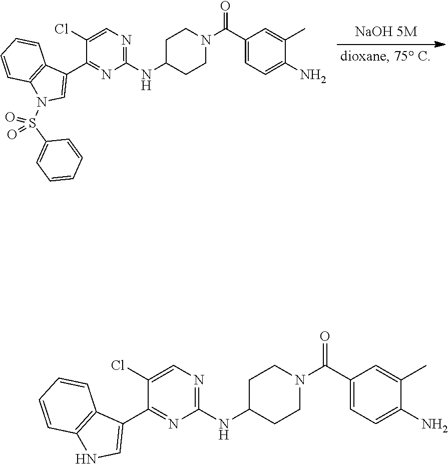 [SMILES: Cc1cc(C(=O)N2CCC(Nc3ncc(Cl)c(-c4cn(S(=O)(=O)c5ccccc5)c5ccccc45)n3)CC2)ccc1N ; Cc1cc(C(=O)N2CCC(Nc3ncc(Cl)c(-c4c[nH]c5ccccc45)n3)CC2)ccc1N]

A solution of (4-amino-3-methylphenyl)(4-(5-chloro-4-(1-(phenylsulfonyl)-1H-indol-3-yl)pyrimidin-2-ylamino)piperidin-1-yl)methanone (258 mg, 0.43 mmol) and 5M NaOH (0.86 mL, 4.29 mmol) in dioxane (2.9 mL) was heated 2 h at 75° C. The cold mixture was concentrated under reduced pressure and water was azeotropically removed with toluene (3×2 mL). The residue was purified by flash chromatography (iPrOH/DCM 0 to 15% gradient) and afforded the title compound (174 mg, 0.38 mmol, 75%) as a white solid.

### (E)-N-(4-(4-(5-chloro-4-(1H-indol-3-yl)pyrimidin-2-ylamino)piperidine-1-carbonyl)-2-methylphenyl)-4-(dimethylamino)but-2-enamide (Compound 113)

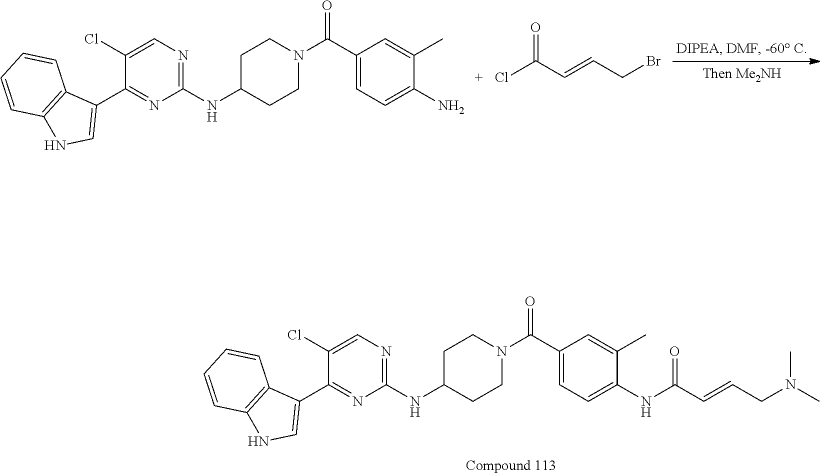

To a cold solution (−60° C.) of (4-amino-3-methylphenyl)(4-(5-chloro-4-(1H-indol-3-yl)pyrimidin-2-ylamino)piperidin-1-yl)methanone (144 mg, 0.312 mmol) and DIPEA (164 μL, 0.94 mmol) in THF (6.2 mL) was added a 55.6 mg/mL solution of (E)-4-bromobut-2-enoyl chloride (1.12 mL, 0.340 mmol) in THF. After 3 h at (−60° C.), a 2M solution of dimethylamine in THF (469 μL, 0.93 mmol) was added and the mixture was stirred 24 h at rt. NMP (2 mL) was added, THF was evaporated under reduced pressure and the residue was purified by prep HPLC (0.1% HCOOH, H₂O/ACN 15 to 65% gradient) and afforded Compound 113 (58 mg, 0.101 mmol, 33%) as a white solid after lyophilization. ¹H NMR (500 MHz, d₆-DMSO) δ 11.85 (s, 1H), 9.43 (s, 1H), 8.59 (s (br), 1H), 8.47 (d, J=3.0 Hz, 1H), 8.27 (s, 1H), 8.27 (s (br), 1H), 7.65 (d, J=8.1 Hz, 1H), 7.49 (d, J=8.0 Hz, 1H), 7.33-7.24 (m, 2H), 7.24-7.14 (m, 2H), 6.74 (dt, J=15.4, 5.9 Hz, 1H), 6.41 (d, J=15.4 Hz, 1H), 4.55-4.33 (m, 1H), 4.18-3.98 (m, 1H), 3.83-3.63 (m, 1H), 3.44-3.25 (m, 2H), 3.06 (dd, J=5.8, 1.2 Hz, 2H), 2.26 (s, 3H), 2.18 (s, 6H), 2.10-1.90 (m, 2H), 1.59-1.42 (m, 2H); MS (m/z): 572.65 [M+1]⁺.

### Example 15. (E)-N-(4-(4-(5-chloro-4-(1H-indol-3-yl)pyrimidin-2-ylamino)piperidine-1-carbonyl)-3-methylphenyl)-4-(dimethylamino)but-2-enamide (Compound 114)

### N-(4-(4-(5-chloro-4-(1-(phenylsulfonyl)-1H-indol-3-yl)pyrimidin-2-ylamino)piperidine-1-carbonyl)-3-methylphenyl)acetamide

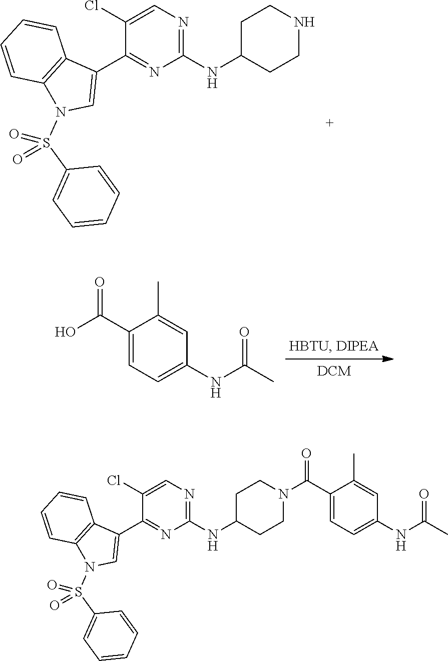 [SMILES: O=S(=O)(c1ccccc1)n1cc(-c2nc(NC3CCNCC3)ncc2Cl)c2ccccc21 ; "+" ; CC(=O)Nc1ccc(C(=O)O)c(C)c1 ; CC(=O)Nc1ccc(C(=O)N2CCC(Nc3ncc(Cl)c(-c4cn(S(=O)(=O)c5ccccc5)c5ccccc45)n3)CC2)c(C)c1]

A solution of 5-chloro-4-(1-(phenylsulfonyl)-1H-indol-3-yl)-N-(piperidin-4-yl)pyrimidin-2-amine (220 mg, 0.47 mmol), 4-acetamido-2-methylbenzoic acid (91 mg, 0.47 mmol), HBTU (357 mg, 0.94 mmol) and DIPEA (0.25 mL, 1.41 mmol) in DCM (3.1 mL) was stirred overnight at 23° C. before being concentrated under reduced pressure. The residue was purified by flash chromatography (Hex/EtOAc 50 to 100% gradient) and afforded the title compound (240 mg, 0.37 mmol, 83%) as a white solid.

### 4-amino-2-methylphenyl)(4-(5-chloro-4-(1-(phenylsulfonyl)-1H-indol-3-yl)pyrimidin-2-ylamino)piperidin-1-yl)methanone

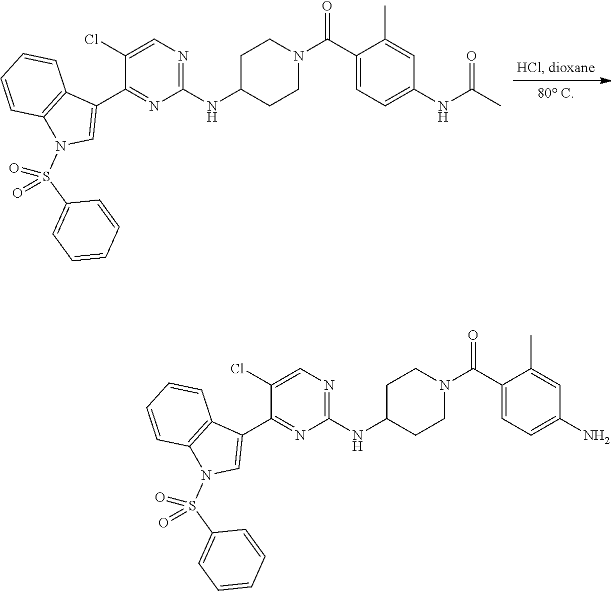 [SMILES: CC(=O)Nc1ccc(C(=O)N2CCC(Nc3ncc(Cl)c(-c4cn(S(=O)(=O)c5ccccc5)c5ccccc45)n3)CC2)c(C)c1 ; Cc1cc(N)ccc1C(=O)N1CCC(Nc2ncc(Cl)c(-c3cn(S(=O)(=O)c4ccccc4)c4ccccc34)n2)CC1]

A solution of N-(4-(4-(5-chloro-4-(1-(phenylsulfonyl)-1H-indol-3-yl)pyrimidin-2-ylamino)piperidine-1-carbonyl)-3-methylphenyl)acetamide (240 mg, 0.37 mmol) in dioxane (7.5 mL) was treated with 4M HCl in dioxane (0.73 mL, 3.64 mmol) and heated 3 h at 80° C. The cold mixture was diluted with DCM (10 mL), washed with sat NaHCO₃ (10 mL), dried (MgSO₄), filtered concentrated under reduced pressure and afforded the title compound (219 mg, 0.36 mmol, 98%) which was used in the next step without further purification.

### (4-amino-2-methylphenyl)(4-(5-chloro-4-(1H-indol-3-yl)pyrimidin-2-ylamino)piperidin-1-yl)methanone (Compound 63)

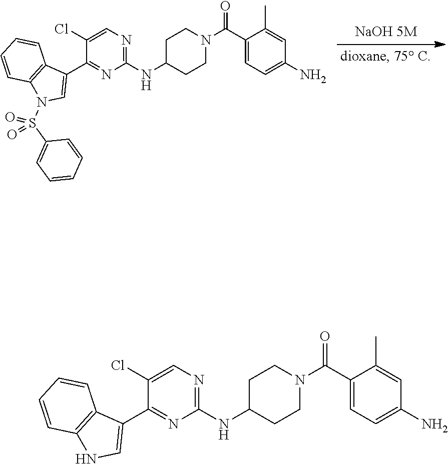 [SMILES: Cc1cc(N)ccc1C(=O)N1CCC(Nc2ncc(Cl)c(-c3cn(S(=O)(=O)c4ccccc4)c4ccccc34)n2)CC1 ; Cc1cc(N)ccc1C(=O)N1CCC(Nc2ncc(Cl)c(-c3c[nH]c4ccccc34)n2)CC1]

A solution of (4-amino-2-methylphenyl)(4-(5-chloro-4-(1-(phenylsulfonyl)-1H-indol-3-yl)pyrimidin-2-ylamino)piperidin-1-yl)methanone (219 mg, 0.36 mmol) and 5M NaOH (0.73 mL, 3.64 mmol) in dioxane (2.4 mL) was heated 2 h at 75° C. The cold mixture was concentrated under reduced pressure and water was azeotropically removed with toluene (3×2 mL). The residue was purified by flash chromatography (iPrOH/DCM 0 to 15% gradient) and afforded the title compound (136 mg, 0.29 mmol, 81%) as a white solid.

### (E)-N-(4-(4-(5-chloro-4-(1H-indol-3-yl)pyrimidin-2-ylamino)piperidine-1-carbonyl)-3-methylphenyl)-4-(dimethylamino)but-2-enamide (Compound 114)

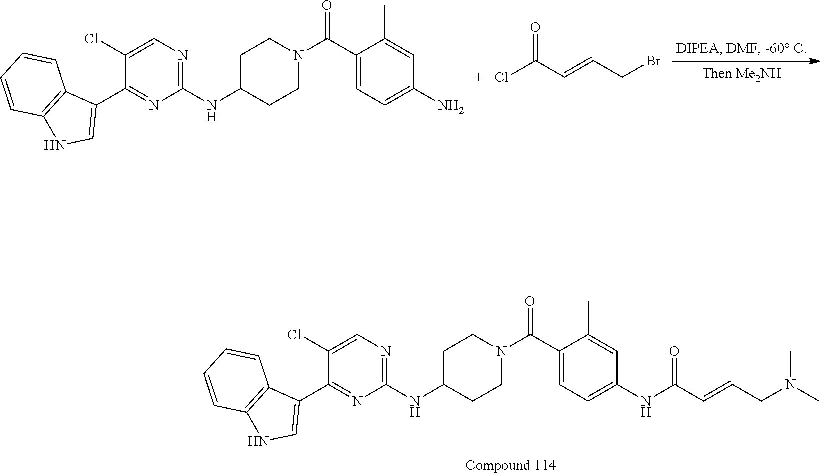

To a cold solution (−60° C.) of (4-amino-2-methylphenyl)(4-(5-chloro-4-(1H-indol-3-yl)pyrimidin-2-ylamino)piperidin-1-yl)methanone (130 mg, 0.282 mmol) and DIPEA (148 μL, 846 mmol) in THF (5.6 mL) was added a 55.6 mg/mL solution of (E)-4-bromobut-2-enoyl chloride (1.01 mL, 0.308 mmol) in THF. After 4 h30 at (−60° C.), a 2M solution of dimethylamine in THF (423 μL, 0.846 mmol) was added and the mixture was stirred 24 h at rt. NMP (2 mL) was added, THF was evaporated under reduced pressure and the residue was purified by prep HPLC-MS (0.1% HCOOH, H₂O/ACN 15 to 55% gradient) and afforded Compound 114 (45 mg, 0.079 mmol, 28%) as a white solid after lyophilization. ¹H NMR (500 MHz, d₆-DMSO) δ 11.85 (s, 1H), 10.10 (s, 1H), 8.74-8.50 (m, 1H), 8.47 (d, J=3.0 Hz, 1H), 8.26 (s, 1H), 7.57 (s, 1H), 7.53 (d, J=8.3 Hz, 1H), 7.49 (d, J=8.1 Hz, 1H), 7.29 (d, J=7.6 Hz, 1H), 7.24-7.19 (m, 1H), 7.19-7.15 (m, 1H), 7.11 (d, J=8.2 Hz, 1H), 6.74 (dt, J=15.4, 5.9 Hz, 1H), 6.27 (dt, J=15.3, 1.5 Hz, 1H), 4.52 (d, J=12.5 Hz, 1H), 4.16-3.98 (m, 1H), 3.19-3.09 (m, 2H), 3.06 (dd, J=5.9, 1.4 Hz, 2H), 3.03-2.89 (m, 1H), 2.31-2.19 (m, 3H), 2.18 (s, 6H), 2.14-2.00 (m, 1H), 1.95-1.78 (m, 1H), 1.59-1.47 (m, 1H), 1.46-1.34 (m, 1H); MS (m/z): 572.59 [M+1]⁺.

### Example 16. (E)-N-(4-(4-(5-chloro-4-(1H-indol-3-yl)pyrimidin-2-ylamino)piperidine-1-carbonyl)-3-fluorophenyl)-4-(dimethylamino)but-2-enamide (Compound 115)

### (4-(5-chloro-4-(1-(phenylsulfonyl)-1H-indol-3-yl)pyrimidin-2-ylamino)piperidin-1-yl)(2-fluoro-4-nitrophenyl)methanone

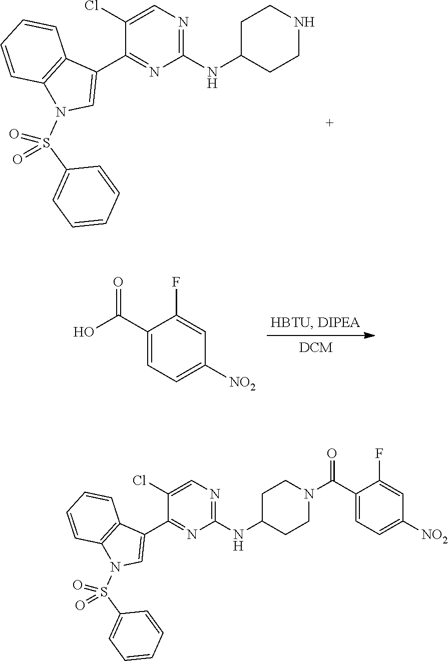 [SMILES: O=S(=O)(c1ccccc1)n1cc(-c2nc(NC3CCNCC3)ncc2Cl)c2ccccc21 ; "+" ; O=C(O)c1ccc([N+](=O)[O-])cc1F ; O=C(c1ccc([N+](=O)[O-])cc1F)N1CCC(Nc2ncc(Cl)c(-c3cn(S(=O)(=O)c4ccccc4)c4ccccc34)n2)CC1]

A solution of 5-chloro-4-(1-(phenylsulfonyl)-1H-indol-3-yl)-N-(piperidin-4-yl)pyrimidin-2-amine (220 mg, 0.47 mmol), 4-nitro-2-fluorobenzoic acid (87 mg, 0.47 mmol), HBTU (357 mg, 0.94 mmol) and DIPEA (0.25 mL, 1.41 mmol) in DCM (3.1 mL) was stirred overnight at 23° C. before being concentrated under reduced pressure. The residue was purified by flash chromatography (Hex/EtOAc 5 to 70% gradient) and afforded the title compound (303 mg, 0.47 mmol, 100%) as a yellowish solid.

### (4-amino-2-fluorophenyl)(4-(5-chloro-4-(1-(phenylsulfonyl)-1H-indol-3-yl)pyrimidin-2-ylamino)piperidin-1-yl)methanone

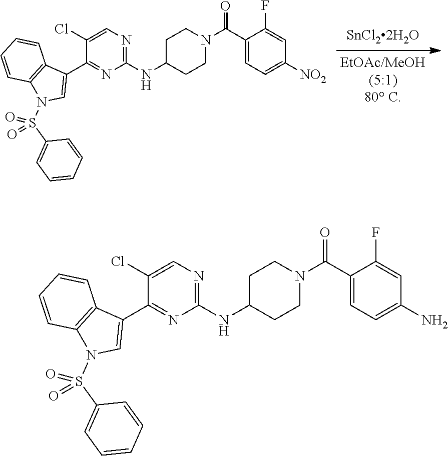 [SMILES: O=C(c1ccc([N+](=O)[O-])cc1F)N1CCC(Nc2ncc(Cl)c(-c3cn(S(=O)(=O)c4ccccc4)c4ccccc34)n2)CC1 ; Nc1ccc(C(=O)N2CCC(Nc3ncc(Cl)c(-c4cn(S(=O)(=O)c5ccccc5)c5ccccc45)n3)CC2)c(F)c1]

A solution of (4-(5-chloro-4-(1-(phenylsulfonyl)-1H-indol-3-yl)pyrimidin-2-ylamino)piperidin-1-yl)(2-fluoro-4-nitrophenyl)methanone (304 mg, 0.48 mmol) in EtOAc/MeOH (5/1, 10 mL) was treated with Tin (II) chloride dihydrate (270 mg, 1.2 mmol) and the mixture was heated 3 h at 80° C. The cold mixture was poured into sat NaHCO₃ (10 mL) and the resulting mixture was stirred 20 min at rt before being extracted with EtOAc (3×20 ml). The combined organics layers were washed with water (10 mL), brine (10 mL), dried (MgSO₄), filtered and concentrated under reduced pressure and afforded the title compound (290 mg, 0.48 mmol, 10%) as a yellow solid which was used in the next step without further purification.

### (4-amino-2-fluorophenyl)(4-(5-chloro-4-(1H-indol-3-yl)pyrimidin-2-ylamino)piperidin-1-yl)methanone (Compound 67)

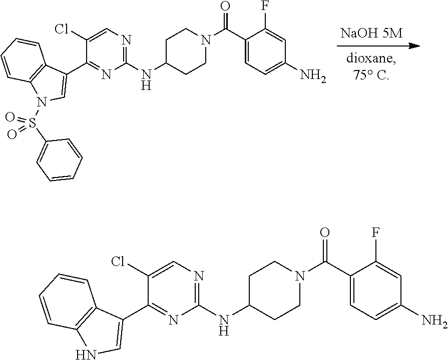 [SMILES: Nc1ccc(C(=O)N2CCC(Nc3ncc(Cl)c(-c4cn(S(=O)(=O)c5ccccc5)c5ccccc45)n3)CC2)c(F)c1 ; Nc1ccc(C(=O)N2CCC(Nc3ncc(Cl)c(-c4c[nH]c5ccccc45)n3)CC2)c(F)c1]

A solution of (4-amino-2-fluorophenyl)(4-(5-chloro-4-(1-(phenylsulfonyl)-1H-indol-3-yl)pyrimidin-2-ylamino)piperidin-1-yl)methanone (290 mg, 0.48 mmol) and 5M NaOH (0.96 mL, 4.79 mmol) in dioxane (3.2 mL) was heated 2 h at 75° C. The cold mixture was concentrated under reduced pressure and water was azeotropically removed with toluene (3×2 mL). The residue was purified by flash chromatography (iPrOH/DCM 0 to 15% gradient) and afforded the title compound (169 mg, 0.36 mmol, 76%) as a white solid.

### (E)-N-(4-(4-(5-chloro-4-(1H-indol-3-yl)pyrimidin-2-ylamino)piperidine-1-carbonyl)-3-fluorophenyl)-4-(dimethylamino)but-2-enamide (Compound 115)

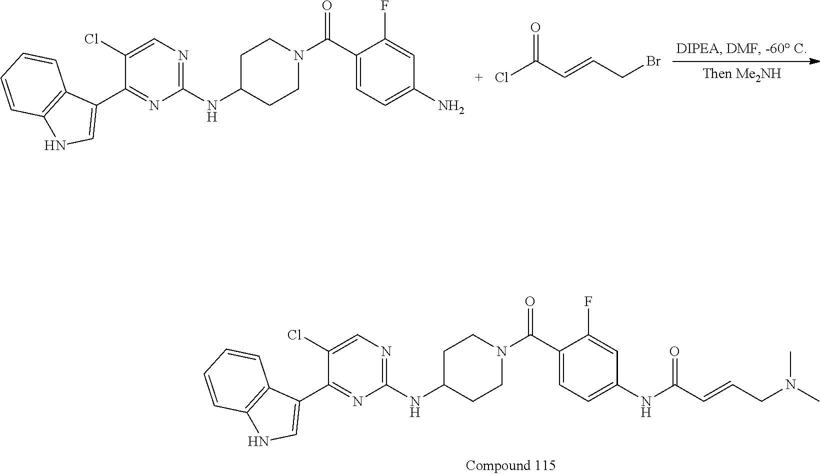

To a cold solution (−60° C.) of (4-amino-2-fluorophenyl)(4-(5-chloro-4-(1H-indol-3-yl)pyrimidin-2-ylamino)piperidin-1-yl)methanone (165 mg, 0.355 mmol) and DIPEA (186 μL, 1.07 mmol) in THF (1.8 mL) was added a 55.6 mg/mL solution of (E)-4-bromobut-2-enoyl chloride (532 μL, 1.07 mmol) in THF. After 1 h30 at (−60° C.), a 2M solution of dimethylamine in THF (532 μL, 1.07 mmol) was added and the mixture was stirred 24 h at rt. NMP (2 mL) was added, THF was evaporated under reduced pressure and the residue was purified by prep HPLC (0.1% HCOOH, H₂O/ACN 15 to 55% gradient) and afforded Compound 115 (59 mg, 0.102 mmol, 29%) as a white solid after lyophilization. ¹H NMR (500 MHz, d₆-DMSO) δ 11.85 (s, 1H), 10.41 (s, 1H), 8.71-8.50 (m, 1H), 8.48 (d, J=3.0 Hz, 1H), 8.27 (s, 1H), 7.76 (dd, J=12.3, 1.7 Hz, 1H), 7.50 (d, J=8.0 Hz, 1H), 7.41 (dd, J=8.4, 1.8 Hz, 1H), 7.38-7.30 (m, 2H), 7.25-7.20 (m, 1H), 7.20-7.14 (m, 1H), 6.79 (dt, J=15.4, 5.8 Hz, 1H), 6.27 (dt, J=15.4, 1.6 Hz, 1H), 4.48 (d, J=11.9 Hz, 1H), 4.17-4.03 (m, 1H), 3.59-3.49 (m, 2H), 3.08 (dd, J=5.9, 1.3 Hz, 2H), 3.05-2.96 (m, 1H), 2.19 (s, 6H), 2.13-2.01 (m, 1H), 2.01-1.87 (m, 1H), 1.59-1.43 (m, 2H); MS (m/z): 576.62 [M+1]⁺.

### Example 17. Synthesis of tert-butyl (4-(4-aminopiperidine-1-carbonyl)phenyl)carbamate (Intermediate 4)

Step 1

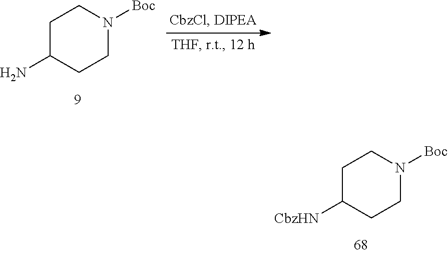 [SMILES: CC(C)(C)OC(=O)N1CCC(N)CC1 ; CC(C)(C)OC(=O)N1CCC(NC(=O)OCc2ccccc2)CC1]

To a solution of 9 (10 g, 50 mmol) in THF (100 mL) was added CbzCl (9.35 g, 55 mmol), DIPEA (12.9 g, 100 mmol) under N₂. The mixture was stirred at rt for 12 h. The mixture was quenched by water, extracted with EA. The organic layer was washed with brine, dried over Na₂SO₄ and concentrated under vacuum to afford 68 (22 g, 66% yield).

Step 2

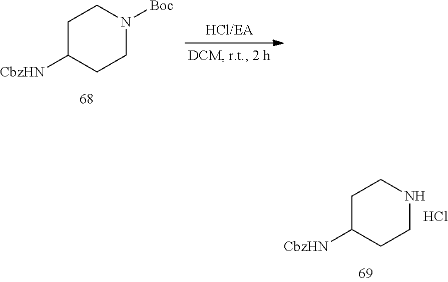 [SMILES: CC(C)(C)OC(=O)N1CCC(NC(=O)OCc2ccccc2)CC1 ; O=C(NC1CCNCC1)OCc1ccccc1 ; Cl]

To a solution of 68 (13 g, 38.9 mmol) in DCM (5 mL) was added HCl/EA (20 mL). The mixture was stirred at rt for 2 h. The mixture was filtered and filter cake was concentrated under vacuum to afford 69 (7.59 g, 83% yield).

Step 3

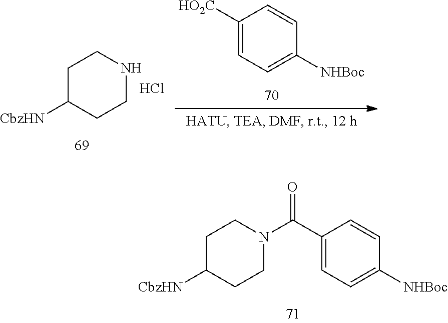 [SMILES: O=C(NC1CCNCC1)OCc1ccccc1 ; Cl ; CC(C)(C)OC(=O)Nc1ccc(C(=O)O)cc1 ; CC(C)(C)OC(=O)Nc1ccc(C(=O)N2CCC(NC(=O)OCc3ccccc3)CC2)cc1]

To a solution of 69 (11.39 g, 49 mmol) in DMF (100 mL) was added 70 (11.61 g, 49 mmol), HATU (28.12 g, 74 mmol), TEA (14.85 g, 147 mmol). The mixture was stirred at rt for 12 h. The mixture was dissolved with brine, filtered and extracted with EA. The organic layer was dried over Na₂SO₄ and concentrated under vacuum. The residue was purified by column chromatography on silica gel to afford 71 (21 g, 95% yield).

Step 4

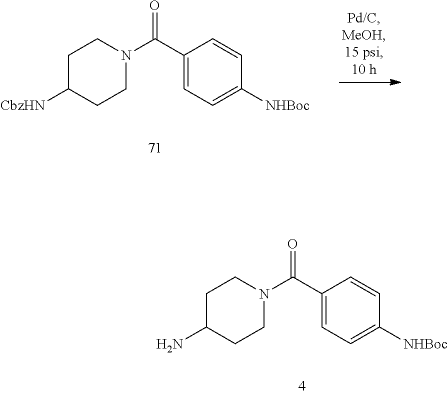 [SMILES: CC(C)(C)OC(=O)Nc1ccc(C(=O)N2CCC(NC(=O)OCc3ccccc3)CC2)cc1 ; CC(C)(C)OC(=O)Nc1ccc(C(=O)N2CCC(N)CC2)cc1]

To a solution of 71 (22 g, 48.6 mmol) in MeOH (220 mL) was added Pd/C (2 g). The mixture was stirred at 15 psi under H₂ for 10 h. The mixture was filtered and the filtrate was concentrated under vacuum to afford intermediate 4 (14 g, 90% yield).

### Example 18. Synthesis of (E)-N-(4-(4-((5-chloro-4-(1H-pyrazol-3-yl)pyrimidin-2-yl)amino)piperidine-1-carbonyl)phenyl)-4-(dimethylamino)but-2-enamide (Compound 117)

Step 1

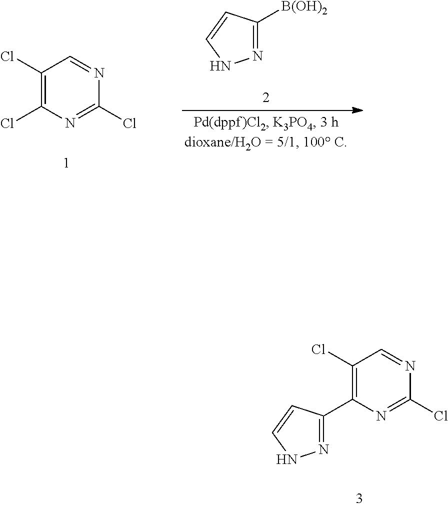 [SMILES: Clc1ncc(Cl)c(Cl)n1 ; OB(O)c1cc[nH]n1 ; Clc1ncc(Cl)c(-c2cc[nH]n2)n1]

Under N₂, to a solution of 1 (3.28 g, 17.9 mmol) in dioxane/H₂O (25 mL) was added 2 (2.0 g, 17.9 mmol), Pd(dppf)Cl₂ (0.66 g, 0.895 mmol) and K₃PO₄ (7.59 g, 35.8 mmol). The mixture was stirred and heated to 100° C. for 3 h. The mixture was dissolved with water, extracted with EA. The organic layer was dried over Na₂SO₄ and evaporated under vacuum. The residue was purified by column chromatography on silica gel to afford 3 (0.7 g, 18% yield).

Step 2.

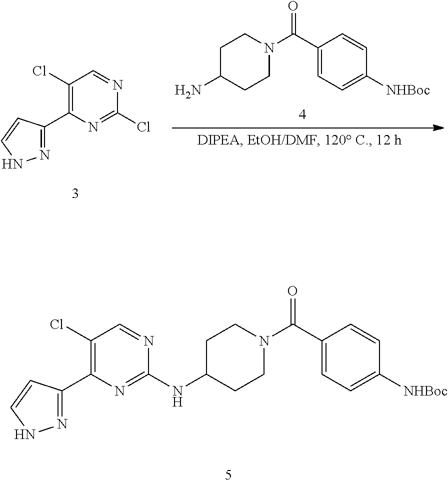 [SMILES: Clc1ncc(Cl)c(-c2cc[nH]n2)n1 ; CC(C)(C)OC(=O)Nc1ccc(C(=O)N2CCC(N)CC2)cc1 ; CC(C)(C)OC(=O)Nc1ccc(C(=O)N2CCC(Nc3ncc(Cl)c(-c4cc[nH]n4)n3)CC2)cc1]

To a solution of 3 (100 mg, 0.465 mmol) in EtOH/DMF (2.5 mL) was added intermediate 4 (Example 17; 148 mg, 0.465 mmol) and DIPEA (120 mg, 0.93 mmol). The mixture was stirred at 120° C. for 12 h. The mixture was concentrated under vacuum and the residue was purified by prep-HPLC to afford 5 (156 mg, 67% yield).

Step 3 (Compound 6).

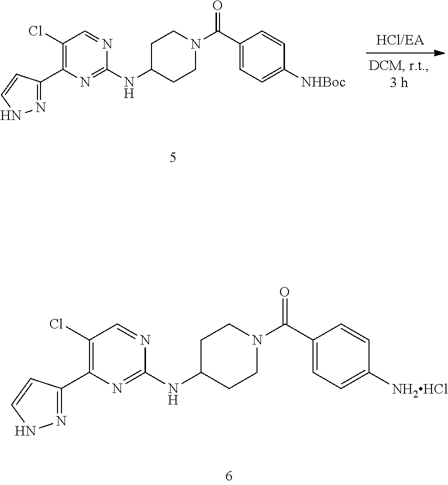

To a solution of 5 (200 mg, 0.402 mmol) in DCM (2 mL) was added HCl/EA (15 mL). The mixture was stirred at rt for 3 h. The mixture was concentrated under vacuum to afford 6 (170 mg, 100% yield).

Step 4.

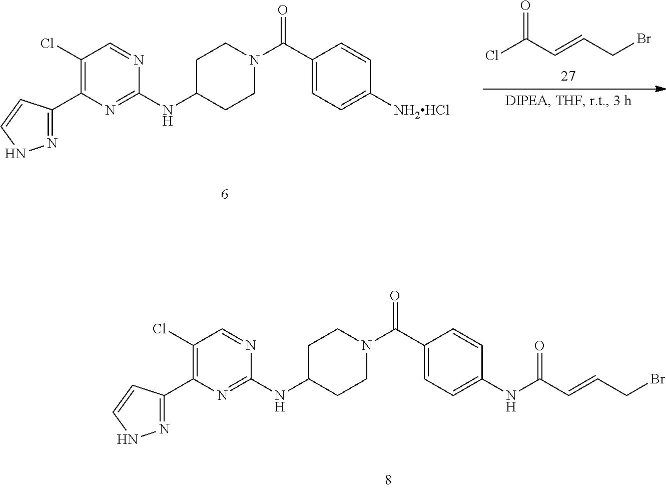

To a solution of 6 (150 mg, 0.226 mmol) in THF (5 mL) was added 27 (41 mg, 0.226 mmol) and DIPEA (117 mg, 0.904 mmol) in THF. The mixture was stirred at rt and used directly in the next step.

Step 5.

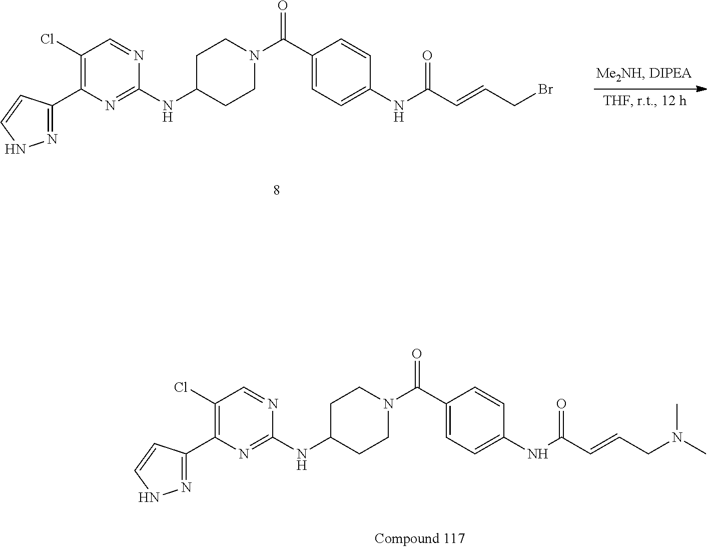 [SMILES: O=C(/C=C/CBr)Nc1ccc(C(=O)N2CCC(Nc3ncc(Cl)c(-c4cc[nH]n4)n3)CC2)cc1 ; CN(C)C/C=C/C(=O)Nc1ccc(C(=O)N2CCC(Nc3ncc(Cl)c(-c4cc[nH]n4)n3)CC2)cc1]

To a solution of 8 (123 mg, 0.226 mmol) in THF (12 mL) cooled to 0° C. was added Me₂NH (0.226 mL, 0.452 mmol). The mixture was stirred at rt for 12 h. The mixture was concentrated under vacuum and the residue was purified by prep-HPLC to afford Compound 117 (10 mg, 10% yield). ¹H NMR: (DMSO; 400 MHz) δ 10.53 (s, 1H), 9.89 (s, 1H), 8.59 (s, 1H), 8.28 (s, 1H), 7.79-7.74 (m, 3H), 7.51 (d, J=7.6 Hz, 1H), 7.4 (d, J=8.4 Hz, 2H), 6.78-6.73 (m, 1H), 6.55-6.45 (m, 2H), 4.43 (s, 2H), 4.20 (s, 1H), 3.96 (s, 2H), 3.08 (s, 1H), 2.81 (s, 6H), 1.87 (s, 2H), 1.68 (s, 2H). MS (m/z): 509 [M+1]⁺.

### Example 19. Synthesis of (E)-N-(4-(4-((5-chloro-4-(2,4-dimethylthiazol-5-yl)pyrimidin-2-yl)amino)piperidine-1-carbonyl)phenyl)-4-(dimethylamino)but-2-enamide (Compound 119)

Step 1.

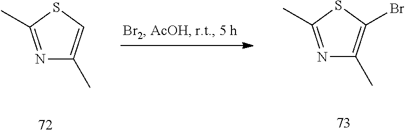

To a solution of compound 72 (1 g, 8.84 mmol) in AcOH (15 mL) was added Br₂ (2.82 g, 17.7 mmol) in AcOH (3 mL) dropwise at 0° C. and stirred at rt for 5 h. The mixture was basified to pH=10 with NaHCO₃ (solid), and then partitioned between H₂O and EA. The organic layer was dried and concentrated to afford 73 (700 mg, 41% yield).

Step 2.

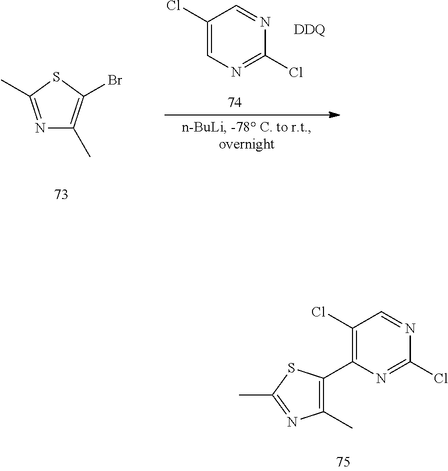

To a solution of 73 (5 g, 26 mmol) in THF (50 mL) was added n-BuLi (11 mL, 27.5 mmol) at −78° C. and stirred at −78° C. for 30 min. Then 74 in THF (15 mL) was added at −78° C., and stirred at rt overnight. H₂O (544 mg, 30 mmol) in THF (5 mL) was added at 0° C., and stirred at rt for 1 h, then DDQ (5.9 g, 26 mmol) in THF (30 mL) was added and stirred at rt overnight. The mixture was partitioned between H₂O and EA and the organic layer was dried and concentrated. The residue was purified by column (PE:EA=10:1) to afford 75 (2.2 g, 32% yield).

Step 3.

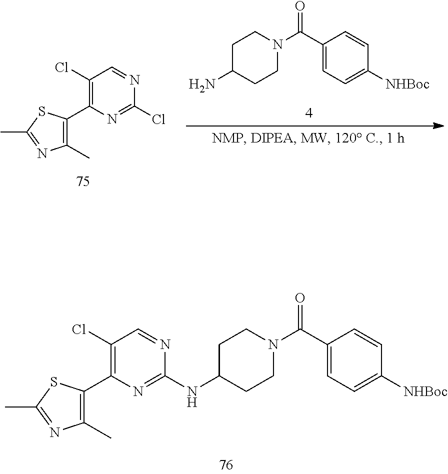 [SMILES: Cc1nc(C)c(-c2nc(Cl)ncc2Cl)s1 ; CC(C)(C)OC(=O)Nc1ccc(C(=O)N2CCC(N)CC2)cc1 ; Cc1nc(C)c(-c2nc(NC3CCN(C(=O)c4ccc(NC(=O)OC(C)(C)C)cc4)CC3)ncc2Cl)s1]

A mixture of 75 (500 mg, 1.92 mmol), 4 (614 mg, 1.92 mmol) and DIPEA (300 mg, 2.3 mmol) in NMP (6 mL) was stirred under mW at 150° C. for 1 h. The mixture was partitioned between H₂O and EA and the organic layer was dried and concentrated. The residue was purified by column (PE:EA=8:1) to afford 76 (500 mg, 49% yield).

Step 4 (Compound 77).

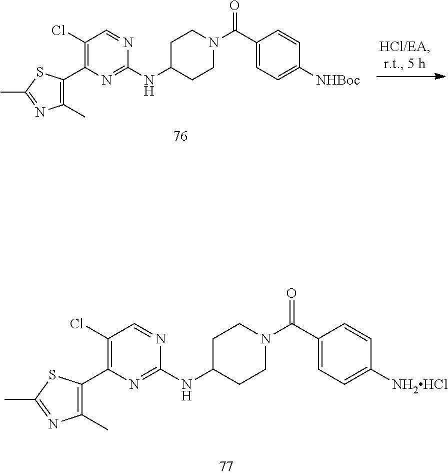

A mixture of 76 (500 mg, 0.92 mmol) in EA (5 mL) was added into a solution of HCl/EA (50 mL) and the reaction mixture was stirred at rt for 5 h. The mixture was concentrated to afford 77 (380 mg, 79% yield).

Step 5.

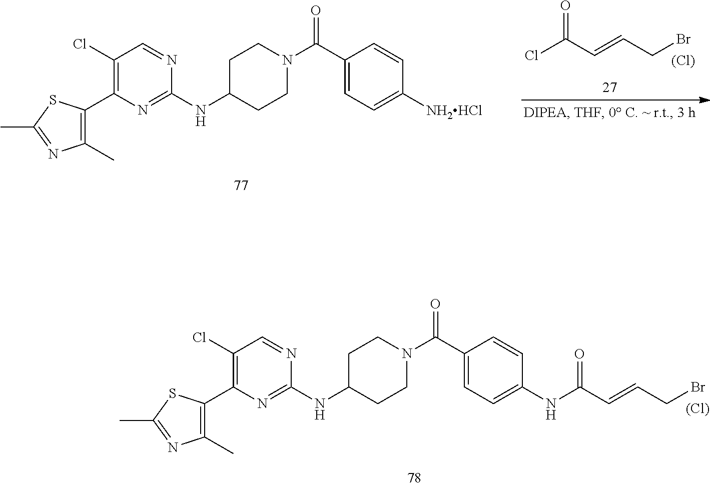

To a solution of 27 (69 mg, 0.42 mmol) in DCM (3 mL) was added oxalyl chloride (63.5 mg, 0.5 mmol) at 0° C. and the reaction mixture was stirred at rt for 1 h. The mixture was concentrated and the residue was dissolved in THF (2 mL) added into a solution of 77 (200 mg, 0.42 mmol) and DIEA (216 mg, 1.67 mmol) in THF (3 mL) and stirred at 20° C. for 3 h. The mixture was concentrated and purified by prep-HPLC to afford 78 (80 mg, 31% yield).

Step 6.

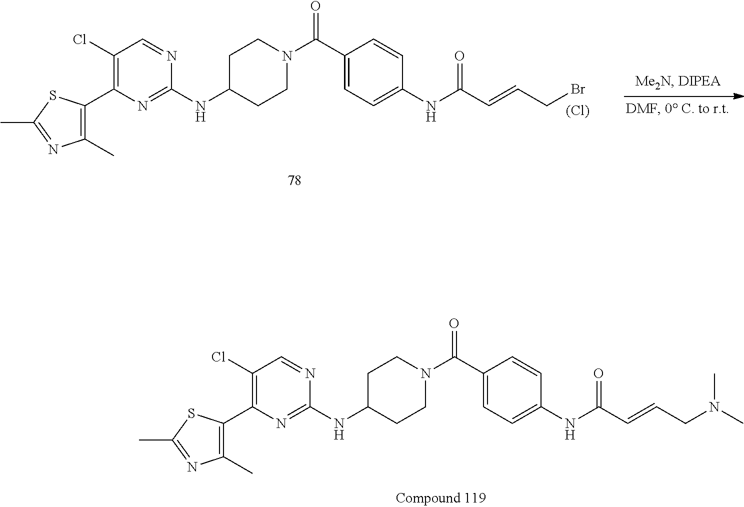

To a solution of 78 (40 mg, 73 umol) and DIEA (10 mg, 73 ul) in DMF (3 mL) was added dimethylamine (73 uL g, 147 umol) at 0° C. and the reaction mixture was stirred at rt for 6 h. The mixture was concentrated and purified by prep-HPLC to afford Compound 119 (11 mg, 27% yield). MS (m/z): [M+H] 554.2. ¹H NMR: (DMSO; 400 MHz): δ 10.39 (s, 1H), 8.38 (s, 1H), 7.74 (d, J=8 Hz, 2H), 7.36 (d, J=8 Hz, 2H), 6.87-6.79 (m, 1H), 6.54 (d, J=16 Hz, 2H), 4.02-3.92 (m, 5H), 3.10-3.06 (m, 2H), 2.80 (s, 6H), 2.65 (s, 3H), 2.44 (s, 3H), 1.95-1.91 (m, 2H), 1.53-1.51 (m, 2H).

### Example 20. Synthesis of Racemic Mixture of N-((1R,3S)-3-((5-chloro-4-(1H-indol-3-yl)pyrimidin-2-yl)amino)cyclopentyl)-4-((E)-4-(dimethylamino)but-2-enamido)benzamide and N-((1S,3R)-3-((5-chloro-4-(1H-indol-3-yl)pyrimidin-2-yl)amino)cyclopentyl)-4-((E)-4-(dimethylamino)but-2-enamido)benzamide (Compound 120)

Step 1.

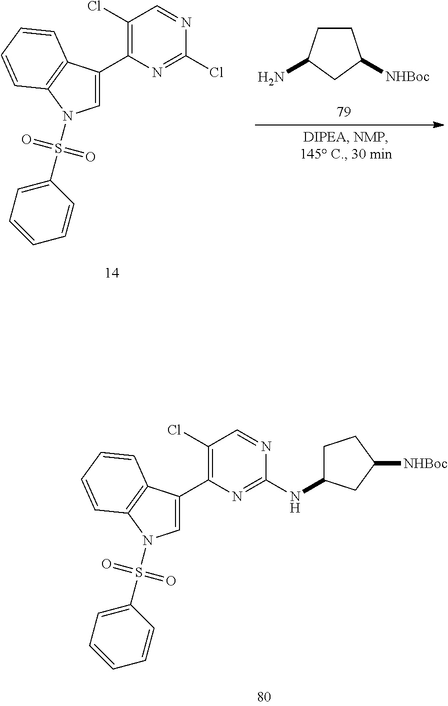

A solution of 14 (200 mg, 0.517 mmol), 79 (113.85 mg, 0.568 mmol) and DIPEA (133.60 mg, 1.034 mmol) was mixed with NMP (4 mL) and heated to 145° C. The mixture was stirred for 30 min by microwave. The reaction was diluted with water, extracted with EtOAc, and the organic phase was dried and concentrated under vacuum. The residue was purified by column to afford 80 (150 mg, 53.3%) as a yellow oil

Step 2.

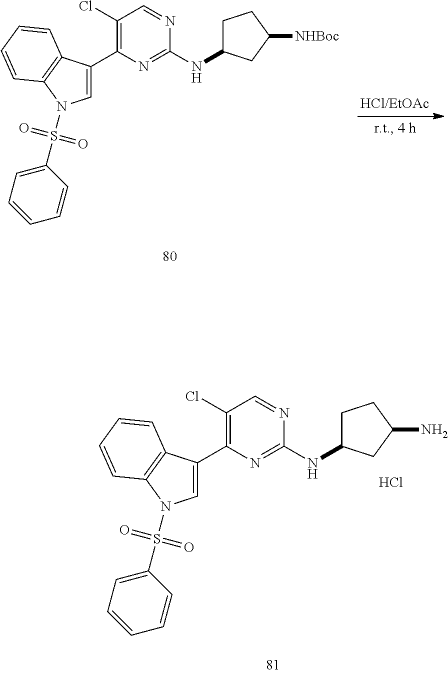

To a solution of 80 (150 mg, 0.264 mmol) in HCl/EtOAc (10 mL) was stirred at room temperature for 4 h. The mixture was concentrated under vacuum to afford 81 (150 mg, crude) as a yellow solid, which was used for next step directly.

Step 3.

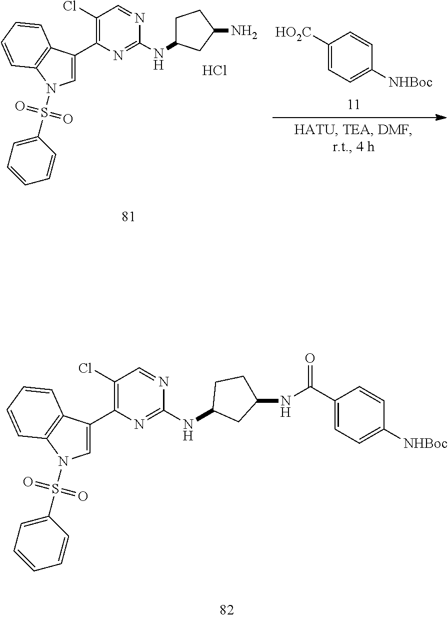

To a solution of 81 (400 mg, 0.855 mmol) in DMF (10 mL) was added 11 (223 mg, 0.94 mmol), TEA (173 mg, 1.71 mmol) and HATU (650 mg, 1.71 mmol), and the mixture was stirred at rt for 4 h. The reaction was diluted with water and extracted with EtOAc, and the organic layer was dried with anhydrous Na₂SO₄ and concentrated. The residue was purified by column to afford 82 (400 mg, 68.2%).

Step 4.

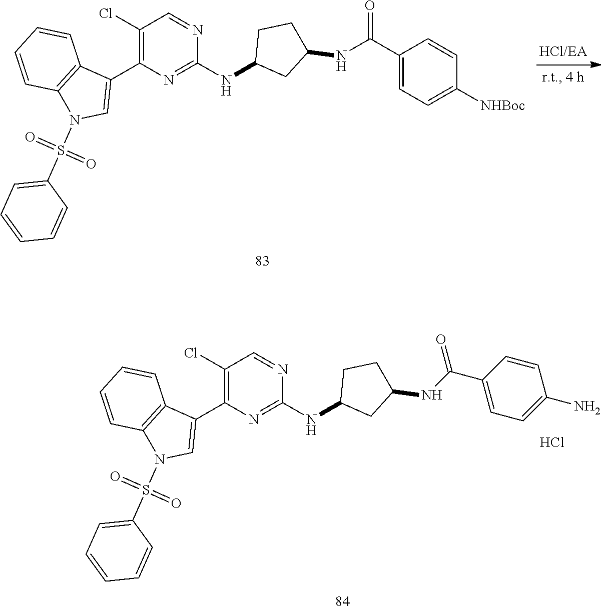

To a solution of compound 83 (400 mg, 0.583 mmol) in HCl/EA (10 mL) was stirred at room temperature for 4 h. The mixture was concentrated under vacuum to give compound 84 (250 mg, 69%) as a yellow solid.

Step 5.

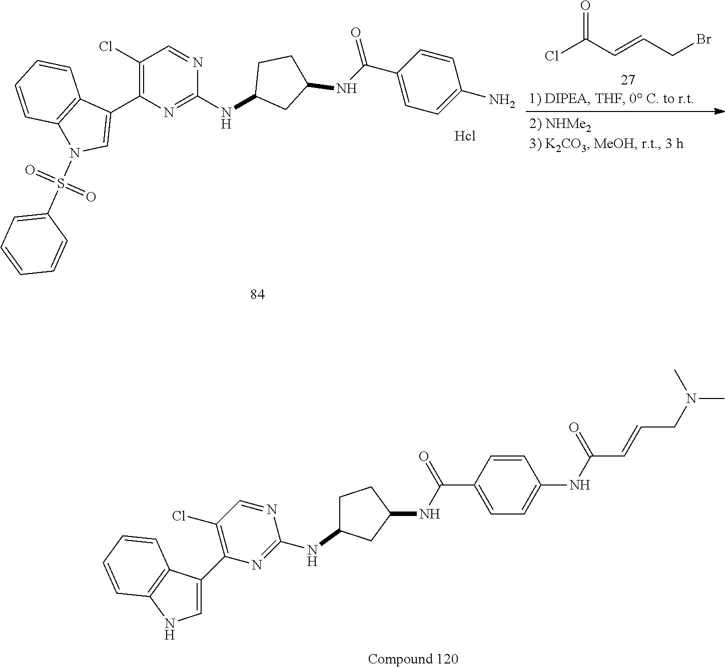

A solution of 84 (100 mg, 0.17 mmol) and 27 (62.07 mg, 0.34 mmol) in THF (3 mL) was stirred for 30 min, then DIPEA (43.9 mg, 0.34 mmol) was added. The reaction was kept stirring for 2 h. Then a solution of NHMe₂ (0.26 mL, 0.51 mmol) in THF (2 mL) was added dropwise and stirred for and additional 2 h, followed by concentration under reduced pressure. The residue was dissolved in MeOH (5 mL), charged with K₂CO₃ (50 mg, 0.36 mmol) and allowed to stir for 3 h at room temperature. The mixture was filtered and the organic phase was concentrated by vacuum. The residue was purified by prep-HPLC to afford Compound 120 (3.5 mg, 3.7%) as yellow solid. LCMS: (M+H⁺): 558. ¹H NMR: (MeOD, 400 MHz); δ 1.83-1.86 (m, 1H), 1.98-2.00 (m, 2H), 2.19-2.22 (m, 2H), 2.71-2.72 (m, 1H), 2.90 (s, 6H), 3.99 (d, J=7.2 Hz, 2H), 4.40-4.46 (m, 2H), 6.57-6.86 (m, 1H), 6.88-6.90 (m, 1H), 7.33-7.35 (m, 2H), 7.53-7.54 (m, 1H), 7.69-7.71 (m, 1H), 7.75-7.77 (m, 1H) 7.83-7.85 (m, 2H), 8.22 (s, 1H), 8.68 (brs, 1H), 8.93 (s, 1H).

### Example 21. Synthesis of (E)-N-(4-(4-((5-chloro-4-(3,5-dimethylisoxazol-4-yl)pyrimidin-2-yl)amino)piperidine-1-carbonyl)phenyl)-4-(dimethylamino)but-2-enamide (Compound 121)

Step 1.

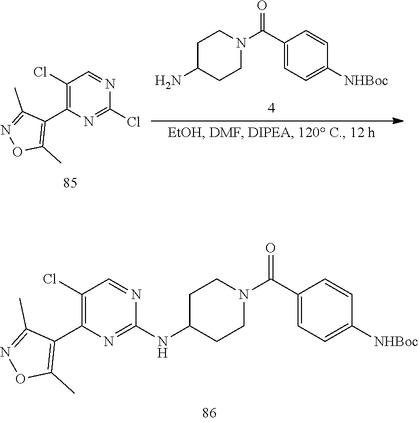 [SMILES: Cc1noc(C)c1-c1nc(Cl)ncc1Cl ; CC(C)(C)OC(=O)Nc1ccc(C(=O)N2CCC(N)CC2)cc1 ; Cc1noc(C)c1-c1nc(NC2CCN(C(=O)c3ccc(NC(=O)OC(C)(C)C)cc3)CC2)ncc1Cl]

A mixture of 85 (500 mg, 2.05 mmol), 4 (980 mg, 3 mmol) and DIPEA (529 mg, 4.1 mmol) in EtOH/DMF (V/v=4/1, 10 mL) was heated to 120° C. and stirred for 12 h. The mixture was diluted with EtOAc, washed with water and brine, and the organic phase was dried and concentrated under reduced pressure. The residue was purified by flash column to afford compound 86 (740 mg, 68.5%).

Step 2 (Compound 87)

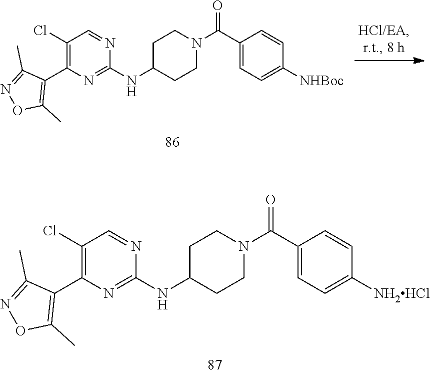

A mixture of 86 (70 mg, 1.41 mmol) in HCl/EtOAc (40 mL) was stirred at rt for 3 h. The mixture was evaporated to dryness to give 87 (20 mg, 32.7%).

Step 3.

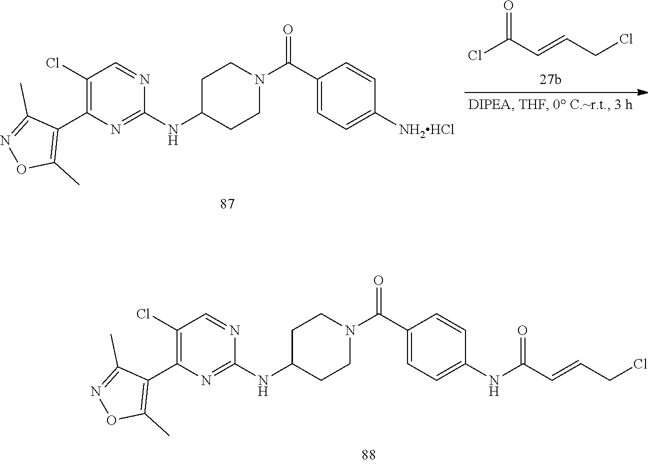

To a mixture of 87 (440 mg, 0.95 mmol) and DIPEA (490 g, 11.3 mmol) in THF (5 mL) was added 27b (183 mg, 1.00 mmol) and the reaction was stirred at rt for 3 h. The mixture was diluted with EtOAc, washed with water and brine, and the organic layer was dried over Na₂SO₄ and concentrated to afford 88 (400 mg, 73.6%).

Step 4.

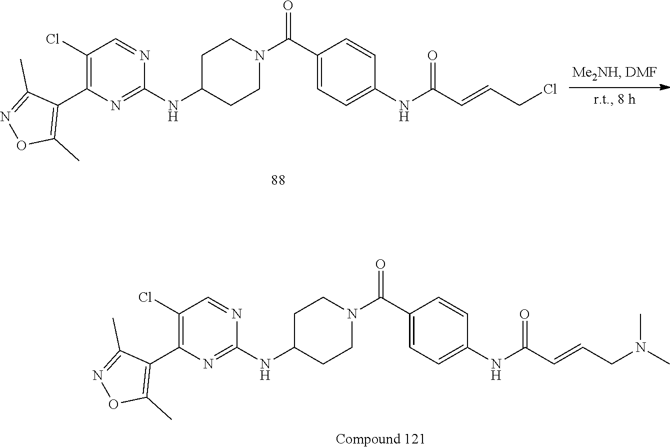 [SMILES: Cc1noc(C)c1-c1nc(NC2CCN(C(=O)c3ccc(NC(=O)/C=C/CCl)cc3)CC2)ncc1Cl ; Cc1noc(C)c1-c1nc(NC2CCN(C(=O)c3ccc(NC(=O)/C=C/CN(C)C)cc3)CC2)ncc1Cl]

A mixture of 88 (290 mg, 0.55 mmol) and Me₂NH (100 mg, 2.2 mmol) in DMF (3 mL) was stirred at rt for 8 h. The mixture was diluted with EtOAc, washed with water and brine, and the organic layer was dried and concentrated. The residue was purified by prep-HPLC to give Compound 121 (10 mg, 3.4%). ¹HNMR: TH12076-027-1A (CDCl₃, 400 MHz): δ1.59 (brs, 3H), 1.93-2.20 (m, 3H), 2.28 (s, 3H), 2.43 (s, 3H), 2.96 (s, 6H), 3.08-3.22 (m, 2H), 3.83 (brs, 1H), 4.02 (d, J=7.28 Hz, 2H), 4.10 (brs, 1H), 4.57 (brs, 1H), 6.57 (d, J=15.06 Hz, 1H), 6.83-6.96 (m, 1H), 7.46 (d, J=8.28 Hz, 2H), 7.78 (d, J=8.03 Hz, 2H), 8.38 (s, 1H).

### Example 22. Synthesis of a Racemic Mixture of N-((1R,3S)-3-((5-chloro-4-(3,5-dimethylisoxazol-4-yl)pyrimidin-2-yl)amino)cyclopentyl)-4-((E)-4-(dimethylamino)but-2-enamido)benzamide and N-((1S,3R)-3-((5-chloro-4-(3,5-dimethylisoxazol-4-yl)pyrimidin-2-yl)amino)cyclopentyl)-4-((E)-4-(dimethylamino)but-2-enamido)benzamide (Compound 122)

Step 1.

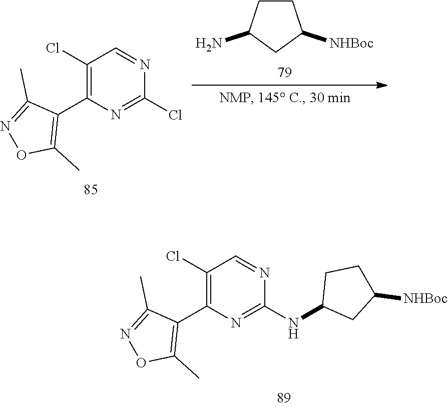

A mixture of 85 (500 mg, 2.05 mmol), 79 (492 mg, 2.46 mmol) and DIPEA (529 mg, 4.1 mmol) in NMP (5 mL) was heated to 145° C. stirred for 30 min (mW). After cooling to rt, the mixture was dissolved in EtOAc, washed with water and brine, and the organic phase was dried and concentrated under reduced pressure. The residue was purified by flash column to afford 89 (628 mg, 73.5%).

Step 2.

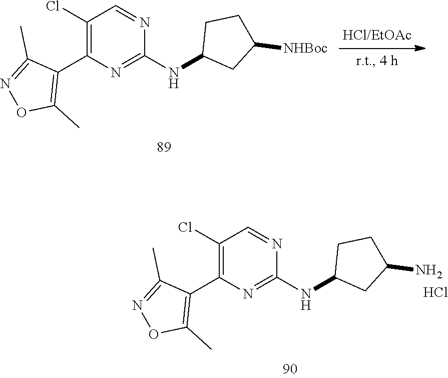

A mixture of 89 (100 mg, 0.25 mmol) in HCL/EtOAc (25 mL) was stirred at room temperature for 4 h. The mixture was evaporated to dryness to give 90 (70 mg, 82.8%).

Step 3.

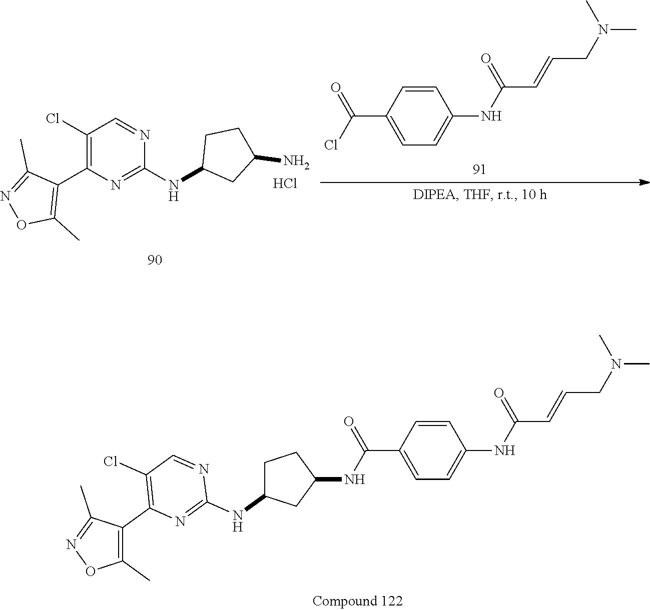

To a mixture of 90 (70 mg, 0.2 mmol) and DIPEA (490 g, 11.3 mmol) in THF (5 mL) was added 91 (183 mg, 1.00 mmol). The mixture was stirred at rt for 10 h, then diluted with EtOAc, washed with water and brine, and the organic layer was dried over Na₂SO₄ and concentrated. The residue was purified by prep-HPLC to afford Compound 122 (11 mg, 10.1%). ¹HNMR: TH06208-031-1 (CDCl₃, 400 MHz): δ 1.60-1.71 (m, 1H), 1.76-1.90 (m, 2H), 2.04-2.18 (m, 2H), 2.29 (s, 3H), 2.43 (s, 3H), 2.52-2.63 (m, 1H), 2.9-2.99 (m, 6H), 4.02 (d, J=7.28 Hz, 2H), 4.27-4.46 (m, 2 H), 6.59 (d, J=15.31 Hz, 1H), 6.90 (dt, J=14.93, 7.34 Hz, 1H), 7.74-7.90 (m, 4H), 8.37 (s, 1H).

### Example 23. Synthesis of a Racemic Mixture of N-((1R,3S)-3-((5-chloro-4-(pyridin-3-yl)pyrimidin-2-yl)amino)cyclopentyl)-4-((E)-4-(dimethylamino)but-2-enamido)benzamide and N-((1S,3R)-3-((5-chloro-4-(pyridin-3-yl)pyrimidin-2-yl)amino)cyclopentyl)-4-((E)-4-(dimethylamino)but-2-enamido)benzamide (Compound 123)

Step 1.

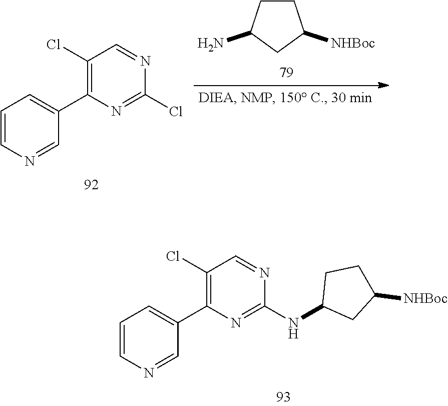

A solution of 92 (200 mg, 0.89 mmol), 79 (196.7 mg, 0.98 mmol) and DIPEA (230 mg, 1.78 mmol) was dissolved in NMP (4 mL), and the mixture was heated to 145° C. and stirred for 30 min by microwave. The mixture was diluted with water and extracted with EtOAc, and the organic phase was dried and concentrated under vacuum. The residue was purified by column to afford 93 (250 mg, 72.2%) as a yellow oil

Step 2.

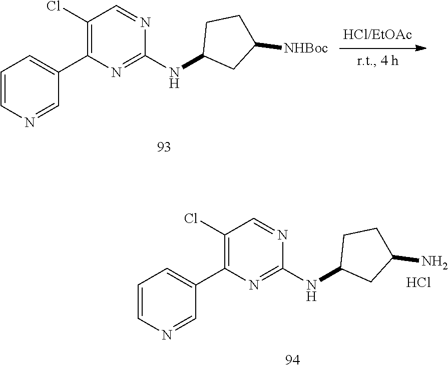

A solution of 93 (250 mg, 0.64 mmol) in HCl/EtOAc (10 mL) was stirred at room temperature for 4 h. The mixture was concentrated under vacuum to give 94 (180 mg, 86.2%) as a yellow solid.

Step 3.

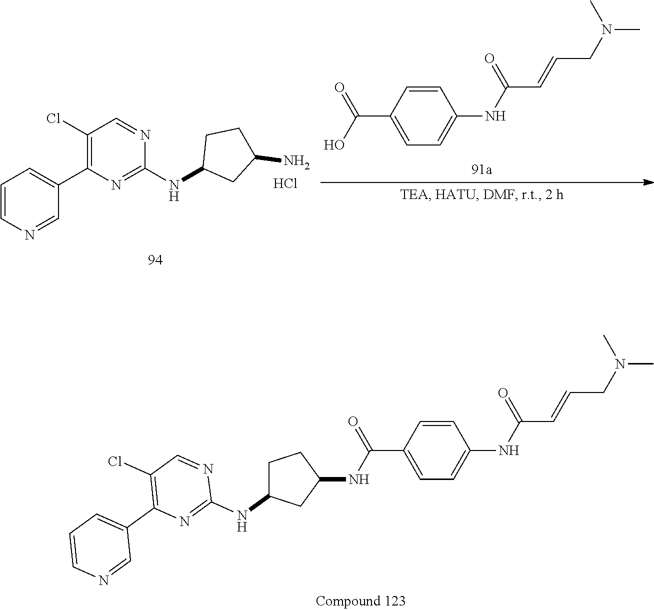

To a solution of 94 (100 mg, 0.308 mmol) in DMF (2 mL) was added 91a (70.5 mg, 0.308 mmol), TEA (62 mg, 0.616 mmol) and HATU (175.5 mg, 0.462 mmol). The mixture was stirred at rt for 2 h, after which it was concentrated under vacuum and the subsequent residue was purified by prep-HPLC to afford Compound 123 (21 mg, 13.1%). LCMS: (M+H+⁺): 520. ¹H NMR: TH06207-043-1 (MeOD, 400 MHz): δ 1.62-1.66 (m, 1H), 1.81-1.84 (m, 2H), 2.08-2.13 (m, 2H), 2.57-2.60 (m, 1H), 2.93 (s, 6H), 3.99 (d, J=6 Hz, 2H), 4.33-4.40 (m, 2H), 6.54-6.58 (m, 1H), 6.84-6.89 (m, 1H), 7.73-7.77 (m, 2H), 7.81-7.83 (m, 2H), 7.83 (brs, 1H), 8.40 (s, 1H), 8.72-8.74 (m, 1H), 8.84 (brs, 1H), 9.19 (br, 1H).

### Example 24. Synthesis of Intermediate 91a

Step 1.

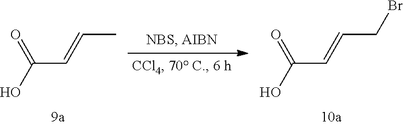

A mixture of 9a (50 g, 581 mmol), NBS (105 g, 592 mmol) and AIBN (1.6 g, 11.6 mmol) in CCl₄ (700 mL) was stirred at reflux for 6 h. The mixture was concentrated and recrystallized with PE to afford 10a (37 g, 39% yield).

Step 2.

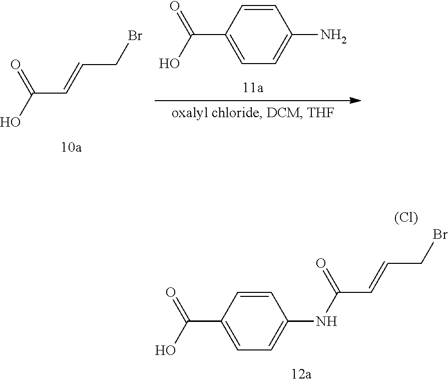

To a solution of 10a (5 g, 30 mmol) in DCM (50 mL) was added oxalyl chloride (4.6 g, 36.4 mmol) in DCM (30 mL) and a drop of DMF. The reaction was stirred at rt for 2 h, then concentrated and dissolved in THF (30 mL). The mixture was then added into a solution of 11a (4.15 g, 30 mmol) in THF (50 mL) and stirred at rt for 5 h, followed by concentration and purification by column (PE:EA=3:1) to afford 12a (1 g, 11% yield).

Step 3.

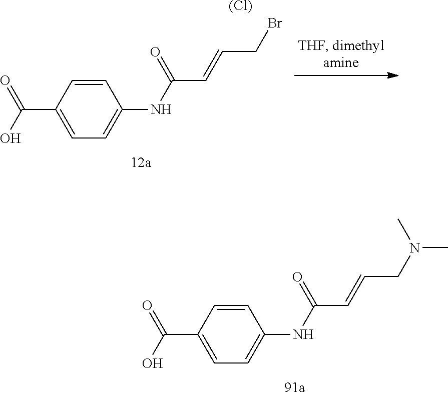

To a mixture of 12a (500 mg, 2.09 mmol) and DIEA (324 mg, 2.5 mmol) in THF (10 mL) was added dimethyl amine (1.25 mL, 2.5 mmol). The reaction was stirred at rt for 3 h, after which the mixture was concentrated and purified by prep-HPLC to afford intermediate 91a (140 mg, 27% yield).

### Example 25. Synthesis of a Racemic Mixture of N-((1R,3S)-3-((5-chloro-4-(1H-pyrazol-3-yl)pyrimidin-2-yl)amino)cyclopentyl)-4-((E)-4-(dimethylamino)but-2-enamido)benzamide and N-((1S,3R)-3-((5-chloro-4-(1H-pyrazol-3-yl)pyrimidin-2-yl)amino)cyclopentyl)-4-((E)-4-(dimethylamino)but-2-enamido)benzamide (Compound 124)

Step 1.

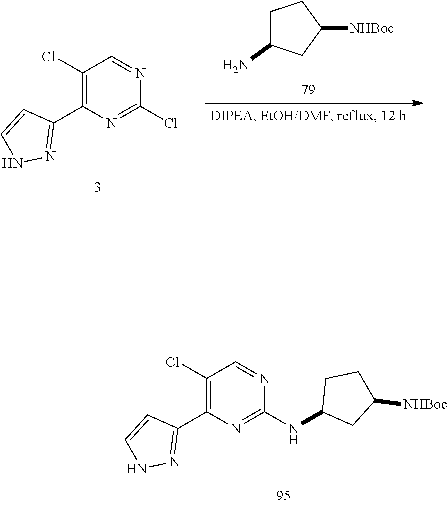

To a solution of 3 (200 mg, 0.93 mmol) in EtOH/DMF (5 mL) was added 79 (280 mg, 1.4 mmol) and DIPEA (240 mg, 1.86 mmol) under N₂. The mixture was stirred at 100° C. for 10 h, and the mixture was concentrated under vacuum. The residue was purified by column chromatography on silica gel to afford 95 (340 mg, 96% yield).

Step 2.

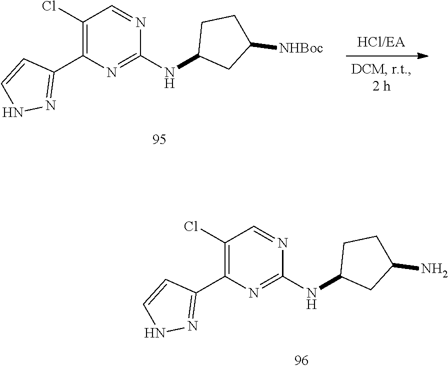

To a solution of 95 (400 mg, 1.055 mmol) in DCM (2 mL) was added HCl/EA (20 mL). The mixture was stirred at rt for 2 h, then filtered, and the filter cake was concentrated under vacuum to afford 96 (275 mg, 94% yield).

Step 3.

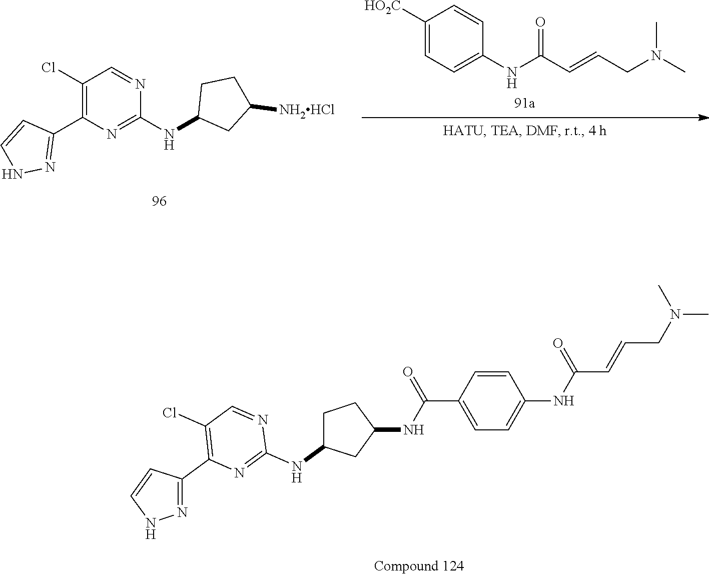

To a solution of 96 (56 mg, 0.2 mmol) in DMF (2 mL) was added 91a (50 mg, 0.2 mmol), TEA (61 mg, 0.6 mmol), and HATU (76 mg, 0.2 mmol). The mixture was stirred at rt for 4 h and concentrated under vacuum, after which the residue was purified by prep-HPLC to afford Compound 124 (20 mg, 19% yield). LCMS: (M+H⁺): 509. ¹H NMR: TH06398-025-1; (DMSO; 400 MHz): δ 10.57 (s, 1H), 10.11 (s, 1H), 8.59 (s, 1H), 8.58 (d, J=0.8 Hz, 1H), 8.27 (s, 1H), 7.86-7.65 (m, 5H), 6.86-6.70 (m, 1H), 6.54-6.50 (m, 2H), 4.58-4.54 (m, 1H), 4.30-4.25 (m, 1H), 4.62-4.57 (m, 1H), 3.94 (d, J=7.2 Hz, 1H), 2.79 (s, 6H), 2.03-1.72 (m, 6H).

### Example 26. Synthesis of (E)-N-(4-(4-((5-chloro-4-(2-methyl-1H-indol-3-yl)pyrimidin-2-yl)amino)piperidine-1-carbonyl)phenyl)-4-(dimethylamino)but-2-enamide (Compound 125)

Step 1

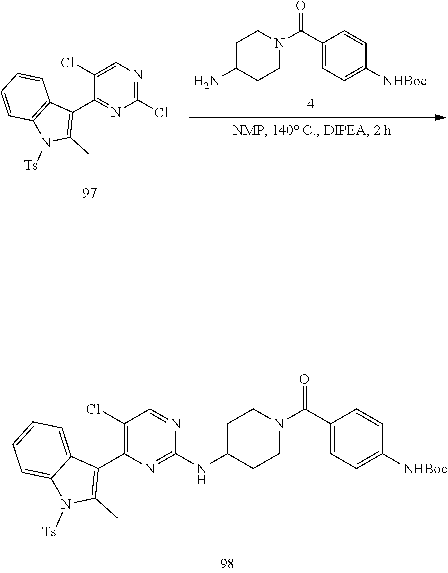 [SMILES: Cc1ccc(S(=O)(=O)n2c(C)c(-c3nc(Cl)ncc3Cl)c3ccccc32)cc1 ; CC(C)(C)OC(=O)Nc1ccc(C(=O)N2CCC(N)CC2)cc1 ; Cc1ccc(S(=O)(=O)n2c(C)c(-c3nc(NC4CCN(C(=O)c5ccc(NC(=O)OC(C)(C)C)cc5)CC4)ncc3Cl)c3ccccc32)cc1]

A mixture of 97 (500 mg, 1.16 mmol), 4 (406 mg, 1.27 mmol) and DIPEA (180 mg, 1.39 mmol) in NMP (8 mL) was stirred under mW at 150° C. for 2 h. The mixture was partitioned between H₂O and EA, and the organic layer was dried and concentrated. The residue was purified by column (PE:EA/5:1) to afford 98 (400 mg, 48% yield).

Step 2.

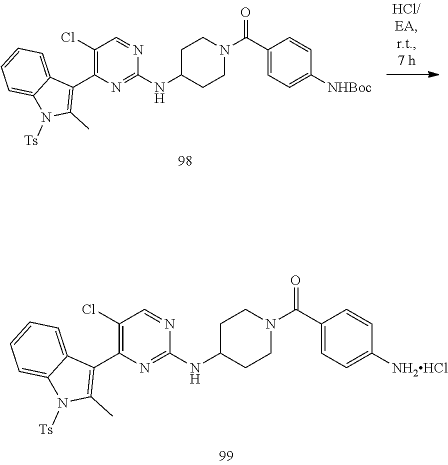

To a solution of HCl/EA (30 mL) was added 98 (420 mg, 0.59 mmol) in EA (3 mL), and the mixture was allowed to stir at rt for 7 h, then concentrated to afford 99 (350 mg, 91% yield).

Step 3 (Compound 1a)

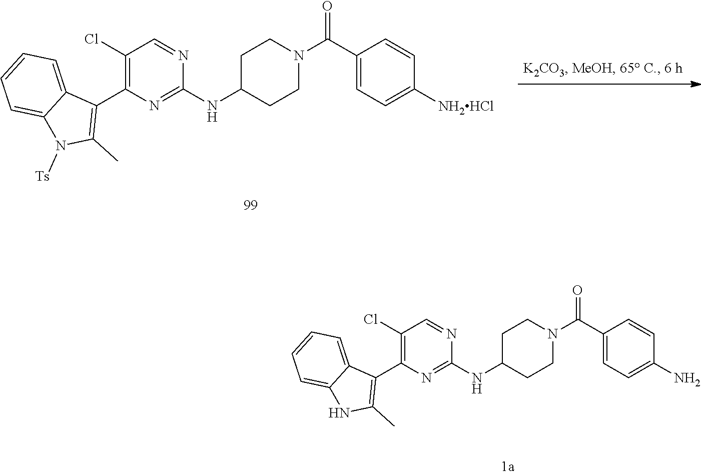

A mixture of 99 (100 mg, 0.153 mmol) and K₂CO₃ (64 mg, 0.46 mmol) in MeOH (3 mL) was stirred at reflux for 6 h. The mixture was filtered and the residue was concentrated to afford 1a (60 mg, 85% yield).

Step 4.

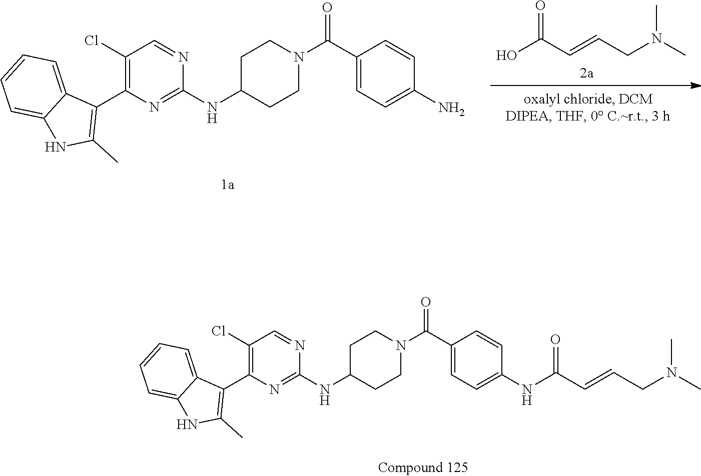

To a solution of 1a (16 mg, 0.124 mmol) in DCM (2 mL) was added oxalyl chloride (16 mg, 0.126 mmol) in DCM (1 mL). The mixture was stirred at rt for 2 h, then added into a solution of 2a (55 mg, 0.12 mmol) and DIPEA (62 mg, 0.48 mmol) in THF (3 mL). After stirring at rt for 3 h, the mixture was concentrated and purified by prep-HPLC to afford Compound 125 (6.5 mg, 9% yield). MS found: [M+H] 572.2. ¹H NMR: (MeOH; 400 MHz): δ 8.35 (s, 1H), 7.78 (d, J=8 Hz, 2H), 7.48-7.44 (m, 3H), 7.35 (d, J=8 Hz, 2H), 7.12-7.05 (m, 2H), 6.89-6.87 (m, 1H), 6.57 (d, J=16 Hz, 2H), 4.65-4.55 (m, 1H), 4.20-4.10 (m, 1H), 4.01 (d, J=8 Hz, 2H), 3.95-3.75 (m, 1H), 2.95 (s, 6H), 2.51 (s, 1H), 2.25-2.0 (m, 2H), 1.7-1.5 (m, 2H).

### Example 27. Synthesis of a Racemic Mixture of N-((1R,3S)-3-((5-chloro-4-(2-methyl-1H-indol-3-yl)pyrimidin-2-yl)amino)cyclopentyl)-4-((E)-4-(dimethylamino)but-2-enamido)benzamide and N-((1S,3R)-3-((5-chloro-4-(2-methyl-1H-indol-3-yl)pyrimidin-2-yl)amino)cyclopentyl)-4-((E)-4-(dimethylamino)but-2-enamido)benzamide (Compound 126)

Step 1.

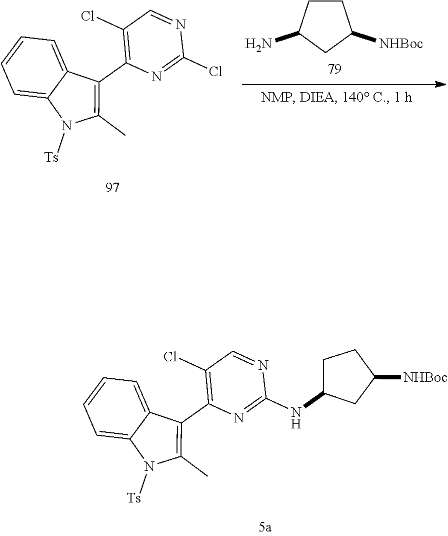

A mixture of 97 (500 mg, 1.16 mmol) and compound 4 (231.6 mg, 1.16 mmol) in NMP (4 mL) was stirred under mW at 140° C. for 1 h. The mixture was partitioned between H₂O and EA and the organic layer was dried and concentrated. The residue was purified by column (PE:EA=3:1) to afford 5a (300 mg, 43% yield).

Step 2.

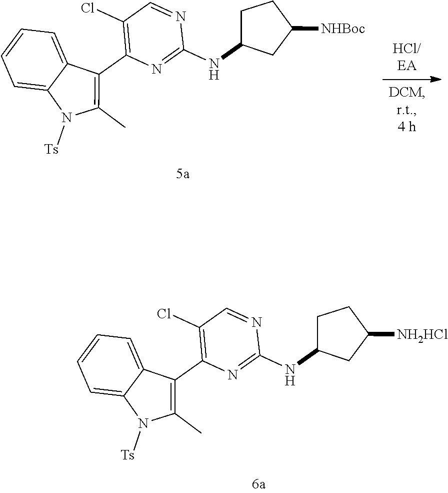

A mixture of 5a (300 mg, 0.5 mmol) and HCl/EA (35 mL) was stirred at rt for 5 h, after which the mixture was concentrated to afford 6a (250 mg, 93% yield).

Step 3.

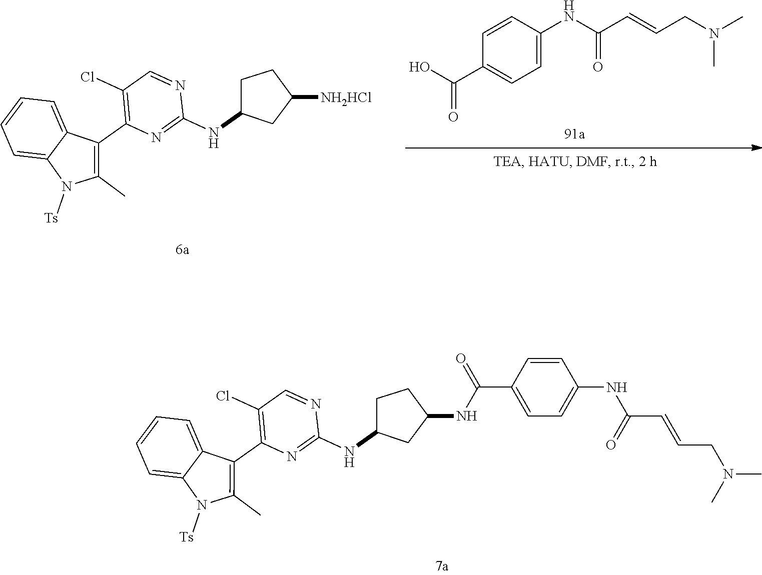

To a solution of 6a (69 mg, 0.42 mmol) in DMF (2 mL) was added 91a (65 mg, 0.262 mmol), HATU (109.6 mg, 0.2888 mmol), and TEA (79.56 mg, 0.786 mmol). The reaction mixture was stirred at 10° C. for 2 h, then concentrated and purified by prep-HPLC to afford 7a (70 mg, 33% yield).

Step 4.

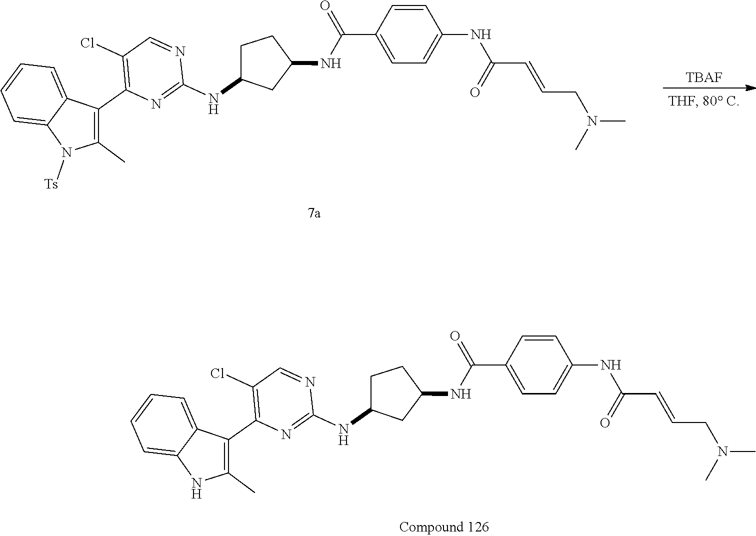

To a solution of 7a (50 mg, 68.84 umol) in THF (2 mL) was added TBAF (36 mg, 137.68 umol), and the reaction mixture was stirred at 80° C. for 6 h. The mixture was concentrated and purified by prep-HPLC to afford Compound 126 (2 mg, 4.98% yield). MS found: [M+H] 572.2. ¹H NMR: (DMSO; 400 MHz): δ 8.34 (s, 1H), 7.83 (d, J=8 Hz, 2H), 7.75 (d, J=8.8 Hz, 2H), 7.48 (d, J=7.6 Hz, 1H), 7.34 (d, J=7.6 Hz, 1H), 7.11-7.06 (m, 2H), 7.04-6.88 (m, 1H), 6.56 (d, J=15.2 Hz, 1H), 4.39-4.37 (m, 2H), 4.00 (d, J=8 Hz, 2H), 2.93 (s, 6H), 2.61-2.51 (m, 4H), 2.15-2.05 (m, 2H), 1.90-1.80 (m, 2H), 1.69-1.65 (m, 1H).

### Example 28. Synthesis of (E)-N-(4-(4-((5-chloro-4-(1H-imidazol-4-yl)pyrimidin-2-yl)amino)piperidine-1-carbonyl)phenyl)-4-(dimethylamino)but-2-enamide (Compound 127)

Step 1.

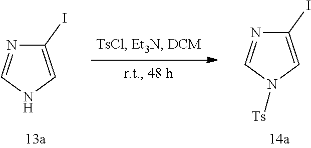 [SMILES: Ic1c[nH]cn1 ; Cc1ccc(S(=O)(=O)n2cnc(I)c2)cc1]

To a mixture of 13a (5 g, 0.026 mol) in DCM (100 mL) was added Et₃N (3.9 g, 0.038 mol) and TsCl (5.87 g, 0.030 mol) at 0° C., and the mixture stirred at rt for 48 h. The reaction was poured into water (100 mL) and extracted with EtOAc (200 mL). The organic layer was dried with Na₂SO₄ and concentrated under reduced pressure. The residue was purified by flash column to afford 14a (6.0 g, 66.8%).

Step 2.

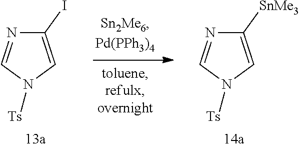

To a mixture of 13a (5 g, 14.3 mmol) in toluene (50 mL) was added Sn₂Me₆ (7 g, 21.5 mmol) and Pd(PPh₃)₄ (1.6 g, 1.43 mmol). The mixture was degassed three times, heated to reflux and stirred overnight. The mixture was concentrated under reduced pressure to afford crude 14a (5 g, crude), which was used for next step directly.

Step 3.

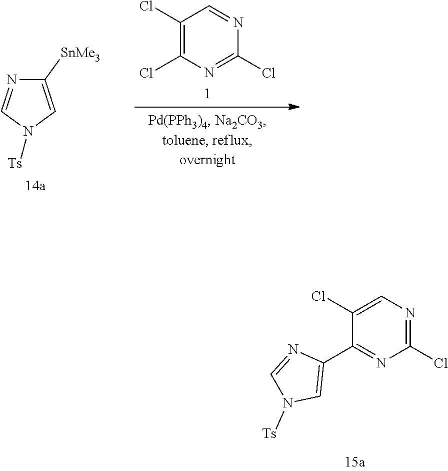

A mixture of 14a (12.2 g, crude), 1 (3.95 g, 21.5 mmol), Pd(PPh₃)₄ (1.6 g, 1.43 mmol) and Na₂CO₃ (3.0 g, 28.7 mmol) in toluene (100 mL) was degassed for three times, heated to reflux, and stirred overnight. The mixture was concentrated under reduced pressure, and the residue was purified by flash column to afford 15a (1.4 g, 18.8% for two steps).

Step 4.

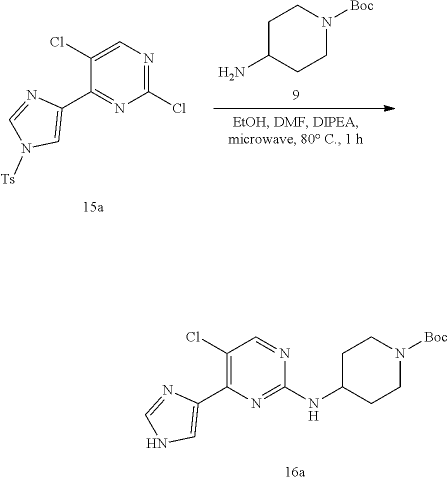 [SMILES: Cc1ccc(S(=O)(=O)n2cnc(-c3nc(Cl)ncc3Cl)c2)cc1 ; CC(C)(C)OC(=O)N1CCC(N)CC1 ; CC(C)(C)OC(=O)N1CCC(Nc2ncc(Cl)c(-c3c[nH]cn3)n2)CC1]

To a solution of 15a (500 mg, 1.35 mmol) in EtOH (4 mL) and DMF (1 mL) was added 9 (406 mg, 2.03 mmol) and DIPEA (350 mg, 2.70 mmol). The mixture was stirred under microwave at 80° C. for 8 h. The reaction mixture was diluted with EtOAc, washed with water and brine, and the organic layer was dried and concentrated. The residue was purified by prep-HPLC to afford 16a (200 mg, 38.9%).

Step 5.

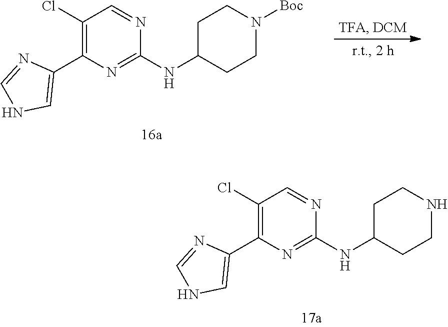 [SMILES: CC(C)(C)OC(=O)N1CCC(Nc2ncc(Cl)c(-c3c[nH]cn3)n2)CC1 ; Clc1cnc(NC2CCNCC2)nc1-c1c[nH]cn1]

A solution of 16a (150 mg, 0.39 mmol) in DCM (2 mL) and TFA (0.5 mL) was stirred at rt for 2 h. The mixture was evaporated under reduced pressure to afford 17a (200 mg, crude), which was used for next step directly.

Step 6.

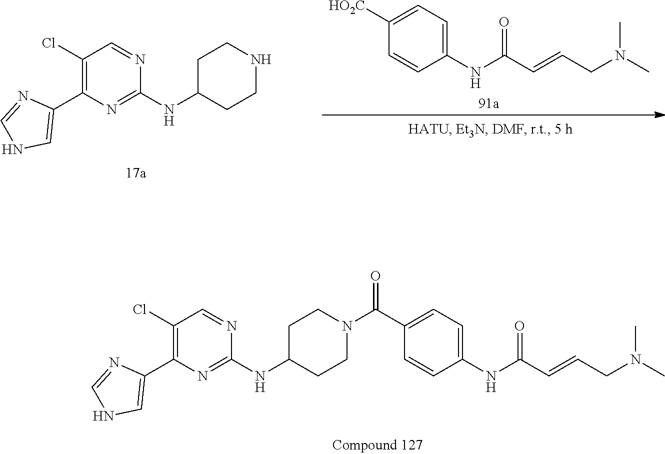 [SMILES: Clc1cnc(NC2CCNCC2)nc1-c1c[nH]cn1 ; CN(C)C/C=C/C(=O)Nc1ccc(C(=O)O)cc1 ; CN(C)C/C=C/C(=O)Nc1ccc(C(=O)N2CCC(Nc3ncc(Cl)c(-c4c[nH]cn4)n3)CC2)cc1]

To a solution of 17a (150 mg, crude) in DMF (2 mL) was added 91a (200 mg, 0.81 mmol), Et₃N (109 mg, 1.07 mmol) and HATU (225 mg, 0.81 mmol). The mixture was stirred at rt for 5 h, then diluted with EtOAc, and washed with water and brine. The organic layer was dried and concentrated and the residue was purified by prep-HPLC to afford Compound 127 (40 mg, 19.8% for two steps). ¹H NMR: (DMSO, 400 MHz); δ10.53 (s, 1H), 9.92 (s, 1H), 8.31-8.52 (m, 3H), 7.75 (d, J=8.28 Hz, 2H), 7.54 (d, J=7.53 Hz, 1H), 7.40 (d, J=8.28 Hz, 2H), 6.69-6.85 (m, 1H), 6.48 (d, J=15.31 Hz, 1H), 4.40 (s, 3H), 4.04 (s, 1H), 4.04 (s, 1H), 3.96 (d, J=6.78 Hz, 2H), 3.72 (s, 2H), 3.14 (brs, 1H), 2.82 (s, 6H), 1.92 (brs, 2H), 1.48 (brs, 2H).

### Example 29. Synthesis of a Racemic Mixture of N-((1R,3S)-3-((5-chloro-4-(2,4-dimethylthiazol-5-yl)pyrimidin-2-yl)amino)cyclopentyl)-4-((E)-4-(dimethylamino)but-2-enamido)benzamide and N-((1S,3R)-3-((5-chloro-4-(2,4-dimethylthiazol-5-yl)pyrimidin-2-yl)amino)cyclopentyl)-4-((E)-4-(dimethylamino)but-2-enamido)benzamide (Compound 128)

Step 1.

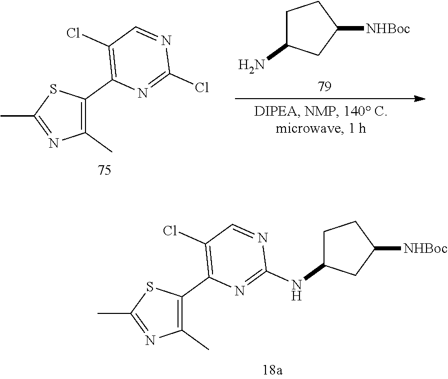 [SMILES: Cc1nc(C)c(-c2nc(Cl)ncc2Cl)s1 ; CC(C)(C)OC(=O)N[C@@H]1CC[C@H](N)C1 ; Cc1nc(C)c(-c2nc(N[C@H]3CC[C@@H](NC(=O)OC(C)(C)C)C3)ncc2Cl)s1]

To a solution of 75 (300 mg, 1.15 mmol) in NMP (2 mL) was added 79 (231 mg, 1.15 mmol) and DIPEA (298 mg, 2.30 mmol) under N₂. The mixture was stirred at 140° C. under microwave for 1 h. The mixture was then dissolved in water and EA and extracted with EA. The organic layer was dried over Na₂SO₄ and concentrated under vacuum. The residue was purified by prep-HPLC to afford 18a (300 mg, 61% yield).

Step 2.

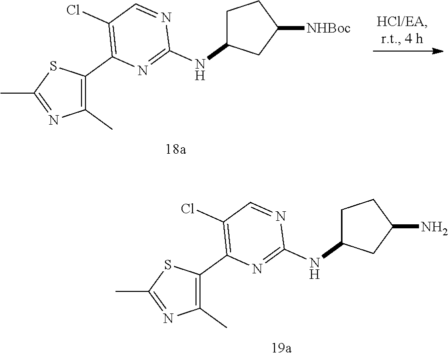 [SMILES: Cc1nc(C)c(-c2nc(N[C@H]3CC[C@@H](NC(=O)OC(C)(C)C)C3)ncc2Cl)s1 ; Cc1nc(C)c(-c2nc(N[C@H]3CC[C@@H](N)C3)ncc2Cl)s1]

To a solution of 18a (300 mg, 0.708 mmol) in DCM (2 mL) was added HCl/EA (20 mL). The mixture was stirred at rt for 4 h, then concentrated under vacuum to afford 19a (240 mg, 94% yield).

Step 3.

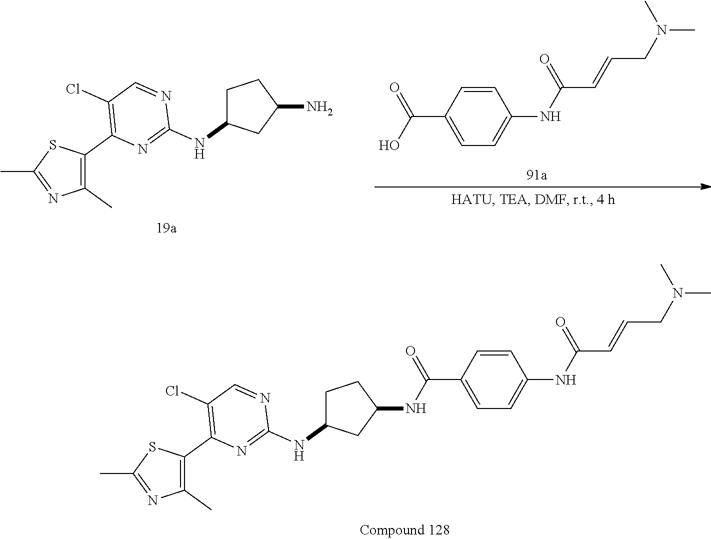

To a solution of 19a (100 mg, 0.309 mmol) in DMF (2 mL) was added 91a (84 mg, 0.339 mmol), TEA (94 mg, 0.926 mmol), and HATU (129 mg, 0.339 mmol). The mixture was stirred at rt for 4 h then concentrated under vacuum. The residue was purified by prep-HPLC to afford Compound 128 (50 mg, 29% yield). LCMS: (M+H⁺): 554. ¹H NMR: (MeOD; 400 MHz): δ 8.33 (s, 1H), 7.83-7.81 (m, 2H), 7.76-7.74 (m, 2H), 6.91-6.84 (m, 1H), 6.56 (d, J=15.2 Hz, 1H), 4.38-4.29 (m, 2H), 4.00 (d, J=7.6 Hz, 2H), 2.93 (s, 6H), 2.73 (s, 3H), 2.58-2.55 (m, 1H), 2.51 (s, 3H), 2.12-2.08 (m, 2H), 1.83-1.80 (m, 2H), 1.64-1.61 (m, 1H).

### Example 30. Synthesis of N-((1R,3S)-3-((5-chloro-4-(1H-indol-3-yl)pyrimidin-2-yl)amino)cyclopentyl)-4-((E)-4-(dimethylamino)but-2-enamido)benzamide (Compound 129) and N-((1S,3R)-3-((5-chloro-4-(1H-indol-3-yl)pyrimidin-2-yl)amino)cyclopentyl)-4-((E)-4-(dimethylamino)but-2-enamido)benzamide (Compound 130)

Step 1.

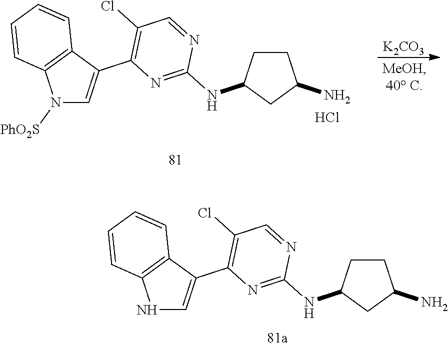

A mixture of 81 (0.7 g, 1.39 mmol) in MeOH (10 mL) was added K₂CO₃ (0.96 g, 6.94 mmol) at rt. Then the reaction mixture was heated to 40° C. and stirred for 1 h. LCMS showed the reaction was completed and the reaction mixture was filtered. The filtrate was concentrated and purified by prep-HPLC to obtain 81a (300 mg, purity: 90% TLC)

Step 2.

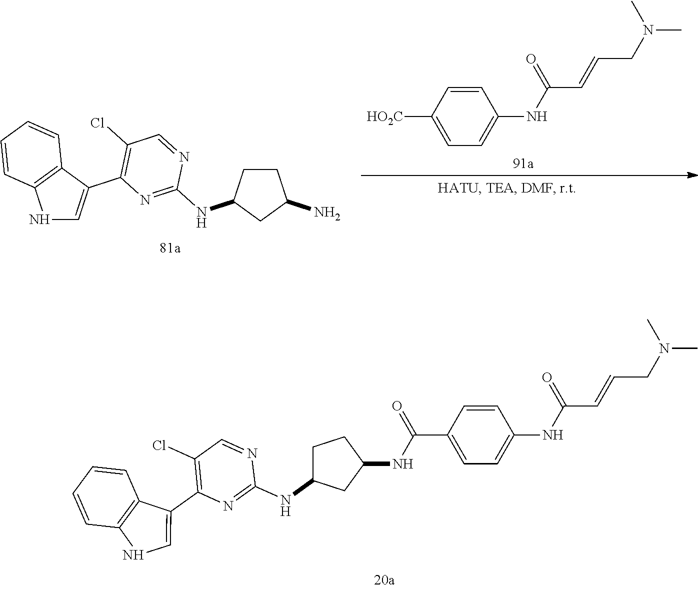

A mixture of 91a (249.94 mg, 1.01 mmol) in DMF (5 mL) was added DIPEA (236.55 mg, 1.83 mmol) and HATU (382.77 mg, 1.01 mmol), then the mixture was stirred at rt for 1 hr. 81a (300.00 mg, 0.91 mmol) was added to the mixture and stirred at rt for 2 hr. The mixture was concentrated and purified by prep-HPLC to afford 20a (120.00 mg, purity: 96% on HPLC). ¹H NMR: (400 MHz; d₆-DMSO): δ ppm 11.84 (s, 1H), 10.27 (s, 1H), 8.62 (s, 1H), 8.45 (d, J=2.4 Hz, 1H), 8.31 (d, J=7.6 Hz, 1H), 8.27 (s, 1H), 7.82 (d, J=8.8 Hz, 2H), 7.72 (d, J=8.8 Hz, 2H), 7.49 (d, J=8 Hz, 1H), 7.41 (d, J=8 Hz, 1H), 7.23-7.16 (m, 2H), 6.79-6.73 (m, 1H), 6.28 (d, J=8.4 Hz, 2H), 4.32 (s, 2H), 3.07 (d, J=5.2 Hz, 2H), 2.46-2.45 (m, 2H), 2.18 (s, 6H), 2.00-1.98 (m, 2H), 1.78-1.73 (m, 2H), 1.66-1.59 (m, 1H).

Step 3.

20a was separated by SFC to obtain the two isomers: Compound 129 (70 mg, purity: 96% on LCMS) and Compound 130 (75 mg, purity: 96% on LCMS). ¹H NMR: (400 MHz; d₆-DMSO): δ ppm 11.84 (s, 1H), 10.27 (s, 1H), 8.62 (s, 1H), 8.45 (d, J=2.4 Hz, 1H), 8.31 (d, J=7.6 Hz, 1H), 8.27 (s, 1H), 7.82 (d, J=8.8 Hz, 2H), 7.72 (d, J=8.8 Hz, 2H), 7.49 (d, J=8 Hz, 1H), 7.41 (d, J=8 Hz, 1H), 7.23-7.16 (m, 2H), 6.79-6.73 (m, 1H), 6.28 (d, J=8.4 Hz, 2H), 4.32 (s, 2H), 3.07 (d, J=5.2 Hz, 2H), 2.46-2.45 (m, 2H), 2.18 (s, 6H), 2.00-1.98 (m, 2H), 1.78-1.73 (m, 2H), 1.66-1.59 (m, 1H).

### Example 31. Synthesis of (E)-N-(3-(5-chloro-4-(1H-indol-3-yl)pyrimidin-2-ylamino)-2,2-dimethylpropyl)-4-(4-hydroxybut-2-enamido)benzamide (Compound 132)

### N1-(5-chloro-4-(1-(phenylsulfonyl)-1H-indol-3-yl)pyrimidin-2-yl)-2,2-dimethylpropane-1,3-diamine

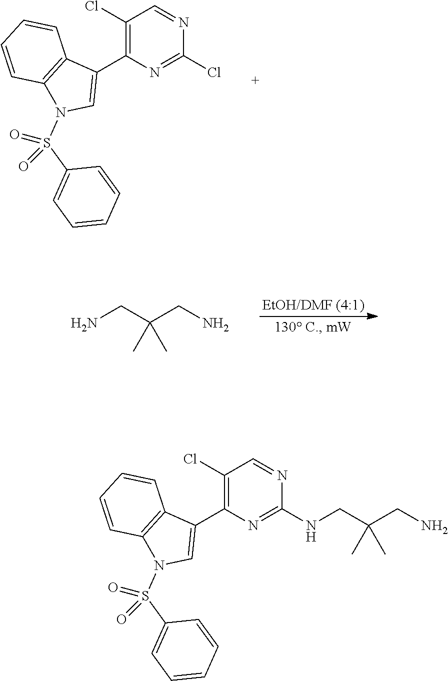

A suspension of 3-(2,5-dichloropyrimidin-4-yl)-1-(phenylsulfonyl)-1H-indole (0.590 g, 1.46 mmol), 3,3-dimethylaminopropyldiamine (149 mg, 1.46 mmol) in EtOH/DMF (4:1, 10 mL) was heated at 130° C. (mW) for 20 min. The mixture was diluted with EtOAc (30 mL), washed with sat. NaHCO₃ (5 mL), brine (5 mL) dried (MgSO₄), filtered and concentrated under reduced pressure to afford the title compound (600 mg, 1.28 mmol, 87%) as a white solid which was used in the next step without any further purification.

### tert-butyl 4-(3-(5-chloro-4-(1-(phenylsulfonyl)-1H-indol-3-yl)pyrimidin-2-ylamino)-2,2-dimethylpropylcarbamoyl)phenylcarbamate

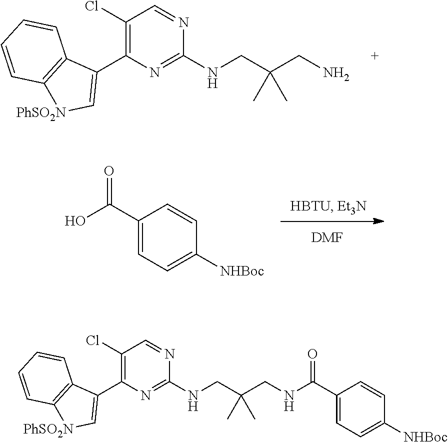

To a solution of N1-(5-chloro-4-(1-(phenylsulfonyl)-1H-indol-3-yl)pyrimidin-2-yl)-2,2-dimethylpropane-1,3-diamine (200 mg, 0.43 mmol), 4-(tert-butoxycarbonylamino)benzoic acid (100 mg, 0.43 mmol) and Et₃N (180 μL, 1.28 mmol) in DMF (4.0 mL) was added HBTU (200 mg, 0.527 mmol). The mixture was stirred overnight at rt, diluted with EtOAc (100 mL), washed with water (20 mL), brine (2×20 mL), dried (MgSO₄), filtered and evaporated to dryness. The residue was purified by SiO₂ chromatography (DCM/EtOAc 10 to 50% gradient) and afforded the title compound (296 mg, 0.43 mmol, 100%) as a white solid.

### 4-amino-N-(3-(5-chloro-4-(1-(phenylsulfonyl)-1H-indol-3-yl)pyrimidin-2-ylamino)-2,2-dimethylpropyl)benzamide.TFA

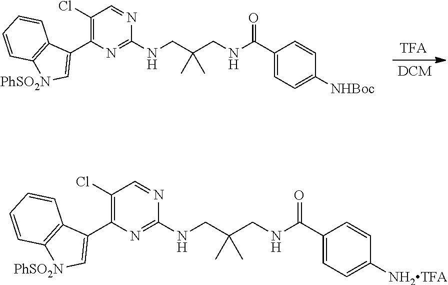

To a solution of tert-butyl 4-(3-(5-chloro-4-(1-(phenylsulfonyl)-1H-indol-3-yl)pyrimidin-2-ylamino)-2,2-dimethylpropylcarbamoyl)phenylcarbamate (90 mg, 0.131 mmol) in DCM (3 mL) was added TFA (1 mL). The resulting mixture was stirred 30 min at rt before evaporation to dryness. The residue was dried under high vacuum and afforded the title compound (92 mg, 0.131 mmol, 100%) as a colorless glue which was used in the next step without further purification.

### (E)-N-(3-(5-chloro-4-(1-(phenylsulfonyl)-1H-indol-3-yl)pyrimidin-2-ylamino)-2,2-dimethylpropyl)-4-(4-(dimethylamino)but-2-enamido)benzamide

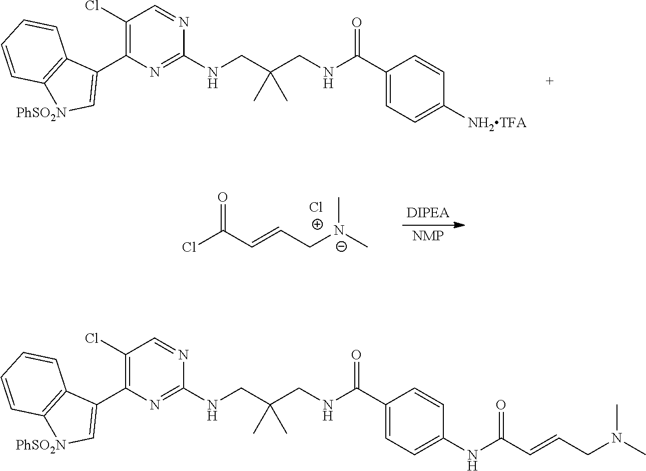

To a 0° C. solution of (E)-N-(3-(5-chloro-4-(1-(phenylsulfonyl)-1H-indol-3-yl)pyrimidin-2-ylamino)-2,2-dimethylpropyl)-4-(4-(dimethylamino)but-2-enamido)benzamide (60 mg, 0.101 mmol) and DIPEA (40 μL, 0.310 mmol) in NMP (2 mL) was slowly added a solution of 54 mg/mL (E)-4-chloro-N,N-dimethyl-4-oxobut-2-en-1-aminium chloride (0.34 mL, 0.101 mmol) in DCM. The mixture is stirred at rt for 2 h, before being diluted with ETOAc (25 mL) and washed with sat. NaHCO₃ (5 mL). The organic layer was dried (MgSO₄), filtered, evaporated to dryness and afforded the title compound (22 mg, 0.031 mmol, 31%) which was used without further purification.

### (E)-N-(3-(5-chloro-4-(1H-indol-3-yl)pyrimidin-2-ylamino)-2,2-dimethylpropyl)-4-(4-hydroxybut-2-enamido)benzamide

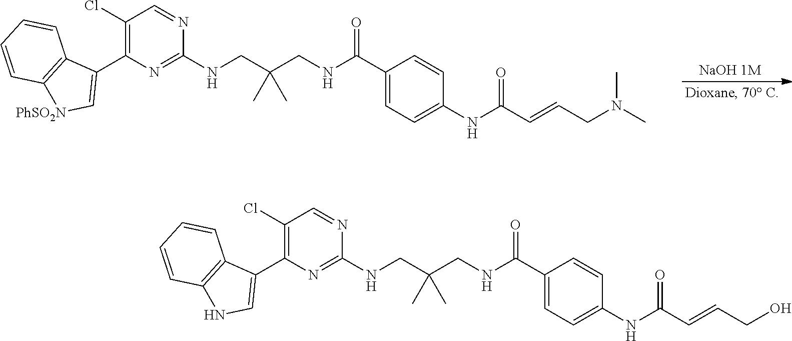

A solution of (E)-N-(3-(5-chloro-4-(1-(phenylsulfonyl)-1H-indol-3-yl)pyrimidin-2-ylamino)-2,2-dimethylpropyl)-4-(4-(dimethylamino)but-2-enamido)benzamide (22 mg, 0.031 mmol) in dioxane (2 mL) and 1M NaOH (400 μL) was heated at 70° C. for 5 h. The cooled mixture was treated with HCO₂H (100 μL) and the volatiles were removed by evaporation. The crude mixture was purified by reverse phase chromatography (C18, H₂O/ACN +0.1% HCO₂H 0 to 100% gradient) and afforded the title compound (3.2 mg, 0.006 mmol, 19%) as a white solid after lyophilization. ¹H NMR (500 MHz, d₆-DMSO) δ 11.84 (s, 1H), 8.72-8.57 (m, 1H), 8.45 (s, 2H), 8.25 (s, 1H), 7.88 (d, J=8.2 Hz, 2H), 7.78-7.66 (m, 1H), 7.49 (d, J=8.4 Hz, 2H), 7.42-7.08 (m, 3H), 6.38 (d, J=8.0 Hz, 2H), 5.81-5.63 (m, 2H), 3.22 (d, J=4.8 Hz, 2H), 2.36 (s, 3H), 1.88 (s, 1H), 1.49-1.16 (m, 4H), 0.93 (s, 2H); MS (m/z): 533.59 [M+1]⁺.

### Example 32. Synthesis of (E)-N-(3-(3-(5-chloro-4-(1H-indol-3-yl)pyrimidin-2-ylamino)phenylamino)-3-oxopropyl)-4-(dimethylamino)but-2-enamide (Compound 131)

### N1-(5-chloro-4-(1-(phenylsulfonyl)-1H-indol-3-yl)pyrimidin-2-yl)benzene-1,3-diamine

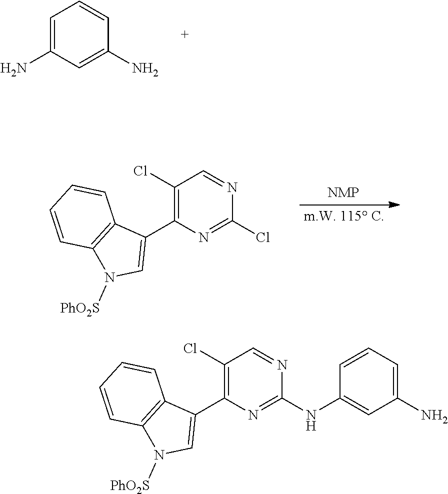

A solution of 3-(2,5-dichloropyrimidin-4-yl)-1-(phenylsulfonyl)-1H-indole (1.5 g, 3.70 mmol) and m-phenylenediamine (400 mg, 3.70 mmol) in NMP (15 mL) was heated 15 min at 175° C. (mW). The cooled mixture was diluted with EtOAc (100 mL) and water (50 mL). The layers were separated and the aqueous layer was extracted with EtOAc (3×50 mL). The combined organics were washed with brine (50 mL) dried (MgSO₄), filtered and evaporated to dryness. The mixture was purified by SiO₂ column (DCM/EtOAc 0 to 30% gradient) and afforded the title compound (606 mg, 1.27 mmol, 34%) as a pale brown solid.

### tert-butyl 3-(3-(5-chloro-4-(1-(phenylsulfonyl)-1H-indol-3-yl)pyrimidin-2-ylamino)phenylamino)-3-oxopropylcarbamate

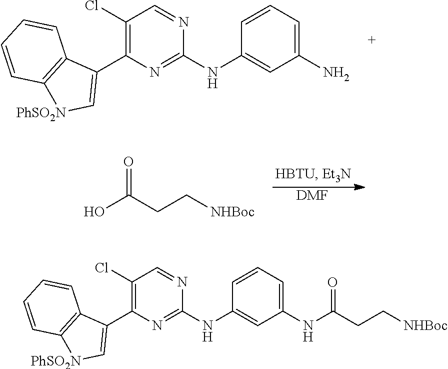

To a solution of N1-(5-chloro-4-(1-(phenylsulfonyl)-1H-indol-3-yl)pyrimidin-2-yl)benzene-1,3-diamine (150 mg, 0.315 mmol), Boc-f3-Ala-OH (72 mg, 0.378 mmol) and Et₃N (131 μL, 0.945 mmol) in DMF (2.1 mL) was added HBTU (179 mg, 0.473 mmol). The mixture was stirred overnight at rt, diluted with EtOAc (20 mL), washed with sat. NaHCO₃ (5 mL), brine (2×5 mL), dried (MgSO₄), filtered and evaporated to dryness which afforded the title compound (204 mg, 0.315 mmol, 100%) as a colorless oil which was used in the next step without further purification.

### tert-butyl 3-(3-(5-chloro-4-(1H-indol-3-yl)pyrimidin-2-ylamino)phenylamino)-3-oxopropylcarbamate

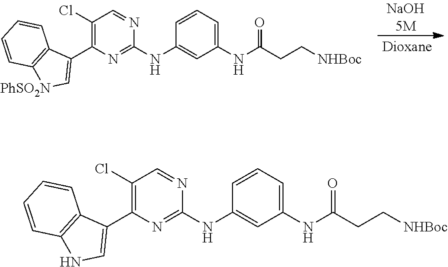

A solution of tert-butyl 3-(3-(5-chloro-4-(1-(phenylsulfonyl)-1H-indol-3-yl)pyrimidin-2-ylamino)phenylamino)-3-oxopropylcarbamate (204 mg, 0.315 mmol) in dioxane (5.2 mL) and 5M NaOH (946 μL, 4.73 mmol) was heated at 70° C. for 2 h. The cooled mixture was diluted with DCM (10 mL) and a sat. solution of NH₄Cl (10 mL). The layers were separated and the aqueous layer was extracted with DCM (3×10 mL). The combined organic layers were dried (MgSO₄), filtered and evaporated to dryness. The residue was purified by SiO₂ chromatography (DCM/EtOAc 0 to 50% gradient) and afforded the title compound (152 mg, 0.299, 95%) as a creamy foam.

### 3-amino-N-(3-(5-chloro-4-(1H-indol-3-yl)pyrimidin-2-ylamino)phenyl) propanamide.TFA (Compound 306 TFA Salt)

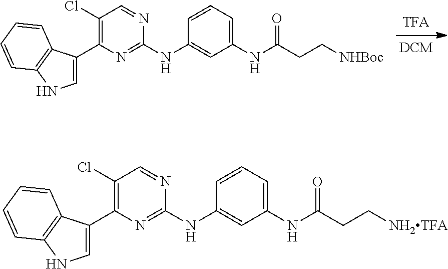

To a tert-butyl 3-(3-(5-chloro-4-(1H-indol-3-yl)pyrimidin-2-ylamino)phenylamino)-3-oxopropylcarbamate (152 mg, 0.300 mmol) in DCM (3 mL) was added TFA (344 μL, 4.50 mmol). The resulting mixture was stirred 90 min at rt before evaporation to dryness. The residue was dried under high vacuum and afforded the title compound (156 mg, 0.300 mmol, 100%) as a brownish glue which was used in the next step without further purification.

### (E)-N-(3-(3-(5-chloro-4-(1H-indol-3-yl)pyrimidin-2-ylamino)phenylamino)-3-oxopropyl)-4-(dimethylamino)but-2-enamide

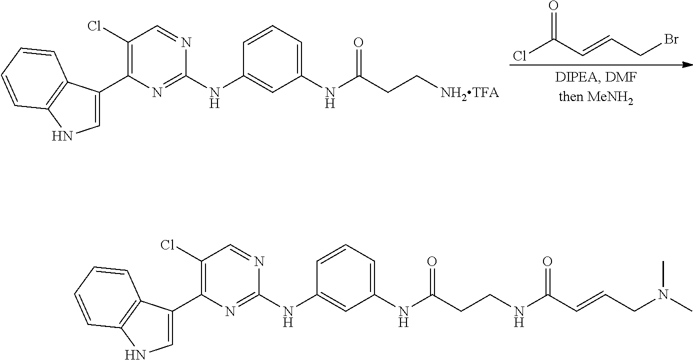

To a −60° C. solution of 3-amino-N-(3-(5-chloro-4-(1H-indol-3-yl)pyrimidin-2-ylamino)phenyl)propanamide.TFA (68 mg, 0.167 mmol) and DIPEA (87 μL, 0.167 mmol) in 1:1 NMP/THF (1.6 mL) was added a 1M solution of (E)-4-bromobut-2-enoyl chloride (167 μL, 0.167 mmol) in DCM. The resulting mixture was stirred 1 h at −60° C. before addition of a 2M solution of dimethylamine in THF (501 μL, 1.00 mmol). The resulting was stirred overnight at −30° C. and warmed to rt before being evaporated to dryness. The residue was purified by reverse phase chromatography (C18, water/ACN +0.1% HCO₂H 15 to 60% gradient) and afforded the title compounds (21.7 mg, 0.042 mmol, 25%) as a white solid after lyophilization. ¹H NMR (500 MHz, d₆-DMSO) δ 11.86 (s, 1H), 10.57 (s, 1H), 9.99 (br s, 1H), 8.65 (s, 1H), 8.47 (d, J=5.0 Hz, 1H) 8.44 (br s, 1H), 8.25 (s, 1H), 8.13 (s, 1H), 7.87 (d, J=8.4 Hz, 1H), 7.77 (d, J=8.0 Hz, 1H), 7.49 (d, J=8.1 Hz, 1H), 7.22-7.08 (m, 2H), 7.00 (s, 1H), 6.82-6.75 (m, 1H), 6.49 (d, J=15.4 Hz, 1H), 3.95 (t, J=5.0 Hz, 2H), 3.22 (d, J=5.4 Hz, 2H), 2.80 (d, J=4.3 Hz, 2H), 2.50 (s, 6H), 0.93 (s, 6H); MS (m/z): 518.66 [M+1]⁺.

### Example 33. Synthesis of (E)-N-(2-(3-(5-chloro-4-(1H-indol-3-yl)pyrimidin-2-ylamino)phenylamino)-2-oxoethyl)-4-(dimethylamino)but-2-enamide (Compound 137)

### tert-butyl 2-(3-(5-chloro-4-(1-(phenylsulfonyl)-1H-indol-3-yl)pyrimidin-2-ylamino)phenylamino)-2-oxoethylcarbamate

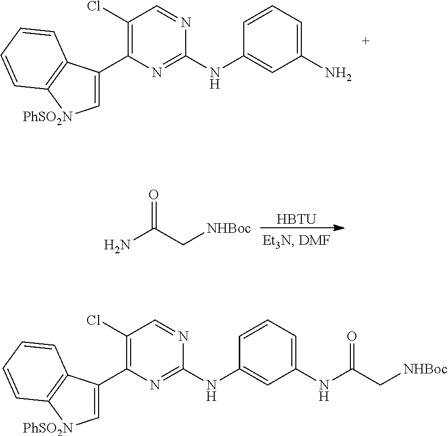

To a solution of N1-(5-chloro-4-(1-(phenylsulfonyl)-1H-indol-3-yl)pyrimidin-2-yl)benzene-1,3-diamine (110 mg, 0.231 mmol) prepared as in Example 32, Boc-gly-OH (49 mg, 0.277 mmol) and Et₃N (97 μL, 0.693 mmol) in DMF (1.5 mL) was added HBTU (132 mg, 0.347 mmol). The mixture was stirred overnight at rt, diluted with EtOAc (20 mL), washed with sat. NaHCO₃ (5 mL), brine (2×5 mL), dried (MgSO₄), filtered and evaporated to dryness which afforded the title compound (146 mg, 0.231 mmol, 100%) as a colorless oil which was used in the next step without further purification.

### tert-butyl 2-(3-(5-chloro-4-(1H-indol-3-yl)pyrimidin-2-ylamino)phenylamino)-2-oxoethylcarbamate

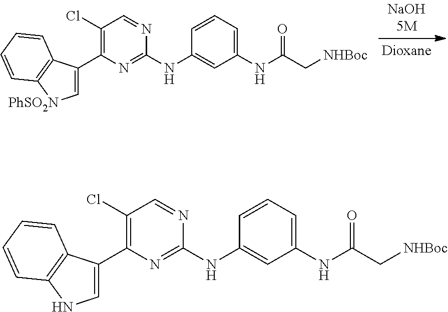

A solution of tert-butyl 2-(3-(5-chloro-4-(1-(phenylsulfonyl)-1H-indol-3-yl)pyrimidin-2-ylamino)phenylamino)-2-oxoethylcarbamate (146 mg, 0.231 mmol) in dioxane (3.8 mL) and 5M NaOH (692 μL, 3.45 mmol) was heated at 70° C. for 2 h. The cooled mixture was diluted with DCM (10 mL) and a sat. solution of NH₄Cl (10 mL). The layers were separated and the aqueous layer was extracted with DCM (3×10 mL). The combined organic layers were dried (MgSO₄), filtered and evaporated to dryness. The residue was purified by SiO₂ chromatography (DCM/EtOAc 0 to 50% gradient) and afforded the title compound (82 mg, 0.166, 72%) as a creamy foam.

### 2-amino-N-(3-(5-chloro-4-(1H-indol-3-yl)pyrimidin-2-ylamino)phenyl)acetamide.TFA (Compound 307 TFA Salt)

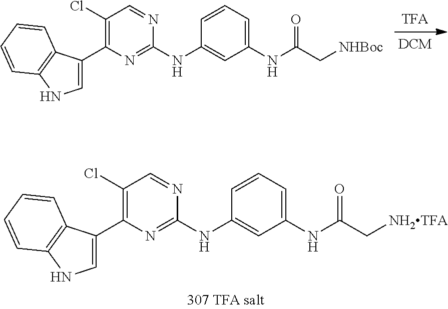

To a solution of tert-butyl 2-(3-(5-chloro-4-(1H-indol-3-yl)pyrimidin-2-ylamino)phenylamino)-2-oxoethylcarbamate (82 mg, 0.166 mmol) in DCM (1.7 mL) was added TFA (191 μL, 2.50 mmol). The resulting mixture was stirred 90 min at rt before evaporation to dryness. The residue was dried under high vacuum and afforded the title compound (84 mg, 0.166 mmol, 100%) as a brownish glue which was used in the next step without further purification.

### (E)-N-(2-(3-(5-chloro-4-(1H-indol-3-yl)pyrimidin-2-ylamino)phenylamino)-2-oxoethyl)-4-(dimethylamino)but-2-enamide

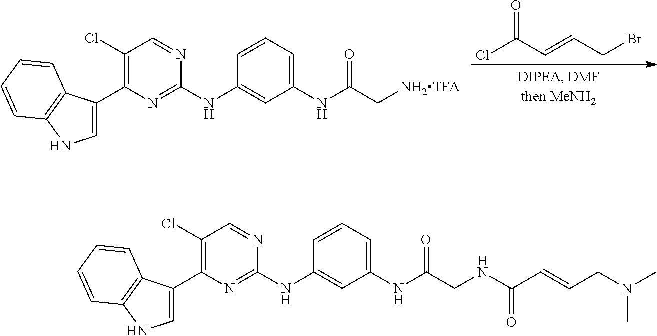

To a −60° C. solution of 2-amino-N-(3-(5-chloro-4-(1H-indol-3-yl)pyrimidin-2-ylamino)phenyl)acetamide.TFA (60 mg, 0.153 mmol) and DIPEA (59 μL, 0.458 mmol) in THF (0.8 mL) was added a 1M solution of (E)-4-bromobut-2-enoyl chloride (153 μL, 0.153 mmol) in DCM. The resulting mixture was stirred 1 h at −60° C. before addition of a 2M solution of dimethylamine in THF (458 μL, 0.916 mmol). The resulting was warmed to rt and evaporated to dryness. The residue was purified by reverse phase chromatography (C18, water/ACN +0.1% HCO₂H 15 to 60% gradient) and afforded the title compounds (30.5 mg, 0.061 mmol, 40%) as a white solid after lyophilization. ¹H NMR (500 MHz, d₆-DMSO) δ 11.91 (s, 1H), 9.97 (s, 1H), 9.64 (s, 1H), 8.63 (d, J=7.9 Hz, 1H), 8.52 (d, J=3.0 Hz, 1H), 8.45 (s, 1H), 8.34 (t, J=5.9 Hz, 1H), 7.92 (s, 1H), 7.50 (d, J=8.1 Hz, 2H), 7.27 (d, J=8.2 Hz, 1H), 7.22 (ddd, J=8.0, 3.8, 2.1 Hz, 2H), 7.12 (t, J=7.1 Hz, 1H), 6.60 (dt, J=15.5, 6.2 Hz, 1H), 6.16 (dt, J=15.5, 1.5 Hz, 1H), 3.95 (d, J=5.9 Hz, 2H), 3.01 (dd, J=6.1, 1.3 Hz, 2H), 2.15 (s, J=34.8 Hz, 6H); MS (m/z): 504.54 [M+1]⁺.

### Example 34. Synthesis of ((1R,3R,5S)-3-(5-chloro-4-(1H-indol-3-yl)pyrimidin-2-ylamino)-8-aza-bicyclo[3.2.1]octan-8-yl)(4-((E)-N-4-(dimethylamino)but-2-enamide)phenyl)methanone (Compound 116)

### tert-Butyl (1R,5S)-3-amino-8-azabicyclo[3.2.1]octane-8-carboxylate

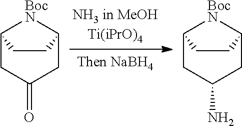 [SMILES: CC(C)(C)OC(=O)N1[C@@H]2CC[C@H]1CC(=O)C2 ; CC(C)(C)OC(=O)N1[C@@H]2CC[C@H]1C[C@@H](N)C2]

A mixture of tert-Butyl (1S,5R)-3-oxo-8-azabicyclo[3.2.1]octane-8-carboxylate (200 mg, 0.89 mmol) and a 7M solution of NH₃ in MeOH (630 μL, 4.44 mmol) was treated with Ti(iPrO)₄ (526 mL, 1.78 mmol) and stirred 6 h at rt. The resulting mixture was treated with NaBH₄ (30 mg, 1.33 mmol) and the mixture was stirred 3 h at rt. The resulting mixture was diluted with a 2M solution of NH₄OH (20 mL) and the resulting solid was filtered and washed with EtOAc (2×25 mL). The layers of the filtrate were separated and the aqueous layer was extracted with EtOAc (2×25 mL). The combined organic layers were washed extracted with a 1M solution of HCl in water (30 mL) and the aqueous layer was washed with EtOAc (50 mL). The aqueous layer was then treated with a 2M solution of NaOH until the pH reached 12 and extracted with EtOAc (3×50 mL). The combined organic layers were washed with brine (50 mL), dried (MgSO₄), filtered and evaporated to dryness affording the title compound (169 mg, 0.748, 84%) as a colorless oil which was used in the next step without further purification.

### (1S,3R,5R)—N-(5-chloro-4-(1-(phenylsulfonyl)-1H-indol-3-yl)pyrimidin-2-yl)-8-(tert-butoxycarbonyl)-aza-bicyclo[3.2.1]octan-3-amine

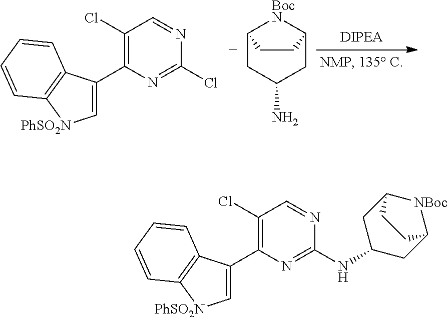

A solution of 3-(2,5-dichloropyrimidin-4-yl)-1-(phenylsulfonyl)-1H-indole (300 mg, 0.74 mmol), tert-Butyl (1R,5S)-3-amino-8-azabicyclo[3.2.1]octane-8-carboxylate (168 mg, 0.74 mmol) and DIPEA (0.13 mL, 0.74 mmol) in NMP (2.0 mL) was heated at 135° C. for 25 min (mW). The cooled mixture was diluted with EtOAc (30 mL), washed with water (3×10 mL), brine (10 mL), dried (MgSO₄), filtered and evaporated to dryness. The residue was purified by SiO₂ chromatography (Hex/EtOAc 0 to 50% gradient) and afforded the title compound (249 mg, 0.420 mmol, 57%) as a white solid.

### (1S,3R,SR)—N-(5-chloro-4-(1-(phenylsulfonyl)-1H-indol-3-yl)pyrimidin-2-yl)-8-aza-bicyclo[3.2.1]octan-3-amine

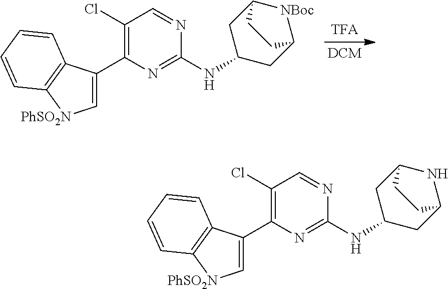

To a (1S,3R,5R)—N-(5-chloro-4-(1-(phenylsulfonyl)-1H-indol-3-yl)pyrimidin-2-yl)-8-(tert-butoxycarbonyl)-aza-bicyclo[3.2.1]octan-3-amine (128 mg, 0.220 mmol) in DCM (1.0 mL) was added TFA (160 μL, 2.15 mmol). The resulting mixture was stirred 1 h at rt before evaporation to dryness. The residue was diluted with DCM (20 mL), washed with a sat. solution of NaHCO₃ (3×5 mL), brine (5 mL), dried (MgSO₄), filtered and evaporated to dryness, affording the title compound (106 mg, 0.215 mmol, 95%) as a colorless oil which was used in the next step without further purification.

### (1S,3R,5R)-tert-butyl-N-(5-chloro-4-(1-(phenylsulfonyl)-1H-indol-3-yl)pyrimidin-2-yl)-8-carbonyl)phenylcarbamate-aza-bicyclo[3.2.1]octan-3-amine

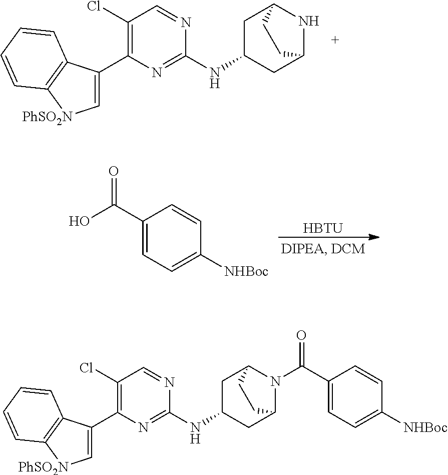

To a solution of (1 S,3R,5R)—N-(5-chloro-4-(1-(phenylsulfonyl)-1H-indol-3-yl)pyrimidin-2-yl)-8-aza-bicyclo[3.2.1]octan-3-amine (106 mg, 0.215 mmol), 4-(tert-butoxycarbonylamino)benzoic acid (51 mg, 0.210 mmol) and DIPEA (220 μL, 1.29 mmol) in DCM (1.5 mL) was added HBTU (163 mg, 0.347 mmol). The mixture was stirred overnight at rt, diluted with EtOAc (20 mL), washed with sat. NaHCO₃ (5 mL), brine (2×5 mL), dried (MgSO₄), filtered and evaporated to dryness which afforded the title compound (153 mg, 0.215 mmol, 100%) as a colorless oil which was used in the next step without further purification.

### (4-aminophenyl)((1R,3R,5S)-3-(5-chloro-4-(1-(phenylsulfonyl)-1H-indol-3-yl)pyrimidin-2-ylamino)-8-aza-bicyclo[3.2.1]octan-8-yl)methanone

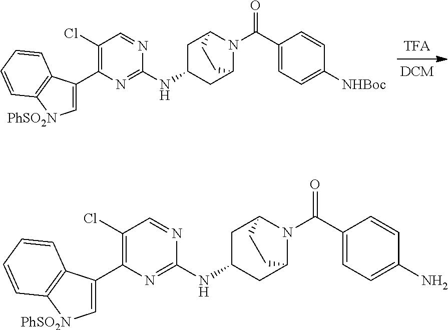

To a solution of (1S,3R,5R)-tert-butyl-N-(5-chloro-4-(1-(phenylsulfonyl)-1H-indol-3-yl)pyrimidin-2-yl)-8-carbonyl)phenylcarbamate-aza-bicyclo[3.2.1]octan-3-amine (153 mg, 0.215 mmol) in DCM (0.9 mL) was added TFA (160 μL, 2.15 mmol). The resulting mixture was stirred 90 min at rt before evaporation to dryness. The residue was diluted with DCM (30 mL), washed with a sat. solution of NaHCO₃ (3×10 mL), brine (10 mL), dried (MgSO₄), filtered and evaporated to dryness, affording the title compound (130 mg, 0.212 mmol, 99%) as a colorless glue which was used in the next step without further purification.

### (4-aminophenyl)((1R,3R,5S)-3-(5-chloro-4-(1H-indol-3-yl)pyrimidin-2-ylamino)-8-aza-bicyclo[3.2.1]octan-8-yl)methanone (Compound 302)

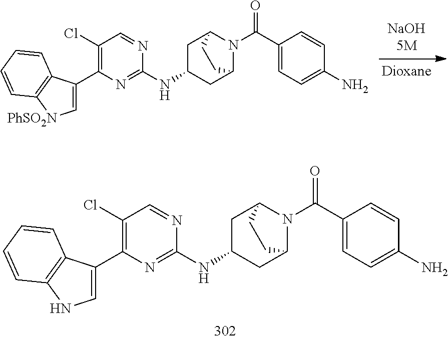

A solution of (4-aminophenyl)((1R,3R,5S)-3-(5-chloro-4-(1-(phenylsulfonyl)-1H-indol-3-yl)pyrimidin-2-ylamino)-8-aza-bicyclo[3.2.1]octan-8-yl)methanone (132 mg, 0.215 mmol) in dioxane (1.4 mL) and 5M NaOH (430 μL, 2.15 mmol) was heated at 75° C. for 2 h. The cooled mixture was evaporated to dryness and the residue was purified by SiO₂ chromatography (DCM/IPA 0 to 15% gradient) affording the title compound (65 mg, 0.137, 64%) as a white solid.

### ((1R,3R,5S)-3-(5-chloro-4-(1H-indol-3-yl)pyrimidin-2-ylamino)-8-aza-bicyclo[3.2.1]octan-8-yl)(4-((E)-N-4-(dimethylamino)but-2-enamide)phenyl)methanone

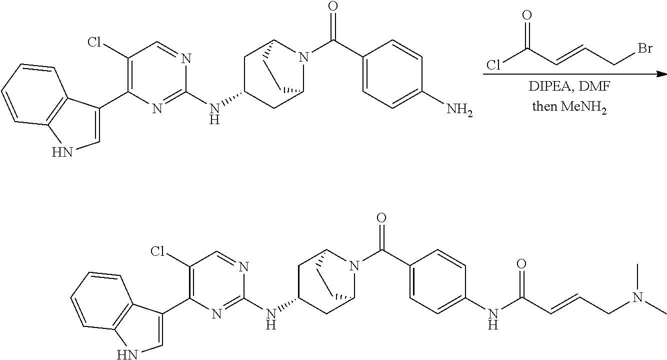 [SMILES: Nc1ccc(C(=O)N2[C@@H]3CC[C@H]2C[C@@H](Nc2ncc(Cl)c(-c4c[nH]c5ccccc45)n2)C3)cc1 ; O=C(Cl)/C=C/CBr ; CN(C)C/C=C/C(=O)Nc1ccc(C(=O)N2[C@@H]3CC[C@H]2C[C@@H](Nc2ncc(Cl)c(-c4c[nH]c5ccccc45)n2)C3)cc1]

To a −60° C. solution of ((1R,3R,5S)-3-(5-chloro-4-(1H-indol-3-yl)pyrimidin-2-ylamino)-8-aza-bicyclo[3.2.1]octan-8-yl)(4-((E)-N-4-(dimethylamino)but-2-enamide)phenyl)methanone (21 mg, 0.044 mmol) and DIPEA (23 μL, 0.133 mmol) in THF (0.9 mL) was added a 1M solution of (E)-4-bromobut-2-enoyl chloride (156 μL, 0.156 mmol) in DCM. The resulting mixture was stirred 1 h at −60° C. before addition of a 2M solution of dimethylamine in THF (67 μL, 0.133 mmol). The resulting was warmed up to rt and evaporated to dryness. The residue was purified by reverse phase chromatography (C18, water/ACN +0.1% HCO₂H 15 to 65% gradient) and afforded the title compound (14 mg, 0.024 mmol, 54%) as a white solid after lyophilisation. ¹H NMR (500 MHz, d₆-DMSO) δ 11.83 (d, J=2.4 Hz, 1H), 10.23 (s, 1H), 8.57 (d, (br) J=4.1 Hz, 1H), 8.46 (d, J=3.0 Hz, 1H), 8.29 (s, 1H), 7.72 (d, J=8.7 Hz, 2H), 7.49-7.45 (m, 1H), 7.47 (d, J=8.6 Hz, 2H), 7.23-7.17 (m, 2H), 7.17-7.12 (m, 1H), 6.76 (dt, J=15.4, 5.8 Hz, 1H), 6.28 (dt, J=15.3, 1.6 Hz, 1H), 4.61 (s (br), 1H), 4.18-4.10 (m, 1H), 4.10-4.03 (m, 1H), 3.06 (dd, J=5.8, 1.4 Hz, 2H), 2.35-2.26 (m, 1H), 2.18 (s, 6H), 2.17-2.12 (m, 3H), 2.07-1.95 (m, 2H), 1.95-1.86 (m, 2H); MS (m/z): 584.63 [M+1]⁺.

### Example 35. Synthesis of 4-acrylamido-N-(6-((5-chloro-4-(1H-indol-3-yl)pyrimidin-2-yl)amino)spiro[3.3]heptan-2-yl)benzamide (Compound 138)

### N2-(5-chloro-4-(1-(phenylsulfonyl)-1H-indol-3-yl)pyrimidin-2-yl)spiro[3.3]heptane-2,6-diamine

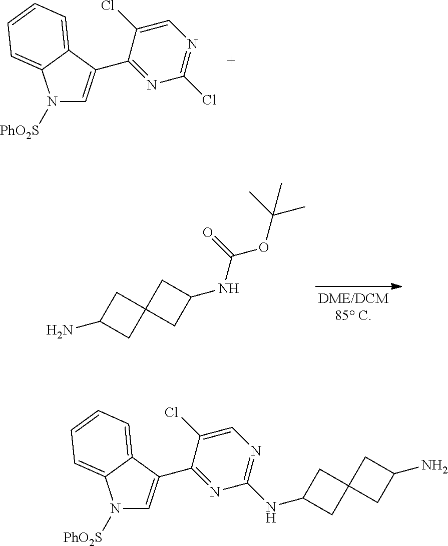

A 20 mL scintillation vial was charged with 3-(2,5-dichloropyrimidin-4-yl)-1-(phenylsulfonyl)-1H-indole (170 mg, 0.42 mmol) and tert-butyl (6-aminospiro[3.3]heptan-2-yl)carbamate (113 mg, 0.5 mmol). Reagents were suspended in a mixture of DME (3.4 mL) and DCM (0.8 mL). The vial was sealed and heated to 85° C. for 16 h. Upon completion (with concomitant Boc deprotection) the reaction was cooled and concentrated. The residue was purified by SiO₂ chromatography (MeOH/DCM gradient 0-15%) to afford the title compound (71.5 mg, 0.145 mmol, 35%) as a white solid.

### N-(6-((5-chloro-4-(1-(phenylsulfonyl)-1H-indol-3-yl)pyrimidin-2-yl)amino)spiro[3.3]heptan-2-yl)-4-nitrobenzamide

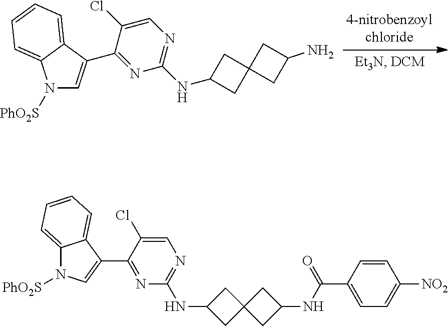

N2-(5-chloro-4-(1-(phenylsulfonyl)-1H-indol-3-yl)pyrimidin-2-yl)spiro[3.3]heptane-2,6-diamine (71.5 mg, 0.145 mmol) was dissolved in DCM (15 mL) in a 20 mL scintillation vial. Et₃N (63 μL, 0.145 mmol) was added, followed by 4-nitrobenzoyl chloride (26.9 mg, 0.145 mmol), and the vial was sealed and stirred for 16 h at rt. The reaction mixture was partitioned between DCM (10 mL) and saturated aqueous NaHCO₃ (25 mL) and extracted with DCM (2×25 mL). Combined organics were washed with brine (25 mL), dried over Na₂SO₄, filtered and concentrated to afford the title compound as a crude residue which was used directly in the next step.

### 4-amino-N-(6-((5-chloro-4-(1-(phenylsulfonyl)-1H-indol-3-yl)pyrimidin-2-yl)amino)spiro[3.3]heptan-2-yl)benzamide

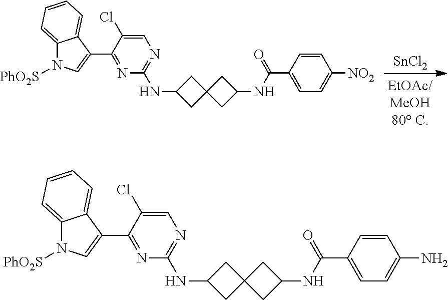

N-(6-((5-chloro-4-(1-(phenylsulfonyl)-1H-indol-3-yl)pyrimidin-2-yl)amino)spiro[3.3]heptan-2-yl)-4-nitrobenzamide was dissolved in EtOAc (5 mL) and MeOH (1 mL) in a 20 mL scintillation vial. Tin (II) chloride (69 mg, 0.363 mmol) was added, the vial sealed and the reaction heated to 80° C. for 3 h. The reaction was cooled and then poured into saturated aqueous NaHCO₃ (25 mL). The aqueous layer was extracted with 4:1 CHCl₃/2-propanol (3×10 mL) and combined organics were washed with water (25 mL) and then brine (25 mL) and dried over Na₂SO₄. The filtrate was concentrated and the residue purified by SiO₂ chromatography (MeOH/DCM gradient 0-15%) to afford the title compound (37 mg, 0.060 mmol, 41% over two steps) as a tan solid.

### 4-amino-N-(6-((5-chloro-4-(1H-indol-3-yl)pyrimidin-2-yl)amino)spiro[3.3]heptan-2-yl)benzamide (Compound 318)

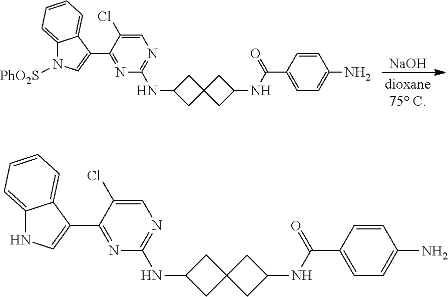

4-amino-N-(6-((5-chloro-4-(1-(phenylsulfonyl)-1H-indol-3-yl)pyrimidin-2-yl)amino)spiro[3.3]heptan-2-yl)benzamide (37 mg, 0.060 mmol) was dissolved in dioxane (400 μL) and a 5M solution of aqueous NaOH was added (120 μL, 0.6 mmol). The mixture was heated to 75° C. for 3 h. The cooled mixture was diluted with DCM (5 mL) and extracted with saturated aqueous NH₄Cl (5 mL). The organic layers were washed with brine (5 mL) and then dried over sodium sulfate, filtered and concentrated. Crude residue was purified by SiO₂ chromatography (MeOH/DCM, 0-20% gradient) to afford the title compound (25.5 mg, 0.054 mmol, 90%) as a white solid. MS (m/z): 473.54 [M+1]⁺.

### 4-acrylamido-N-(6-((5-chloro-4-(1H-indol-3-yl)pyrimidin-2-yl)amino)spiro[3.3]heptan-2-yl)benzamide (Compound 138)

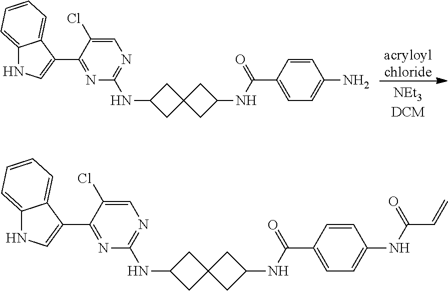 [SMILES: Nc1ccc(C(=O)NC2CC3(C2)CC(Nc2ncc(Cl)c(-c4c[nH]c5ccccc45)n2)C3)cc1 ; "acryloylchloride" ; C=CC(=O)Nc1ccc(C(=O)NC2CC3(C2)CC(Nc2ncc(Cl)c(-c4c[nH]c5ccccc45)n2)C3)cc1]

4-amino-N-(6-((5-chloro-4-(1H-indol-3-yl)pyrimidin-2-yl)amino)spiro[3.3]heptan-2-yl)benzamide (23.7 mg, 0.050 mmol) was dissolved in DCM (1 mL) and Et₃N (14 μL, 0.10 mmol) was added followed by acryloyl chloride (6.2 uL, 0.075 mmol). The mixture was stirred for 2 h at rt, diluted with DCM (5 mL) and quenched by saturated aqueous NaHCO₃. The aqueous layer was extracted with DCM (2×5 mL) and combined organics were washed with brine (10 mL), dried over sodium sulfate, filtered and concentrated. Crude residue was purified by SiO₂ chromatography (MeOH/DCM gradient 0-20%) to afford the title compound (4.8 mg, 0.009 mmol, 18%) as an off-white solid. MS (m/z): 527.55 [M+1]⁺

### Example 36. Synthesis of (E)-N-(4-(4-((5-chloro-4-(pyridin-3-yl)pyrimidin-2-yl)amino)piperidine-1-carbonyl)phenyl)-4-(dimethylamino)but-2-enamide (Compound 118)

### tert-butyl (4-(4-((5-chloro-4-(pyridin-3-yl)pyrimidin-2-yl)amino)piperidine-1-carbonyl)phenyl)carbamate

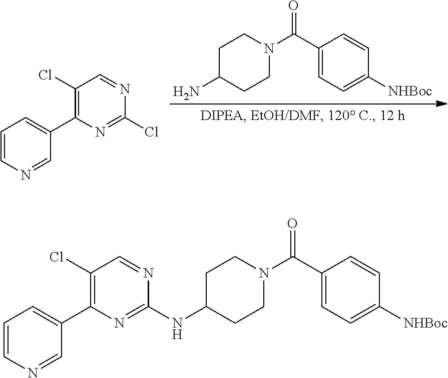 [SMILES: Clc1ncc(Cl)c(-c2cccnc2)n1 ; CC(C)(C)OC(=O)Nc1ccc(C(=O)N2CCC(N)CC2)cc1 ; CC(C)(C)OC(=O)Nc1ccc(C(=O)N2CCC(Nc3ncc(Cl)c(-c4cccnc4)n3)CC2)cc1]

To a solution of 2,5-dichloro-4-(pyridin-3-yl)pyrimidine (100 mg, 0.44 mmol) in EtOH/DMF (2.5 mL) was added tert-butyl (4-(4-aminopiperidine-1-carbonyl)phenyl)carbamate (140 mg, 0.44 mmol) and DIPEA (114 mg, 0.88 mmol). The mixture was stirred at 120° C. for 12 h. The mixture was concentrated under vacuum and the resulting residue was purified by prep-HPLC to afford tert-butyl (4-(4-((5-chloro-4-(pyridin-3-yl)pyrimidin-2-yl)amino)piperidine-1-carbonyl)phenyl)carbamate (200 mg, 88% yield).

### (4-aminophenyl)(4-((5-chloro-4-(pyridin-3-yl)pyrimidin-2-yl)amino)piperidin-1-yl)methanone hydrochloride (Compound 22a)

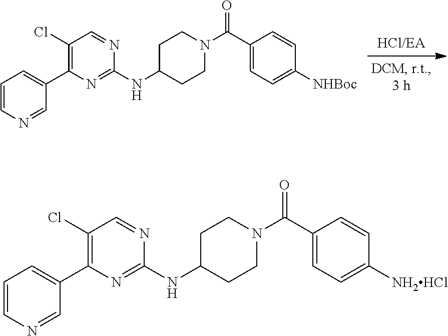

To a solution of tert-butyl (4-(4-((5-chloro-4-(pyridin-3-yl)pyrimidin-2-yl)amino)piperidine-1-carbonyl)phenyl)carbamate (200 mg, 0.402 mmol) in DCM (2 mL) was added HCl/EA (15 mL). The mixture was stirred at rt for 3 h. The mixture was concentrated under vacuum to afford (4-aminophenyl)(4-((5-chloro-4-(pyridin-3-yl)pyrimidin-2-yl)amino)piperidin-1-yl)methanone hydrochloride (170 mg, 100% yield).

### (E)-4-bromo-N-(4-(4-((5-chloro-4-(pyridin-3-yl)pyrimidin-2-yl)amino)piperidine-1-carbonyl)phenyl)but-2-enamide

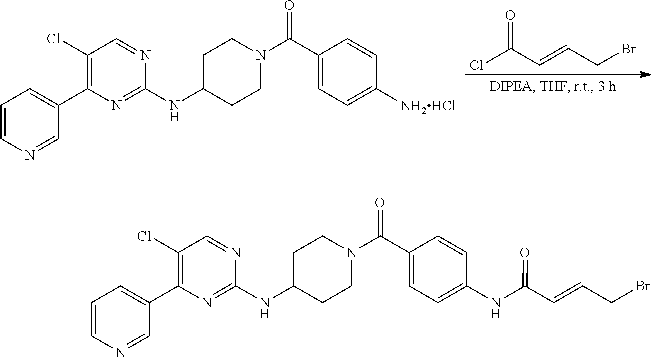

To a solution of (4-aminophenyl)(4-((5-chloro-4-(pyridin-3-yl)pyrimidin-2-yl)amino)piperidin-1-yl)methanone hydrochloride (210 mg, 60%, 0.308 mmol) in THF (10 mL) was added (E)-4-bromobut-2-enoyl chloride (56 mg, 0.308 mmol) and DIPEA (159 mg, 1.232 mmol). The mixture was stirred at rt for 3 h to afford (E)-4-bromo-N-(4-(4-((5-chloro-4-(pyridin-3-yl)pyrimidin-2-yl)amino)piperidine-1-carbonyl)phenyl)but-2-enamide and the mixture was used directly in next step.

### (E)-N-(4-(4-((5-chloro-4-(pyridin-3-yl)pyrimidin-2-yl)amino)piperidine-1-carbonyl)phenyl)-4-(dimethylamino)but-2-enamide

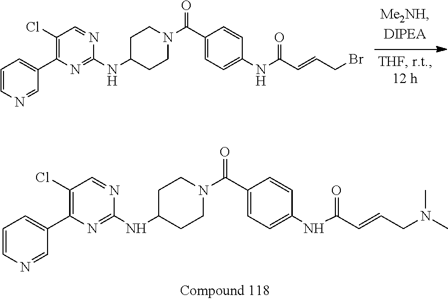 [SMILES: O=C(/C=C/CBr)Nc1ccc(C(=O)N2CCC(Nc3ncc(Cl)c(-c4cccnc4)n3)CC2)cc1 ; CN(C)C/C=C/C(=O)Nc1ccc(C(=O)N2CCC(Nc3ncc(Cl)c(-c4cccnc4)n3)CC2)cc1]

To a solution of (E)-4-bromo-N-(4-(4-((5-chloro-4-(pyridin-3-yl)pyrimidin-2-yl)amino)piperidine-1-carbonyl)phenyl)but-2-enamide (171 mg, 0.308 mmol) in THF (12 mL) cooled to 0° C. was added Me₂NH in THF (0.308 mL, 0.616 mmol, 2M) and DIPEA (79 mg, 0.616 mmol). The mixture was stirred at rt for 12 h. The mixture was concentrated under vacuum and the residue was purified by prep-HPLC to afford Compound 118 (10 mg, 8% yield). ¹H NMR: (400 MHz, d₆-DMSO): δ 10.51 (s, 1H), 9.79 (s, 1H), 8.89 (s, 1H), 8.71 (d, J=4.8 Hz, 1H), 8.47 (s, 1H), 7.74 (s, 1H), 7.72-7.57 (m, 3H), 7.56 (d, J=5.2 Hz, 1H), 7.38 (d, J=8.0 Hz, 1H), 6.79-6.44 (m, 2H), 4.00 (s, 1H), 3.95 (s, 2H), 3.08 (s, 2H), 2.81 (d, J=3.2 Hz, 6H), 1.94 (s, 2H), 1.45 (s, 2H). MS (m/z): 520 [M+1]⁺.

### Example 37. Synthesis of (E)-N-[3-[[4-[[5-chloro-4-(1H-indol-3-yl)pyrimidin-2-yl]amino]-1-piperidyl]methyl]phenyl]-4-(dimethylamino)but-2-enamide (Compound 141)

### Tert-butyl N-[1-[(4-nitrophenyl) methyl]-4-piperidyl]carbamate

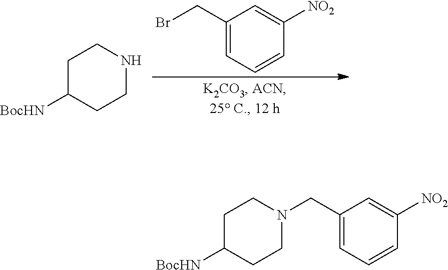 [SMILES: CC(C)(C)OC(=O)NC1CCNCC1 ; O=[N+]([O-])c1cccc(CBr)c1 ; CC(C)(C)OC(=O)NC1CCN(Cc2cccc([N+](=O)[O-])c2)CC1]

To a mixture of tert-butyl N-(4-piperidyl)carbamate (5.00 g, 24.97 mmol) and 1-(bromomethyl)-4-nitro-benzene (5.39 g, 24.97 mmol) in ACN (25 mL) was added K₂CO₃ (6.90 g, 49.94 mmol) at 25° C., The mixture was stirred for 12 h. The mixture was poured into water, extracted with EA, and the organic phase was washed with saturated brine, dried with anhydrous Na₂SO₄, and concentrated. The residue was purified by silica gel to afford the title compound (7.50 g, 89.5%) as a white solid.

### 1-[(4-nitrophenyl) methyl]piperidin-4-amine

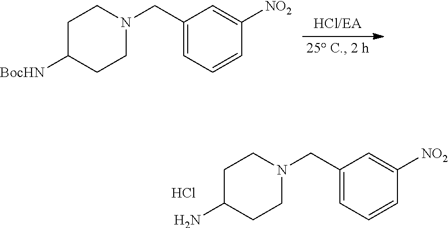 [SMILES: CC(C)(C)OC(=O)NC1CCN(Cc2cccc([N+](=O)[O-])c2)CC1 ; Cl ; NC1CCN(Cc2cccc([N+](=O)[O-])c2)CC1]

A solution of tert-butyl N-[1-[(4-nitrophenyl) methyl]-4-piperidyl]carbamate (5 g, 14.91 mmol) in HCl/EA (20 mL) was stirred at 25° C. for 2 h. The mixture was filtered and the solid was collected and concentrated under vacuum to give the title compound (4 g, 91.2%), which was used for next step directly.

### 5-chloro-4-(1Hindol-3-yl)-N-[1-[(3-nitrophenyl) methyl]-4-piperidyl]pyrimidin-2-amine

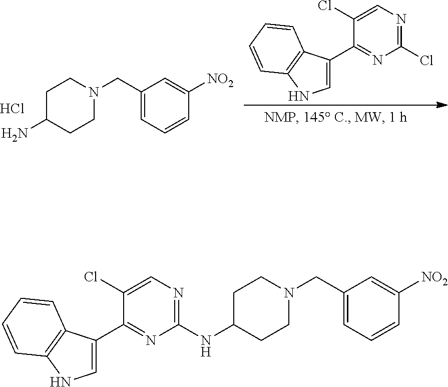 [SMILES: Cl ; NC1CCN(Cc2cccc([N+](=O)[O-])c2)CC1 ; Clc1ncc(Cl)c(-c2c[nH]c3ccccc23)n1 ; O=[N+]([O-])c1cccc(CN2CCC(Nc3ncc(Cl)c(-c4c[nH]c5ccccc45)n3)CC2)c1]

A mixture of 1-[(4-nitrophenyl) methyl]piperidin-4-amine (501 mg, 2.13 mmol), 3-(2,5-dichloropyrimidin-4-yl)-1H-indole (281 mg, 1.07 mmol) and DIPEA (413 mg, 3.20 mmol) in NMP (10 mL) was heated to 145° C. and stirred for 1 h (mW). The mixture was poured into water, extracted with EA, and the organic phase was washed with saturated brine, dried over anhydrous Na₂SO₄, and concentrated. The residue was purified by silica gel chromatography to afford the title compound (200 mg, 40.6%) as a yellow solid.

### N-[1-[(3-aminophenyl)methyl]-4-piperidyl]-5-chloro-4-(1H-indol-3-yl)pyrimidin-2-amine (Compound 319)

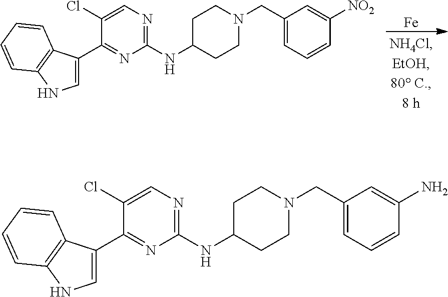

To a mixture of [3-[[4-[[5-chloro-4-(1H-indol-3-yl) pyrimidin-2-yl] amino]-1-piperidyl] methyl]phenyl]azinate (500 mg, 1.08 mmol) in EtOH (20 mL) and NH₄Cl solution (5 mL) was added Fe (302 mg, 5.40 mmol) and the mixture was heated to 80° C. for 8 h. The mixture was filtered and the organic phase diluted with water, extracted with EA, dried over anhydrous Na₂SO₄, and concentrated to give the title compound (430 mg, 82.7%) as a yellow solid.

### (E)-N-[3-[[4-[[5-chloro-4-(1H-indol-3-yl)pyrimidin-2-yl]amino]-1-piperidyl]methyl]phenyl]-4-(dimethylamino)but-2-enamide (Compound 141)

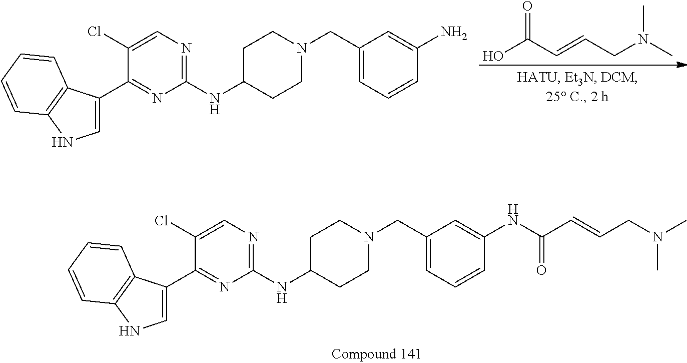

To a mixture of N-[1-[(3-aminophenyl)methyl]-4-piperidyl]-5-chloro-4-(1H-indol-3-yl)pyrimidin-2-amine (60 mg, 0.14 mmol) and (E)-4-(dimethylamino)but-2-enoic acid (36 mg, 0.28 mmol) in DCM (10 mL) was added Et₃N (42 mg, 0.41 mmol) and HATU (106 mg, 0.28 mmol) at 25° C. and the mixture was stirred for 2 h. The volatiles were evaporated and the residue was purified by prep-HPLC to afford the title compound (45 mg, 59.7%) as a white solid.

¹H NMR: (MeOD, 400 MHz): δ 1.64-1.73 (m, 2H), 2.12 (d, J=14.55 Hz, 2H), 2.26 (br. s., 2H), 2.31 (s, 6H), 2.95-3.03 (m, 2H), 3.19 (dd, J=6.62, 1.32 Hz, 2H), 3.61 (s, 2H), 3.95 (br. s., 1H), 6.29 (dt, J=15.44, 1.54 Hz, 1H), 6.91 (dt, J=15.33, 6.45 Hz, 1H), 7.10-7.16 (m, 2H), 7.22 (td, J=7.61, 1.10 Hz, 1H), 7.30-7.34 (m, 1H), 7.45 (d, J=8.38 Hz, 1H), 7.59 (d, J=8.38 Hz, 1H), 7.64 (s, 1H), 8.13-8.17 (m, 1H), 8.45-8.49 (m, 1H), 8.60 (d, J=7.94 Hz, 1H). MS (m/z): 544.2 [M+1]⁺.

### Example 38. Synthesis of (E)-N-[4-[[4-[[5-chloro-4-(1H-indol-3-yl)pyrimidin-2-yl]amino]-1-piperidyl]methyl]phenyl]-4-(methylamino)but-2-enamide (Compound 142)

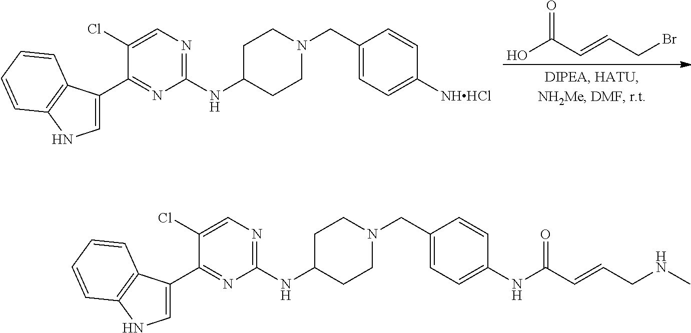

To a stirred solution of N-[1-[(4-aminophenyl)methyl]-4-piperidyl]-5-chloro-4-(1H-indol-3-yl)pyrimidin-2-amine (Compound 48, HCl salt; 200 mg, 426.07 umol) and (E)-4-bromobut-2-enoic acid (84.36 mg, 511.28 umol) in DMF (5 mL) was added HATU (194.41 mg, 511.28 umol) and DIPEA (165.20 mg, 1.28 mmol) at rt. Then the reaction mixture was stirred at 6° C. After 2 hr the MeNH₂ (5 mL, 10 mmol) was added to the reaction mixture, which was allowed to continue to stir for 2 hr. The mixture was concentrated and purified by prep-HPLC twice to obtain the title compound (5.00 mg, 9.43 umol, 2.21% yield). ¹H NMR: (400 MHz; MeOD): δ ppm 8.61 (d, J=8 Hz, 1H), 8.47 (s, 1H), 8.14 (s, 1H), 7.62 (d, J=8.4 Hz, 2H), 7.45 (d, J=8 Hz, 1H), 7.34 (d, J=8 Hz, 2H), 7.21 (d, J=8 Hz, 1H), 7.14 (d, J=7.6 Hz, 1H), 6.95-6.91 (m, 1H), 6.25 (d, J=15.2 Hz, 1H), 3.93 (s, 1H), 3.58 (s, 2H), 3.39 (d, J=5.6 Hz, 2H), 2.97 (d, J=11.2 Hz, 2H), 2.41 (s, 3H), 2.29-2.23 (m, 2H), 2.10 (s, 2H), 1.70-1.62 (M, 2H).

### Example 39. Synthesis of (E)-N-(4-((4-((5-chloro-4-(1H-indol-3-yl)pyrimidin-2-yl)amino) piperidin-1-yl)methyl)phenyl)-4-((2,3-dihydroxypropyl)(methyl)amino)but-2-enamide (Compound 143)

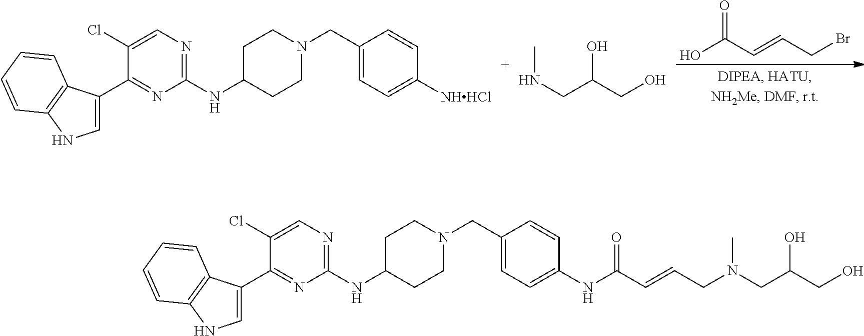

To a stirred solution of N-[1-[(4-aminophenyl)methyl]-4-piperidyl]-5-chloro-4-(1H-indol-3-yl) pyrimidin-2-amine (Compound 48, HCl salt; 200 mg, 426.07 umol) and DIPEA (165.2 mg, 1.28 mmol) in DMF (5 mL) was added (E)-4-bromobut-2-enoic acid (105.4 mg, 639.10 umol) and HATU (243.01 mg, 639.10 umol) at rt, then the reaction mixture was stirred for 2 hr. Once the starting material was consumed (as shown by LCMS), 3-(methylamino)propane-1,2-diol (134.38 mg, 1.28 mmol) was added, then the mixture was stirred for another 2 hr. The reaction was monitored by LCMS. The mixture was concentrated and purified by prep-HPLC to give the title compound (30 mg, 49.66 umol, 11.65% yield). ¹H NMR: (400 MHz; MeOD): δ ppm 8.60 (d, J=8 Hz, 1H), 8.47 (s, 1H), 8.16 (s, 1H), 7.66 (d, J=8 Hz, 2H), 7.45 (d, J=8 Hz, 1H), 7.37 (d, J=8.4 Hz, 2H), 7.24-7.22 (m, 1H), 7.21-7.16 (m, 1H), 7.14-6.91 (m, 1H), 6.32 (d, J=15.6 Hz, 1H), 3.99 (s, 1H), 3.82 (s, 1H), 3.75 (s, 2H), 3.55-3.51 (m, 3H), 3.38-3.37 (m, 2H), 3.10 (d, J=11.2 Hz, 2H), 2.59-2.53 (m, 3H), 2.40 (s, 3H), 2.16 (d, J=10.2 Hz, 2H), 1.72 (d, J=11.2 Hz, 2H).

### Example 40. Synthesis of (R,E)-N-(4-((3-((5-chloro-4-(1H-indol-3-yl) pyrimidin-2-yl)amino)pyrrolidin-1-yl)methyl)phenyl)-4-(dimethylamino)but-2-enamide (E)-4-(dimethylamino)but-2-enoate (Compound 144)

### (R)-tert-butyl (1-(4-acetamidobenzyl)pyrrolidin-3-yl) carbamate

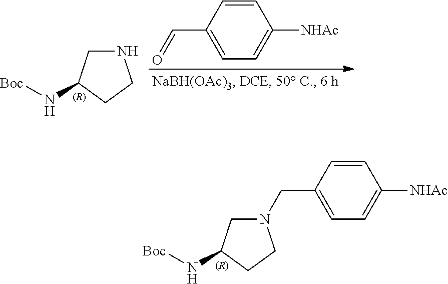

A mixture of (R)-tert-butyl pyrrolidin-3-ylcarbamate (5.0 g, 26.85 mmol), N-(4-formylphenyl)acetamide (4.38 g, 26.85 mmol) and NaBH(OAc)₃ (11.38 g, 63.70 mmol) in DCE (120 mL) was stirred at 50° C. for 12 h. The mixture was partitioned between H₂O and DCM. The organic layer was dried and concentrated. The residue was purified by column to afford the title compound (5 g, 55.9% yield).

### (R)—N-(4-((3-aminopyrrolidin-1-yl)methyl)phenyl)acetamide

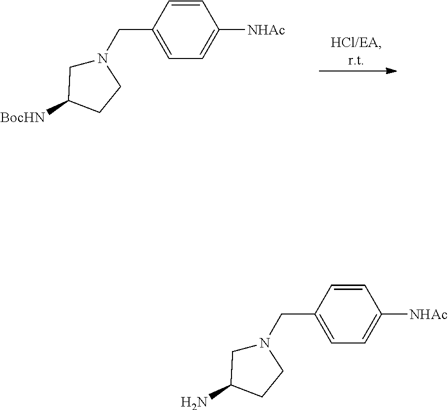 [SMILES: CC(=O)Nc1ccc(CN2CC[C@@H](NC(=O)OC(C)(C)C)C2)cc1 ; CC(=O)Nc1ccc(CN2CC[C@@H](N)C2)cc1]

A solution of (R)-tert-butyl (1-(4-acetamidobenzyl)pyrrolidin-3-yl)carbamate (5.0 g, 15.0 mmol) in HCl/EA (100 mL) was stirred at 15° C. for 8 h. The mixture was concentrated to afford (R)—N-(4-((3-aminopyrrolidin-1-yl)methyl)phenyl)acetamide (3.2 g, 91.4% yield).

### 3-(2-(((R)-1-(4-acetamidobenzyl)pyrrolidin-3-yl)amino)-5-chloropyrimidin-4-yl)-1H-indol-1-yl benzenesulfinate

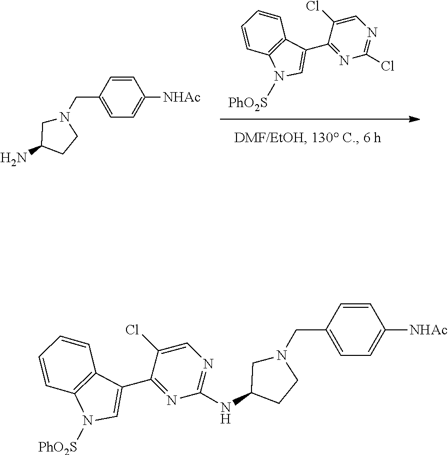

A mixture of (R)—N-(4-((3-aminopyrrolidin-1-yl)methyl)phenyl)acetamide (1.5 g, 6.43 mmol), 3-(2,5-dichloropyrimidin-4-yl)-1-(phenylsulfonyl)-1H-indole (2.26 g, 6.43 mmol) and DIPEA (2.49 g, 19.29 mmol) in DMF/EtOH (15:15 mL) was stirred at 130° C. for 6 h. The mixture was concentrated and purified by column (PE:EA=5:1) to afford the title compound (1.9 g, 49.2% yield).

### (R)—N-(1-(4-aminobenzyl)pyrrolidin-3-yl)-5-chloro-4-(1-(phenylsulfonyl)-1H-indol-3-yl)pyrimidin-2-amine

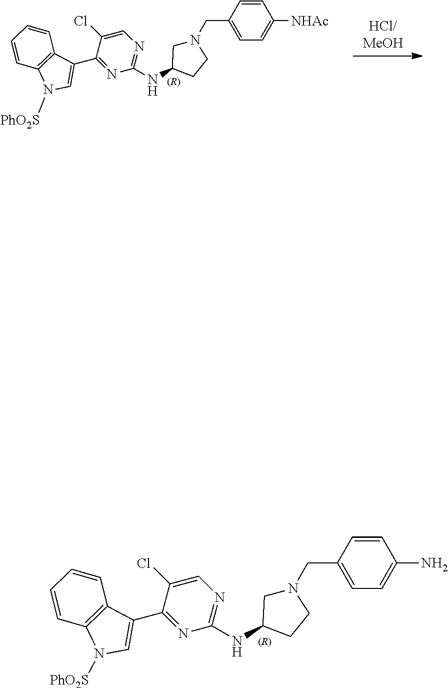

A solution of 3-(2-(((R)-1-(4-acetamidobenzyl)pyrrolidin-3-yl)amino)-5-chloropyrimidin-4-yl)-1H-indol-1-yl benzenesulfinate (2.0 g, 3.33 mmol) in HCl/MeOH (50 mL) was stirred at 15° C. for 6 h. The mixture was concentrated to afford the title compound (1.5 g, crude).

### (R)—N-(1-(4-aminobenzyl)pyrrolidin-3-yl)-5-chloro-4-(1H-indol-3-yl)pyrimidin-2-amine (Compound 320)

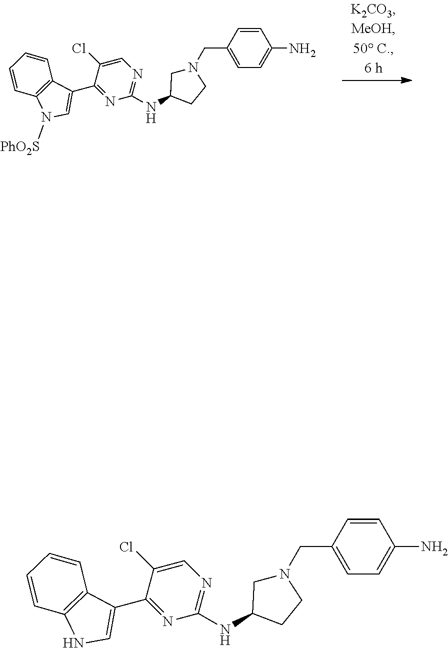

A mixture of (R)—N-(1-(4-aminobenzyl)pyrrolidin-3-yl)-5-chloro-4-(1-(phenylsulfonyl)-1H-indol-3-yl)pyrimidin-2-amine (1.5 g, 2.68 mmol) and K₂CO₃ (1.11 g, 8.05 mmol) in MeOH (200 mL) was stirred at 50° C. for 6 h. The mixture was filtered and concentrated to afford the title compound (850 mg, 75.7% yield).

### (R,E)-N-(4-((3-((5-chloro-4-(1H-indol-3-yl)pyrimidin-2-yl)amino pyrrolidin-1-yl)methyl)phenyl)-4-(dimethylamino)but-2-enamide-(E)-4-(dimethylamino)but-2-enoate (Compound 144)

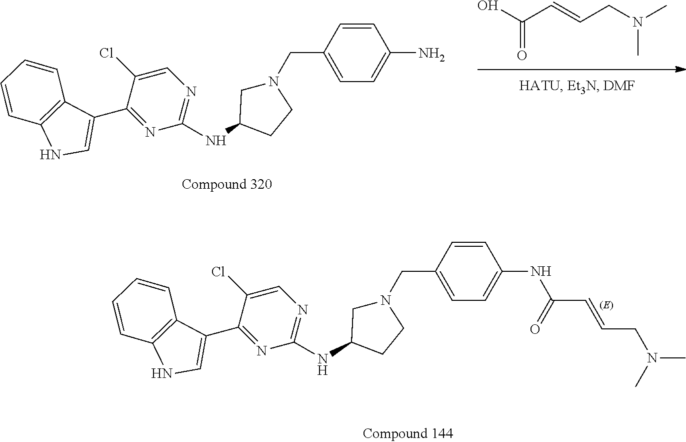

To a solution of (R)—N-(1-(4-aminobenzyl)pyrrolidin-3-yl)-5-chloro-4-(1H-indol-3-yl)pyrimidin-2-amine (50 mg, 119.35 umol) and (E)-4-(dimethylamino)but-2-enoic acid (16.96 mg, 131.29 mmol) in DMF (1 mL) was added HATU (54.46 mg, 143.22 μmol). The reaction mixture was stirred at 20° C. for 20 min, then Et₃N (48.31 mg, 477.4 μmol) was added, and the mixture was stirred at 20° C. for 2.5 h. The mixture was purified by prep-HPLC to afford the title compound (3 mg, 4.7% yield). ¹H NMR: (DMSO; 400 MHz): δ 9.09 (br, 1H), 8.49 (s, 1H), 8.33 (s, 1H), 7.76-7.50 (m, 5H), 6.82 (br, 1H), 6.52 (d, J=16 Hz, 1H), 4.36 (s, 2H), 3.91 (s, 4H), 2.93 (s, 3H), 2.76 (d, J=8 Hz, 8H), 1.95 (s, 2H). MS (m/z): 530.2 [M+H]+.

### Example 41. Synthesis of (E)-N-[3-[[4-[[5-chloro-4-(1H-indol-3-yl)pyrimidin-2-yl]amino]-1-piperidyl]methyl]phenyl]-4-(dimethylamino)-N-methyl-but-2-enamine (Compound 147)

### 5-chloro-4-(1H-indol-3-yl)-N-[1-[[3-(methylami)phenyl]methyl]-4-piperidyl]pyrimidin-2-amine (Compound 321)

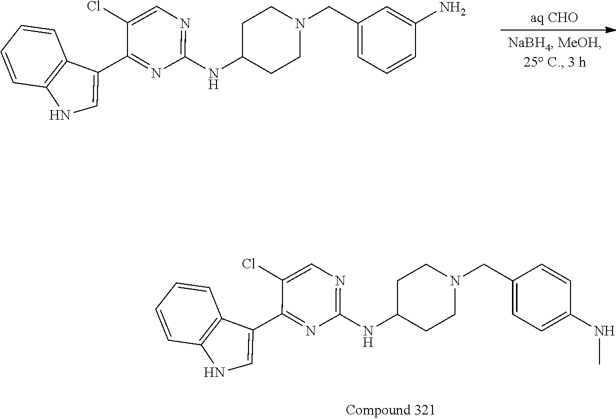 [SMILES: Nc1cccc(CN2CCC(Nc3ncc(Cl)c(-c4c[nH]c5ccccc45)n3)CC2)c1 ; CNc1ccc(CN2CCC(Nc3ncc(Cl)c(-c4c[nH]c5ccccc45)n3)CC2)cc1]

To a solution of N-[1-[(3-aminophenyl)methyl]-4-piperidyl]-5-chloro-4-(1H-indol-3-yl)pyrimidin-2-amine (500 mg, 1.15 mmol) in MeOH (15 mL) was added dropwise a solution of formaldehyde (35 mg, 1.15 mmol) in MeOH (5 mL) at 25° C. and the mixture was stirred for 1 h. NaBH₃CN (145 mg, 2.30 mmol) was then added and the mixture was stirred for an additional 2 h. The mixture was concentrated under reduced pressure, and the residue was purified by prep-HPLC to afford the title compound (150 mg, 17.5%) as a yellow solid.

### (E)-N-[3-[[4-[[5-chloro-4-(1H-indol-3-yl)pyrimidin-2-yl]amino]-1-piperidyl]methyl]phenyl]-4-(dimethylamino)-N-methyl-but-2-enamide (Compound 147)

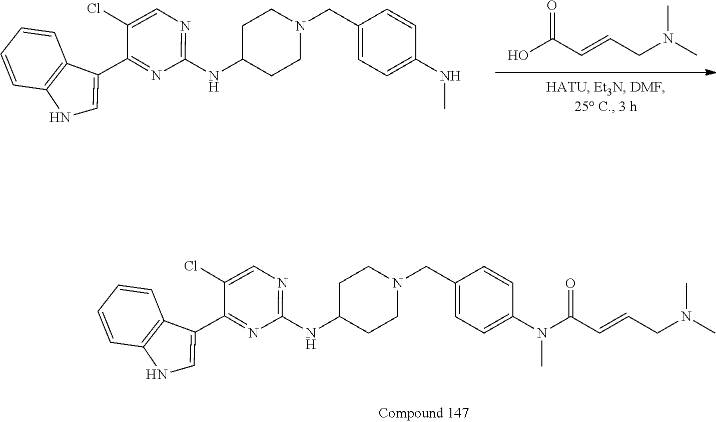

To a solution of 5-chloro-4-(1H-indol-3-yl)-N-[1-[[3-(methylamino)phenyl]methyl]-4-piperidyl]pyrimidin-2-amine (200 mg, 0.45 mmol) and (E)-4-(dimethylamino)but-2-enoic acid (87 mg, 0.67 mmol) in DCM (10 mL) was added HATU (340 mg, 0.59 mmol) and Et₃N (136 mg, 1.34 mmol) at 25° C. and the mixture was stirred for 3 h. Then the mixture was concentrated under vacuum and purified by prep-HPLC to afford the title compound (25 mg, 10%). ¹H NMR: (d₆-DMSO, 400 MHz): δ 1.58 (d, J=11.29 Hz, 2H), 1.86-2.18 (m, 10H), 2.86 (br. s., 4H), 3.25 (s, 3H), 3.55 (br. s., 2H), 3.83 (br. s., 1H), 5.88 (br. s., 1H), 6.62 (br. s., 1H), 7.08-7.25 (m, 5H), 7.33 (br. s., 1H), 7.43 (br. s., 1H), 7.49 (d, J=8.78 Hz, 1H), 8.24 (br. s., 1H), 8.44-8.65 (m, 2H), 11.85 (br. s., 1H). MS (m/z): 558.3 [M+H]⁺.

### Example 42. Synthesis of (E)-4-((2-amino-2-oxoethyl)(methyl)amino)-N-(4-((4-((5-chloro-4-(1H-indol-3-yl) pyrimidin-2-yl)amino)piperidin-1-yl)methyl)phenyl)but-2-enamide (Compound 146)

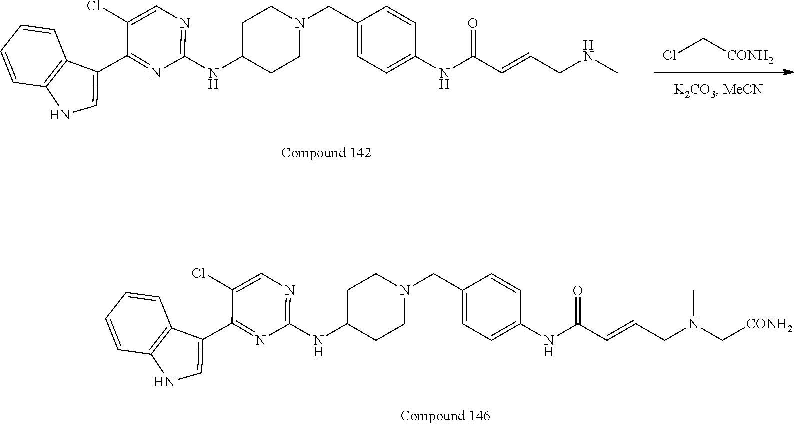 [SMILES: CNC/C=C/C(=O)Nc1ccc(CN2CCC(Nc3ncc(Cl)c(-c4c[nH]c5ccccc45)n3)CC2)cc1 ; NC(=O)CCl ; CN(C/C=C/C(=O)Nc1ccc(CN2CCC(Nc3ncc(Cl)c(-c4c[nH]c5ccccc45)n3)CC2)cc1)CC(N)=O]

To a stirred solution of (E)-N-[4-[[4-[[5-chloro-4-(1H-indol-3-yl)pyrimidin-2-yl]amino]-1-piperidyl]methyl]phenyl]-4-(methylamino)but-2-enamide (Compound 142; 30.00 mg, 56.60 umol) in MeCN (2 mL) was added 2-chloroacetamide (26.46 mg, 282.99 umol) and K₂CO₃ (39.11 mg, 282.99 umol). The reaction mixture was stirred at rt for 12 hr, filtered, concentrated, and purified by prep-HPLC to give the title compound (5.00 mg, 8.52 umol, 15.05% yield). ¹H NMR: (400 MHz; MeOD); δ ppm 8.60 (d, J=8 Hz, 1H), 8.47 (s, 1H), 8.14 (s, 1H), 7.62 (d, J=8.4 Hz, 2H), 7.45 (d, J=8.4 Hz, 1H), 7.33 (d, J=8 Hz, 2H), 7.22-7.20 (m, 1H), 7.15-7.13 (m, 1H), 6.94-6.90 (m, 1H), 6.30 (d, J=15.6 Hz, 1H), 3.92 (s, 1H), 3.56 (s, 2H), 3.06 (s, 2H), 2.99-2.94 (m, 3H), 2.35 (s, 3H), 2.33-2.22 (m, 2H), 2.11-2.08 (m, 2H), 1.69-1.64 (s, 3H).

### Example 43. Synthesis of 2-[[(E)-4-[4-[[4-[[5-chloro-4-(1H-indol-3-yl)pyrimidin-2-yl]amino]-1-piperidyl]methyl]anilino]-4-oxo-but-2-enyl]-methyl-amino]acetic acid (Compound 148)

### (E)-tert-butyl 2-((4-((4-((4-((5-chloro-4-(1H-indol-3-yl)pyrimidin-2-yl)amino)piperidin-1-yl)methyl)phenyl)amino)-4-oxobut-2-en-1-yl)(methyl)amino)acetate

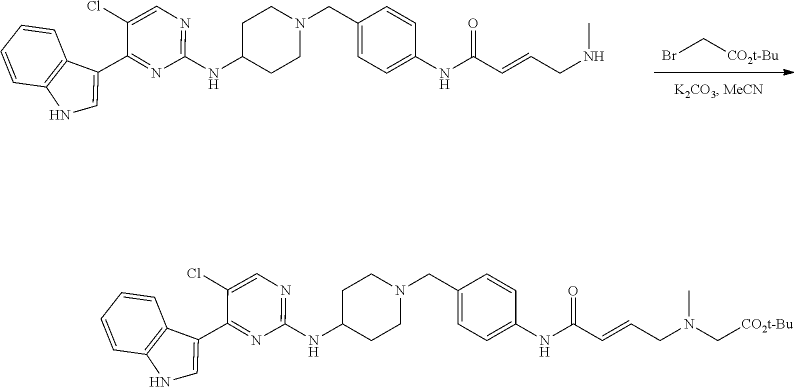 [SMILES: CNC/C=C/C(=O)Nc1ccc(CN2CCC(Nc3ncc(Cl)c(-c4c[nH]c5ccccc45)n3)CC2)cc1 ; CC(C)(C)OC(=O)CBr ; CN(C/C=C/C(=O)Nc1ccc(CN2CCC(Nc3ncc(Cl)c(-c4c[nH]c5ccccc45)n3)CC2)cc1)CC(=O)OC(C)(C)C]

To a stirred solution of (E)-N-[4-[[4-[[5-chloro-4-(1H-indol-3-yl)pyrimidin-2-yl]amino]-1-piperidyl]methyl]phenyl]-4-(methylamino)but-2-enamide (100.00 mg, 188.66 umol) and K₂CO₃ (52.15 mg, 377.32 umol) in MeCN (2 mL) was added tert-butyl 2-bromoacetate (73.60 mg, 377.32 umol), then the mixture was stirred at rt for 2 hr. The mixture was filtered and the filtrate was purified by prep-HPLC to afford the title compound (25.00 mg, 38.81 umol, 20.57% yield).

### 2-[[(E)-4-[4-[[4-[[5-chloro-4-(1H-indol-3-yl)pyrimidin-2-yl]amino]-1-piperidyl]methyl]anilino]-4-oxo-but-2-enyl]-methyl-amino]acetic acid

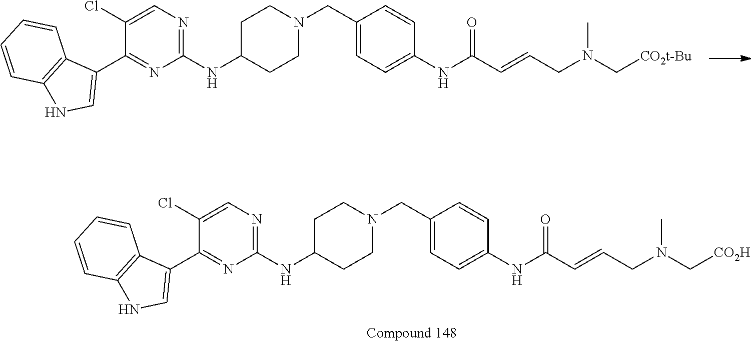 [SMILES: CN(C/C=C/C(=O)Nc1ccc(CN2CCC(Nc3ncc(Cl)c(-c4c[nH]c5ccccc45)n3)CC2)cc1)CC(=O)OC(C)(C)C ; CN(C/C=C/C(=O)Nc1ccc(CN2CCC(Nc3ncc(Cl)c(-c4c[nH]c5ccccc45)n3)CC2)cc1)CC(=O)O]

To a stirred solution of tert-butyl 2-[[(E)-4-[4-[[4-[[5-chloro-4-(1H-indol-3-yl)pyrimidin-2-yl]amino]-1-piperidyl]methyl]anilino]-4-oxo-but-2-enyl]-methyl-amino]acetate (25.00 mg, 38.81 umol, 1.00 Eq) in DCM (2 mL) was added TFA (2 mL) at rt. Then the reaction mixture was stirred at rt overnight. The mixture was concentrated and purified by prep-HPLC to afford the title compound (5.50 mg, 8.81 umol, 22.70% yield). ¹H NMR: (400 MHz; MeOD): δ ppm 8.94 (s, 1H), 8.59 (s, 1H), 8.30 (s, 1H), 7.84 (d, J=8.4 Hz, 2H), 7.60-7.58 (m, 3H), 7.36 (s, 2H), 6.94-6.89 (m, 1H), 6.61 (d, J=15.2 Hz, 1H), 4.41 (s, 2H), 4.17-4.11 (m, 4H), 3.65 (d, J=12.4 Hz, 2H), 3.46 (s, 2H), 3.00 (s, 3H), 2.45-2.35 (m, 3H), 2.04-2.01 (m, 2H).

### Example 44. Synthesis of N-((+/−cis)-5-((5-chloro-4-(1H-indol-3-yl)pyrimidin-2-yl)amino)piperidin-3-yl)-4-((E)-4-(dimethylamino)but-2-enamido)benzamide (Compound 149) and N-((+/−trans)-5-((5-chloro-4-(1H-indol-3-yl)pyrimidin-2-yl)amino)piperidin-3-yl)-4-((E)-4-(dimethylamino)but-2-enamido)benzamide (Compound 150)

### Dimethylpyridine-3,5-dicarboxylate

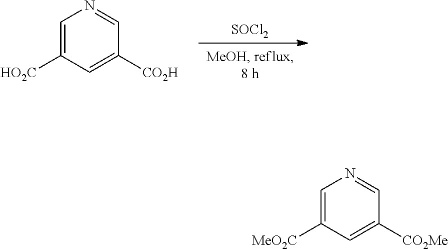 [SMILES: O=C(O)c1cncc(C(=O)O)c1 ; "SOCl2" ; COC(=O)c1cncc(C(=O)OC)c1]

To a solution of pyridine-3,5-dicarboxylic acid (85.0 g, 508.62 mmol) in MeOH (1 L) was added SOCl₂ (61.80 g, 519.46 mmol) dropwise. The reaction mixture was stirred and heated to 60° C. for 12 hr. The reaction mixture was concentrated under vacuum to afford the title compound (120.00 g, crude) as a white solid.

### Dimethylpiperidine-3,5-dicarboxylate

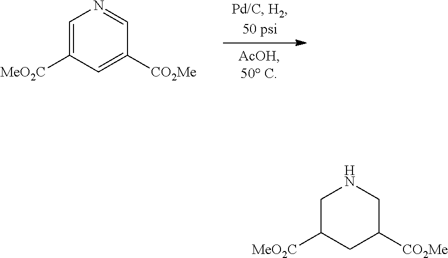 [SMILES: COC(=O)c1cncc(C(=O)OC)c1 ; "Pd/C, H2,50 psi" ; COC(=O)C1CNCC(C(=O)OC)C1]

To a solution of dimethyl pyridine-3,5-dicarboxylate (120.0 g, 614.85 mmol) in AcOH (500 mL) and MeOH (300 mL) was added Pd/C (10.0 g) under N₂. The suspension was degassed under vacuum and purged with H₂ several times. The mixture was stirred under H₂ (45 psi) at 50° C. for 48 hr. The reaction mixture was filtered and the filter was concentrated under vacuum to give title compound (140.0 g, crude) as a light yellow oil.

### 1-benzyl 3,5-dimethyl piperidine-1,3,5-tricarboxylate

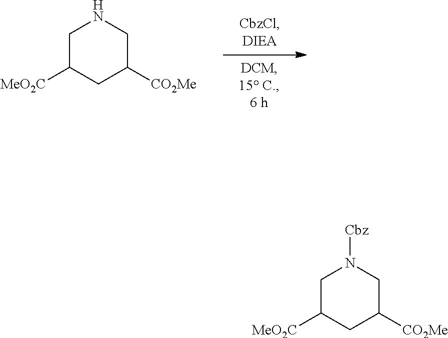 [SMILES: COC(=O)C1CNCC(C(=O)OC)C1 ; "CbzCl,DIEA" ; COC(=O)C1CC(C(=O)OC)CN(C(=O)OCc2ccccc2)C1]

To a solution of dimethyl piperidine-3,5-dicarboxylate (40 g, 198.85 mmol) and DIEA (51.38 g, 397.6 mmol) in DCM (500 mL) was added CbzCl (35.61 g, 208.7 mmol) dropwise and the mixture was stirred at 15° C. for 6 h. The mixture was and concentrated and purified by column to afford the title compound (20 g, crude).

### 1-((benzyloxy)carbonyl)-5-(methoxycarbonyl)piperidine-3-carboxylic acid

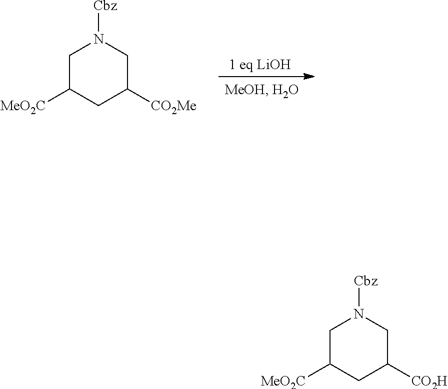 [SMILES: COC(=O)C1CC(C(=O)OC)CN(C(=O)OCc2ccccc2)C1 ; "1 eq LiOH" ; COC(=O)C1CC(C(=O)O)CN(C(=O)OCc2ccccc2)C1]

A mixture of 1-benzyl 3,5-dimethyl piperidine-1,3,5-tricarboxylate (20 g, 59.64 mmol) and LiOH.H₂O (2.5 g, 59.64 mmol) in H₂O (90 mL) and MeOH (180 mL) was stirred at 10° C. for 6 h. The mixture was concentrated and extracted with EA and the aqueous layer was adjusted to pH=2, then extracted with EA once again. The organic layer was dried and concentrated. The residue was purified by prep-HPLC under acidic conditions to afford the title compound (3.2 g, 16.7% yield).

### 1-benzyl 3-methyl 5-((tert-butoxycarbonyl)amino)piperidine-1,3-dicarboxylate

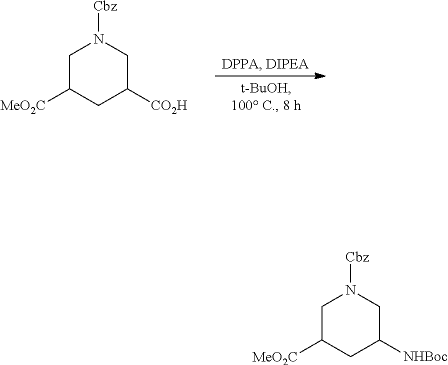 [SMILES: COC(=O)C1CC(C(=O)O)CN(C(=O)OCc2ccccc2)C1 ; "DPPA, DIPEA" ; COC(=O)C1CC(NC(=O)OC(C)(C)C)CN(C(=O)OCc2ccccc2)C1]

A mixture of 1-((benzyloxy) carbonyl)-5-(methoxycarbonyl) piperidine-3-carboxylic acid (3.5 g, 10.89 mmol), DPPA (3 g, 10.89 mmol) and DIEA (2.82 g, 21.87 mmol) in t-BuOH (50 mL) was stirred at 100° C. for 8 h. The mixture was then concentrated and purified by column (PE:EA=8:1) to afford 1-benzyl 3-methyl-5-((tert-butoxycarbonyl)amino)piperidine-1,3-dicarboxylate (3 g, 70.02% yield).

### 1-((benzyloxy) carbonyl)-5-((tert-butoxycarbonyl)amino)piperidine-3-carboxylic acid

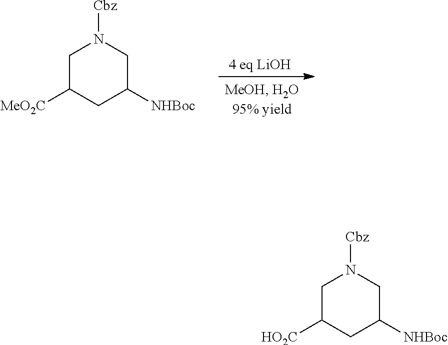 [SMILES: COC(=O)C1CC(NC(=O)OC(C)(C)C)CN(C(=O)OCc2ccccc2)C1 ; "4 eq LiOH" ; CC(C)(C)OC(=O)NC1CC(C(=O)O)CN(C(=O)OCc2ccccc2)C1]

A mixture of 1-benzyl 3-methyl 5-((tert-butoxycarbonyl)amino)piperidine-1,3-dicarboxylate (1 g, 2.5 mmol) in MeOH (20 mL) and H₂O (5 ml) was added LiOH.H₂O (0.4 g, 10 mmol). The reaction mixture was stirred at 25° C. for 5 h. The mixture was adjusted to pH=4 and extracted with EA. The organic layer was separated, dried, and concentrated to afford the title compound (900 mg, 95% yield).

### benzyl 3,5-bis((tert-butoxycarbonyl)amino)piperidine-1-carboxylate

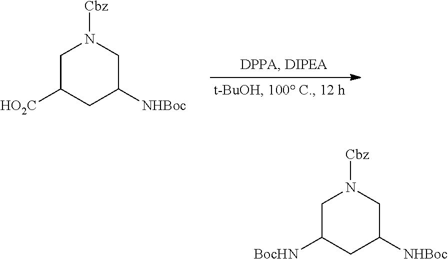 [SMILES: CC(C)(C)OC(=O)NC1CC(C(=O)O)CN(C(=O)OCc2ccccc2)C1 ; CC(C)(C)OC(=O)NC1CC(NC(=O)OC(C)(C)C)CN(C(=O)OCc2ccccc2)C1]

To a solution of 1-((benzyloxy)carbonyl)-5-((tert-butoxycarbonyl)amino)piperidine-3-carboxylic acid (0.9 g, 2.37 mmol) in t-BuOH (20 mL) was added DPPA (0.65 g, 2.37 mmol) and DIPEA (0.61 g, 4.74 mmol). The reaction mixture was stirred at rt for 0.5 h under a N₂ atmosphere. Then the solution was heated at reflux overnight, then cooled to room temperature, washed with saturated NaHCO₃ and extracted with EA. The organic layer was separated, dried, and concentrated. The residue was purified by column to afford the title compound (0.43 g, 40% yield)

### benzyl 3,5-bis((tert-butoxycarbonyl)amino)piperidine-1-carboxylate

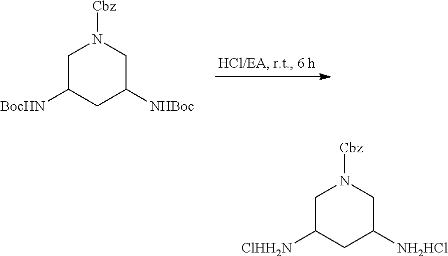

A mixture of benzyl 3,5-bis((tert-butoxycarbonyl)amino)piperidine-1-carboxylate (500 mg, 1.11 mmol) and HCl/EA (15 mL) was stirred at 15° C. for 6 h. The mixture was concentrated and used directly.

### 3-amino-5-((5-chloro-4-(1-(phenylsulfonyl)-H-indol-3-yl)pyrimidin-2-yl)amino) piperidine-1-carboxylate

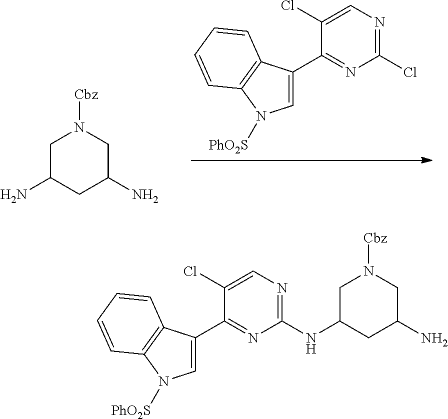

A mixture of benzyl 3,5-diaminopiperidine-1-carboxylate (797.07 mg, 2.47 mmol), 3-(2,5-dichloropyrimidin-4-yl)-1-(phenylsulfonyl)-1H-indole (1.0 g, 2.47 mmol), and DIEA (1.6 g, 12.37 mmol) in NMP (12 mL) was stirred at 140° C. (mW) for 1 h. The mixture was partitioned between EA and H₂O, and the organic layer was dried and concentrated to afford the title compound (1.0 g, crude).

### benzyl 3-amino-5-((5-chloro-4-(1H-indol-3-yl)pyrimidin-2-yl)amino)piperidine-1-carboxylate

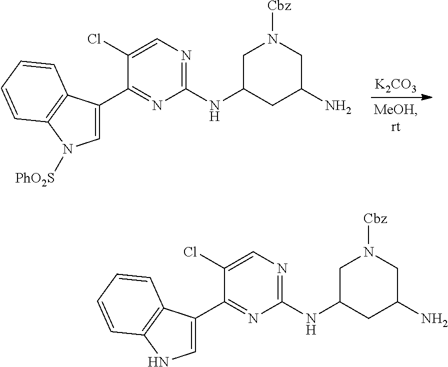

A mixture of benzyl 3-amino-5-((5-chloro-4-(1-(phenylsulfonyl)-1H-indol-3-yl)pyrimidin-2-yl) amino) piperidine-1-carboxylate (900 mg, 1.46 mmol) and K₂CO₃ (604.69 mg, 4.38 mmol) in EtOH (15 mL) was stirred at 60° C. for 6 h. The mixture was concentrated and purified by prep-HPLC to afford the title compound (110 mg, 15.8% yield for two steps).

### N-((+/−cis)-5-((5-chloro-4-(1H-indol-3-yl)pyrimidin-2-yl)amino)piperidin-3-yl)-4-((E)-4-(dimethylamino)but-2-enamido)benzamide and N-((+/−trans)-5-((5-chloro-4-(1H-indol-3-yl)pyrimidin-2-yl)amino)piperidin-3-yl)-4-((E)-4-(dimethylamino)but-2-enamido)benzamide

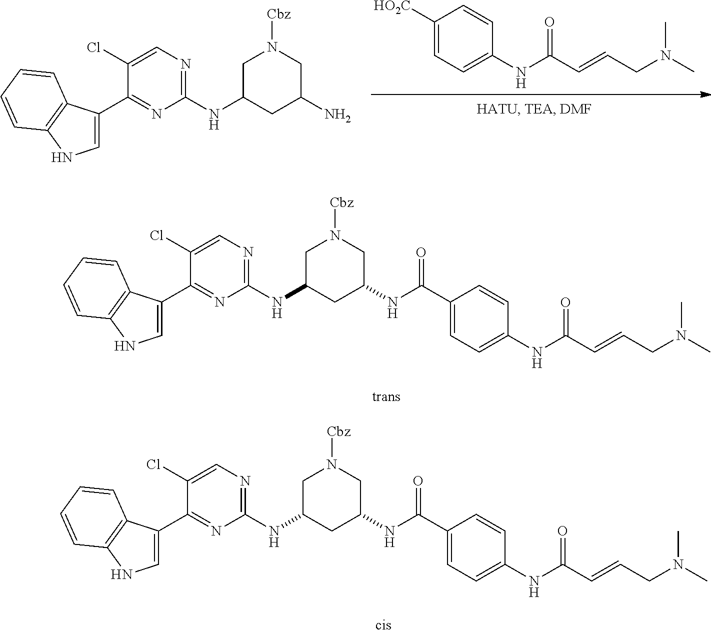

A mixture of benzyl-3-amino-5-((5-chloro-4-(1H-indol-3-yl)pyrimidin-2-yl)amino) piperidine-1-carboxylate (150 mg, 314.49 umol), HATU (143.5 mg, 377.39 ummol) and TEA (95.47 mg, 943.47 umol) in DMF (5 mL) was stirred at 15° C. for 8 h. The mixture was purified by prep-HPLC to afford the title compounds peak 1 (35 mg, 15.74% yield) and peak two (25 mg, 11.24% yield).

### N-((+/−cis)-5-((5-chloro-4-(1H-indol-3-yl)pyrimidin-2-yl)amino)piperidin-3-yl)-4-((E)-4-(dimethylamino)but-2-enamido)benzamide

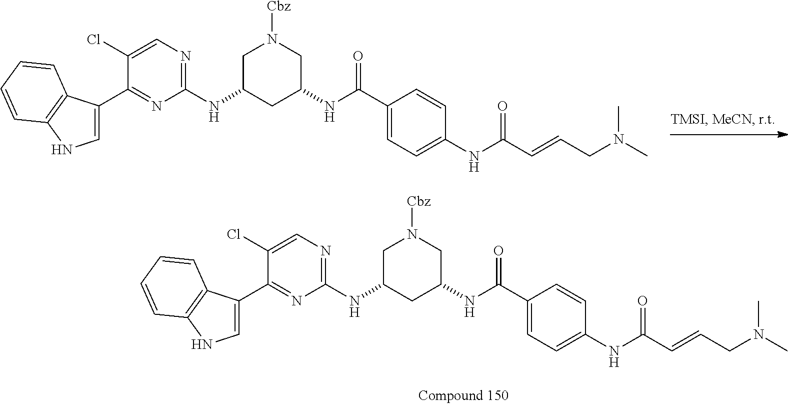

A mixture of (+/−cis)-benzyl 3-((5-chloro-4-(1H-indol-3-yl)pyrimidin-2-yl)amino)-5-(4-((E)-4-(dimethylamino)but-2-enamido)benzamido)piperidine-1-carboxylate (25 mg, 35.35 umol) in MeCN (2 mL) was added TMSI (28.28 mg, 141.40 umol) at rt. The mixture was stirred at rt for 3 h. The mixture was purified by prep-HPLC (HCl acid) to afford the title compound (2.3 mg, 11.35% yield). ¹H NMR: (CDCl₃; 400 MHz): δ 8.90 (s, 1H), 8.59 (br, 1H), 8.38 (s, 1H), 7.94 (d, J=8.8 Hz, 2H), 7.81 (d, J=8.8 Hz, 2H), 7.55 (d, J=7.2 Hz, 1H), 7.38-7.36 (m, 1H), 7.34-7.31 (m, 2H), 6.94-6.92 (m, 1H), 6.63 (d, J=16 Hz, 1H), 4.69 (br, 1H), 4.04 (d, J=7.2 Hz, 2H), 3.70-3.55 (m, 4H), 3.01-2.88 (m, 7H), 2.04 (br, 2H). MS (m/z): 573.2 [M+1]⁺.

### N-((+/−trans)-5-((5-chloro-4-(1H-indol-3-yl)pyrimidin-2-yl)amino)piperidin-3-yl)-4-((E)-4-(dimethylamino)but-2-enamido)benzamide

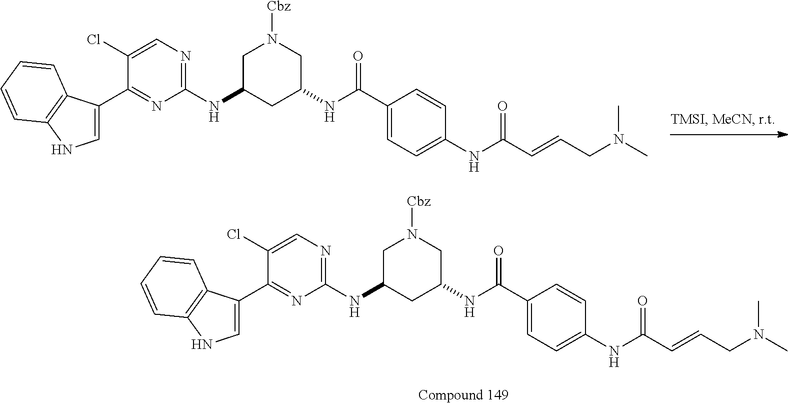

To a solution of (+/−trans)-benzyl 3-[[5-chloro-4-(1H-indol-3-yl)pyrimidin-2-yl] amino]-5-[[4-[[(E)-4-(dimethylamino)but-2-enoyl]amino]benzoyl]amino]piperidine-1-carboxylate (35.00 mg, 49.49 umol) in MeCN (2 mL) was added TMSI (39.61 mg, 197.96 umol) at rt and the mixture was stirred at rt for 3 h. The mixture was purified by prep-HPLC to afford the title compound (2.40 mg, 4.19 umol, 8.46% yield) as an HCl salt. ¹H NMR: (CDCl₃; 400 MHz): δ 8.89 (s, 1H), 8.59-8.57 (br, 1H), 8.35 (s, 1H), 7.88 (d, J=8.8 Hz, 2H), 7.79 (d, J=8.8 Hz, 2H), 7.55 (d, J=7.2 Hz, 1H), 7.46 (m, 1H), 7.35 (m, 1H), 6.88 (m, 1H), 6.58 (d, J=16 Hz, 1H), 4.63 (br, 1H), 4.00 (d, J=7.2 Hz, 2H), 3.70-3.55 (m, 4H), 3.15-3.12 (m, 1H), 3.05-2.93 (m, 7H), 2.69 (m, 1H), 2.06 (m, 1H). MS (m/z): 573.2 [M+1]⁺.

### Example 45. Synthesis of (+/−cis)-N-(4-acrylamidobenzyl)-5-((5-chloro-4-(1H-indol-3-yl)pyrimidin-2-yl) amino)piperidine-3-carboxamide (Compound 152) and (+/−trans)-N-(4-acrylamidobenzyl)-5-((5-chloro-4-(1H-indol-3-yl) pyrimidin-2-yl)amino)piperidine-3-carboxamide (Compound 151)

### 1-tert-butyl 3,5-dimethyl piperidine-1,3,5-tricarboxylate

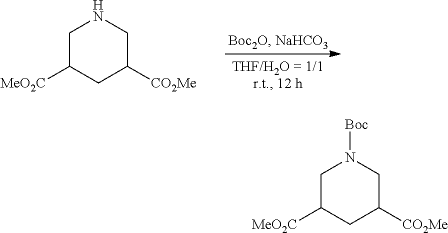 [SMILES: COC(=O)C1CNCC(C(=O)OC)C1 ; COC(=O)C1CC(C(=O)OC)CN(C(=O)OC(C)(C)C)C1]

To a solution of dimethyl piperidine-3,5-dicarboxylate (150.00 g, 745.45 mmol) in THF (500 mL) was added sat. NaHCO₃ solution (400 mL) to pH 7.0-8.0. Then Boc₂O (162.70 g, 745.45 mmol) in THF (500 mL) was added dropwise. The mixture was stirred at 25° C. for 12 hr. The reaction mixture was concentrated under vacuum. The residue was dissolved with EA (200 mL) and extracted with EA (2×300 mL). The combined organic layer was dried over Na₂SO₄ and concentrated under vacuum to afford the title compound (157.00 g, crude) as a light yellow oil.

### 1-(tert-butoxycarbonyl)-5-(methoxycarbonyl)piperidine-3-carboxylic acid

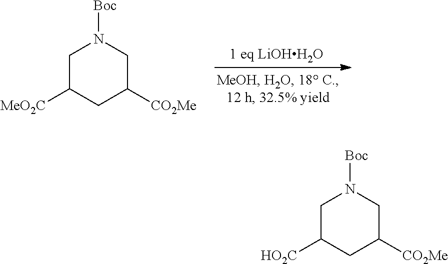 [SMILES: COC(=O)C1CC(C(=O)OC)CN(C(=O)OC(C)(C)C)C1 ; COC(=O)C1CC(C(=O)O)CN(C(=O)OC(C)(C)C)C1]

To a solution of 1-tert-butyl 3,5-dimethyl piperidine-1,3,5-tricarboxylate (2.00 g, 6.64 mmol) in MeOH (10 mL) and H₂O (10 mL) was added LiOH.H₂O (278.64 mg, 6.64 mmol). The mixture was stirred at 18° C. for 12 hr. The reaction mixture was adjusted to pH 4.0˜5.0, extracted with EA twice. The organic layer was dried over Na₂SO₄ and concentrated under vacuum to afford the title compound (620.00 mg, 2.16 mmol, 32.50% yield) as a light yellow oil.

### 1-tert-butyl 3-methyl 5-aminopiperidine-1,3-dicarboxylate

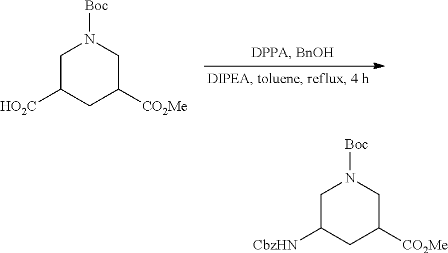 [SMILES: COC(=O)C1CC(C(=O)O)CN(C(=O)OC(C)(C)C)C1 ; COC(=O)C1CC(NC(=O)OCc2ccccc2)CN(C(=O)OC(C)(C)C)C1]

To a solution of 1-(tert-butoxycarbonyl)-5-(methoxycarbonyl) piperidine-3-carboxylic acid (5 g, 0.017 mol) and DIPEA (5 g, 0.0187 mol) in toluene (100 mL) was added BnOH (0.0187 g) and DPPA (0.0187 mol) with stirred at 120° C. for 4 hrs under N₂ atmosphere. After prep-TLC purification, the solution was washed with aq of NaHCO₃ and extracted with EA. The organic layer was separated, dried, and concentrated to afford the crude product. After column purification, title compound was obtained as an oil (2.8 g, 44% yield).

### 1-tert-butyl 3-methyl 5-aminopiperidine-1,3-dicarboxylate

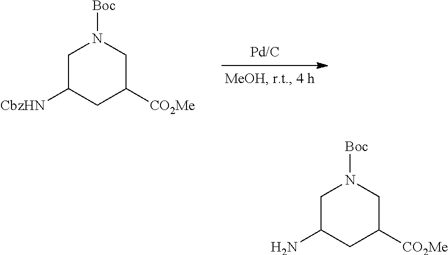 [SMILES: COC(=O)C1CC(NC(=O)OCc2ccccc2)CN(C(=O)OC(C)(C)C)C1 ; COC(=O)C1CC(N)CN(C(=O)OC(C)(C)C)C1]

To a solution of 1-tert-butyl 3-methyl 5-(((benzyloxy)carbonyl)amino)piperidine-1,3-dicarboxylate (2.80 g, 7.13 mmol) in MeOH (80 mL) was added Pd/C (0.50 g) under H₂ (45 psi) and the mixture was stirred at rt for 4 h. The mixture was filtered and filtrate was concentrated under vacuum to afford 1-tert-butyl 3-methyl 5-aminopiperidine-1,3-dicarboxylate (1.80 g, 97.7% yield) as a light yellow oil.

### 1-tert-butyl 3-methyl 5-((5-chloro-4-(1-(phenylsulfonyl)-1H-indol-3-yl)pyrimidin-2-yl)amino)piperidine-1,3-dicarboxylate

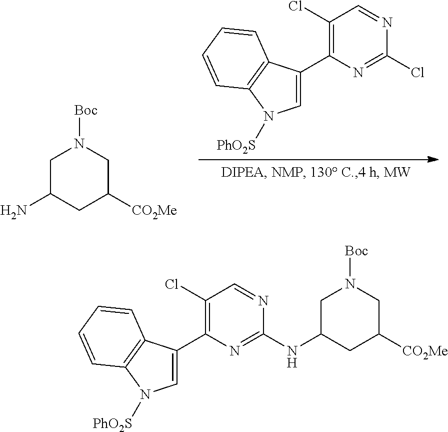

3-(2,5-dichloropyrimidin-4-yl)-1-(phenylsulfonyl)-1H-indole (2.00 g, 4.95 mmol), 1-tert-butyl 3-methyl 5-aminopiperidine-1,3-dicarboxylate (1.50 g, 5.79 mmol) and DIPEA (1.28 g, 9.89 mmol) were taken up into a microwave tube in NMP (10 mL) and the sealed tube was heated at 130° C. for 4 h under microwave. After cooling to rt, EA (100 mL) and 5% Na₂CO₃ (100 mL) were added. The aqueous layer was extracted with EA (2×100 mL). The combined organic layers were washed with brine (100 mL), dried over Na₂SO₄ and concentrated under vacuum to afford the title compound (2.19 g, 3.50 mmol, 70.66% yield) as a yellow solid.

### 1-(tert-butoxycarbonyl)-5-((5-chloro-4-(1H-indol-3-yl)pyrimidin-2-yl)amino)piperidine-3-carboxylic acid

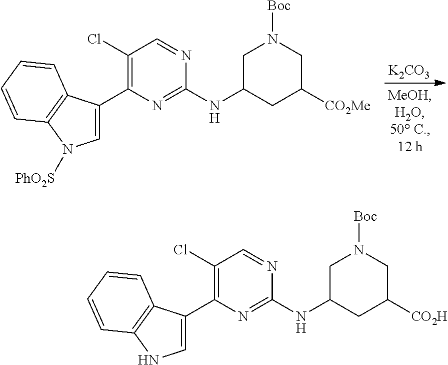

To a solution of 1-tert-butyl 3-methyl 5-((5-chloro-4-(1-(phenylsulfonyl)-1H-indol-3-yl)pyrimidin-2-yl)amino)piperidine-1,3-dicarboxylate (2.59 g, 4.14 mmol) in MeOH (40 mL) was added K₂CO₃ (1.14 g, 8.28 mmol, 2.00 Eq). The mixture was stirred at 50° C. for 12 hr. Then the mixture was cooled to 20° C. and H₂O (10 mL) was added, the mixture was stirred at 20° C. for 4 hr. The mixture was adjusted to pH 5.0-6.0 and extracted with EA three times. The combined organic layer were washed brine, dried over Na₂SO₄, and concentrated under vacuum to afford the title compound (94.17% yield) as a yellow solid.

### Tert-butyl 3-((4-acrylamidobenzyl)carbamoyl)-5-((5-chloro-4-(1H-indol-3-yl)pyramidin-2-yl)amino)piperidine-1-carboxylate

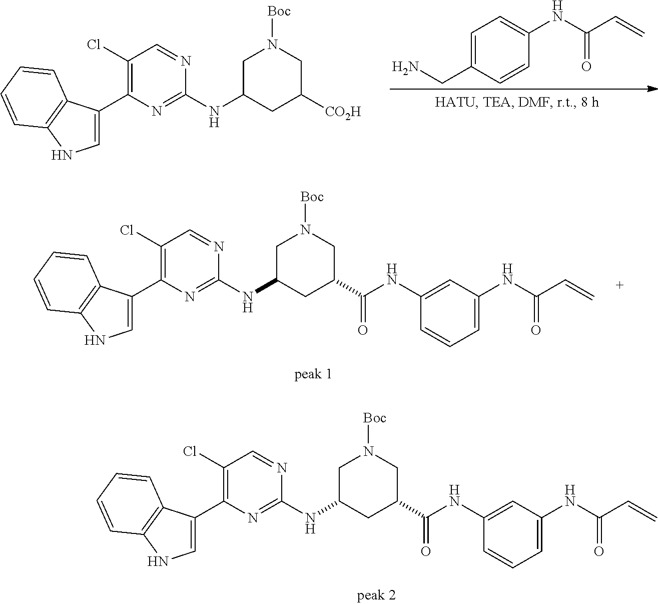

To a solution of 1-(tert-butoxycarbonyl)-5-((5-chloro-4-(1H-indol-3-yl)pyrimidin-2-yl)amino)piperidine-3-carboxylic acid (300.00 mg, 635.68 umol) in DMF (10 mL) was added N-(4-(aminomethyl)phenyl)acrylamide (168.00 mg, 953.41 umol), TEA (193.00 mg, 1.91 mmol) and HATU (242.00 mg, 635.68 umol). The reaction mixture was stirred at 20° C. for 12 hr. The reaction mixture was concentrated under vacuum and the residue was purified by prep-HPLC to title compound peak 1 (140.00 mg, 222.17 umol, 34.95% yield) as a yellow solid and peak 2 (100.0 mg, 158.69 umol, 24.96% yield) as a yellow solid

### (+/−cis)-N-(4-acrylamidobenzyl)-5-((5-chloro-4-(1H-indol-3-yl)pyrmidin-2-yl)amino)piperidine-3-carboxamide

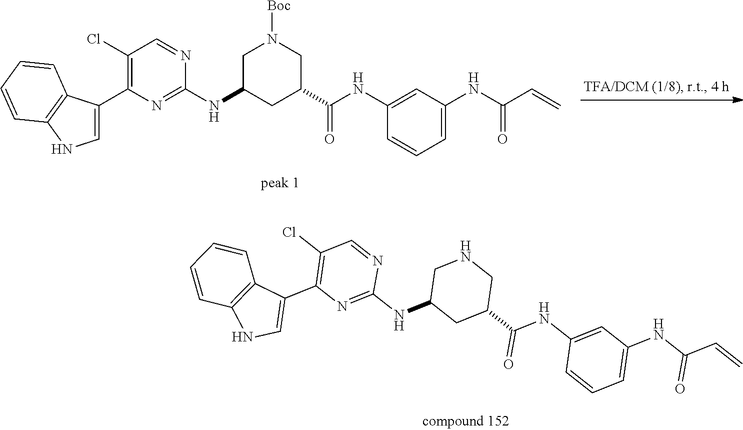

A solution of (+/−cis)-tert-butyl 3-((4-acrylamidobenzyl)carbamoyl)-5-((5-chloro-4-(1H-indol-3-yl)pyrimidin-2-yl)amino)piperidine-1-carboxylate (115.00 mg, 182.50 umol) in TFA/DCM (1:8) (5 mL) was stirred at 20° C. for 4 hr. The mixture was concentrated under vacuum and the residue was exchanged by AMBERLYST® A21 to pH 7.0˜8.0 in MeOH (50 mL), filtered and filtrate was concentrated under vacuum to afford the title compound (95.00 mg, 179.24 umol, 98.21% yield) as a light yellow solid. ¹H NMR (400 MHz, MeOD) δ 8.55 (s, 1H), 8.43 (s, 1H), 8.24 (s, 1H), 7.57 (d, J=8.40 Hz, 2H), 7.46 (d, J=7.60 Hz, 1H), 7.23-7.19 (m, 4H), 6.45-6.32 (m, 2H), 5.77-5.75 (m, 1H), 4.34 (d, J=4.00 Hz, 2H), 3.65 (d, J=12.00 Hz, 1H), 3.44 (d, J=9.20 Hz, 1H), 3.17-3.11 (m, 1H), 2.94-2.91 (m, 2H), 2.70 (s, 1H), 2.43 (d, J=12.80 Hz, 1H), 1.95-1.86 (m, 1H). MS (m/z): 530.2 [M+1]⁺.

### (+/−trans)-N-(4-acrylamidobenzyl)-5-((5-chloro-4-(1H-indol-3-yl)pyramidin-2-yl)amino)piperidine-3-carboxamide

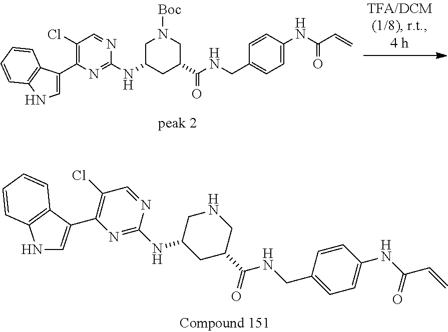

A solution of (3R,5S)-tert-butyl 3-((4-acrylamidobenzyl)carbamoyl)-5-((5-chloro-4-(1H-indol-3-yl)pyrimidin-2-yl)amino)piperidine-1-carboxylate (100.00 mg, 158.69 umol) in TFA/DCM (1/8) (5 mL) was stirred at 20° C. for 4 hr. The mixture was concentrated under vacuum. The residue was exchanged by AMBERLYST® A21 to pH 7.0-8.0 in MeOH, filtered and filtrate was concentrated under vacuum to afford (3R,5S)—N-(4-acrylamidobenzyl)-5-((5-chloro-4-(1H-indol-3-yl)pyrimidin-2-yl)amino)piperidine-3-carboxamide (67.00 mg, 126.41 umol, 79.66% yield) as a light yellow solid. ¹H NMR (400 MHz, MeOD) δ 8.56 (s, 1H), 8.46 (s, 1H), 8.23 (s, 1H), 7.52 (d, J=8.00 Hz, 2H), 7.45 (s, 1H), 7.22 (s, 4H), 6.44-6.31 (m, 2H), 5.75 (d, J=6.80 Hz, 1H), 4.31 (s, 2H), 3.32 (s, 2H), 3.17 (s, 2H), 3.01 (s, 1H), 2.69 (s, 1H), 2.25 (s, 1H), 2.13 (s, 1H). MS (m/z): 530.2 [M+1]⁺.

### Example 46. Synthesis of N-[4-(aminomethyl)phenyl]prop-2-enamide

### Tert-butyl N-[(4-aminophenyl)methyl]carbamate

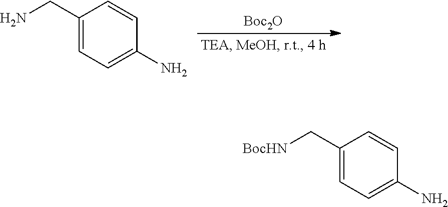 [SMILES: "Boc2O" ; NCc1ccc(N)cc1 ; CC(C)(C)OC(=O)NCc1ccc(N)cc1]

To a solution of 4-(aminomethyl)aniline (5.90 g, 48.29 mmol) in MeOH (100 mL) was added Boc₂O (10.54 g, 48.29 mmol) and TEA (9.77 g, 96.58 mmol). The mixture was stirred at 17° C. for 4 hr. The mixture was concentrated under vacuum. The residue was purified by column chromatography on silica gel to afford the title compound (8.64 g, 38.87 mmol, 80.49% yield) as a light yellow solid.

### Tert-butyl N-[[4-(prop-2-enoylamino)phenyl]methyl]carbamate

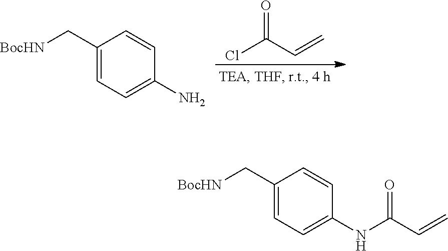

To a solution of tert-butyl N-[(4-aminophenyl)methyl]carbamate (2.00 g, 9.00 mmol) and TEA (1.82 g, 18.00 mmol) in THF (20 mL) under N₂ was added prop-2-enoyl chloride (896.05 mg, 9.90 mmol) dropwise. The mixture was stirred at 18° C. for 4 hr. The mixture was extracted with EA and washed with brine. The organic layer was dried over Na₂SO₄ and concentrated under vacuum. The residue was purified by column chromatography on silica gel to afford the title compound (1.92 g, 6.95 mmol, 77.20% yield) as a yellow solid.

### N-[4-(aminomethyl)phenyl]prop-2-enamide

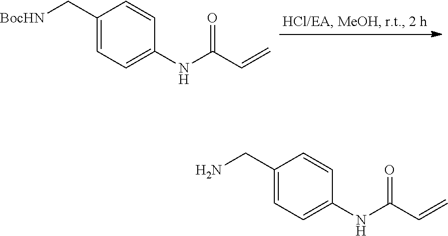 [SMILES: C=CC(=O)Nc1ccc(CNC(=O)OC(C)(C)C)cc1 ; C=CC(=O)Nc1ccc(CN)cc1]

To a solution of tert-butyl N-[[4-(prop-2-enoylamino)phenyl]methyl]carbamate (1.70 g, 6.15 mmol) in MeOH (5 mL) was added HCl/EA (50 mL). The mixture was stirred at 15° C. for 2 hr. The mixture was concentrated under vacuum to afford N-[4-(aminomethyl)phenyl]prop-2-enamide (1.45 g, crude, HCl salt) as a yellow solid.

### Example 47. Synthesis of (+/−cis)-N-(4-acrylamidophenyl)-5-((5-chloro-4-(1H-indol-3-yl)pyrimidin-2-yl) amino)piperidine-3-carboxamide (Compound 153) and (+/−trans)-N-(4-acrylamidophenyl)-5-((5-chloro-4-(1H-indol-3-yl)pyrimidin-2-yl) amino)piperidine-3-carboxamide (Compound 154)

### 1-(tert-butoxycarbonyl)-5-((5-chloro-4-(1H-indol-3-yl)pyrimidin-2-yl)amino)piperidine-3-carboxylic isobutyric anhydride

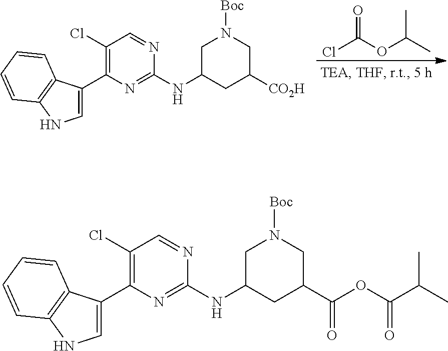

To a mixture of 1-tert-butoxycarbonyl-5-[[5-chloro-4-(1H-indol-3-yl)pyrimidin-2-yl]amino]piperidine-3-carboxylic acid (500.00 mg, 1.06 mmol) and TEA (429.05 mg, 4.24 mmol) in THF (5 mL) under N₂ was added a solution of isopropyl carbonochloridate (129.84 mg, 1.06 mmol) in THF (1 mL) dropwise. The reaction mixture was stirred at 20° C. for 4 h. The reaction mixture was used directly in next step.

### Tert-butyl 3-((4-acrylamidophenyl)carbamoyl)-5-((5-chloro-4-(1H-indol-3-yl)pyrimidin-2-yl)amino)piperidine-1-carboxylate

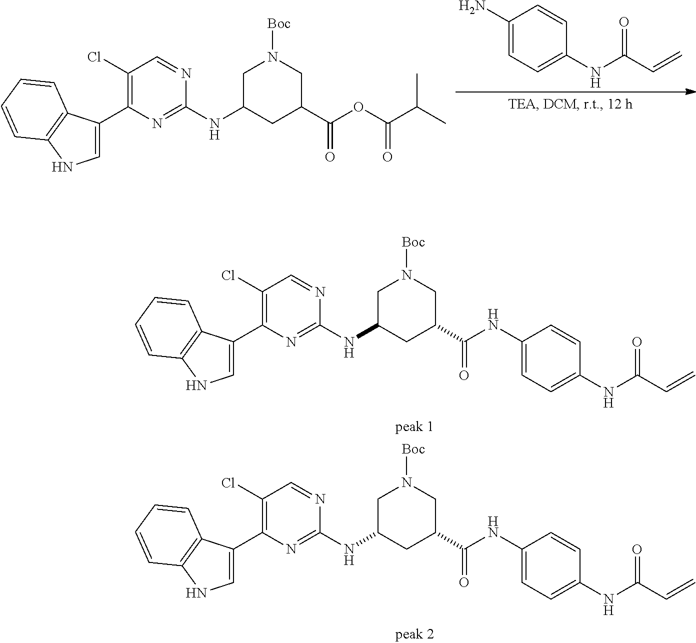

To a mixture of 1-(tert-butoxycarbonyl)-5-((5-chloro-4-(1H-indol-3-yl)pyrimidin-2-yl)amino)piperidine-3-carboxylic isobutyric anhydride (574.15 mg, 1.06 mmol) and TEA (429.05 mg, 4.24 mmol) in THF (5 m 1) under N2 was added a solution of N-(4-aminophenyl)prop-2-enamide (257.75 mg, 1.59 mmol) in DCM (5 mL) was added dropwise, the reaction mixture was stirred at 20° C. for 12 hr. The reaction mixture was concentrated under vacuum and the residue was purified by prep-HPLC to afford peak 1) (+/−trans)-tert-butyl 3-((4-acrylamidophenyl)carbamoyl)-5-((5-chloro-4-(1H-indol-3-yl)pyrimidin-2-yl)amino)piperidine-1-carboxylate (26.00 mg, 42.20 umol, 3.98% yield) as a yellow solid and (peak 2) (+/−trans)-tert-butyl-3-((4-acrylamidophenyl)carbamoyl)-5-((5-chloro-4-(1H-indol-3-yl)pyrimidin-2-yl)amino)piperidine-1-carboxylate (24.00 mg, 38.95 umol, 3.67% yield) as a yellow solid.

### (+/−cis)-N-(4-acrylamidophenyl)-5-((5-chloro-4-(1H-indol-3-yl)pyrimidin-2-yl) amino)piperidine-3-carboxamide

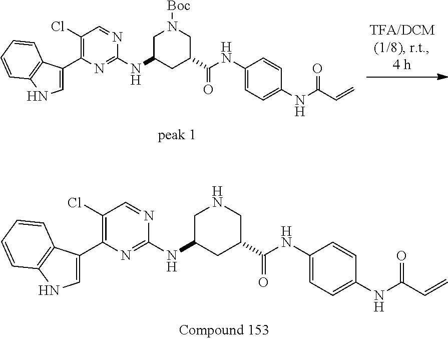

A solution of (+/−trans)-tert-butyl 3-((4-acrylamidophenyl)carbamoyl)-5-((5-chlor o-4-(1H-indol-3-yl)pyrimidin-2-yl)amino)piperidine-1-carboxylate (26.00 mg, 42.20 umol) in TFA/DCM (1:8) (5 mL) was stirred at 20° C. for 4 hr. The reaction mixture was concentrated under vacuum. The residue was purified by prep-HPLC to afford the title compound (10.00 mg, 19.40 umol, 45.91% yield) as a yellow solid HCl salt. ¹H NMR (400 MHz, MeOD) δ 8.81 (s, 1H), 8.58 (s, 1H), 8.27 (s, 1H), 7.58-7.54 (m, 5H), 7.40-7.33 (m, 2H), 6.46-6.32 (m, 2H), 5.76 (d, J=9.60 Hz, 1H), 4.62 (s, 1H), 3.74 (d, J=10.8 Hz, 1H), 3.56 (d, J=12.0 Hz, 1H), 3.21 (s, 3H), 2.55 (d, J=11.6 Hz, 1H), 2.20 (s, 1H). MS (m/z): 516.2 [M+1]⁺.

### (+/−trans)-N-(4-acrylamidophenyl)-5-((5-chloro-4-(1H-indol-3-yl)pyrimidin-2-yl)amino)piperidine-3-carboxamide

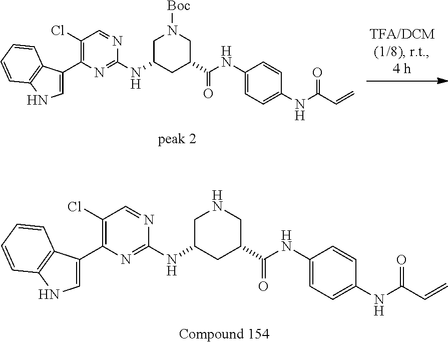

A solution of (+/−trans)-tert-butyl 3-((4-acrylamidophenyl)carbamoyl)-5-((5-chloro-4-(1H-indol-3-yl)pyrimidin-2-yl)amino)piperidine-1-carboxylate (20.00 mg, 32.46 umol, 1.00 Eq) in TFA/DCM (1:8) (5 mL) was stirred at 20° C. for 3 hr. The reaction mixture was concentrated under vacuum. The residue was exchanged by amberlyst-21 in MeOH to lyophilization afford the title compound (15.00 mg, 29.07 umol, 89.56% yield) as a light yellow solid free base. ¹H NMR (400 MHz, MeOD) δ 8.59 (d, J=7.20 Hz, 1H), 8.45 (s, 1H), 8.21 (s, 1H), 7.57-7.44 (m, 5H), 7.21-7.19 (m, 2H), 6.42-6.35 (m, 2H), 5.76-5.74 (m, 1H), 4.40 (s, 1H), 3.24-2.96 (m, 5H), 2.23-2.19 (m, 2H). MS (m/z): 516.2 [M+1]⁺.

### Example 48. Synthesis of N-(4-aminophenyl)prop-2-enamide

### Tert-butyl N-[4-(prop-2-enoylamino)phenyl]carbamate

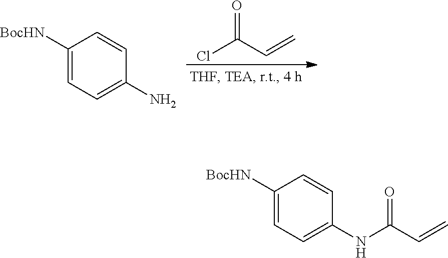

To a solution of tert-butyl N-(4-aminophenyl)carbamate (2.00 g, 9.60 mmol) and TEA (1.94 g, 19.20 mmol) in THF (20 mL) under N₂ was added prop-2-enoyl chloride (912.34 mg, 10.08 mmol) dropwise at 17° C. The reaction mixture was stirred at 17° C. for 4 hr. The mixture reaction was concentrated under vacuum. The residue was purified by column chromatography on silica gel to afford the title (2.22 g, 8.46 mmol, 88.16% yield) as a pale white solid.

### N-(4-aminophenyl)prop-2-enamide

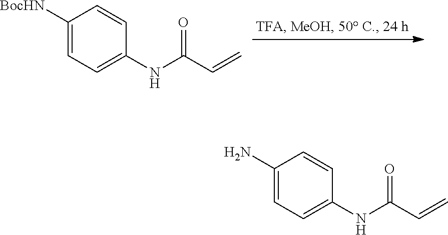 [SMILES: C=CC(=O)Nc1ccc(NC(=O)OC(C)(C)C)cc1 ; C=CC(=O)Nc1ccc(N)cc1]

To a solution of tert-butyl N-[4-(prop-2-enoylamino)phenyl]carbamate (1.80 g, 6.86 mmol) in MeOH (20 mL) was added TFA (10 mL). The reaction mixture was stirred at 50° C. for 24 hr. Most of starting material was consumed by TLC. The reaction mixture was concentrated under vacuum. The residue was dissolved in aq. Na₂CO₃ solution, extracted with EA. The organic layer was dried over Na₂SO₄ and concentrated under vacuum to afford the title compound (1.70 g, crude) as a brown solid.

### Example 49. Synthesis of (+/−trans)-N-(4-acrylamidobenzyl)-5-((5-chloro-4-(1H-indol-3-yl)pyrimidin-2-yl)amino)-1-methylpiperidine-3-carboxamide (Compound 155) and (+/−cis)-N-(4-acrylamidobenzyl)-5-((5-chloro-4-(1H-indol-3-yl)pyrimidin-2-yl)amino)-1-methylpiperidine-3-carboxamide (Compound 157)

### 5-[[5-chloro-4-(1H-indol-3-yl)pyrimidin-2-yl]amino]piperidine-3-carboxylic acid

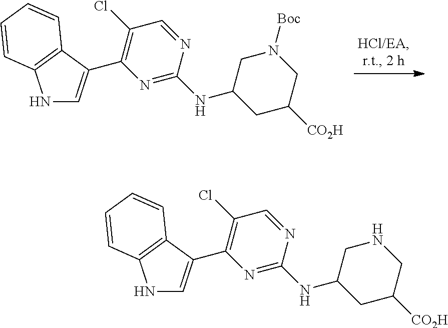 [SMILES: CC(C)(C)OC(=O)N1CC(Nc2ncc(Cl)c(-c3c[nH]c4ccccc34)n2)CC(C(=O)O)C1 ; O=C(O)C1CNCC(Nc2ncc(Cl)c(-c3c[nH]c4ccccc34)n2)C1]

To a solution of 1-tert-butoxycarbonyl-5-[[5-chloro-4-(1H-indol-3-yl)pyrimidin-2-yl]amino]piperidine-3-carboxylic acid (900.00 mg, 1.91 mmol) in DCM (2 mL) was added HCl/EA (20 mL). The reaction mixture was stirred at 25° C. for 2 hr. The reaction mixture was concentrated under vacuum to afford the title compound (790.00 mg, crude) as a yellow solid.

### 5-((5-chloro-4-(1H-indol-3-yl)pyrimidin-2-yl)amino)-1-methylpiperidine-3-carboxylic acid

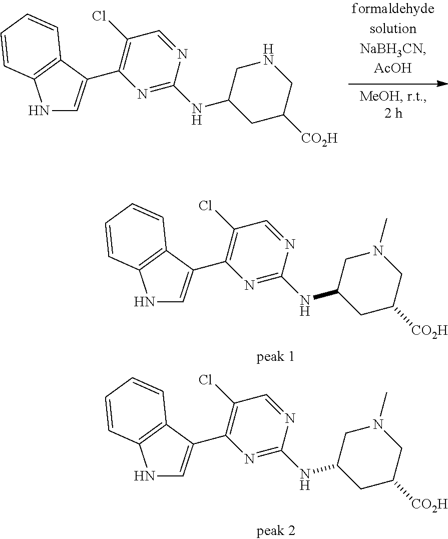

To a solution of 5-[[5-chloro-4-(1H-indol-3-yl)pyrimidin-2-yl]amino]piperidine-3-carboxylic acid (790.00 mg, 1.93 mmol) in MeOH (10 mL) was added formaldehyde (183.53 mg, 1.83 mmol) and followed by AcOH (57.95 mg, 965.00 umol). After stirred at 25° C. for 30 min, then NaBH₃CN (242.56 mg, 3.86 mmol) was added in portions and the reaction mixture was stirred at 25° C. for 2 hr. The reaction mixture was concentrated under vacuum and the residue was purified by prep-HPLC to afford the title compounds peak 1 (200.00 mg, 518.34 umol, 26.86% yield) and peak 2 (180.00 mg, 466.50 umol, 24.17% yield) as a light yellow solid.

### (+/−trans)-N-(4-acrylamidobenzyl)-5-((5-chloro-4-(1H-indol-3-yl)pyrimidin-2-yl)amino)-1-methylpiperidine-3-carboxamide

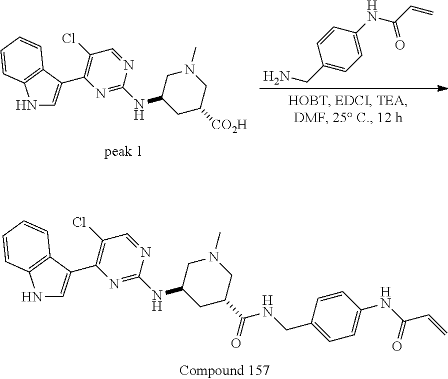

To a mixture of (+/−trans)-5-((5-chloro-4-(1H-indol-3-yl)pyrimidin-2-yl)amino)-1-methylpiperidine-3-carboxylic acid (80.00 mg, 207.34 umol) and N-(4-(aminomethyl)phenyl)acrylamide (54.80 mg, 311.00 umol) in DMF (2 mL), was added TEA (83.92 mg, 829.35 umol), EDCI (47.70 mg, 248.81 umol), and HOBT (33.62 mg, 248.81 umol). The reaction mixture was stirred at 25° C. for 12 hr. The reaction mixture was concentrated under vacuum and the residue was purified by prep-HPLC to afford the title compound (40.00 mg, 68.91 umol, 33.23% yield) as a light brown solid HCl salt. ¹H NMR (400 MHz, MeOD) δ 8.92 (s, 1H), 8.55 (s, 1H), 8.31 (s, 1H), 7.58-7.54 (m, 4H), 7.35 (d, J=6.80 Hz, 1H), 7.23 (d, J=8.00 Hz, 2H), 6.43-6.32 (m, 2H), 5.76 (d, J=9.20 Hz, 1H), 4.34-4.29 (m, 2H), 3.86 (d, J=9.20 Hz, 1H), 3.67 (s, 1H), 3.18 (m, 2H), 3.00 (s, 3H), 2.52 (s, 1H), 1.87 (d, J=11.20 Hz, 1H). MS (m/z): 544.2 [M+1]⁺.

### (+/−cis)-N-(4-acrylamidobenzyl)-5-((5-chloro-4-(1H-indol-3-yl)pyrimidin-2-yl) amino)-1-methylpiperidine-3-carboxamide

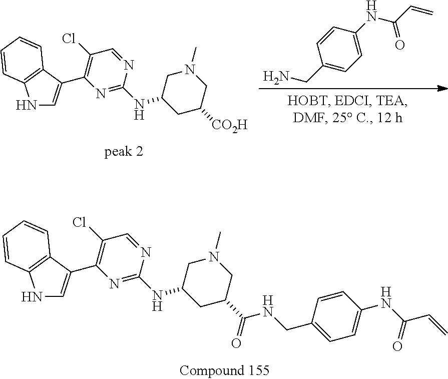

To a mixture of (+/−cis)-5-((5-chloro-4-(1H-indol-3-yl)pyrimidin-2-yl)amino)-1-methylpiperidine-3-carboxylic acid (80.00 mg, 207.33 umol) and N-(4-(aminomethyl)phenyl)acrylamide (54.80 mg, 311.00 umol) in DMF (2 mL) was added TEA (83.92 mg, 829.34 umol), EDCI (47.70 mg, 248.80 umol), followed by HOBT (33.62 mg, 248.80 umol). The reaction mixture was stirred at 25° C. for 12 hr. The reaction mixture was concentrated under vacuum and the residue was purified by prep-HPLC to afford the title compound (52.00 mg, 95.58 umol, 46.10% yield) as yellow solid HCl salt. ¹H NMR (400 MHz, MeOD) δ 9.00 (d, J=12.40 Hz, 1H), 8.46 (s, 1H), 8.38-8.35 (m, 1H), 7.57-7.50 (m, 3H), 7.36-7.26 (m, 3H), 7.04 (s, 1H), 6.41-6.34 (m, 2H), 5.76-5.74 (m, 1H), 4.34-4.26 (m, 2H), 3.78 (d, J=12.00 Hz, 1H), 3.26-3.10 (m, 3H), 3.01 (s, 2H), 2.91 (s, 1H), 2.45-2.36 (m, 1H), 2.24-2.14 (m, 1H). MS (m/z): 544.2 [M+1]⁺.

### Example 50. Synthesis of (E)-N-(4-((4-((5-chloro-4-(1H-indol-3-yl) pyrimidin-2-yl)amino)piperidin-1-yl)methyl)phenyl)-4-(2,2,2-trifluoroacetamido)but-2-enamide (Compound 156)

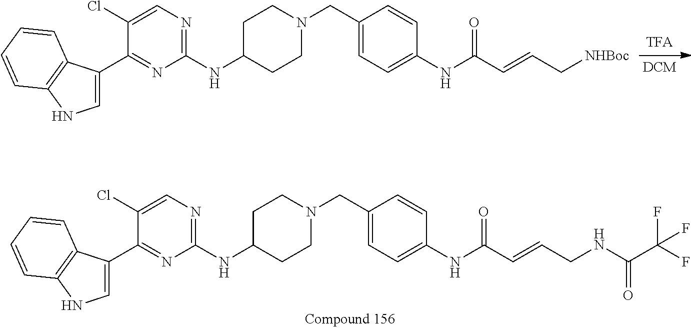 [SMILES: CC(C)(C)OC(=O)NC/C=C/C(=O)Nc1ccc(CN2CCC(Nc3ncc(Cl)c(-c4c[nH]c5ccccc45)n3)CC2)cc1 ; O=C(/C=C/CNC(=O)C(F)(F)F)Nc1ccc(CN2CCC(Nc3ncc(Cl)c(-c4c[nH]c5ccccc45)n3)CC2)cc1]

A mixture of (E)-tert-butyl (4-((4-((4-((5-chloro-4-(1H-indol-3-yl)pyrimidin-2-yl)amino) piperidin-1-yl)methyl)phenyl)amino)-4-oxobut-2-en-1-yl)carbamate (10.00 mg, 16.23 umol) in DCM (2 mL) was added TFA (1.85 mg, 16.23 umol) at 20° C., and then the reaction mixture was stirred at this temperature for 2 hr. The reaction mixture was concentrated and purified by acidic prep-HPLC to give the title compound (7.00 mg, 10.79 umol, 66.48% yield) as HCl salt. ¹H NMR: (400 MHz; MeOD) δ ppm 8.99 (br. s., 1H), 8.57 (br. s., 1H), 8.29 (s, 1H), 7.80 (d, J=8.03 Hz, 2H), 7.58 (d, J=7.53 Hz, 3H), 7.36 (br. s., 2H), 6.85-6.94 (m, 1H), 6.26 (d, J=15.31 Hz, 1H), 4.40 (br. s., 2H), 4.11 (br. s., 2H), 3.65 (d, J=11.29 Hz, 2H), 2.44 (br. s., 2H), 2.34 (br. s., 1H), 2.06 (d, J=11.29 Hz, 2H). MS (m/z): 612.2 [M+1]⁺.

### Example 51. Synthesis of (E)-N-(4-((4-((5-chloro-4-(1H-indol-3-yl)pyrimidin-2-yl)amino)piperidin-1-yl)methyl)phenyl)-4-(methylsulfonamido)but-2-enamide (Compound 158)

### (E)-methyl 4-azidobut-2-enoate

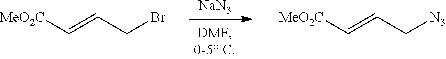 [SMILES: COC(=O)/C=C/CBr ; COC(=O)/C=C/CN=[N+]=[N-] ; "NaN3"]

NaN₃ (6.70 g, 103.06 mmol) was added in small potions to a solution of (E)-methyl 4-bromobut-2-enoate (10.00 g, 55.86 mmol) and NH₄Cl (597.59 mg, 11.17 mmol) in DMF (150 mL) at 0-5° C. The reaction mixture was stirred at 0-5° C. for 3 hr. The reaction mixture was poured into water, the solution was adjusted to pH=9, and then it was extracted with MTBE three times. The organic layers were washed with water and brine, dried over Na₂SO₄, concentrated to obtain the title compound (7.50 g, 53.14 mmol, 95.14% yield) in a solution of 400 mL MTBE. The aqueous phase was diluted and quenched with NaCl.

### (E)-methyl 4-aminobut-2-enoate hydrochloride

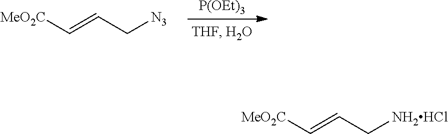

To a stirred solution of (E)-methyl 4-azidobut-2-enoate (7.00 g, 49.60 mmol) in THF (70 mL) and H₂O (5 mL) was added P(OEt)₃ (9.89 g, 59.52 mmol) dropwise at 0° C.

After the addition, the reaction mixture was allowed to rt and stirred overnight, then the mixture was concentrated and re-dissolved in toluene, treated with hydrochloride gas for about 1 hr, then the solution was stirred at rt overnight. The mixture was filtered and the product was collected. It was dried under reduced pressure to obtain the title compound (3.40 g, crude).

### Methyl (E)-4-(tert-butoxycarbonylamino)but-2-enoate

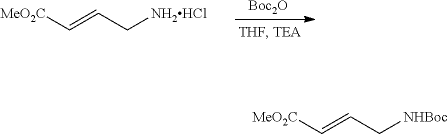

Methyl (E)-4-aminobut-2-enoate (500.00 mg, 3.30 mmol) and DIPEA (852.56 mg, 6.60 mmol) in a mixed solvent of DIOXANE (5 mL) and H₂O (5 mL) was added Boc₂O (719.86 mg, 3.30 mmol) in portions at 0° C. Then the reaction mixture was stirred at 20° C. overnight. The reaction was monitored by LCMS. After concentrating under reduced pressure, the aqueous solution was extracted with EtOAc three times. Combined organic layers were washed with water and 10% aqueous citric acid, dried over Na₂SO₄, and evaporated to obtain the title compound (600.00 mg, crude)

### (E)-4-(tert-butoxycarbonylamino)but-2-enoic acid

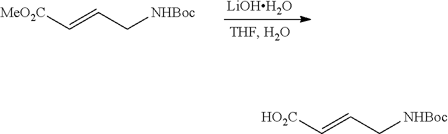 [SMILES: "LiOH·H2O" ; COC(=O)/C=C/CNC(=O)OC(C)(C)C ; CC(C)(C)OC(=O)NC/C=C/C(=O)O]

A mixture of methyl (E)-4-(tert-butoxycarbonylamino)but-2-enoate (600.00 mg, 2.79 mmol,) and LiOH.H₂O (175.60 mg, 4.18 mmol) in a mixed solvent of THF (5 mL) and H₂O (5 mL) was stirred at 20° C. for 12 hr. The reaction was monitored by TLC. After the THF was removed under reduced pressure, the residue was washed with MTBE and acidified with 10% aqueous citric acid, then extracted with EtOAc three times. Combined EtOAc solution was washed with brine and dried over Na₂SO₄. Removal of solvent afforded the (E)-4-(tert-butoxycarbonylamino)but-2-enoic acid (400.00 mg, 1.99 mmol, 71.25% yield) as a white solid.

### Tert-butyl-N-[(E)-4-[4-[[4-[[5-chloro-4-(1H-indol-3-yl)pyrimidin-2-yl]amino]-1-piperidyl] methyl]anilino]-4-oxo-but-2-enyl]carbamate

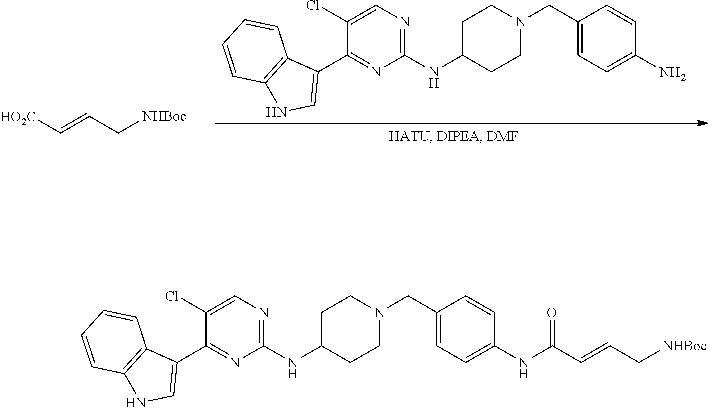 [SMILES: Nc1ccc(CN2CCC(Nc3ncc(Cl)c(-c4c[nH]c5ccccc45)n3)CC2)cc1 ; CC(C)(C)OC(=O)NC/C=C/C(=O)O ; CC(C)(C)OC(=O)NC/C=C/C(=O)Nc1ccc(CN2CCC(Nc3ncc(Cl)c(-c4c[nH]c5ccccc45)n3)CC2)cc1]

To a stirred solution of N-[1-[(4-aminophenyl)methyl]-4-piperidyl]-5-chloro-4-(1H-indol-3-yl)pyrimidin-2-amine (300.00 mg, 692.92 umol) and (E)-4-(tertbutoxycarbonylamino)but-2-enoic acid (139.43 mg, 692.92 umol) in DMF (10 mL) was added DIPEA (179.11 mg, 1.39 mmol) and HATU (289.82 mg, 762.21 umol) at rt Then the reaction mixture was stirred at r.t for 3 hr. The mixture was concentrated directly and purified by prep-HPLC to give the title compound (180.00 mg, 246.52 umol, 35.58% yield) as a TFA salt.

### (E)-4-amino-N-(4-((4-((5-chloro-4-(1H-indol-3-yl) pyrimidin-2-yl)amino)piperidin-1-yl)methyl)phenyl)but-2-enamide hydrochloride

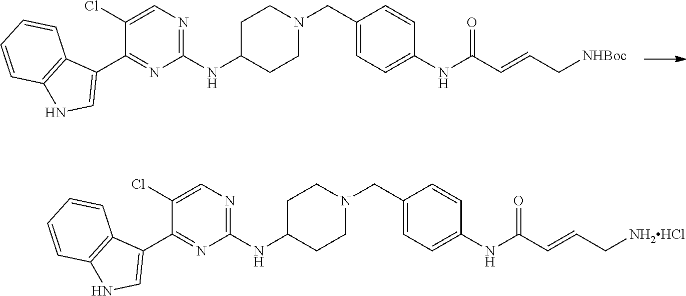

A mixture of tert-butyl N-[(E)-4-[4-[[4-[[5-chloro-4-(1H-indol-3-yl)pyrimidin-2-yl]amino]-1-piperidyl]methyl]anilino]-4-oxo-but-2-enyl]carbamate (90.00 mg, 146.07 umol) in HCL/ACOET (5 mL) was stirred at 25° C. for 1 hr. The reaction mixture was concentrated and purified by acidic prep-HPLC to obtain the title compound (35.00 mg, 63.35 umol, 43.37% yield) as a yellow solid.

### (E)-N-(4-((4-((5-chloro-4-(1H-indol-3-yl)pyrimidin-2-yl)amino)piperidin-1-yl)methyl)phenyl)-4-(methylsulfonamido)but-2-enamide

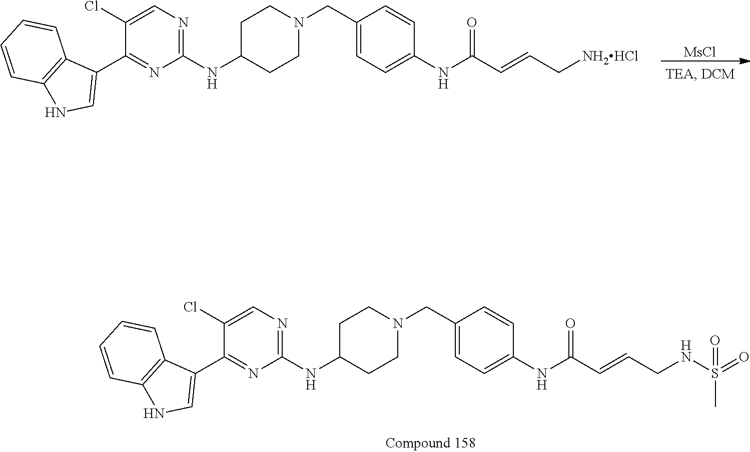

To a stirred solution of (E)-4-amino-N-(4-((4-((5-chloro-4-(1H-indol-3-yl)pyrimidin-2-yl)amino)piperidin-1-yl)methyl)phenyl)but-2-enamide hydrochloride (34.00 mg, 61.54 umol, 1.00 Eq) and TEA (12.45 mg, 123.08 umol, 2.00 Eq) in DCM (2 mL) was added MsCl (10.57 mg, 92.31 umol, 1.50 Eq) at 0° C. under N₂. Then the reaction mixture was allowed to warm to 25° C. and stirred for 4 hr. The mixture was concentrated and purified by prep-HPLC to give the title compound (5.00 mg, 7.93 umol, 12.88% yield) as HCl salt. ¹H NMR: ET251-219-P1A; (400 MHz; MeOD) δ ppm 8.89 (s, 1H), 8.57 (s, 1H), 8.28 (s, 1H), 7.82-7.77 (m, 2H), 7.56-7.54 (m, 3H), 7.33-7.30 (m, 2H), 6.96-6.91 (m, 1H), 6.41 (d, J=15.2 Hz, 1H), 4.39 (s, 2H), 3.92 (d, J=2.8 Hz, 2H), 3.64 (d, J=12.4 Hz, 2H), 3.44 (d, J=12.4 Hz, 2H), 3.23 (d, J=14 Hz, 1H), 2.98 (s, 3H), 2.46 (d, J=11.6 Hz, 2H), 2.02-1.95 (m, 2H). MS (m/z): 594 [M+1]⁺.

### Example 52. Synthesis of (Trans)-N-(4-acrylamidophenyl)-5-((5-chloro-4-(1H-indol-3-yl)pyrimidin-2-yl) amino)-1-methylpiperidine-3-carboxamide (Compound 159) and (Cis)-N-(4-acrylamidophenyl)-5-((5-chloro-4-(1H-indol-3-yl)pyrimidin-2-yl)amino)-1-methylpiperidine-3-carboxamide (Compound 160)

### tert-butyl (4-((trans)-5-((5-chloro-4-(1H-indol-3-yl)pyrimidin-2-yl)amino)-1-methylpiperidine-3-carboxamido)phenyl)carbamate

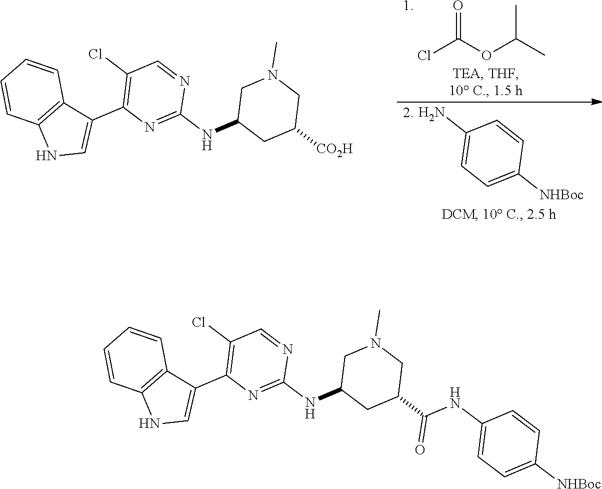

To a mixture of (trans)-5-((5-chloro-4-(1H-indol-3-yl)pyrimidin-2-yl)amino)-1-methylpiperidine-3-carboxylic acid (70 mg, 0.181 mmol) and TEA (73 mg, 0.726 mmol) in THF (2 mL) was added a solution of isopropyl carbonochloridate (27 mg, 0.218 mmol) in THF (0.5 mL) dropwise under N₂ at 10° C. After stirring for 1.5 h, tert-butyl (4-aminophenyl)carbamate (41.56 mg, 0.199 mmol) in DCM (1 mL) was added and the reaction mixture was stirred for 2.5 h. The reaction mixture was concentrated under vacuum and the residue was purified by prep-HPLC to afford the title compound (27 mg, 25.8%) as a yellow solid.

### (trans)-N-(4-aminophenyl)-5-((5-chloro-4-(H-indol-3-yl)pyrimidin-2-yl)amino)-1-methylpiperidine-3-carboxamide (Compound 323)

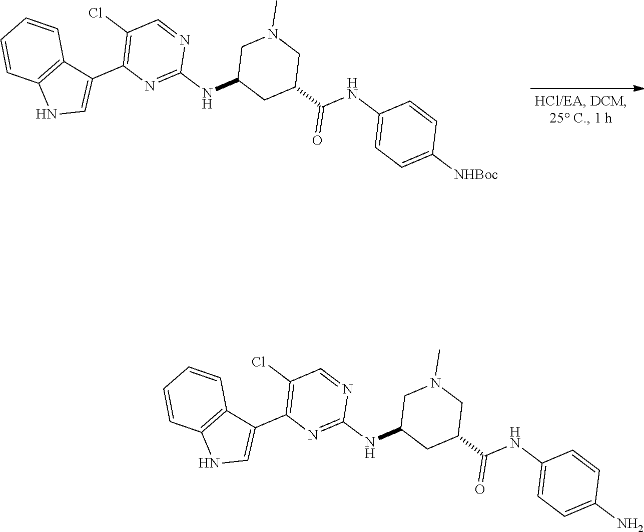

A solution of tert-butyl (4-((trans)-5-((5-chloro-4-(1H-indol-3-yl)pyrimidin-2-yl)amino)-1-methylpiperidine-3-carboxamido)phenyl)carbamate (27 mg, 0.047 mmol) in DCM (1 mL) and HCl/EA (10 mL) was stirred at 25° C. for 1 h. The reaction mixture was concentrated under vacuum to afford the title compound (25 mg, 100%) as a yellow solid. MS (m/z): 476.2 [M+1]⁺.

### (trans)-N-(4-aminophenyl)-5-((5-chloro-4-(1H-indol-3-yl)pyrimidin-2-yl)amino)-1-methylpiperidine-3-carboxamide

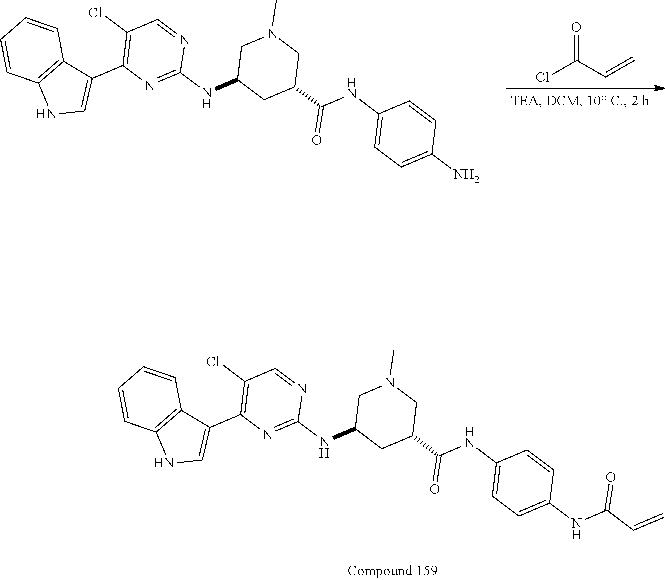

To a mixture of (trans)-N-(4-aminophenyl)-5-((5-chloro-4-(1H-indol-3-yl) pyrimidin-2-yl)amino)-1-methylpiperidine-3-carboxamide (25 mg, 0.053 mmol) and TEA (27 mg, 0.263 mmol) in DCM (1 mL) was added a solution of prop-2-enoyl chloride (5 mg, 0.053 mmol) in DCM (1 mL) dropwise at 10° C. and the reaction mixture was stirred for 2 h. The reaction mixture was concentrated under vacuum, and the residue was purified by prep-HPLC to afford the title compound (3 mg, 10.8%) as a off-white solid. ¹H NMR: ET266-223-P1 (400 MHz, MeOD) δ 8.59 (d, J=4.80 Hz, 1H), 8.42 (s, 1H), 8.19 (s, 1H), 7.59-7.50 (m, 5H), 7.23-7.21 (m, 2H), 6.41-6.32 (m, 2H), 5.77-5.74 (m, 1H), 4.29 (s, 1H), 3.36-3.26 (m, 1H), 3.08-3.06 (m, 1H), 2.89-2.86 (m, 1H), 2.44 (s, 3H), 2.33 (d, J=13.60 Hz, 2H), 2.06 (s, 1H), 1.68-1.65 (m, 1H). MS (m/z): 530.2 [M+1]⁺.

(Cis)-N-(4-acrylamidophenyl)-5-((5-chloro-4-(1H-indol-3-yl)pyrimidin-2-yl)amino)-1-methylpiperidine-3-carboxamide (Compound 160). The title compound was prepared from (3R,5S)-5-((5-chloro-4-(1H-indol-3-yl)pyrimidin-2-yl)amino)-1-methylpiperidine-3-carboxylic acid as described above in this example. ¹H NMR: (400 MHz, MeOD) δ 8.59 (s, 1H), 8.45 (s, 1H), 8.19 (s, 1H), 7.55-7.44 (m, 5H), 7.22-7.13 (m, 2H), 6.41-6.31 (m, 2H), 5.74 (d, J=9.60 Hz, 1H), 4.43 (s, 1H), 2.94-2.87 (m, 2H), 2.78 (s, 1H), 2.62 (s, 1H), 2.48 (s, 1H), 2.38 (s, 3H), 2.13 (s, 1H), 1.95 (s, 1H). MS (m/z): 530.2 [M+1]⁺.

### Example 53. Synthesis of N-((3S,5R)-5-((5-chloro-4-(1H-indol-3-yl)pyrimidin-2-yl)amino)-1-methylpiperidin-3-yl)-4-((E)-4-(dimethylamino)but-2-enamido)benzamide (Compound 162) and N-((3R,5S)-5-((5-chloro-4-(1H-indol-3-yl)pyrimidin-2-yl)amino)-1-methylpiperidin-3-yl)-4-((E)-4-(dimethylamino)but-2-enamido)benzamide (Compound 161)

### 1-benzyl 2-tert-butyl hydrazine-1,2-dicarboxylate

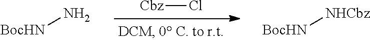 [SMILES: O=C(Cl)OCc1ccccc1 ; CC(C)(C)OC(=O)NN ; CC(C)(C)OC(=O)NNC(=O)OCc1ccccc1]

To a stirred solution of tert-butyl hydrazinecarboxylate (50 g, 0.38 mol) in DCM (500 mL) was added benzyl carbonochloridate (64.54 g, 0.38 mol) dropwise over 30 min at 0° C. After the addition, the mixture was stirred at rt overnight. The mixture was poured into NaHCO₃ solution. The organic layers were separated and washed with brine, dried over Na₂SO₄, evaporated and purified by column to afford the title compound (25 g, purity: 90% on TLC).

### (E)-1-benzyl 2-tert-butyl diazene-1,2-dicarboxylate

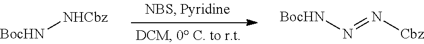 [SMILES: CC(C)(C)OC(=O)NNC(=O)OCc1ccccc1 ; CC(C)(C)OC(=O)N/N=N/C(=O)OCc1ccccc1]

To a stirred solution of 1-benzyl 2-tert-butyl hydrazine-1,2-dicarboxylate (20 g, 75.11 mmol) in DCM (200 mL) was added NBS (13.37 g, 75.11 mmol) and Pyridine (6.13 mL) at 0° C. Then the reaction mixture was allowed to warm to rt and stirred overnight. The mixture was washed with water and the pooled aqueous washes were back extracted with DCM. The pooled organic layers were dried over Na₂SO₄ and evaporated to afford the crude product. The crude product was used directly in next step.

### 2-benzyl 3-tert-butyl 2,3-diazabicyclo[2.2.1]hept-5-ene-2,3-dicarboxylate

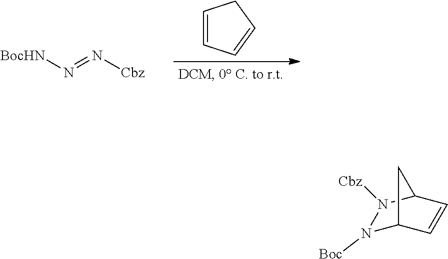 [SMILES: C1=CCC=C1 ; CC(C)(C)OC(=O)N/N=N/C(=O)OCc1ccccc1 ; CC(C)(C)OC(=O)N1C2C=CC(C2)N1C(=O)OCc1ccccc1]

A mixture of (E)-1-benzyl 2-tert-butyl diazene-1,2-dicarboxylate (18 g, 68.11 mmol) in DCM (200 mL) was cooled to 0° C., cyclopenta-1,3-diene (9 g, 136.2 mmol) was added dropwise. After addition, the mixture was allowed warm to room temperature and stirred overnight. The mixture was diluted with DCM, washed with NaHCO₃ solution, dried over Na₂SO₄, concentrated, and purified by column to afford the title compound (20.5 g, purity: 90% on TLC)

### 6-benzyl 7-(tert-butyl) 3-methyl-3,6,7-triazabicyclo[3.2.1]octane-6,7-dicarboxylate

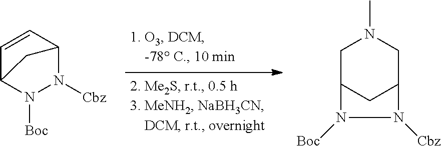 [SMILES: CC(C)(C)OC(=O)N1C2C=CC(C2)N1C(=O)OCc1ccccc1 ; CN1CC2CC(C1)N(C(=O)OC(C)(C)C)N2C(=O)OCc1ccccc1]

Ozone was bubbled into a solution of 2-benzyl 3-tert-butyl 2,3-diazabicyclo[2.2.1]hept-5-ene-2,3-dicarboxylate (10.00 g, 30.27 mmol, 1.00 Eq) in DCM (200 mL) at −78° C. for 30 minutes. The color of solution turned to dark blue. After excess O₃ was purged by N₂, Me₂S (10 g, 160 mmol) was added at 0° C. The reaction was stirred at 25° C. for 12 h. The reaction mixture was concentrated to give the crude product. The crude product was dissolved in DCM (200 mL), then AcOH (1.82 g, 30.27 mmol) and methyl amine (15 mL) was added to the solution. After 10 mins, NaBH(OAc)₃ (6.4 g, 30.27 mmol) was added to the reaction. After another 4 hrs, the solution was concentrated. The residue was purified by prep-HPLC to give title compound (2.80 g, 7.75 mmol, 25.59% yield) as a yellow oil.

### tert-butyl 3-methyl-3,6,7-triazabicyclo[3.2.1]-octane-6-carboxylate

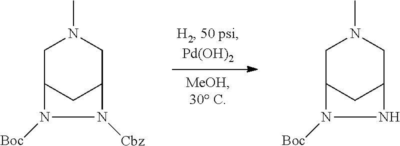 [SMILES: CN1CC2CC(C1)N(C(=O)OC(C)(C)C)N2C(=O)OCc1ccccc1 ; CN1CC2CC(C1)N(C(=O)OC(C)(C)C)N2]

To a solution of 6-benzyl 7-(tert-butyl) 3-methyl-3,6,7-triazabicyclo[3.2.1]octane-6,7-dicarboxylate (2.80 g, 7.75 mmol, 1.00 Eq) in MeOH (100 ml) was added Pd(OH)₂/C (10%, wet, 1 g) under N₂. The suspension was degassed under vacuum and purged with H₂ several times. The mixture was stirred under H₂ (50 psi) at 25° C. for 12 hours. The reaction mixture was filtered and the filtrate was concentrated to afford the crude title compound (1.90 g, crude) as a yellow oil. It was used directly for the next step.

### tert-butyl N-[(3R,5S)-5-amino-1-methyl-3-piperidyl]carbonate

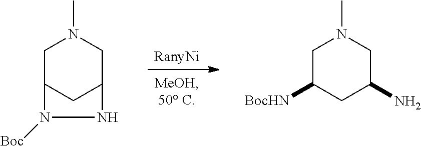 [SMILES: CN1CC2CC(C1)N(C(=O)OC(C)(C)C)N2 ; CN1C[C@@H](N)C[C@@H](NC(=O)OC(C)(C)C)C1]

To a solution of tert-butyl 3-methyl-3,6,7-triazabicyclo[3.2.1]octane-6-carboxylate (1.90 g, 8.36 mmol, 1.00 Eq) in MeOH (50 mL) was added Rany-Ni (1 g) under N₂. The suspension was degassed under vacuum and purged with H₂ several times. The mixture was stirred under H₂ (50 psi) at 50° C. for 4 hours. The reaction mixture was filtered and the filtrate was concentrated to give title compound (1.90 g, 8.29 mmol, 99.11% yield) as a yellow oil.

### tert-butyl N-[(3S)-5-[[4-[1-(benzenesulfonyl)indol-3-yl]-5-chloropyrimidin-2-yl]amino]-1-methyl-3-piperidyl] carbonate

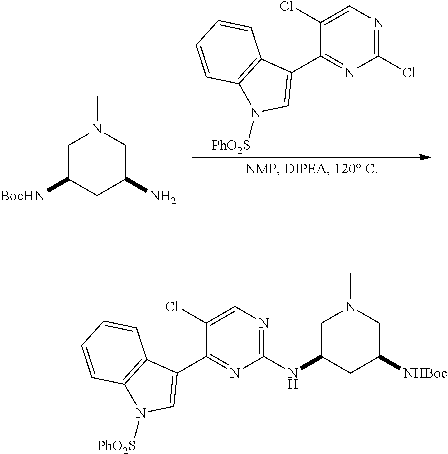

A solution of tert-butyl N-[(3R,5S)-5-amino-1-methyl-3-piperidyl]carbamate (1 g, 4.36 mmol) and 1-(benzenesulfonyl)-3-(2,5-dichloropyrimidin-4-yl)indole (1.76 g, 4.36 mmol) in DMF/EtOH (6/6 mL) was added DIEA (2.82 g, 21.8 mmol). The mixture was purged by N₂. The mixture was heated to 120° C. and stirred for 30 min. The mixture was first purified by flash column to afford (800 mg) crude product. Then the crude product was purified by prep-HPLC to afford the title compound (0.5 g, 19.2% yield) as a yellow solid.

### tert-butylN-[(3S)-5-[[5-chloro-4-(1H-indol-3-yl)pyrimidin-2-yl]amino]-1-methyl-3-piperidyl]carbamate

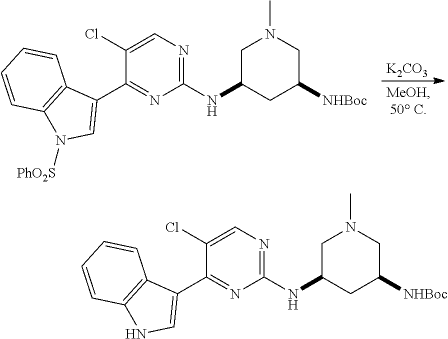

A solution of tert-butyl N-[(3S)-5-[[4-[1-(benzenesulfonyl)indol-3-yl]-5-chloro-pyrimidin-2-yl]amino]-1-methyl-3-piperidyl]carbamate (500 mg, 0.84 mmol) in MeOH (50 mL) was added K₂CO₃ (231 mg, 1.68 mmol). The mixture was heated to 50° C. and stirred for 3 h. Then the mixture was poured with water and extracted with EA. The organic phase was dried with anhydrous Na₂SO4 and concentrated under vacuum to afford the title compound (350 mg, 91.5% yield) as a yellow solid.

### (3S)—N5-[5-chloro-4-(1H-indol-3-yl)pyrimidin-2-yl]-1-methyl-piperidine-3,5-diamine;hydrochloride

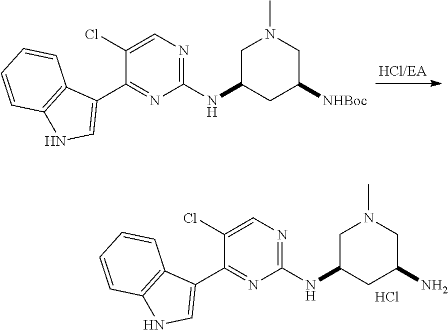 [SMILES: CN1C[C@@H](NC(=O)OC(C)(C)C)C[C@@H](Nc2ncc(Cl)c(-c3c[nH]c4ccccc34)n2)C1 ; CN1C[C@@H](N)C[C@@H](Nc2ncc(Cl)c(-c3c[nH]c4ccccc34)n2)C1 ; Cl]

A solution of tert-butyl N-[(3S)-5-[[5-chloro-4-(1H-indol-3-yl)pyrimidin-2-yl]amino]-1-methyl-3-piperidyl]carbamate (350 mg, 0.76 mmol) in HCl/EA (30 mL) was stirred for 3 h at 28° C. Then the mixture was concentrated under vacuum to afford the title compound (300 mg, 99.6% yield) as a yellow solid.

### N-[(3S)-5-[[5-chloro-4-(1H-indol-3-yl)pyrimidin-2-yl]amino]-1-methyl-3-piperidyl]-4-[[(E)-4-(dimethylamino)but-2-enoyl]amino]benzamide

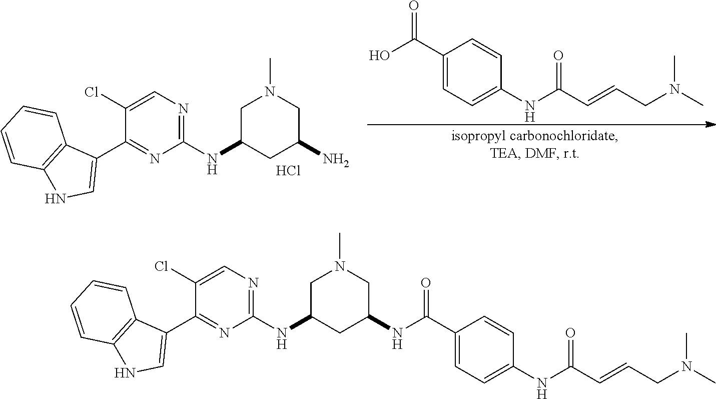 [SMILES: CN1C[C@@H](N)C[C@@H](Nc2ncc(Cl)c(-c3c[nH]c4ccccc34)n2)C1 ; Cl ; CN(C)C/C=C/C(=O)Nc1ccc(C(=O)O)cc1 ; CN(C)C/C=C/C(=O)Nc1ccc(C(=O)N[C@H]2C[C@@H](Nc3ncc(Cl)c(-c4c[nH]c5ccccc45)n3)CN(C)C2)cc1]

A solution of (3S)—N5-[5-chloro-4-(1H-indol-3-yl)pyrimidin-2-yl]-1-methylpiperidine-3,5-diamine;hydrochloride (200 mg, 0.51 mmol) in DMF (5 mL) was added a solution of isopropoxycarbonyl 4-[[(E)-4-(dimethylamino)but-2-enoyl]amino]benzoate (170 mg, 0.51 mmol) in THF (2 mL) dropwise under N₂ protection at 25° C. for 1 h. Then the mixture was concentrated under vacuum to afford the crude residue. Then the residue was purified by prep-HPLC to afford the title compound (33 mg, 10.4%) as yellow solid. ¹H-NMR: (MeOD, 400 MHz) 1.91-2.03 (m, 1H), 2.63 (d, J=9.70 Hz, 1H), 2.85-2.98 (m, 6H), 3.03-3.12 (m, 4H), 3.18-3.26 (m, 1H), 3.76 (d, J=9.70 Hz, 1H), 3.85 (d, J=9.26 Hz, 1H), 3.96-4.04 (m, 2H), 4.62-4.78 (m, 2H), 6.56-6.62 (m, 1H), 6.83-6.92 (m, 1H), 7.29-7.34 (m, 1H), 7.51 (d, J=7.94 Hz, 2H), 7.75 (d, J=8.82 Hz, 2H), 7.81-7.89 (m, 2H), 8.24 (s, 1H), 8.48-8.57 (m, 1H), 8.84 (br. s., 1H). MS (m/z): 587.2 [M+1]⁺.

Separation of N-((3S,5R)-5-((5-chloro-4-(1H-indol-3-yl)pyrimidin-2-yl)amino)-1-methylpiperidin-3-yl)-4-((E)-4-(dimethylamino)but-2-enamido)benzamide (Compound 162) and N-((3R,5S)-5-((5-chloro-4-(1H-indol-3-yl)pyrimidin-2-yl)amino)-1-methylpiperidin-3-yl)-4-((E)-4-(dimethylamino)but-2-enamido)benzamide (Compound 161).

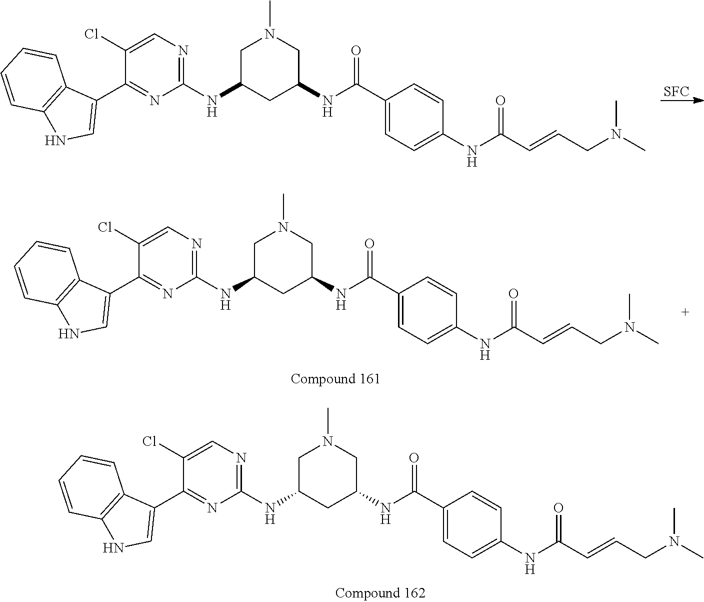

N-[(3S)-5-[[5-chloro-4-(1H-indol-3-yl)pyrimidin-2-yl]amino]-1-methyl-3-piperidyl]-4-[[(E)-4-(dimethylamino)but-2-enoyl]amino]benzamide (25 mg) was run under SFC separation to afford N-((3S,5R)-5-((5-chloro-4-(1H-indol-3-yl)pyrimidin-2-yl)amino)-1-methylpiperidin-3-yl)-4-((E)-4-(dimethylamino)but-2-enamido)benzamide (5 mg) and N-((3R,5S)-5-((5-chloro-4-(1H-indol-3-yl)pyrimidin-2-yl)amino)-1-methylpiperidin-3-yl)-4-((E)-4-(dimethylamino)but-2-enamido)benzamide (6 mg).

Compound 161: ¹HNMR: (MeOD, 400 MHz) 1.13-1.34 (m, 1H), 1.83 (br. s., 1H), 2.49 (br. s., 1H), 2.67-3.03 (m, 9H), 3.49-3.75 (m, 3H), 3.94 (br. s., 2H), 4.47 (br. s., 2H), 6.55 (d, J=14.05 Hz, 1H), 6.85 (br. s., 1H), 7.19 (br. s., 2H), 7.41 (br. s., 1H), 7.65-7.95 (m, 4H), 8.09-8.26 (m, 1H), 8.40 (s, 1H), 8.54 (br. s., 1H). MS (m/z): 587.2 [M+1]⁺.

Compound 162: ¹HNMR: (MeOD, 400 MHz). 1.15-1.28 (m, 1H), 2.43-2.57 (m, 2H), 2.83 (br. s., 9H), 3.51-3.57 (m, 3H), 3.88 (br. s., 2H), 4.42 (br. s., 2H), 6.54 (br. s., 1H), 6.85 (br. s., 1H), 7.19 (br. s., 2H), 7.42 (br. s., 1H), 7.57-7.91 (m, 4H), 8.05-8.22 (m, 1H), 8.41 (br. s., 1H), 8.54 (br. s., 1H). MS (m/z): 587.2 [M+1]⁺.

### Example 54. Synthesis of (E)-N-[3-[[4-[[5-chloro-4-(1H-indol-3-yl)pyrimidin-2-yl]amino]-1-piperidyl]methyl]phenyl]-4-(dimethylamino)-N-methyl-but-2-enamide (Compound 145)

### 5-chloro-4-(1H-indol-3-yl)-N-(1-(3-(methylamino)benzyl)piperidin-4-yl)pyrimidin-2-amine

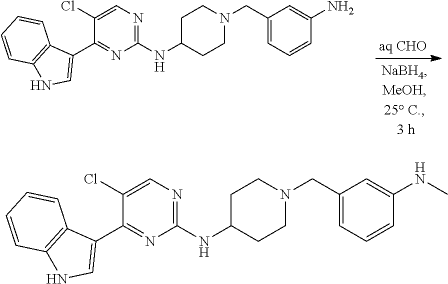 [SMILES: Nc1cccc(CN2CCC(Nc3ncc(Cl)c(-c4c[nH]c5ccccc45)n3)CC2)c1 ; CNc1cccc(CN2CCC(Nc3ncc(Cl)c(-c4c[nH]c5ccccc45)n3)CC2)c1]

To a solution of N-[1-[(3-aminophenyl)methyl]-4-piperidyl]-5-chloro-4-(1H-indol-3-yl)-pyrimidin-2-amine (500 mg, 1.15 mmol) in MeOH (15 mL) was added a solution of formaldehyde (35 mg, 1.15 mmol) in MeOH (5 mL) at 25° C. drop-wised and the mixture was stirred for 1 h. Then NaBH₃CN (145 mg, 2.30 mmol) was added and the mixture was stirred for 2 h. The mixture was concentrated under reduced pressure, and the residue was purified by prep-HPLC to afford the title compound (150 mg, 17.5%) as a yellow solid.

### (E)-N-[3-[[4-[[5-chloro-4-(1H-indol-3-yl)pyrimidin-2-yl]amino]-1-piperidyl]methyl]phenyl]-4-(dimethylamino)-N-methyl-but-2-enamide

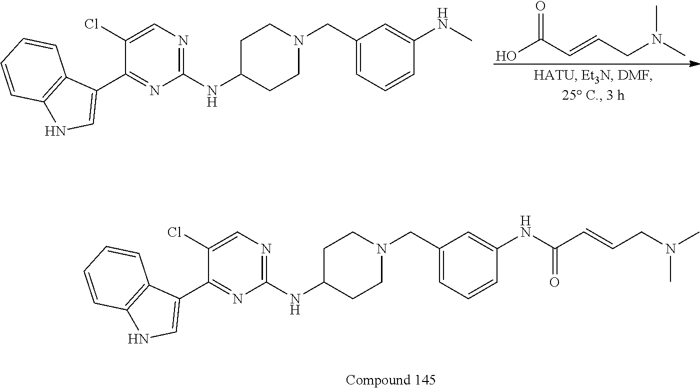

To a solution of 5-chloro-4-(1H-indol-3-yl)-N-[1-[[3-(methylamino)phenyl]methyl]-4-piperidyl]pyrimidin-2-amine (200 mg, 0.45 mmol) and (E)-4-(dimethylamino)but-2-enoic acid (87 mg, 0.67 mmol) in DCM (10 mL) was added HATU (340 mg, 0.59 mmol) and Et₃N (136 mg, 1.34 mmol) at 25° C. and the mixture was stirred for 3 h. Then the mixture was concentrated under vacuum and purified by prep-HPLC to afford the title compound (25 mg, 10%). ¹H NMR: (DMSO, 400 MHz) δ 11.85 (br. s., 1H), 8.44-8.65 (m, 2H), 8.24 (br. s., 1H), 7.49 (d, J=8.78 Hz, 1H), 7.43 (br. s., 1H), 7.33 (br. s., 1H), 7.08-7.25 (m, 5H), 6.62 (br. s., 1H), 5.88 (br. s., 1H), 3.83 (br. s., 1H), 3.55 (br. s., 2H), 3.25 (s, 3H), 2.86 (br. s., 4H), 1.86-2.18 (m, 10H), 1.58 (d, J=11.29 Hz, 2H). MS (m/z): 558.3 [M+1]⁺.

### Example 55. Synthesis of (E)-N-(4-(5-(5-chloro-4-(1H-indol-3-yl)pyrimidin-2-ylamino)-1-(4-methoxybenzyl)-1H-benzo[d]imidazol-2-yl)phenyl)-4-(dimethylamino)but-2-enamide (Compound 133)

### 5-chloro-N-(4-fluoro-3-nitrophenyl)-4-(1-(phenylsulfonyl)-1H-indol-3-yl)pyrimidin-2-amine

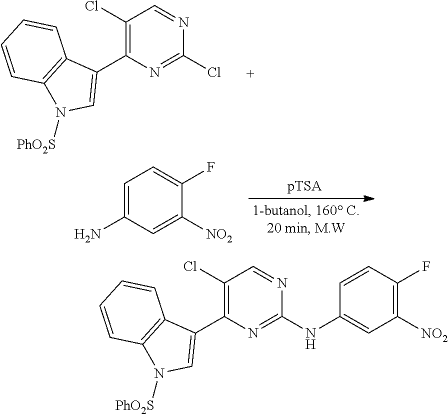

A solution of 2-(2,5-dichloropyrimidin-4-yl)-1-(phenylsulfonyl)-1H-indole (622 mg, 1.50 mmol), 3-nitro-4-fluoroaniline (240 mg, 1.50 mmol) and pTSOH. H₂O (585 mg, 3.10 mmol) in 1-butanol (10 mL) was heated at 160° C. (mW) for 20 min. The mixture was diluted with Et₂O (20 mL) and the resulting suspension was filtered and afforded the title compound (800 mg, 1.53 mmol, 99%) as a yellowish powder.

### N4-(5-chloro-4-(1-(phenylsulfonyl)-1H-indol-3-yl)pyrimidin-2-yl)-N1-(4-methoxybenzyl)-2-nitrobenzene-1,4-diamine

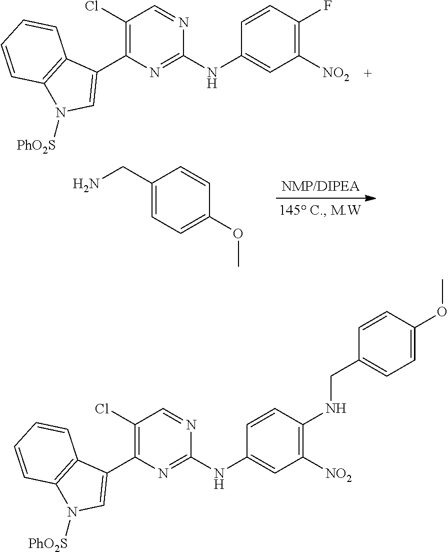

A solution of the 5-chloro-N-(4-fluoro-3-nitrophenyl)-4-(1-(phenylsulfonyl)-1H-indol-3-yl)pyrimidin-2-amine (701 mg, 1.34 mmol), DIPEA (0.47 μL, 2.68 mmol) and p-methoxybenzylamine (175 μL, 1.34 mmol) in NMP (10 mL) was heated 20 min at 145° C. (mW). The cooled mixture was diluted with EtOAc (20 mL), washed with sat. NaHCO₃ (5 mL), dried (MgSO₄), filtered and concentrated under reduced pressure. The residue was purified by SiO₂ chromatography (Hex/EtOAc 20-100% gradient) and afforded the title compound (665 mg, 1.04 mmol, 78%) as a red solid.

### N4-(5-chloro-4-(1-(phenylsulfonyl)-1H-indol-3-yl)pyrimidin-2-yl)-N1-(4-methoxybenzyl)benzene-1,2,4-triamine

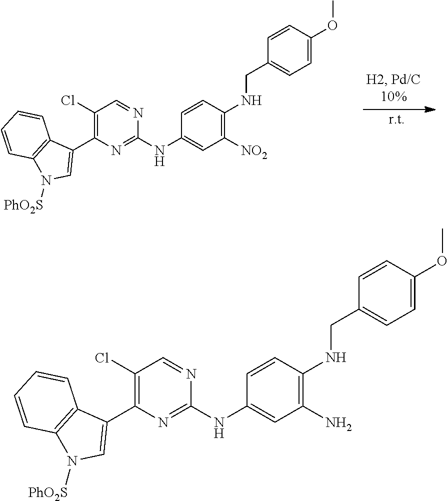

To a degassed solution of N4-(5-chloro-4-(1-(phenylsulfonyl)-1H-indol-3-yl)pyrimidin-2-yl)-N1-(4-methoxybenzyl)-2-nitrobenzene-1,4-diamine (554 mg, 0.86 mmol) in THF/MeOH 2:1 (15 mL) was added the 10% Pd/C (80 mg). The resulting was stirred 1 h under H₂ (1 atm.), filtered over Celite (EtOAc) and the solvents were removed under reduced pressure. The residue was purified by SiO₂ chromatography (Hex/EtOAc 20 to 80% gradient) and afforded the title compound (300 mg, 0.491 mmol, 57%) as a yellow solid.

### N-(4-(5-(5-chloro-4-(1-(phenylsulfonyl)-1H-indol-3-yl)pyrimidin-2-ylamino)-1-(4-methoxybenzyl)-1H-benzo[d]imidazol-2-yl)phenyl)acetamide

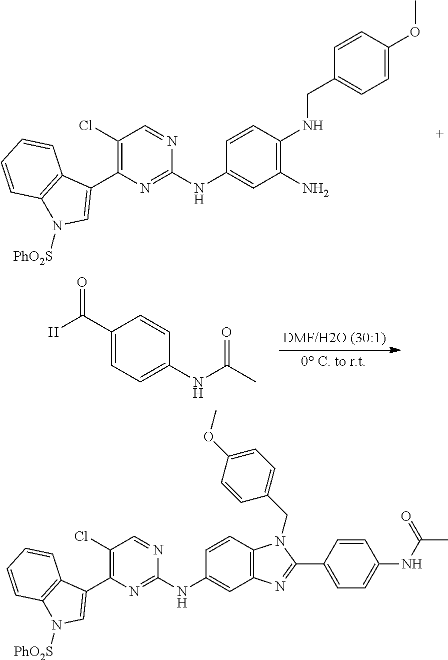

A cooled (0° C.) solution of N4-(5-chloro-4-(1-(phenylsulfonyl)-1H-indol-3-yl)pyrimidin-2-yl)-N1-(4-methoxybenzyl)benzene-1,2,4-triamine (301 mg, 0.49 mmol), 4-acetamidobenzaldehyde (84 mg, 0.52 mmol) in DMF (6 mL) and de-ionized water (0.2 mL) was treated with Oxone (1967 mg, 1.10 mmol). The resulting mixture was stirred 30 min at 0° C. and diluted with EtOAc (30 mL), washed with sat. NaHCO₃, dried (MgSO₄), filtered and concentrated under reduced pressure. The residue was purified by SiO₂ chromatography (DCM/I MeOH 0 to 10% gradient) and afforded the title compound (340 mg, 0.451 mmol, 92%) as an orange solid.

### 2-(4-aminophenyl)-N-(5-chloro-4-(1H-indol-3-yl)pyrimidin-2-yl)-1-(4-methoxybenzyl)-1H-benzo[d]imidazol-5-amine

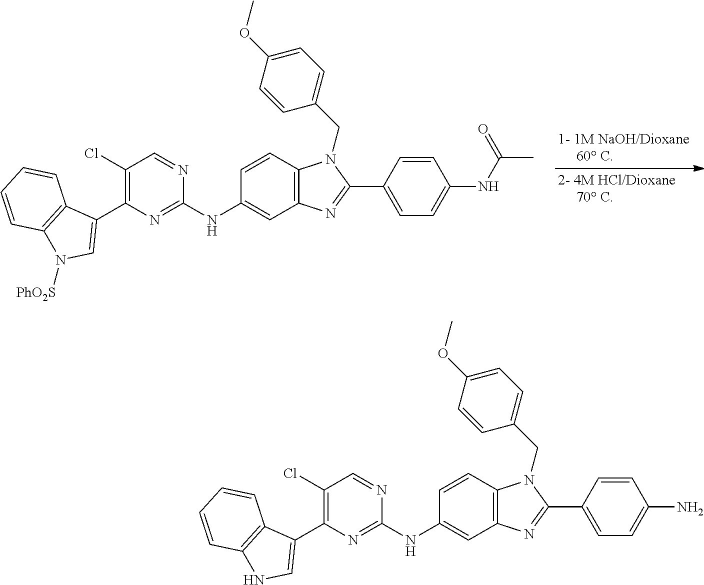

A solution of N-(4-(5-(5-chloro-4-(1-(phenylsulfonyl)-1H-indol-3-yl)pyrimidin-2-ylamino)-1-(4-methoxybenzyl)-1H-benzo[d]imidazol-2-yl)phenyl)acetamide (340 mg, 0.451 mmol) and 1M NaOH (3 mL, 3.16 mmol) in dioxane (5 mL) was heated 3 h at 70° C. The cooled mixture was diluted with DCM (20 mL), washed with water (5 mL), dried (MgSO₄), filtered and concentrated under reduced pressure. The resulting solid was dissolved with 4M HCl in dioxane (1.5 mL) and water (300 μL) and heated overnight at 80° C. The cooled mixture was diluted with DCM (20 mL), washed with sat. NaHCO₃, dried (MgSO₄), filtered and concentrated under reduced pressure. The mixture was triturated with Et₂O and afforded the title compound (200 mg, 0.350 mmol, 78%) as an orange solid.

### (E)-N-(4-(5-(5-chloro-4-(1H-indol-3-yl)pyrimidin-2-ylamino)-1-(4-methoxybenzyl)-1H-benzo[d]imidazol-2-yl)phenyl)-4-(dimethylamino)but-2-enamide (Compound 133)

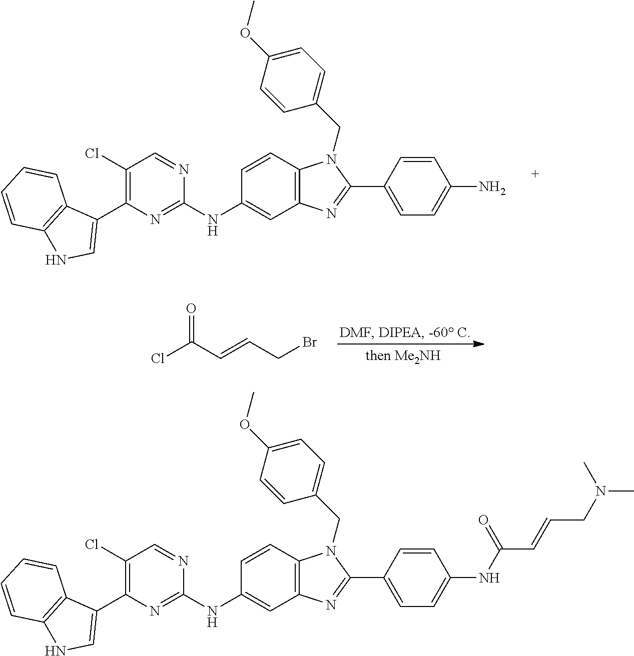

To a −30° C. solution of 2-(4-aminophenyl)-N-(5-chloro-4-(1H-indol-3-yl)pyrimidin-2-yl)-1-(4-methoxybenzyl)-1H-benzo[d]imidazol-5-amine (25 mg, 0.09 mmol) and DIPEA (23 pl, 0.26 mmol) in DMF (1 mL) was slowly added a 34 mg/mL solution of (E)-4-bromobut-2-enoyl chloride in DCM (256 μL, 0.09 mmol). After 30 min at −30° C. a 2M solution of dimethylamine in THF (900 μL, 1.80 mmol) was added and the mixture was stirred 1 h at rt before being concentrated under reduced pressure. The mixture was purified by prep HPLC (Water/CH₃CN 20 to 100% gradient) and afforded the title compound (10 mg, 0.015 mmol, 16%) as white solid after lyophilization. ¹H NMR (500 MHz, DMSO) δ 11.88 (s, 1H), 10.32 (d, J=8.3 Hz, 1H), 10.20 (s, 1H), 9.64-9.51 (m, 1H), 8.52-8.48 (m, 1H), 8.44 (d, J=2.4 Hz, 1H), 8.11-8.05 (m, 1H), 7.85-7.79 (m, 1H), 7.75-7.69 (m, 1H), 7.63-7.31 (m, 3H), 7.22-7.15 (m, 1H), 7.14-7.03 (m, 2H), 7.02-6.93 (m, 2H), 6.86 (dt, J=9.2, 6.0 Hz, 1H), 6.82-6.73 (m, 1H), 6.52 (s, 1H), 6.37-6.27 (m, 1H), 6.04 (s, 1H), 5.75 (d, J=1.8 Hz, 1H), 5.57-5.45 (m, 1H), 3.70 (d, J=3.7 Hz, 2H), 2.63-2.51 (m, 6H), 2.32-2.14 (m, 3H); MS (m/z): 560.63 [M+1]⁺.

### Example 56. Synthesis of Additional Compounds of the Invention

Additional compounds of the invention were synthesized using modification to or one or more of the foregoing examples. In the table below, the specific examples and modifications are indicated for each compound, as well as the ¹H NMR and MS characterization data. Compound numbers (“Cmpd #”) correspond to the compound numbers in FIG. 1A-FIG. 1H.

| Cmpd # | Synthetic Protocol | ¹H NMR | [M + 1]⁺ |
|---|---|---|---|
| 134 | Starting from 3-(2,5- | ¹H NMR (500 MHz, DMSO-d6) δ 11.90 (s, | 546.63 |
| | dichloropyrimidin-4- | 1H), 9.87 (s, 1H), 9.60 (s, 1H), 8.61 (d, J = 7.7 | |
| | yl)-1- | Hz, 1H), 8.52 (d, J = 3.1 Hz, 1H), 8.44 (s, 1H), | |
| | (phenylsulfonyl)- | 7.95 (s, 1H), 7.50 (d, J = 8.1 Hz, 1H), 7.46 (d, | |
| | 1H-indole and m- | J = 9.0 Hz, 1H), 7.27 (d, J = 8.7 Hz, 1H), 7.25- | |
| | phenylenediamine | 7.16 (m, 1H), 7.10 (t, J = 7.5 Hz, 1H), 6.87 (d, | |
| | using the same | J = 16.1 Hz, 1H), 6.67-6.52 (m, 1H), 4.48-4.41 | |
| | synthetic sequence | (m, 2H), 4.24-3.96 (m, 2H), 3.89-3.58 (m, 2H), | |
| | as Example 32 | 3.21-2.98 (m, 2H), 2.85-2.55 (m, 6H), 1.93- | |
| | | 1.71 (m, 2H), 1.71-1.39 (m, 2H) | |
| 135 | Starting from 3-(2,5- | ¹H NMR (500 MHz, DMSO) δ 11.90 (s, 1H), | 447.55 |
| | dichloropyrimidin-4- | 10.01 (s, 1H), 9.61 (s, 1H), 8.60 (d, J = 8.2 Hz, | |
| | yl)-1- | 1H), 8.52 (d, J = 3.0 Hz, 1H), 8.44 (s, 1H), | |
| | (phenylsulfonyl)- | 7.99 (s, 1H), 7.48 (dd, J = 11.7, 8.6 Hz, 2H), | |
| | 1H-indole and m- | 7.32 (d, J = 8.0 Hz, 1H), 7.20 (ddd, J = 11.1, | |
| | phenylenediamine | 9.6, 4.6 Hz, 2H), 7.09 (t, J = 7.1 Hz, 1H), 6.72 | |
| | using the same | (dt, J = 15.4, 6.0 Hz, 1H), 6.27 (d, J = 15.4 Hz, | |
| | synthetic sequence | 1H), 3.04 (dd, J = 6.0, 1.4 Hz, 2H), 2.50 (s, | |
| | as Example 32 | 3H), 2.17 (s, 3H) | |

### Example 57. Kinase Activity

Compounds of the invention were assayed for activity against CDK7 at Life Technologies™ (Grand Island, N.Y.) using their commercially available Adapta® kinase assay services. Test compounds were tested at concentrations ranging from 10 μM down to 0.514 nM in a series of 3-fold serial dilutions. Details of the assay, including the substrate used for CDK7 kinase, are available on the Life Technologies web site (http://www.lifetechnologies.com/us/en/homellife-science/drug-discovery/target-and-lead-identification-and-validation/kinasebiology/kinase-activity-assays.html).

The results of this assay are shown below in Table 2. “A” represents a calculated IC₅₀ of less than 100 nM; “B” represents a calculated IC₅₀ of between 100 nM and 1 μM; and “C” represents a calculated IC₅₀ of greater than 1 μM. The co-factors used for the assay were cyclin H and MNAT1.

TABLE 2
Activity of Selected Compounds of the Invention against CDK7.

| Compound | CDK7 |
|---|---|
| No. | Inhibition |
| 100 | A |
| 101 | B |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | B |
| 117 | C |
| 118 | B |
| 119 | B |
| 120 | B |
| 121 | C |
| 122 | B |
| 123 | B |
| 124 | C |
| 125 | A |
| 126 | A |
| 127 | B |
| 128 | B |
| 129 | A |
| 131 | B |
| 132 | C |
| 133 | B |
| 134 | A |
| 135 | A |
| 136 | A |
| 137 | A |
| 138 | B |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | B |
| 156 | B |
| 157 | A |
| 158 | B |
| 159 | B |
| 160 | A |
| 161/162* | A |
| 24 | B |
| 26 | B |
| 37 | A |
| 35 | A |
| 32 | A |
| 47 | B |
| 48 | B |
| 301 | B |
| 55 | A |
| 59 | A |
| 63 | B |
| 67 | B |
| 302 | C |
| 303 | C |
| 304 | B |
| 305 | A |
| 306 | A |
| 307 | A |
| 308 | A |
| 309 | B |
| 310 | C |
| 311 | A |
| 312 | A |
| 313 | C |
| 322 | B |

*A mixture of the two enantiomers - Compound 161 and 162 - was tested.

Exemplary compounds of the invention were further tested for inhibitory activity against CDK7 using an assay developed using a Caliper/LabChip EZ Reader (Perkin Elmer, Waltham, Mass.). In this protocol, the concentration of phosphorylated peptide substrate produced as a fraction of total peptide activity is monitored following an incubation period (30 minutes), which was selected such that the total fraction of phosphorylated peptide produced was less than 20% for the uninhibited kinase. Compounds of the invention were assayed at concentrations ranging from 10 μM to 0.514 nM in a series of 3-fold serial dilutions, and were incubated with CDK7/Cyclin H/MAT1 trimeric complex (10 nM), ATP (2 mM), and “FAM-CDK7tide” peptide substrate (2 μM, synthesized fluorophore-labeled peptide with the following sequence: 5-FAM-YSPTSPSYSPTSPSYSPTSPSKKKK) in a buffer comprising 20 mM MES, pH 6.75; 6 mM MgCl₂; 0.01% Tween 20; and 0.05 mg/mL BSA. IC₅₀ values were recorded for selected test compounds and are reported in Table 3, wherein “A” represents a calculated IC₅₀ of less than 100 nM, “B” represents a calculated IC₅₀ of between 100 nM and 1 μM, and “C” represents a calculated IC₅₀ of greater than 1 μM.

TABLE 3
Calculated IC₅₀ values of exemplary compounds
of the invention against CDK7

| Compound No. | CDK7 IC₅₀ |
|---|---|
| 114 | B |

### Example 58 Inhibition of Cell Proliferation

Representative compounds of the invention were tested at different concentrations (from 10 μM to 316 pM; 0.5 log serial dilutions) for their ability to inhibit the proliferation of various cancer cell lines. Known CDK inhibitors flavopiridol and triptolide were used as positive controls. Cells were grown in the indicated media below. All cell lines were supplemented with FBS (Life Technologies) and 100 U·mL⁻¹ penicillin, 100 μg·mL⁻¹ streptomycin (Invitrogen) and cultured at 37° C. in a humidified chamber in the presence of 5% CO₂. Proliferation assays were conducted over a 72 hour time period. CellTiter-Glo® (Promega Corporation, Madison, Wis. USA) was used to assess the anti-proliferative effects of the compounds following manufacturer's directions and utilizing the reagents supplied with the CellTiter-Glo® kit.

The following cancer cell lines were tested with the media conditions indicated: Blood Cancer Cell Lines
Jurkat—RPMI 1640+10% FBS+1% Glutamax
HL-60—RPMI 1640+10% FBS+1% Glutamax
THP-1—RPMI 1640+10% FBS+1% Glutamax+0.05 mM 2-Mercaptoethanol
MV4-11—RPMI 1640+10% FBS+1% Glutamax
RS4-11—RPMI 1640+10% FBS+1% Glutamax

### Breast Cancer Cell Lines

hTERT-HME1—Mammary Epithelial Cell Basal Medium (500 mL; Lonza CC-3151)+2 mL BPE+0.5 mL hEGF+0.5 mL Hydrocortisone+0.5 mL GA-1000+0.5 mL insulin (Lonza CC-4136)+100 ng/mL cholera toxin.
MDA-MB231—Leibovitz's L-15 Medium+10% FBS+1% Glutamax
MCF-7—RPMI 1640+10% FBS+1% Glutamax
MCF-10A—Mammary Epithelial Cell Basal Medium (500 mL; Lonza CC-3151)+2 mL BPE+0.5 mL hEGF+0.5 mL Hydrocortisone+0.5 mL GA-1000+0.5 mL insulin (Lonza CC-4136)+100 ng/mL cholera toxin.
SKBR-3—McCoy's 5a Medium Modified+10% FBS
T47D—RPMI 1640+10% FBS+1% Glutamax+0.2 Units/ml bovine insulin

### Osteosarcoma Cell Lines

143B—EMEM+10% FBS+15 ug/ml Bromo-deoxy Uridine (BUdR)+2 mM Glutamine+1% Non Essential Amino Acids (NEAA)
MNNG-Hos Cl#5—EMEM+10% FBS
SAOS—McCoy's 5a Medium Modified+10% FBS+2 mM L-Glut
MG-63—EMEM+10% FBS

### Ewing's Sarcoma Cell Lines

Hs863T—DMEM (4 mM L-Glut, 4.5 g/L Glucose, 1 mM pyruvate, 1.5 g/l bicarb)+10% FBS
Hs822T—DMEM (4 mM L-Glut, 4.5 g/L Glucose, 1 mM pyruvate, 1.5 g/l bicarb)+10% FBS
A673—DMEM (4 mM L-Glut, 4.5 g/L Glucose, 1 mM pyruvate, 1.5 g/l bicarb)+10% FBS
SKES-1—McCoy's 5a Medium Modified (modified—1.5 mM L-glut, 2.2 g/L bicarb)+15% FBS
RD-ES—RPMI 1640+15% FBS.

The results of these assays are set forth in Tables 4A, 4B and 4C, below. In these tables, “A” represents an IC₅₀ of less than 500 nM; “B” an IC₅₀ of between 500 nM and 5 μM; and “C” an IC₅₀ of greater than 5 μM

TABLE 4A
Inhibition of Proliferation of Various Cancer Cell Lines by Compounds of the Invention.

| | | | | | Breast Cancer/Breast | | | | | |
|---|---|---|---|---|---|---|---|---|---|---|
| | Blood Cancer | | | | hTERT- | MDA- | | | | |
| Compound | HL60 | THP-1 | MV4;11 | RS4;11 | HME1 | MB231 | MCF7 | MCF10A | T47D | SKBR3 |
| 100 | A | A | A | A | A | A | A | A | C | C |
| 101 | C | C | A | B | B | C | C | B | C | C |
| 102 | A | A | A | A | A | A | A | A | C | C |
| 103 | A | A | A | A | A | A | C | A | C | C |
| 104 | A | A | A | A | A | A | B | A | C | C |
| Flavopiridol | A | A | A | A | A | A | B | A | C | A |
| Triptolide | A | A | A | A | A | A | A | A | C | A |

TABLE 4B
Inhibition of Proliferation of Various Cancer Cell Lines by Compounds of the
Invention.

| | Ewing's Sarcoma | | | | | Osteosarcoma | | |
|---|---|---|---|---|---|---|---|---|
| | | | | | SK- | | MNNG-HOS | |
| Compound | A673 | Hs822T | Hs863T | RD-ES | ES | SAOS | Cl#5 | 143B |
| 100 | A | B | C | A | A | A | B | C |
| 101 | B | C | C | C | B | C | C | C |
| 102 | A | B | C | A | A | A | C | C |
| 103 | A | C | C | A | A | A | B | C |
| 104 | A | B | C | A | A | A | C | C |
| Flavopiridol | A | C | C | A | A | A | B | B |
| Triptolide | A | B | C | A | A | A | A | A |

TABLE 4C
Inhibition of Proliferation of Jurkat
Cells by Compounds of the Invention.

| Compound | Jurkat |
|---|---|
| 100 | A |
| 101 | B |
| 102 | A |
| 103 | A |
| 104 | A |
| 105 | B |
| 106 | A |
| 107 | A |
| 108 | A |
| 109 | A |
| 110 | A |
| 111 | A |
| 112 | A |
| 113 | A |
| 114 | A |
| 115 | A |
| 116 | B |
| 117 | C |
| 118 | B |
| 119 | B |
| 120 | A |
| 121 | B |
| 122 | A |
| 123 | A |
| 124 | C |
| 125 | A |
| 126 | A |
| 127 | C |
| 128 | B |
| 129 | A |
| 130 | A |
| 131 | B |
| 132 | C |
| 133 | B |
| 134 | B |
| 135 | B |
| 136 | B |
| 137 | B |
| 138 | B |
| 139 | B |
| 140 | A |
| 141 | A |
| 142 | A |
| 143 | A |
| 144 | A |
| 145 | A |
| 146 | C |
| 147 | C |
| 148 | B |
| 149 | B |
| 150 | A |
| 151 | A |
| 152 | B |
| 153 | A |
| 154 | A |
| 155 | B |
| 156 | B |
| 157 | A |
| 24 | C |
| 26 | B |
| 37 | B |
| 35 | B |
| 32 | C |
| 47 | B |
| 48 | B |
| 301 | B |
| 55 | B |
| 59 | A |
| 63 | C |
| 67 | B |
| 302 | B |
| 303 | C |
| 304 | B |
| 305 | B |
| 306 | B |
| 307 | B |
| 308 | B |
| 310 | C |
| 311 | A |
| 312 | B |
| 314 | B |
| 315 | C |
| 316 | B |
| 318 | C |
| 322 | C |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-32. (canceled)

33. A compound having the structural formula I: 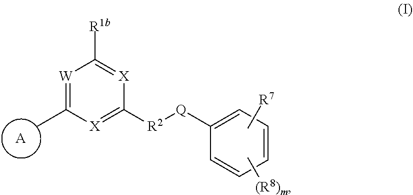
or a pharmaceutically acceptable salt, solvate, hydrate, tautomer, or stereoisomer thereof, wherein:
ring A is an optionally substituted heteroaryl ring of any one of the Formulae (i-1)-(i-6): 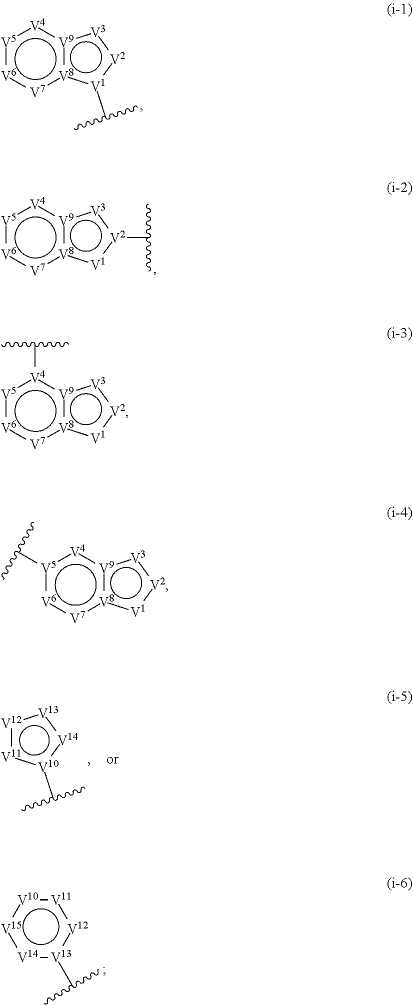
wherein:
each instance of V¹, V², V³, V⁴, V⁵, V⁶, V⁷, V⁸, V⁹, V¹⁰, V¹¹, V¹², V¹³, V¹⁴ and V¹⁵ is independently O, S, N, N(R^{A1}), C, or C(R^{A2}), and wherein at least one ring atom in ring A is selected from O, S or N;
each instance of R^{A1} is independently selected from hydrogen, deuterium, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;
each instance of R^{A2} is independently selected from hydrogen, deuterium, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a} wherein each occurrence of R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring;
each X is independently selected from N and CH, wherein at least one X is N;
W is selected from N and C(R^{1a});
each of R^{1a}, if present, and R^{1b} is independently selected from hydrogen, deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B1a}, —N(R^{B1a})₂, and —SR^{B1a}, wherein each occurrence of R^{B1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
R^{1a} and R^{1b} are joined to form an optionally substituted carbocyclic, optionally substituted heterocyclic, optionally substituted aryl, or optionally substituted heteroaryl ring;
R² is an optionally substituted C₁-C₄ alkylene or an optionally substituted C₂-C₄ alkenylene or alkynylene, wherein one or more methylene units of the alkylene, alkenylene or alkynylene are optionally and independently replaced with —O—, —S—, or —N(R⁶)—;
Q is selected from: 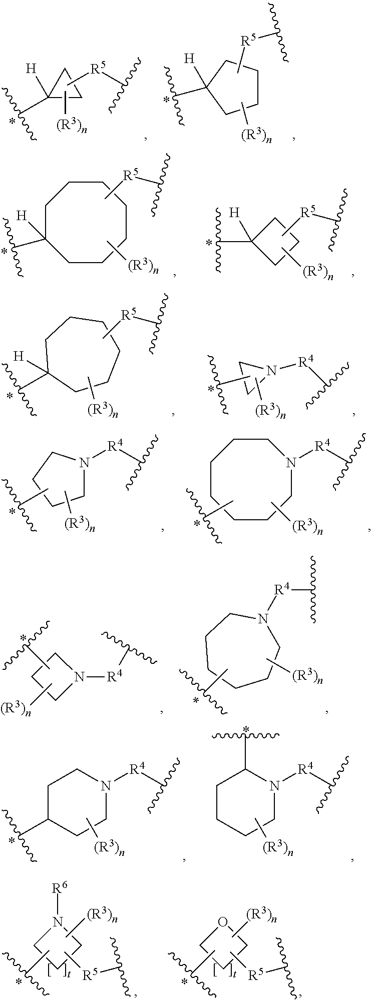
and a 4-14 membered, divalent, fused or spirofused bicyclic ring system comprising a total of 0 to 4 ring heteroatoms independently selected from N, O and S, and optionally substituted with 1 to 6 independently selected R³, wherein:
each ring in the bicyclic ring system is independently selected from heterocyclyl, carbocyclyl, aromatic or heteroaromatic,
one atom in each ring of the bicyclic ring system is attached to the rest of the compound, and
t is 0, 1, 2, 3, or 4;
wherein each [symbol] represents a portion of Q bound to the rest of the compound; and “*” represents a portion of Q bound to R²;
each instance of R³, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1})₂, and —SR^{C1}, wherein each occurrence of R^{C1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
two R³ groups bound to the same ring carbon atom are taken together to form ═O, or
two R³ groups bound to the same or different ring carbon atoms are joined to form an optionally substituted carbocyclyl, optionally substituted heterocyclyl, optionally substituted aryl, or optionally substituted heteroaryl ring;
R⁴ is selected from a bond, an optionally substituted C₁-C₄ alkylene, and an optionally substituted C₂-C₄ alkenylene or alkynylene, wherein:
one or more methylene units of the alkylene, alkenylene or alkynylene other than a methylene unit bound to a nitrogen atom is optionally and independently replaced with —O—, —S—, —N(R⁶)—, or —S(═O)₂—, and
two substituents on either the same or adjacent carbon atoms in the alkylene, alkenylene or alkynylene are taken together to form an optionally substituted carbocyclic or optionally substituted heterocyclic ring;
R⁵ is selected from a bond, an optionally substituted C₁-C₄ alkylene, and an optionally substituted C₂-C₄ alkenylene or alkynylene, wherein:
one or more methylene units of the alkylene, alkenylene or alkynylene is optionally and independently replaced with —O—, —S—, —N(R⁶)—, or —S(═O)₂—, and
two substituents on either the same or adjacent carbon atoms in the alkylene, alkenylene or alkynylene are optionally taken together to form an optionally substituted carbocyclic or optionally substituted heterocyclic ring;
each R⁶ is independently selected from hydrogen, and —C₁-C₆ alkyl;
R⁷ is any one of the Formulae (ii-1)-(ii-20): 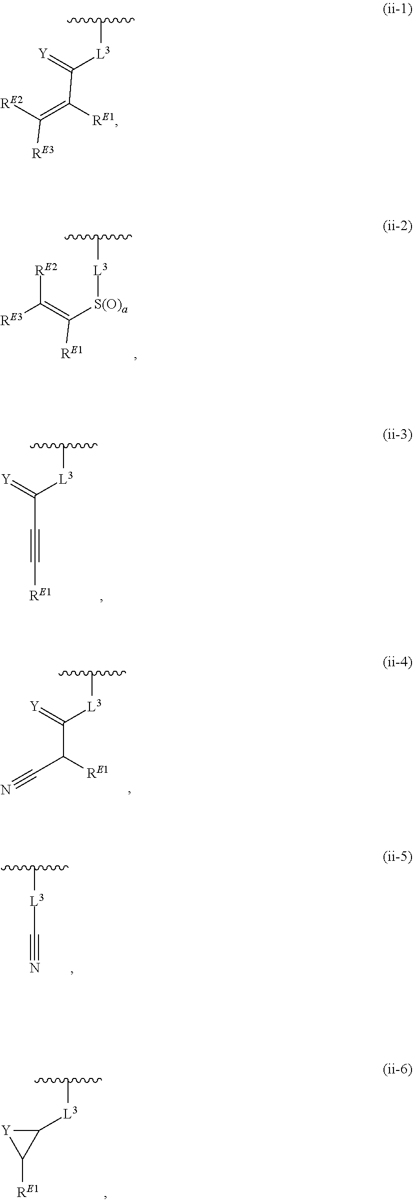 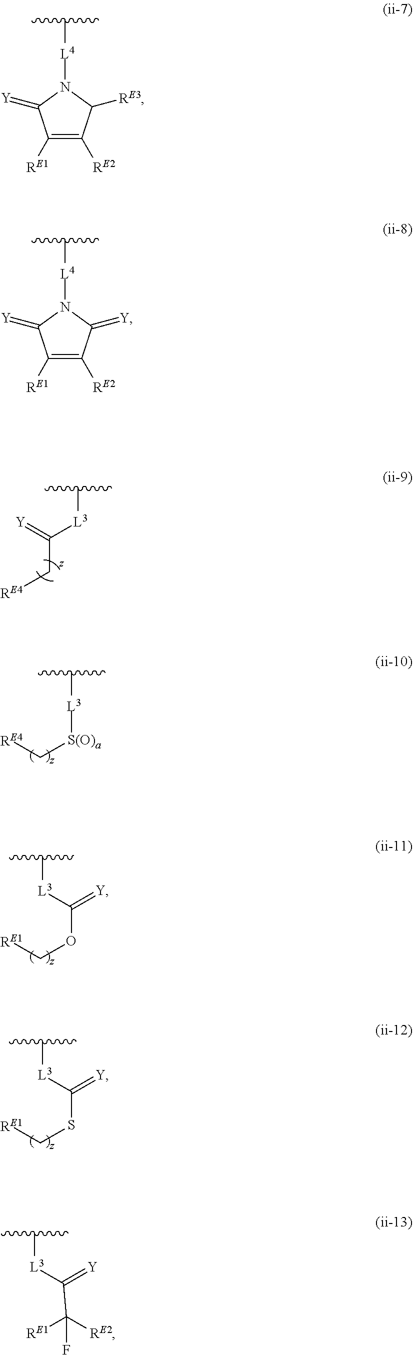 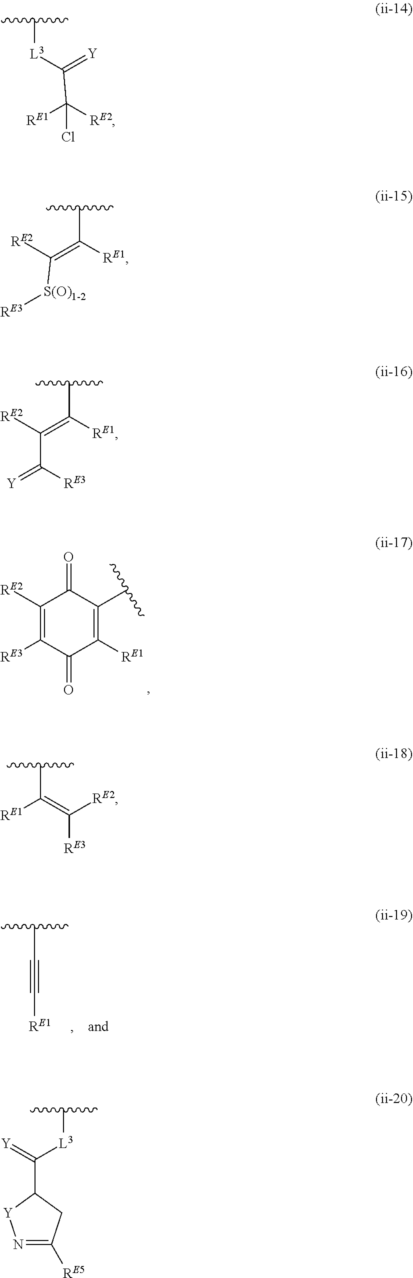
wherein:
R⁷ and Q are para or meta to each other;
L³ is a bond, an optionally substituted C₁-C₇ alkylene, or an optionally substituted C₂-C₅ alkenylene or alkynylene, wherein one or more methylene units of the alkylene, alkenylene or alkynylene are optionally and independently replaced with —O—, —S—, —S(O)—, —S(O)₂, or —N(R⁶)—;
L⁴ is a bond, an optionally substituted C₁-C₄ alkylene, or an optionally substituted C₂-C₄ alkenylene or alkynylene;
each of R^{E1}, R^{E2} and R^{E3} is independently selected from hydrogen, deuterium, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CH₂OR⁹, —CH₂N(R⁹)₂, —CH₂SR⁹, —CN, —OR⁹, —N(R⁹)₂, and —SR⁹, wherein each occurrence of R⁹ is independently selected from hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E5a}, —CH₂N(R^{E5a})₂, —CH₂SR^{E5a}, —OR^{E5a}, —N(R^{E5a})₂, and —SR^{E5a}, wherein each occurrence of R^{E5a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E5a} groups are joined to form an optionally substituted heterocyclic ring;
Y is O, S, or N(R^{E6}); wherein R^{E6} is hydrogen, substituted or unsubstituted C₁₋₆ alkyl, or a nitrogen protecting group;
z is 0, 1, 2, 3, 4, 5, or 6;
a is 1 or 2;
each instance of R⁸, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{D1}, —N(R^{D1})₂, and —SR^{D1}, wherein each occurrence of R^{D1} is independently selected from hydrogen, optionally substituted acyl, optionally substituted alkyl, optionally substituted alkenyl, optionally substituted alkynyl, optionally substituted carbocyclyl, optionally substituted heterocyclyl, and optionally substituted aryl, optionally substituted heteroaryl, or
two R⁸ groups are joined to form an optionally substituted carbocyclyl, optionally substituted heterocyclyl, optionally substituted aryl, or optionally substituted heteroaryl ring;
m is 0, 1, 2, 3 or 4;
n is 0, 1, 2, 3, 4, 5, 6, 7, 8, 9, 10, 11, 12, 13 or 14; and
wherein the compound is other than:

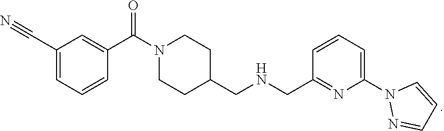 [SMILES: N#Cc1cccc(C(=O)N2CCC(CNCc3cccc(-n4cccn4)n3)CC2)c1]

34. The compound of claim 33, wherein W is C(R^{1a}); and each X is N.

35. The compound of claim 34, wherein R^{1a} is selected from chloro and —CN.

36. The compound of claim 33, wherein ring A is selected from

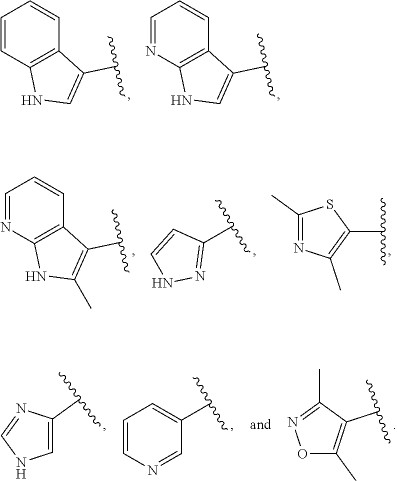

37. The compound of claim 33, wherein R² is selected from —NH—, and —NH—CH₂—**, wherein “**” represents a portion of R² bound to Q.

38. The compound of claim 33, wherein Q is selected from 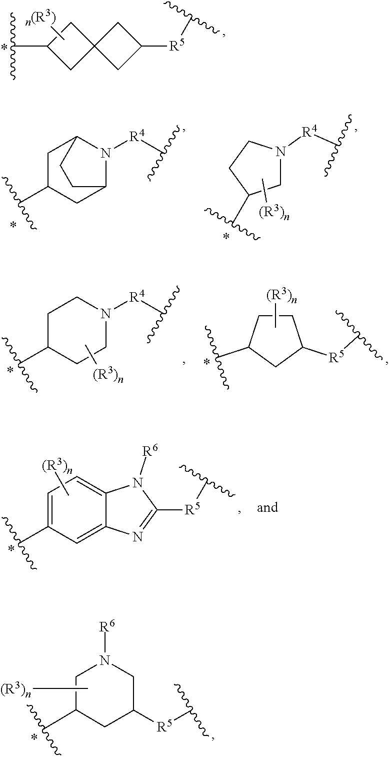
wherein:
“*” represents a portion of Q bound to R²;
n is 0, 1, 2, 3, 4, 5, 6, 7, 8, 9, or 10;
R⁵, when present, is selected from a bond, †—N(R⁶)—C(O)—, †—C(O)—N(R⁶)—, and †—CH₂—, wherein “†” represents a portion of R⁵ bound to Q; and
R⁴, when present, is selected from —C(O)—, —S(O)₂ and —CH₂—.

39. The compound of claim 33, wherein n is 0.

40. The compound of claim 33, wherein each R⁶ is independently hydrogen or —CH₃.

41. The compound of claim 33, wherein R⁷ is located meta or para to Q and is selected from CH₂N(CH₃)C(O)CH═CHN(CH₃)₂, —CH₂NHC(O)CH═CHN(CH₃)₂, —N(CH₃)C(O)CH═CHCH₂N(CH₃)₂, —NHC(O)(CH₂)₄NHC(O)CH═CHCH₂N(CH₃)₂, —NHC(O)CH═CH₂, —NHC(O)CH═CHCH₂N(CH₃)₂, —NHC(O)CH═CHCH₂N(CH₃)CH₂CH(OH)CH₂OH, —NHC(O)CH═CHCH₂N(CH₃)CH₂CH₂OH, —NHC(O)CH═CHCH₂N(CH₃)CH₂C(O)NH₂, —NHC(O)CH═CHCH₂N(CH₃)CH₂C(O)OH, —NHC(O)CH═CHCH₂NHC(O)CF₃, —NHC(O)CH═CHCH₂NHS(O)₂CH₃, —NHC(O)CH═CHCH₂OH, —NHC(O)CH═CHN(CH₃)₂, —NHC(O)CH═CHNHCH₃, —NHC(O)CH₂CH₂NHC(O)CH═CHCH₂N(CH₃)₂, —NHC(O)CH₂NHC(O)CH═CHCH₂N(CH₃)₂,

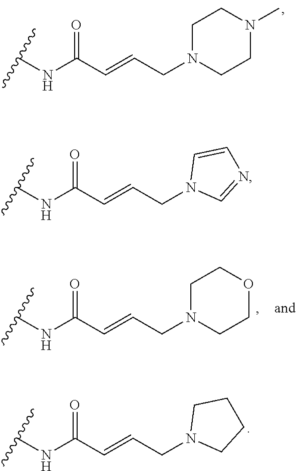 [SMILES: *NC(=O)/C=C/CN1CCN(C)CC1 ; *NC(=O)/C=C/Cn1ccnc1 ; *NC(=O)/C=C/CN1CCOCC1 ; *NC(=O)/C=C/CN1CCCC1]

42. The compound of claim 33, wherein m is 0 or 1; and the single R⁸, if present, is selected C₁-C₄ alkyl and halogen.

43. The compound of claim 33, selected from any one of Compounds 100-162.

44. A compound of Formula (II): 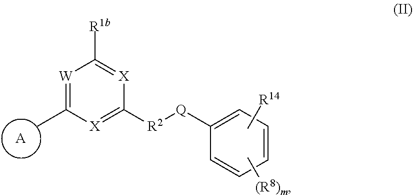
and pharmaceutically acceptable salts, solvates, hydrates, tautomers, stereoisomers, and isotopically labeled derivatives thereof, wherein:
ring A is an optionally substituted heteroaryl ring of any one of the Formulae (i-1)-(i-6): 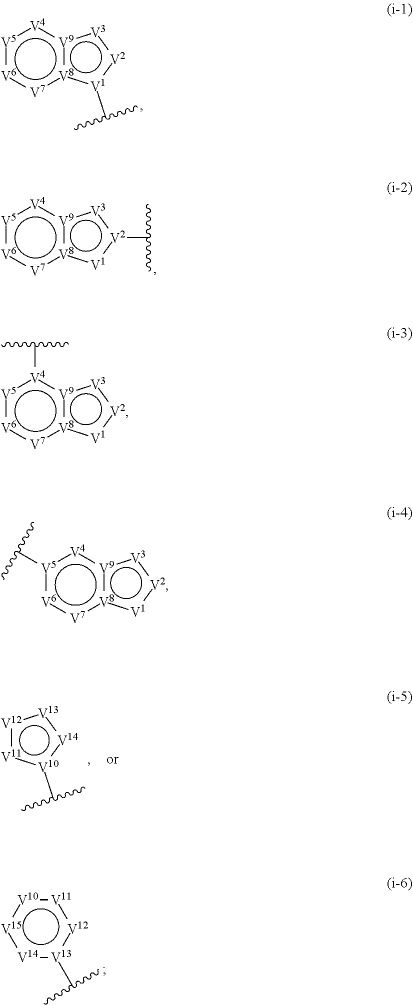
wherein:
each instance of V¹, V², V³, V⁴, V⁵, V⁶, V⁷, V⁸, V⁹, V¹⁰, V¹¹, V¹², V¹³, V¹⁴ and V¹⁵ is independently O, S, N, N(R^{A1}), C, or C(R^{A2}), and wherein at least one ring atom in ring A is selected from O, S or N;
each instance of R^{A1} is independently selected from hydrogen, deuterium, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl;
each instance of R^{A2} is independently selected from hydrogen, deuterium, halogen, —CN, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2}, —N(R^{A2a})₂, and —SR^{A2a}, wherein each occurrence of R^{A2a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
any two R^{A1}, any two R^{A2}, or one R^{A1} and one R^{A2} are joined to form an optionally substituted carbocyclic, optionally substituted heterocyclic, optionally substituted aryl, or optionally substituted heteroaryl ring;
each X is independently selected from N and CH, wherein at least one X is N;
W is selected from N and C(R^{1a});
each of R^{1a}, if present, and R^{1b} is independently selected from hydrogen, deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{B1a}, —N(R^{B1a})₂, and —SR^{B1a}, wherein each occurrence of R^{B1a} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
R^{1a} and R^{1b} are joined to form an optionally substituted carbocyclic, optionally substituted heterocyclic, optionally substituted aryl, or optionally substituted heteroaryl ring;
R² is an optionally substituted C₁-C₄ alkylene or an optionally substituted C₂-C₄ alkenylene or alkynylene, wherein one or more methylene units of the alkylene, alkenylene or alkynylene are optionally and independently replaced with —O—, —S—, or —N(R⁶)—;
Q is selected from: R⁵, 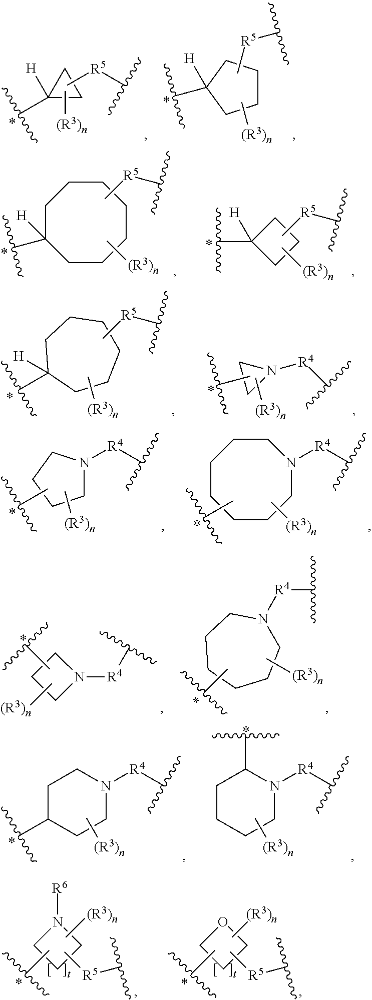
and a 4-14 membered, divalent, fused or spirofused bicyclic ring system comprising a total of 0 to 4 ring heteroatoms independently selected from N, O and S, and optionally substituted with 1 to 6 independently selected R³, wherein:
each ring in the bicyclic ring system is independently selected from heterocyclyl, carbocyclyl, aromatic or heteroaromatic,
one atom in each ring of the bicyclic ring system is attached to the rest of the compound, and
t is 0, 1, 2, 3, or 4;
wherein each [symbol] represents a portion of Q bound to the rest of the compound; and “*” represents a portion of Q bound to R²;
each instance of R³, if present, is independently selected from deuterium, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{C1}, —N(R^{C1})₂, and —SR^{C1} wherein each occurrence of R^{C1} is independently selected from hydrogen, optionally substituted acyl, optionally substituted alkyl, optionally substituted alkenyl, optionally substituted alkynyl, optionally substituted carbocyclyl, optionally substituted heterocyclyl, optionally substituted aryl, and optionally substituted heteroaryl, or
two R³ groups bound to the same ring carbon atom are taken together to form ═O, or two R³ groups bound to the same or different ring carbon atoms are joined to form an optionally substituted carbocyclyl, optionally substituted heterocyclyl, optionally substituted aryl, or optionally substituted heteroaryl ring;
R⁴ is selected from a bond, an optionally substituted C₁-C₄ alkylene, and an optionally substituted C₂-C₄ alkenylene or alkynylene, wherein:
one or more methylene units of the alkylene, alkenylene or alkynylene other than a methylene unit bound to a nitrogen atom is optionally and independently replaced with —O—, —S—, —N(R⁶)—, or —S(═O)₂—, and
two substituents on either the same or adjacent carbon atoms in the alkylene, alkenylene or alkynylene are taken together to form an optionally substituted carbocyclic or optionally substituted heterocyclic ring;
R⁵ is selected from a bond, an optionally substituted C₁-C₄ alkylene, and an optionally substituted C₂-C₄ alkenylene or alkynylene, wherein:
one or more methylene units of the alkylene, alkenylene or alkynylene is optionally and independently replaced with —O—, —S—, —N(R⁶)—, or —S(═O)₂—, and
two substituents on either the same or adjacent carbon atoms in the alkylene, alkenylene or alkynylene are optionally taken together to form an optionally substituted carbocyclic or optionally substituted heterocyclic ring;
each R⁶ is independently selected from hydrogen, and —C₁-C₆ alkyl; and
R¹⁴ is selected from —C₁-C₈ alkyl, —O—C₁-C₈ alkyl, —NH₂, —NH(C₁-C₈ alkyl), —N(C₁-C₈ alkyl)₂, wherein each alkyl in R¹⁴ is independently selected and optionally and independently substituted.

45. The compound of claim 44, wherein R¹⁴ is selected from —NH₂, —NH—CH₃, —NH—C(O)—CH₃, —NH—C(O)—(CH₂)₃—N(CH₃)₂, —NH—C(O)—(CH₂)₄—NH₂, —NH—C(O)—(CH₂)₂—NH₂, and —NH—C(O)—CH₂—NH₂.

46. A pharmaceutical composition comprising a compound of claim 33 and a pharmaceutically acceptable excipient.

47. The compound of claim 33, wherein R^{1b} is H.
